SEQUENCE LISTING (523 sequences)

SEQ ID NO: 1 - protein, 98 aa, synthetic construct
  REGION 1..98: note = Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 2 - protein, 15 aa, synthetic construct
  REGION 1..15: note = Synthetic
EPKSCDKTHTCPPCP

SEQ ID NO: 3 - protein, 110 aa, synthetic construct
  REGION 1..110: note = Synthetic
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAK

SEQ ID NO: 4 - protein, 107 aa, synthetic construct
  REGION 1..107: note = Synthetic
GQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 5 - protein, 98 aa, synthetic construct
  REGION 1..98: note = Synthetic
ASTKGPSVFPLAPCSRSTSESTAALGCLVKDYFPEPVTVSWNSGALTSGVHTFPAVLQSSGLYSLSSVVTVPSSNFGTQTYTCNVDHKPSNTKVDKTV

SEQ ID NO: 6 - protein, 12 aa, synthetic construct
  REGION 1..12: note = Synthetic
ERKCCVECPPCP

SEQ ID NO: 7 - protein, 109 aa, synthetic construct
  REGION 1..109: note = Synthetic
APPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTK

SEQ ID NO: 8 - protein, 107 aa, synthetic construct
  REGION 1..107: note = Synthetic
GQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 9 - protein, 98 aa, synthetic construct
  REGION 1..98: note = Synthetic
ASTKGPSVFPLAPCSRSTSGGTAALGCLVKDYFPEPVTVSWNSGALTSGVHTFPAVLQSSGLYSLSSVVTVPSSSLGTQTYTCNVNHKPSNTKVDKRV

SEQ ID NO: 10 - protein, 62 aa, synthetic construct
  REGION 1..62: note = Synthetic
ELKTPLGDTTHTCPRCPEPKSCDTPPPCPRCPEPKSCDTPPPCPRCPEPKSCDTPPPCPRCP

SEQ ID NO: 11 - protein, 110 aa, synthetic construct
  REGION 1..110: note = Synthetic
APELLGGPSVFLFPPKPKDTLMISRTPEVTCVVVDVSHEDPEVQFKWYVDGVEVHNAKTKPREEQYNSTFRVVSVLTVLHQDWLNGKEYKCKVSNKALPAPIEKTISKTK

SEQ ID NO: 12 - protein, 107 aa, synthetic construct
  REGION 1..107: note = Synthetic
GQPREPQVYTLPPSREEMTKNQVSLTCLVKGFYPSDIAVEWESSGQPENNYNTTPPMLDSDGSFFLYSKLTVDKSRWQQGNIFSCSVMHEALHNRFTQKSLSLSPGK

SEQ ID NO: 13 - protein, 98 aa, synthetic construct
  REGION 1..98: note = Synthetic
ASTKGPSVFPLAPCSRSTSESTAALGCLVKDYFPEPVTVSWNSGALTSGVHTFPAVLQSSGLYSLSSVVTVPSSSLGTKTYTCNVDHKPSNTKVDKRV

SEQ ID NO: 14 - protein, 12 aa, synthetic construct
  REGION 1..12: note = Synthetic
ESKYGPPCPSCP

SEQ ID NO: 15 - protein, 110 aa, synthetic construct
  REGION 1..110: note = Synthetic
APEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAK

SEQ ID NO: 16 - protein, 107 aa, synthetic construct
  REGION 1..107: note = Synthetic
GQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 17 - protein, 102 aa, synthetic construct
  REGION 1..102: note = Synthetic
ASPTSPKVFPLSLCSTQPDGNVVIACLVQGFFPQEPLSVTWSESGQGVTARNFPPSQDASGDLYTTSSQLTLPATQCLAGKSVTCHVKHYTNPSQDVTVPCP

SEQ ID NO: 18 - protein, 101 aa, synthetic construct
  REGION 1..101: note = Synthetic
CCHPRLSLHRPALEDLLLGSEANLTCTLTGLRDASGVTFTWTPSSGKSAVQGPPERDLCGCYSVSSVLPGCAEPWNHGKTFTCTAAYPESKTPLTATLSKS

SEQ ID NO: 19 - protein, 131 aa, synthetic construct
  REGION 1..131: note = Synthetic
GNTFRPEVHLLPPPSEELALNELVTLTCLARGFSPKDVLVRWLQGSQELPREKYLTWASRQEPSQGTTTFAVTSILRVAAEDWKKGDTFSCMVGHEALPLAFTQKTIDRLAGKPTHVNVSVVMAEVDGTCY

SEQ ID NO: 20 - protein, 102 aa, synthetic construct
  REGION 1..102: note = Synthetic
ASPTSPKVFPLSLDSTPQDGNVVVACLVQGFFPQEPLSVTWSESGQNVTARNFPPSQDASGDLYTTSSQLTLPATQCPDGKSVTCHVKHYTNPSQDVTVPCP

SEQ ID NO: 21 - protein, 101 aa, synthetic construct
  REGION 1..101: note = Synthetic
CCHPRLSLHRPALEDLLLGSEANLTCTLTGLRDASGATFTWTPSSGKSAVQGPPERDLCGCYSVSSVLPGCAQPWNHGETFTCTAAHPELKTPLTANITKS

SEQ ID NO: 22 - protein, 131 aa, synthetic construct; identical to SEQ ID NO: 19
  REGION 1..131: note = Synthetic

SEQ ID NO: 23 - protein, 110 aa, synthetic construct
  REGION 1..110: note = Synthetic
VALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKST

SEQ ID NO: 24 - protein, 110 aa, synthetic construct
  REGION 1..110: note = Synthetic
VALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSC

SEQ ID NO: 25 - protein, 130 aa, synthetic construct
  REGION 1..130: note = Synthetic
VALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 26 - protein, 214 aa, synthetic construct
  REGION 1..214: note = Synthetic
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 27 - protein, 450 aa, synthetic construct
  REGION 1..450: note = Synthetic
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 28 - protein, 107 aa, synthetic construct [sequence omitted]

SEQ ID NO: 29 - protein, 120 aa, synthetic construct [sequence omitted]

SEQ ID NO: 30 - protein, 463 aa, synthetic construct
  REGION 1..463: note = Synthetic
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSAVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 441 aa, synthetic construct
  REGION 1..441: note = Synthetic
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 231 aa, synthetic construct
  REGION 1..231: note = Synthetic
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSAVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKST

SEQ ID NO: 33 - protein, 455 aa, synthetic construct
  REGION 1..455: note = Synthetic
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSARTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 217 aa, synthetic construct
  REGION 1..217: note = Synthetic
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKST

SEQ ID NO: 35 - protein, 107 aa, synthetic construct [sequence omitted]

SEQ ID NO: 36 - protein, 330 aa, synthetic construct
  REGION 1..330: note = Synthetic
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 37 - protein, 10 aa, synthetic construct
  REGION 1..10: note = Synthetic
DKTHTCPPCP

SEQ ID NO: 38 - protein, 107 aa, synthetic construct [sequence omitted]

SEQ ID NO: 39 - protein, 123 aa, synthetic construct [sequence omitted]

SEQ ID NO: 40 - protein, 130 aa, synthetic construct
  REGION 1..130: note = Synthetic
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARSPNPYYYDSSGYYYPGAFDIWGQGTMVTVSSA

SEQ ID NO: 41 - protein, 110 aa, synthetic construct
  REGION 1..110: note = Synthetic
QPGLTQPPSVSVAPGQTARITCGGNNIGSKSVHWYQQKPGQAPVLVVYDDSDRPSGIPERFSGSNSGNTATLTISRVEAGDEADYYCQVWDSSSDHYVFGTGTKVTVLSS

SEQ ID NO: 42 - protein, 106 aa, synthetic construct
  REGION 1..106: note = Synthetic
SVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 43 - protein, 107 aa, synthetic construct
  REGION 1..107: note = Synthetic
GQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 44 - protein, 107 aa, synthetic construct
  REGION 1..107: note = Synthetic
GQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 45 - protein, 214 aa, synthetic construct
  REGION 1..214: note = Synthetic
DIQMTQSPSSLSASVGDRVTITCSASQDISNYLNWYQQKPGKAPKVLIYFTSSLHSGVPSRFSGSGSGTDFTLTISSLQPEDFATYYCQQYSTVPWTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 46 - protein, 453 aa, synthetic construct
  REGION 1..453: note = Synthetic
EVQLVESGGGLVQPGGSLRLSCAASGYTFTNYGMNWVRQAPGKGLEWVGWINTYTGEPTYAADFKRRFTFSLDTSKSTAYLQMNSLRAEDTAVYYCAKYPHYYGSSHWYFDVWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPCRDELTKNQVSLWCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 463 aa, synthetic construct
  REGION 1..463: note = Synthetic
QVQLVQSGAEVKKPGASVKVSCKASGYTFTGYYMHWVRQAPGQGLEWMGWINPNSGGTNYAQKFQGRVTMTRDTSISTAYMELSRLRSDDTAVYYCARSPNPYYYDSSGYYYPGAFDIWGQGTMVTVSSASVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGECDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVCTLPPSRDELTKNQVSLSCAVKGFYPSDIAVEWESNGQPENNYKTTPPVLDSDGSFFLVSKLTVDKSRWQQGNVFSCSVMHEALHNHYTQKSLSLSPGK

SEQ ID NO: 48 - protein, 213 aa, synthetic construct
  REGION 1..213: note = Synthetic
QPGLTQPPSVSVAPGQTARITCGGNNIGSKSVHWYQQKPGQAPVLVVYDDSDRPSGIPERFSGSNSGNTATLTISRVEAGDEADYYCQVWDSSSDHYVFGTGTKVTVLSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 49 - protein, 220 aa, synthetic construct
  REGION 1..220: note = Synthetic
QPGLTQPPSVSVAPGQTARITCGGNNIGSKSVHWYQQKPGQAPVLVVYDDSDRPSGIPERFSGSNSGNTATLTISRVEAGDEADYYCQVWDSSSDHYVFGTGTKVTVLSSVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKST

SEQ ID NO: 50 - protein, 220 aa, synthetic construct
  REGION 1..220: note = Synthetic
QPGLTQPPSVSVAPGQTARITCGGNNIGSKSVHWYQQKPGQAPVLVVYDDSDRPSGIPERFSGSNSGNTATLTISRVEAGDEADYYCQVWDSSSDHYVFGTGTKVTVLSSVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSC

SEQ ID NO: 51 - protein, 240 aa, synthetic construct
  REGION 1..240: note = Synthetic
QPGLTQPPSVSVAPGQTARITCGGNNIGSKSVHWYQQKPGQAPVLVVYDDSDRPSGIPERFSGSNSGNTATLTISRVEAGDEADYYCQVWDSSSDHYVFGTGTKVTVLSSVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 52 - protein, 5 aa, synthetic construct
  REGION 1..5: note = Synthetic
EPKSC

SEQ ID NO: 53 - protein, 112 aa, synthetic construct
  REGION 1..112: note = Synthetic
VALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGK

SEQ ID NO: 54 - protein, 109 aa, synthetic construct
  REGION 1..109: note = Synthetic
HRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGK

SEQ ID NO: 55 - protein, 18 aa, synthetic construct
  REGION 1..18: note = Synthetic
PTLYNVSLVMSDTAGTCY

SEQ ID NO: 56 - protein, 110 aa, synthetic construct
  REGION 1..110: note = Synthetic
VALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSI

SEQ ID NO: 57 - protein, 110 aa, synthetic construct
  REGION 1..110: note = Synthetic
VALHRPDVCLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSI

SEQ ID NO: 58 - protein, 110 aa, synthetic construct
  REGION 1..110: note = Synthetic
VALHRPDVYLLPPAREQLNLRESATITCLVTGFSPADVFVQWMQRGQPLSPEKYVTSAPMPEPQAPGRYCAHSILTVSEEEWNTGETYTCVVAHEALPNRVTERTVDKSI

SEQ ID NO: 59 - protein, 110 aa, synthetic construct
  REGION 1..110: note = Synthetic
VALHRPDVYLLPPARECLNLRESATITCLVTGFSPADVFVQWMQRGQPLSPEKYVTSAPMPEPQAPGRYFAHSILTVSEEEWNTGETYTCVVAHEALPNRVTERTVDKSI

SEQ ID NO: 60 - protein, 98 aa, synthetic construct
  REGION 1..98: note = Synthetic
ASTKGPSVFPLAPSSKSTSGGTACLGCLVKDYFPEPVTVSWNSGALTSGVHTFPAVLQSSGLYSLSSVVTVPSSSLGTQTYICNVNHKPSNTKVDKKV

SEQ ID NO: 61 - protein, 98 aa, synthetic construct
  REGION 1..98: note = Synthetic
ASTKGPSVFPLAPSSKSTSGGTAALGCLVKDYFPEPVTVSWNSGALTSGVCTFPAVLQSSGLYSLSSVVTVPSSSLGTQTYICNVNHKPSNTKVDKKV

SEQ ID NO: 62 - protein, 98 aa, synthetic construct
  REGION 1..98: note = Synthetic
ASTKGPSVCPLAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 63 - protein, 98 aa, synthetic construct; identical to SEQ ID NO: 1
  REGION 1..98: note = Synthetic

SEQ ID NO: 64 - protein, 217 aa, synthetic construct
  REGION 1..217: note = Synthetic
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSC

SEQ ID NO: 65 - protein, 213 aa, synthetic construct
  REGION 1..213: note = Synthetic
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKGQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 66 - protein, 106 aa, synthetic construct
  REGION 1..106: note = Synthetic
GQPKANPTVTLFPPSSEELQANKATLVCLISDFYPGAVTVAWKADGSPVKAGVETTKPSKQSNNKYAASSYLSLTPEQWKSHRSYSCQVTHEGSTVEKTVAPTECS

SEQ ID NO: 67 - protein, 107 aa, synthetic construct
  REGION 1..107: note = Synthetic
RTVAAPSVFIFPPCDEQLKSGTASVVCLLNNFYPREAKVQWKVDNALQSGNSQESVTEQDSKDSTYSLSSTLTLSKADYEKHKVYACEVTHQGLSSPVTKSFNRGEA

SEQ ID NO: 68 - protein, 107 aa, synthetic construct
  REGION 1..107: note = Synthetic
RTVAAPSVFIFPPSDECLKSGTASVVCLLNNFYPREAKVQWKVDNALQSGNSQESVTEQDSKDSTYSLSSTLTLSKADYEKHKVYACEVTHQGLSSPVTKSFNRGEA

SEQ ID NO: 69 - protein, 107 aa, synthetic construct
  REGION 1..107: note = Synthetic
RTVAAPSVFIFPPSDEQLKSGTASVVCLLNNFYPREAKVQWKVDNALQSGNSCESVTEQDSKDSTYSLSSTLTLSKADYEKHKVYACEVTHQGLSSPVTKSFNRGEA

SEQ ID NO: 70 - protein, 107 aa, synthetic construct
  REGION 1..107: note = Synthetic
RTVAAPSVCIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEA

SEQ ID NO: 71 - protein, 105 aa, synthetic construct
  REGION 1..105: note = Synthetic
VAAPSVFIFPPSDEQLKSGTACVVCLLNNFYPREAKVQWKVDNALQSGNSQESVTEQDSKDSTYSLSSTLTLSKADYEKHKVYACEVTHQGLSSPVTKSFNRGEI

SEQ ID NO: 72 - protein, 105 aa, synthetic construct
  REGION 1..105: note = Synthetic
VAAPSVFIFPPSDEQLKSGTASVVCLLNNFYPREAKVQWKVDNALQSGNCQESVTEQDSKDSTYSLSSTLTLSKADYEKHKVYACEVTHQGLSSPVTKSFNRGEI

SEQ ID NO: 73 - protein, 105 aa, synthetic construct
  REGION 1..105: note = Synthetic
VAAPSVCIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEI

SEQ ID NO: 74 - protein, 110 aa, synthetic construct
  REGION 1..110: note = Synthetic
VALHRPDVCLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKST

SEQ ID NO: 75 - protein, 110 aa, synthetic construct
  REGION 1..110: note = Synthetic
VALHRPDVYLLPPAREQLNLRESATITCLVTGFSPADVFVQWMQRGQPLSPEKYVTSAPMPEPQAPGRYCAHSILTVSEEEWNTGETYTCVVAHEALPNRVTERTVDKST

SEQ ID NO: 76 - protein, 110 aa, synthetic construct
  REGION 1..110: note = Synthetic
VALHRPDVYLLPPAREQLNLRESACITCLVTGFSPADVFVQWMQRGQPLSPEKYVTSAPMPEPQAPGRYFAHSILTVSEEEWNTGETYTCVVAHEALPNRVTERTVDKST

SEQ ID NO: 77 - protein, 110 aa, synthetic construct
  REGION 1..110: note = Synthetic
VALHRPDVYLLPPARECLNLRESATITCLVTGFSPADVFVQWMQRGQPLSPEKYVTSAPMPEPQAPGRYFAHSILTVSEEEWNTGETYTCVVAHEALPNRVTERTVDKST

SEQ ID NO: 78 - protein, 110 aa, synthetic construct
  REGION 1..110: note = Synthetic
VALHRPDVYLCPPAREQLNLRESATITCLVTGFSPADVFVQWMQRGQPLSPEKYVTSAPMPEPQAPGRYFAHSILTVSEEEWNTGETYTCVVAHEALPNRVTERTVDKST

SEQ ID NO: 79 - protein, 103 aa, synthetic construct
  REGION 1..103: note = Synthetic
ASTKGPSVFPLAPSSKSTSGGTACLGCLVKDYFPEPVTVSWNSGALTSGVHTFPAVLQSSGLYSLSSVVTVPSSSLGTQTYICNVNHKPSNTKVDKKVEPKSI

SEQ ID NO: 80 - protein, 103 aa, synthetic construct
  REGION 1..103: note = Synthetic
ASTKGPSVFPLAPSSKSTSGGTAALGCLVKDYFPEPVTVSWNSGALTSGVCTFPAVLQSSGLYSLSSVVTVPSSSLGTQTYICNVNHKPSNTKVDKKVEPKSI

SEQ ID NO: 81 - protein, 103 aa, synthetic construct
  REGION 1..103: note = Synthetic
ASTKGPSVCPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSI

SEQ ID NO: 82 - protein, 103 aa, synthetic construct
  REGION 1..103: note = Synthetic
ASTKGPSVFPCAPSSKSTSGGTAALGCLVKDYFPEPVTVSWNSGALTSGVHTFPAVLQSSGLYSLSSVVTVPSSSLGTQTYICNVNHKPSNTKVDKKVEPKSI

SEQ ID NO: 83 - protein, 98 aa, synthetic construct
  REGION 1..98: note = Synthetic
ASTKGPSVFPCAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 84 - protein, 98 aa, synthetic construct
  REGION 1..98: note = Synthetic
ASTKGPSVFPLAPSSKSTSGGTAALCCLVKDYFPEPVTVSWNSGALTSGVHTFPAVLQSSGLYSLSSVVTVPSSSLGTQTYICNVNHKPSNTKVDKKV

SEQ ID NO: 85 - protein, 103 aa, synthetic construct
  REGION 1..103: note = Synthetic
ASTKGPSVFPLAPSSKSTSGGTAALCCLVKDYFPEPVTVSWNSGALTSGVHTFPAVLQSSGLYSLSSVVTVPSSSLGTQTYICNVNHKPSNTKVDKKVEPKSI

SEQ ID NO: 86 - protein, 110 aa, synthetic construct
  REGION 1..110: note = Synthetic
VALHRPCVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSI

SEQ ID NO: 87 - protein, 110 aa, synthetic construct
  REGION 1..110: note = Synthetic
VALHRPDVYLCPPAREQLNLRESATITCLVTGFSPADVFVQWMQRGQPLSPEKYVTSAPMPEPQAPGRYFAHSILTVSEEEWNTGETYTCVVAHEALPNRVTERTVDKSI

SEQ ID NO: 88 - protein, 110 aa, synthetic construct
  REGION 1..110: note = Synthetic
VALHRPDVYLLPPAREQLNLRESATITCLVTGFSPADVFVQWMQRGQPLSPEKYCTSAPMPEPQAPGRYFAHSILTVSEEEWNTGETYTCVVAHEALPNRVTERTVDKSI

SEQ ID NO: 89 - protein, 110 aa, synthetic construct
  REGION 1..110: note = Synthetic
VALHRPDVYLLPPAREQLNLRESATITCCVTGFSPADVFVQWMQRGQPLSPEKYVTSAPMPEPQAPGRYFAHSILTVSEEEWNTGETYTCVVAHEALPNRVTERTVDKSI

SEQ ID NO: 90 - protein, 110 aa, synthetic construct
  REGION 1..110: note = Synthetic
VALHRPDVCLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSA

SEQ ID NO: 91 - protein, 110 aa, synthetic construct
  REGION 1..110: note = Synthetic
VALHRPDVCLLAPAREQLNLRESATITCLVTGFSPADVFVQWMQRGQPLSPEKYVTSAPMPEPQAPGRYFAHSILTVSEEEWNTGETYTCVVAHEALPNRVTERTVDKSI

SEQ ID NO: 92 - protein, 110 aa, synthetic construct
  REGION 1..110: note = Synthetic
VALHRPDVCLLPPAREQLNLRESATITCLVYGFSPADVFVQWMQRGQPLSPEKYVTSAPMPEPQAPGRYFAHSILTVSEEEWNTGETYTCVVAHEALPNRVTERTVDKSI

SEQ ID NO: 93 - protein, 217 aa, synthetic construct; identical to SEQ ID NO: 64
  REGION 1..217: note = Synthetic

SEQ ID NO: 94 - protein, 217 aa, synthetic construct
  REGION 1..217: note = Synthetic
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKVALHRPDVCLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSI

SEQ ID NO: 95 - protein, 217 aa, synthetic construct
  REGION 1..217: note = Synthetic
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKVALHRPDVCLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSA

SEQ ID NO: 96 - protein, 217 aa, synthetic construct
  REGION 1..217: note = Synthetic
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKVALHRPDVCLLAPAREQLNLRESATITCLVTGFSPADVFVQWMQRGQPLSPEKYVTSAPMPEPQAPGRYFAHSILTVSEEEWNTGETYTCVVAHEALPNRVTERTVDKSI

SEQ ID NO: 97 - protein, 217 aa, synthetic construct
  REGION 1..217: note = Synthetic
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKVALHRPDVCLLPPAREQLNLRESATITCLVYGFSPADVFVQWMQRGQPLSPEKYVTSAPMPEPQAPGRYFAHSILTVSEEEWNTGETYTCVVAHEALPNRVTERTVDKSI

SEQ ID NO: 98 - protein, 217 aa, synthetic construct
  REGION 1..217: note = Synthetic
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKVALHRPCVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSI

SEQ ID NO: 99 - protein, 217 aa, synthetic construct
  REGION 1..217: note = Synthetic
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKVALHRPDVYLCPPAREQLNLRESATITCLVTGFSPADVFVQWMQRGQPLSPEKYVTSAPMPEPQAPGRYFAHSILTVSEEEWNTGETYTCVVAHEALPNRVTERTVDKSI

SEQ ID NO: 100 - protein, 217 aa, synthetic construct
  REGION 1..217: note = Synthetic
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKVALHRPDVYLLPPAREQLNLRESATITCLVTGFSPADVFVQWMQRGQPLSPEKYCTSAPMPEPQAPGRYFAHSILTVSEEEWNTGETYTCVVAHEALPNRVTERTVDKSI

SEQ ID NO: 101 - protein, 217 aa, synthetic construct
  REGION 1..217: note = Synthetic
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKVALHRPDVYLLPPAREQLNLRESATITCLVTGFSPADVFVQWMQRGQPLSPEKYVTSAPMPEPQAPGRYCAHSILTVSEEEWNTGETYTCVVAHEALPNRVTERTVDKSI

SEQ ID NO: 102 - protein, 217 aa, synthetic construct
  REGION 1..217: note = Synthetic
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKVALHRPDVYLLPPARECLNLRESATITCLVTGFSPADVFVQWMQRGQPLSPEKYVTSAPMPEPQAPGRYFAHSILTVSEEEWNTGETYTCVVAHEALPNRVTERTVDKSI

SEQ ID NO: 103 - protein, 217 aa, synthetic construct
  REGION 1..217: note = Synthetic
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKVALHRPDVYLLPPAREQLNLRESATITCCVTGFSPADVFVQWMQRGQPLSPEKYVTSAPMPEPQAPGRYFAHSILTVSEEEWNTGETYTCVVAHEALPNRVTERTVDKSI

SEQ ID NO: 104 - protein, 98 aa, synthetic construct
  REGION 1..98: note = Synthetic
ASTKGPSVFPLAPSSKSTSGGCAALGCLVKDYFPEPVTVSWNSGALTSGVHTFPAVLQSSGLYSLSSVVTVPSSSLGTQTYICNVNHKPSNTKVDKKV

SEQ ID NO: 105 - protein, 98 aa, synthetic construct
  REGION 1..98: note = Synthetic
ASTKGPSVFPLCPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 106 - protein, 98 aa, synthetic construct; identical to SEQ ID NO: 83
  REGION 1..98: note = Synthetic

SEQ ID NO: 107 - protein, 98 aa, synthetic construct
  REGION 1..98: note = Synthetic
ASTKGPSVFPLAPSSKSTSGGTAALGCLVKDYFPEPVTVSWNSGALTSGVHTFPAVLQCSGLYSLSSVVTVPSSSLGTQTYICNVNHKPSNTKVDKKV

SEQ ID NO: 108 - protein, 98 aa, synthetic construct; identical to SEQ ID NO: 61
  REGION 1..98: note = Synthetic

SEQ ID NO: 109 - protein, 98 aa, synthetic construct; identical to SEQ ID NO: 62
  REGION 1..98: note = Synthetic

SEQ ID NO: 110 - protein, 450 aa, synthetic construct
  REGION 1..450: note = Synthetic
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTACLGCLVKDYFPEPVTVSWNSGALTSGVHTFPAVLQSSGLYSLSSVVTVPSSSLGTQTYICNVNHKPSNTKVDKKVEPKSIDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 111 - protein, 450 aa, synthetic construct
  REGION 1..450: note = Synthetic
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGCAALGCLVKDYFPEPVTVSWNSGALTSGVHTFPAVLQSSGLYSLSSVVTVPSSSLGTQTYICNVNHKPSNTKVDKKVEPKSIDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 112 - protein, 450 aa, synthetic construct
  REGION 1..450: note = Synthetic
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLCPSSKSTSGGTAALGCLVKDYFPEPVTVSWNSGALTSGVHTFPAVLQSSGLYSLSSVVTVPSSSLGTQTYICNVNHKPSNTKVDKKVEPKSIDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - protein, 450 aa, synthetic construct
  REGION 1..450: note = Synthetic
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPCAPSSKSTSGGTAALGCLVKDYFPEPVTVSWNSGALTSGVHTFPAVLQSSGLYSLSSVVTVPSSSLGTQTYICNVNHKPSNTKVDKKVEPKSIDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 114 - protein, 450 aa, synthetic construct
  REGION 1..450: note = Synthetic
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVHTFPAVLQCSGLYSLSSVVTVPSSSLGTQTYICNVNHKPSNTKVDKKVEPKSIDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 115 - protein, 450 aa, synthetic construct
  REGION 1..450: note = Synthetic
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPLAPSSKSTSGGTAALGCLVKDYFPEPVTVSWNSGALTSGVCTFPAVLQSSGLYSLSSVVTVPSSSLGTQTYICNVNHKPSNTKVDKKVEPKSIDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 116 - protein, 450 aa, synthetic construct
  REGION 1..450: note = Synthetic
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVCPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSIDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 117 - protein, 15 aa, synthetic construct
  REGION 1..15: note = Synthetic
EPKSADKTHTCPPCP

SEQ ID NO: 118 - protein, 217 aa, synthetic construct
  REGION 1..217: note = Synthetic
APELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 119 - protein, 110 aa, synthetic construct
  REGION 1..110: note = Synthetic
VALHRPDVCVLPPAREQLNLRESATITCLVYGFSPPDVFVQWMQRGQPLSPEKYVTSAPMPEPQAPGRYFAHSILTVSEEEWNTGETYTCVVAHEALPNRVTERTVDKSA

SEQ ID NO: 120 - protein, 110 aa, synthetic construct
  REGION 1..110: note = Synthetic
VALHRPDVCVLPPAREQLNLRESATITCLITGFSPPDVFVQWMQRGQPLSPEKYVTSAPMPEPQAPGRYFAHSILTVSEEEWNTGETYTCVVAHEALPNRVTERTVDKSA

SEQ ID NO: 121 - protein, 110 aa, synthetic construct
  REGION 1..110: note = Synthetic
VALHRPDVCVLPPAREQLNLRESATITCLVTGFSPPDVFVQWMQRGQPLSPEKYVTSAPMPEPQAPGRYFAHSILTVSEEEWNTGETYTCVVAHEALPNRVTERTVDKSA

SEQ ID NO: 122 - protein, 110 aa, synthetic construct
  REGION 1..110: note = Synthetic
VALHRPDVCVLPPAREQLNLRESATITCLIYGFSPADVFVQWMQRGQPLSPEKYVTSAPMPEPQAPGRYFAHSILTVSEEEWNTGETYTCVVAHEALPNRVTERTVDKSA

SEQ ID NO: 123 - protein, 110 aa, synthetic construct
  REGION 1..110: note = Synthetic
VALHRPDVCVLPPAREQLNLRESATITCLVTGFSPPDVFVQWMQRGQPLSPEKYVTSAPMPEPQAPGRYFAHSILTVSEEEWNTGETYTCVVAHEALPNRVTQRTVDKSA

SEQ ID NO: 124 - protein, 110 aa, synthetic construct
  REGION 1..110: note = Synthetic
VALHRPDVCLLPPAREQLDLRESATITCLVTGFSPADVFVQWMQRGQPLSPEKYVTSAPMPEPQAPGRYFAHSILTVSEQEWNTGETYTCVVAHEALPNRVTERTIDKSI

SEQ ID NO: 125 - protein, 110 aa, synthetic construct
  REGION 1..110: note = Synthetic
VALHRPDVCVLPPAREQLNLRESATITCLITGFSPADVFVQWMQRGQPLSPEKYVTSAPMPEPQAPGRYFAHSILTVSEEEWNTGETYTCVVAHEALPNRVTERTVDKSA

SEQ ID NO: 126 - protein, 110 aa, synthetic construct
  REGION 1..110: note = Synthetic
VALHRPDICVLPPAREQLNLRESATITCLVYGFSPADVFVQWMQRGQPLSPEKYVTSAPMPEPQAPGRYFAHSILTVSEEEWNTGETYTCVVAHEALPNRVTERTVDKSA

SEQ ID NO: 127 - protein, 110 aa, synthetic construct
  REGION 1..110: note = Synthetic
VALHRPDVCVLPPAREQLNLRESATITCLVYGFSPADVFVQWMQRGQPLSPEKYVTSAPMPEPQAPGRYFAHSILTVSEEEWNTGETYTCVVAHEALPNRVTERTVDKSA

SEQ ID NO: 128 - protein, 110 aa, synthetic construct
  REGION 1..110: note = Synthetic
VALHRPDVCVLAPAREQLNLRESATITCLVTGFSPPDVFVQWMQRGQPLSPEKYVTSAPMPEPQAPGRYFAHSILTVSEEEWNTGETYTCVVAHEALPNRVTERTVDKSA

SEQ ID NO: 129 - protein, 110 aa, synthetic construct
  REGION 1..110: note = Synthetic
VALHRPDVCVLPPAREQLNLRESATITCLVTGFSPPDVFVQWMQRGQPLSPEKYVTSAPMPEPQAPGRYFAHSILTVSEQEWNTGETYTCVVAHEALPNRVTERTVDKSA

SEQ ID NO: 130 - protein, 110 aa, synthetic construct
  REGION 1..110: note = Synthetic
VALHRPDVCVLPPAREQLNLRESATITCLVYGFSPADVFVQWMQRGQPLSPEKYVTSAPMPEPQAPGRYFAHSILTVSEEEWNTGETYTCVVAHEALPNRVTQRTVDKSA

SEQ ID NO: 131 - protein, 110 aa, synthetic construct
  REGION 1..110: note = Synthetic
VALHRPDICVLPPAREQLNLRESATITCLITGFSPADVFVQWMQRGQPLSPEKYVTSAPMPEPQAPGRYFAHSILTVSEEEWNTGETYTCVVAHEALPNRVTERTVDKSA

SEQ ID NO: 132 - protein, 110 aa, synthetic construct
  REGION 1..110: note = Synthetic
VALHRPDICVLPPAREQLNLRESATITCLVTGFSPPDVFVQWMQRGQPLSPEKYVTSAPMPEPQAPGRYFAHSILTVSEEEWNTGETYTCVVAHEALPNRVTERTVDKSA

SEQ ID NO: 133 - protein, 110 aa, synthetic construct
  REGION 1..110: note = Synthetic
VALHRPDVCVLPPAREQLNLRESATITCLITGFSPADVFVQWMQRGQPLSPEKYVTSAPMPEPQAPGRYFAHSILTVSEEEWNTGETYTCVVAHEALPNRVTQRTVDKSA

SEQ ID NO: 134 - protein, 110 aa, synthetic construct
  REGION 1..110: note = Synthetic
VALHRPDVCVLPPAREQLNLRESATITCLVYGFSPADVFVQWMQRGQPLSPEKYVTSAPMPEPQAPGRYFAHSILTVSEQEWNTGETYTCVVAHEALPNRVTERTVDKSA

SEQ ID NO: 135 - protein, 110 aa, synthetic construct
  REGION 1..110: note = Synthetic
VALHRPDVCVLPPAREQLNLRESATITCLVTGFSPADVFVQWMQRGQPLSPEKYVTSAPMPEPQAPGRYFAHSILTVSEEEWNTGETYTCVVAHEALPNRVTQRTVDKST

SEQ ID NO: 136 - protein, 110 aa, synthetic construct
  REGION 1..110: note = Synthetic
VALHRPDVCVLAPAREQLNLRESATITCLITGFSPADVFVQWMQRGQPLSPEKYVTSAPMPEPQAPGRYFAHSILTVSEEEWNTGETYTCVVAHEALPNRVTERTVDKSA

SEQ ID NO: 137 - protein, 110 aa, synthetic construct
  REGION 1..110: note = Synthetic
VALHRPDVCVLPPAREQLNLRESATITCLITGFSPADVFVQWMQRGQPLSPEKYVTSAPMPEPQAPGRYFAHSILTVSLEEWNTGETYTCVVAHEALPNRVTERTVDKSI

SEQ ID NO: 138 - protein, 110 aa, synthetic construct
  REGION 1..110: note = Synthetic
VALHRPDVCVLPPAREQLNLRESATITCLVTGFSPADVFVQWMQRGQPLSPEKYVTSAPMPEPQAPGRYFAHSILTVSEEEWNTGETYTCVVAHEALPNRVTQRTVDKSA

SEQ ID NO: 139 - protein, 110 aa, synthetic construct
  REGION 1..110: note = Synthetic
VALHRPDVCVLAPAREQLNLRESATITCLVYGFSPADVFVQWMQRGQPLSPEKYVTSAPMPEPQAPGRYFAHSILTVSEEEWNTGETYTCVVAHEALPNRVTERTVDKSA

SEQ ID NO: 140 - protein, 110 aa, synthetic construct
  REGION 1..110: note = Synthetic
VALHRPDVCVLPPAREQLNLRESATITCLITGFSPADVFVQWMQRGQPLSPEKYVTSAPMPEPQAPGRYFAHSILTVSEQEWNTGETYTCVVAHEALPNRVTERTVDKSA

SEQ ID NO: 141 - protein, 110 aa, synthetic construct
  REGION 1..110: note = Synthetic
VALHRPDVCVLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSA

SEQ ID NO: 142 - protein, 110 aa, synthetic construct
  REGION 1..110: note = Synthetic
VALHRPDICVLPPAREQLNLRESATITCLVTGFSPADVFVQWMQRGQPLSPEKYVTSAPMPEPQAPGRYFAHSILTVSEEEWNTGETYTCVVAHEALPNRVTQRTVDKSA

SEQ ID NO: 143 - protein, 110 aa, synthetic construct
  REGION 1..110: note = Synthetic
VALHRPDVCVLPPAREQLNLRESATITCLVTGFSPADVFVQWMQRGQPLSPEKYVTSAPMPEPQAPGRYFAHSILTVSEQEWNTGETYTCVVAHEALPNRVTQRTVDKSA

SEQ ID NO: 144 - protein, 110 aa, synthetic construct
  REGION 1..110: note = Synthetic
VALHRPDVCVLPPAREQLNLRESATITCLVTGFSPADVFVQWMQRGQPLSPEKYVTSAPMPEPQAPGRYFAHSILTVSEQEWNTGETYTCVVAHEALPNRVTERTVDKSA

SEQ ID NO: 145 - protein, 110 aa, synthetic construct
  REGION 1..110: note = Synthetic
VALHRPDICVLPPAREQLNLRESATITCLVTGFSPADVFVQWMQRGQPLSPEKYVTSAPMPEPQAPGRYFAHSILTVSEQEWNTGETYTCVVAHEALPNRVTERTVDKSA

SEQ ID NO: 146 - protein, 110 aa, synthetic construct
  REGION 1..110: note = Synthetic
VALHRPDICVLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSA

SEQ ID NO: 147 - protein, 110 aa, synthetic construct
  REGION 1..110: note = Synthetic
VALHRPDVCVLAPAREQLNLRESATITCLVTGFSPADVFVQWMQRGQPLSPEKYVTSAPMPEPQAPGRYFAHSILTVSEEEWNTGETYTCVVAHEALPNRVTERTVDKSA

SEQ ID NO: 148 - protein, 110 aa, synthetic construct
  REGION 1..110: note = Synthetic
VALHRPDVCLLPPPREQLNLRESATITCLVTGFSPPDVFVQWMQRGQPLSPEKYVTSAPMPEPQAPGRYFAHSILTVSEEEWNTGETYTCVVAHEALPNHVTERTVDKSI

SEQ ID NO: 149 - protein, 110 aa, synthetic construct
  REGION 1..110: note = Synthetic
VALHRPDVCVLAPAREQLNLRESATITCLVTGFSPADVFVQWMQRGQPLSPEKYVTSAPMPEPQAPGRYFAHSILTVSEEEWNTGETYTCVVAHEALPNRVTQRTVDKSA

SEQ ID NO: 150 - protein, 110 aa, synthetic construct
  REGION 1..110: note = Synthetic
VALHRPDVCLFPPAREQLNLRESATITCLITGFSPADVFVQWMQRGQPVSPEKYVTSAPMPEPQAPGRYFAHSILTVSEEEWNTGETYTCVVAHEALPNRVTERTVDKSI

SEQ ID NO: 151 - protein, 110 aa, synthetic construct
  REGION 1..110: note = Synthetic
VALHRPDVCLLPPAREQLNLRESATITCLVTGFSPADVFVQWMQRGQPLSPEKYVTSAPMPEPQAPGRYFAHSILTVSEEEWNTGDTYTCVVAHEALPNRVTQRTVDRSI

SEQ ID NO: 152 - protein, 110 aa, synthetic construct
  REGION 1..110: note = Synthetic
VALHRPDVCLFPPAREQLNLRESATITCLVSGFSPADVFVQWMQRGQPLSPEKYVTSAPMPEPQAPGRYFAHSILTVSEEEWNTGETYTCVVAHEALPNRVTERTVDKST

SEQ ID NO: 153 - protein, 110 aa, synthetic construct
  REGION 1..110: note = Synthetic
VALHRPDVCLLPPAREQLNLRESATITCVVTGFSPADVFVQWMQRGQPLSPEKYVTSAPMPEPQAPGRYFAHSILTISEEEWNTGETYTCVVAHEALPNRVTERTVDKSS

SEQ ID NO: 154 - protein, 110 aa, synthetic construct
  REGION 1..110: note = Synthetic
VALHRPDICVLAPAREQLNLRESATITCLVTGFSPADVFVQWMQRGQPLSPEKYVTSAPMPEPQAPGRYFAHSILTVSEEEWNTGETYTCVVAHEALPNRVTERTVDKSA

SEQ ID NO: 155 - protein, 110 aa, synthetic construct
  REGION 1..110: note = Synthetic
VALHRPDVCVLAPAREQLNLRESATITCLVTGFSPADVFVQWMQRGQPLSPEKYVTSAPMPEPQAPGRYFAHSILTVSEQEWNTGETYTCVVAHEALPNRVTERTVDKSA

SEQ ID NO: 156 - protein, 110 aa, synthetic construct
  REGION 1..110: note = Synthetic
VALHRPDVCLLPPAREQLDLRESATITCLVTGFSPPDVFVQWMQRGQPLSPEKYVTSAPMPEPQAPGRYFAHSILTVSLEEWNTGETYTCVVAHEALPNRVTERTVDKSI

SEQ ID NO: 157 - protein, 110 aa, synthetic construct
  REGION 1..110: note = Synthetic
VALHAPDVCLLPPAREQLNLRESATITCVVTGFSPADVFVQWMQRGQPLSPEKYVTSAPMPEPQAPGAYFAHSILTVSEEEWNTGETYTCVVAHEALPNRVTERTVDKSI

SEQ ID NO: 158 - protein, 110 aa, synthetic construct
  REGION 1..110: note = Synthetic
VALHRPDVCLLPPAREQLNLRESATLTCLVTGFSPADVFVQWMQRGQPLSPEKYVTSAPMPEPQAPGRYFAHSILTVSEQEWNTGETYTCVVAHEALPNRVTERTVDRSI

SEQ ID NO: 159 - protein, 110 aa, synthetic construct
  REGION 1..110: note = Synthetic
VALHRPDVCLLPPAREQLNLRESATITCLVTGFSPPDVFVQWMQRGQPLSPEKYVTSAPMPEPQAPGRFFAHSILTVSEEEWNTGETYTCVVAHHALPNRVTERTVDKSI

SEQ ID NO: 160 - protein, 110 aa, synthetic construct
  REGION 1..110: note = Synthetic
VALHRPDVCLLPPAREQLNLRESATITCLVSGFSPADVFVQWMQRGQPLSPEKYVTSAPMPEPQAPGRYFAHSILTISEEEWNTGETYTCMVAHEALPNRVTERTVDKSI

SEQ ID NO: 161 - protein, 110 aa, synthetic construct
  REGION 1..110: note = Synthetic
VALHRPDVCLLPPAREQLLLRESATITCLVTGFSPADVFVQWMQRGQPLSPEKYVTSAPMPEPQAPGRYFAHSSLTVSEEEWNTGETYTCMVAHEALPNRVTERTVDKSI

SEQ ID NO: 162 - protein, 110 aa, synthetic construct
  REGION 1..110: note = Synthetic
VALHAPDVCLLPPAREQLNLRESATLTCLVSGFSPADVFVQWMQRGQPLSPEKYVTSAPMPEPQAPGRYFAHSILTVSEEEWNTGETYTCVVAHEALPNRVTERTVDKSI

SEQ ID NO: 163 - protein, 110 aa, synthetic construct
  REGION 1..110: note = Synthetic
VALHRPDVCLLPPAREQLNLRESATLTCLVTGFSPADVFVQWMQRGQPLSPEKYVTSAPMPEPQAPGRFFAHSSLTVSEEEWNTGETYTCVVAHEALPNRVTERTVDKSI

SEQ ID NO: 164 - protein, 110 aa, synthetic construct
  REGION 1..110: note = Synthetic
VALHRPDVCVLPPAREQLNLRESATVTCLVTGFSPADVFVQWMQRGQPLSPEKYVTSAPMPEPQAPGRYFAHSILTVAEEEWNTGETYTCVVAHEALPNRVTERTVDKSI

SEQ ID NO: 165 - protein, 110 aa, synthetic construct
  REGION 1..110: note = Synthetic
VALHRPDVCLFPPAREQLNLRESATVTCLVTGFSPADVFVQWMQRGQPLSPEKYVTSAPMPEPQAPGRYFAHSILTVSEEEWNTGETYTCVVAHEALPNRVTQRTVDKSI

SEQ ID NO: 166 - protein, 110 aa, synthetic construct
  REGION 1..110: note = Synthetic
VALHAPDVCLLPPAREQLNLRESATITCLVTGFSPADVFVQWMQRGQPLSPEKYVTSAPMREPQAPGRYFAHSILTVAEEEWNTGETYTCVVAHEALPNRVTERTVDKSI

SEQ ID NO: 167 - protein, 110 aa, synthetic construct
  REGION 1..110: note = Synthetic
VALHRPDVCLLPPAREQLNLKESATITCLVTGFSPADVFVQWMQRGQPLSPEKYVTSAPMPEPQAPGRYFAHSILTVSEEDWNTGETYTCVVAHEALPNRVAERTVDKSI

SEQ ID NO: 168 - protein, 110 aa, synthetic construct
  REGION 1..110: note = Synthetic
VALHRPDVCLLPPAREQLNLKESATITCLVTGFSPADVFVQWMQRGQPVSPEKYVTSAPMPEPQAPGRYFAHSILTVSEEEWNTGETYYCVVAHEALPNRVTERTVDKSI

SEQ ID NO: 169 - protein, 110 aa, synthetic construct
  REGION 1..110: note = Synthetic
VALHRPDVCLLPPAREQLLLRESATITCLVTGFSPADVFVQWMQRGQPLSPEKYVTSAPMPEPQAPGRYFAHSILTVSEEEWNTGDTYTCVVAHHALPNRVTERTVDKSI

SEQ ID NO: 170 - protein, 110 aa, synthetic construct
  REGION 1..110: note = Synthetic
VALHRPDVCLLPPAREQLLLRESATITCLVTGFSPADVFVQWMQRGQPVSPEKYVTIAPMPEPQAPGRYFAHSILTVSEEEWNTGETYTCVVAHEALPNRVTERTVDKSI

SEQ ID NO: 171 - protein, 110 aa, synthetic construct
  REGION 1..110: note = Synthetic
VALHRPDVCLLPPAREQLNLRESATITCLVTGFSPADVFVQWMQRGQPLSPEKYVTSAPMREPQAPGRYFAHSSLTVSEEEWNTGETYTCVVAHEALPNHVTERTVDKSI

SEQ ID NO: 172 - protein, 110 aa, synthetic construct
  REGION 1..110: note = Synthetic
VALHRPDVCLLPPAREQLNLRESATITCLVTGFSPADVFVQWMQRGQPLSPEKYVTTAPMPEPQAPGRYFAHSILTISEEEWNTGETYTCVVAHEALPNRVTERTIDKSI

SEQ ID NO: 173 - protein, 110 aa, synthetic construct
  REGION 1..110: note = Synthetic
VALHRPDVCLLPPAREQLNLRESATITCLVTGFSPADVFVQWMQRGQPLSPEKYVTSAPMPEPQAPGAYFAHSILTVSEEEWNTGDTYTCVVAHEALPNRVTEKTVDKSI

SEQ ID NO: 174 - protein, 110 aa, synthetic construct
  REGION 1..110: note = Synthetic
VALHRPDVCLLPPAREQLNLRESATITCLVTSFSPADVFVQWMQRGQPLSPEKYVTSAPMPEPQAPGRYFAHSILTVSEEDWNTGETYTCVVAHEALPNRVTEKTVDKSI

SEQ ID NO: 175 - protein, 110 aa, synthetic construct
  REGION 1..110: note = Synthetic
VALHRPDVCLLPPAREQLDLRESATITCLVTSFSPADVFVQWMQRGQPLSPEKYVTIAPMPEPQAPGRYFAHSILTVSEEEWNTGETYTCVVAHEALPNRVTERTVDKSI

SEQ ID NO: 176 - protein, 110 aa, synthetic construct
  REGION 1..110: note = Synthetic
VALHRPDVCLLPPAREQLNLRESATITCLVTSFSPADVFVQWMQRGQPLSPEKYVTSAPMPEPQAPGRYFAHSILTVSEEEWNTGETYTCVVAHEALPNRVAERTVDKSS

SEQ ID NO: 177 - protein, 110 aa, synthetic construct
  REGION 1..110: note = Synthetic
VALHRPDVCLLPPPREQLNLKESATITCLITGFSPADVFVQWMQRGQPLSPEKYVTSAPMPEPQAPGRYFAHSILTVSEEEWNTGETYTCVVAHEALPNRVTERTVDKSI

SEQ ID NO: 178 - protein, 110 aa, synthetic construct
  REGION 1..110: note = Synthetic
VALHRPDVCLLPPAREQLNLRESATITCLVTGFSPADVFVQWMQRGQPLSPEKYVTSAPMREPQAPGRYFAHSILTVSEEEWNTGETYTCVVAHEAVPNRVAERTVDKSI

SEQ ID NO: 179 - protein, 110 aa, synthetic construct
  REGION 1..110: note = Synthetic
VALHRPDVCLLPPAREQLNLRESATITCLVTGFSPADVFVQWMQRGQPLSPEKYVTTAPMPEPQAPGRYFAHSILTVSEEEWNTGETYTCVVAHEALPNHVTEKTVDKSI

SEQ ID NO: 180 - protein, 110 aa, synthetic construct
  REGION 1..110: note = Synthetic
VALHRPDVCLLPPAREQLNLRESATITCLVTGFSPADVFVQWMQRGQPLSPEKYVTSAPMPEPQAPGRYFAHSILTVAEREWNTGETYTCVVAHEAVPNRVTERTVDKSI

SEQ ID NO: 181 - protein, 110 aa, synthetic construct
  REGION 1..110: note = Synthetic
VALHRPDVCLLPPAREQLNLRESATITCLVTGFSPADVFVQWMQRGQPLSPEKYVTSAPMPEPQAPGRFFAHSILTVSLEEWNTGETYTCVVAHEAVPNRVTERTVDKSI

SEQ ID NO: 182 - protein, 110 aa, synthetic construct
  REGION 1..110: note = Synthetic
VALHRPDVCLLPPPREQLNLRESATITCLVTGFSPADVFVQWMQRGQPLSPEKYVTSAPMPEPQAPGRYFAHSILTVSEEEWNTGETYYCVVAHEALPNRVTERTVDKST

SEQ ID NO: 183 - protein, 110 aa, synthetic construct
  REGION 1..110: note = Synthetic
VALHRPDVCLLPPAREQLNLRESATITCVVTGFSPADVFVQWMQRGQPLSPEKYVTIAPMPEPQAPGRYFAHSILTVSEEEWNTGETYTCVVAHEALPNRVTERTIDKSI

SEQ ID NO: 184 - protein, 110 aa, synthetic construct
  REGION 1..110: note = Synthetic
VALHRPDVCLLPPAREQLNLRESATITCLVTGFSPADVFVQWMQRGQPLSPEKYVTTAPMPEPQAPGAYFAHSILTVSEEDWNTGETYTCVVAHEALPNRVTERTVDKSI

SEQ ID NO: 185 - protein, 110 aa, synthetic construct
  REGION 1..110: note = Synthetic
VALHRPDVCLLPPAREQLNLRESATITCLVTGFSPADVFVQWMQRGQPLSPEKYVTSAPMPEPQAPGRYFAHSILTVSEREWNTGETYYCVVAHEALPNRVTERTVDRSI

SEQ ID NO: 186 - protein, 110 aa, synthetic construct
  REGION 1..110: note = Synthetic
VALHRPDVCLLPPAREQLNLRESATVTCLVTGFSPADVFVQWMQRGQPLSPEKYVTSAPMPEPQAPGRYFAHSILTVSEREWNTGETYTCVVAHEALPNRVTERTVDKSS

SEQ ID NO: 187 - protein, 110 aa, synthetic construct
  REGION 1..110: note = Synthetic
VALHRPDVCLLPPAREQLNLRESATITCLVTGFSPADVFVQWMQRGQPLSPEKYVTSAPMPEPQAPGRYFAHSILTVSEQEWNTGETYTCMVAHHALPNRVTERTVDKSI

SEQ ID NO: 188 - protein, 105 aa, synthetic construct
  REGION 1..105: note = Synthetic
VALHRPDVYLLPPAREQLNLRESATITCLVTGFSPADVFVQWMQRGQPLSPEKYVTSAPMPEPQAPGRYFAHSILTVSEEEWNTGETYTCVVAHEATERTVDKSA

SEQ ID NO: 189 - protein, 107 aa, synthetic construct
  REGION 1..107: note = Synthetic
VALHRPDVYLLPPAREQLNLRESATITCLVTGFSPADVFVQWMQRGQPLSPEKYVTSAPMPEPQAPGRYFAHSILTVSEEEWNTGETYTCVVAHEALPTERTVDKSA

SEQ ID NO: 190 - protein, 110 aa, synthetic construct
  REGION 1..110: note = Synthetic
VALHRPDVYLLPPAREQLNLRESATITCLVTGFSPADVFVQWMQRGQPLSPEKYVTSAPMPEPRAPGRYFAHSILTVSEEEWNTGETYTCVVAHEALPNRVTERTVDKSA

SEQ ID NO: 191 - protein, 214 aa, synthetic construct
  REGION 1..214: note = Synthetic
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPCDEQLKSGTASVVCLLNNFYPREAKVQWKVDNALQSGNSQESVTEQDSKDSTYSLSSTLTLSKADYEKHKVYACEVTHQGLSSPVTKSFNRGEA

SEQ ID NO: 192 - protein, 214 aa, synthetic construct
  REGION 1..214: note = Synthetic
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDECLKSGTASVVCLLNNFYPREAKVQWKVDNALQSGNSQESVTEQDSKDSTYSLSSTLTLSKADYEKHKVYACEVTHQGLSSPVTKSFNRGEA

SEQ ID NO: 193 - protein, 214 aa, synthetic construct
  REGION 1..214: note = Synthetic
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVVCLLNNFYPREAKVQWKVDNALQSGNSCESVTEQDSKDSTYSLSSTLTLSKADYEKHKVYACEVTHQGLSSPVTKSFNRGEA

SEQ ID NO: 194 - protein, 463 aa, synthetic construct
  REGION 1..463: note = Synthetic
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSAVALHRPDVCLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTEPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 195 - protein, 463 aa, synthetic construct
  REGION 1..463: note = Synthetic
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSAVALHRPDVYLLPPAREQLNLRESATITCLVTGFSPADVFVQWMQRGQPLSPEKYVTSAPMPEPQAPGRYCAHSILTVSEEEWNTGETYTCVVAHEALPNRVTERTVDKSTEPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 196 - protein, 19 aa, synthetic construct
  REGION 1..19: note = Synthetic
VPSTPPTPSPSTPPTPSPS

SEQ ID NO: 197 - protein, 6 aa, synthetic construct
  REGION 1..6: note = Synthetic
VPPPPP

SEQ ID NO: 198 - protein, 15 aa, synthetic construct
  REGION 1..15: note = Synthetic
EPKSIDKTHTCPPCP

SEQ ID NO: 199 - protein, 15 aa, synthetic construct
  REGION 1..15: note = Synthetic
EPKSTDKTHTCPPCP

SEQ ID NO: 200 - protein, 644 aa, synthetic construct
MLLFVLTCLLAVFPAISTKSPIFGPEEVNSVEGNSVSITCYYPPTSVNRHTRKYWCRQGARGGCITLISSEGYVSSKYAGRANLTNFPENGTFVVNIAQLSQDDSGRYKCGLGINSRGLSFDVSLEVSQGPGLLNDTKVYTVDLGRTVTINCPFKTENAQKRKSLYKQIGLYPVLVIDSSGYVNPNYTGRIRLDIQGTGQLLFSVVINQLRLSDAGQYLCQAGDDSNSNKKNADLQVLKPEPELVYEDLRGSVTFHCALGPEVANVAKFLCRQSSGENCDVVVNTLGKRAPAFEGRILLNPQDKDGSFSVVITGLRKEDAGRYLCGAHSDGQLQEGSPIQAWQLFVNEESTIPRSPTVVKGVAGSSVAVLCPYNRKESKSIKYWCLWEGAQNGRCPLLVDSEGWVKAQYEGRLSLLEEPGNGTFTVILNQLTSRDAGFYWCLTNGDTLWRTTVEIKIIEGEPNLKVPGNVTAVLGETLKVPCHFPCKFSSYEKYWCKWNNTGCQALPSQDEGPSKAFVNCDENSRLVSLTLNLVTRADEGWYWCGVKQGHFYGETAAVYVAVEERKAAGSRDVSLAKADAAPDEKVLDSGFREIENKAIQDPRLFAEEKAVADTRDQADGSRASVDSGSSEEQGGSSRHHHHHH

SEQ ID NO: 201 - protein, 440 aa, synthetic construct
LPQKRPHPRWLWEGSLPSRTHLRAMGTLRPSSPLCWREESSFAAPNSLKGSRLVSGEPGGAVTIQCHYAPSSVNRHQRKYWCRLGPPRWICQTIVSTNQYTHHRYRDRVALTDFPQRGLFVVRLSQLSPDDIGCYLCGIGSENNMLFLSMNLTISAGPASTLPTATPAAGELTMRSYGTASPVANRWTPGTTQTLGQGTAWDTVASTPGTSKTTASAEGRRTPGATRPAAPGTGSWAEGSVKAPAPIPESPPSKSRSMSNTTEGVWEGTRSSVTNRARASKDRREMTTTKADRPREDIEGVRIALDAAKKVLGTIGPPALVSETLAWEILPQATPVSKQQSQGSIGETTPAAGMWTLGTPAADVWILGTPAADVWTSMEAASGEGSAAGDLDAATGDRGPQATLSQTPAVGPWGPPGKESSVKRTFPEDESSSRHHHHHH

SEQ ID NO: 202 - protein, 245 aa, synthetic construct
RILPEVKVEGELGGSVTIKCPLPEMHVRIYLCREMAGSGTCGTVVSTTNFIKAEYKGRVTLKQYPRKNLFLVEVTQLTESDSGVYACGAGMNTDRGKTQKVTLNVHSEYEPSWEEQPMPETPKWFHLPYLFQMPAYASSSKFVTRVTTPAQRGKVPPVHHSSPTTQITHRPRVSRASSVAGDKPRTFLPSTTASKISALEGLLKPQTPSYNHHTRLHRQRALDYGSQSGREGQGHHYPYDVPDYA

SEQ ID NO: 203 - protein, 573 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSGSASAPTLFPLVSCENSPSDTSSVAVGCLAQDFLPDSITFSWKYKNNSDISSTRGFPSVLRGGKYAATSQVLLPSKDVMQGTDEHVVCKVQHPNGNKEKNVPLPVIAELPPKVSVFVPPRDGFFGNPRKSKLICQATGFSPRQIQVSWLREGKQVGSGVTTDQVQAEAKESGPTTYKVTSTLTIKESDWLSQSMFTCRVDHRGLTFQQNASSMCVPDQDTAIRVFAIPPSFASIFLTKSTKLTCLVTDLTTYDSVTISWTRQNGEAVKTHTNISESHPNATFSAVGEASICEDDWNSGERFTCTVTHTDLPSPLKQTISRPKGVALHRPDVYLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTGKPTLYNVSLVMSDTAGTCY

SEQ ID NO: 204 - protein, 137 aa, synthetic construct
QEDERIVLVDNKCKCARITSRIIRSSEDPNEDIVERNIRIIVPLNNRENISDPTSPLRTRFVYHLSDLCKKCDPTEVELDNQIVTATQSNICDEDSATETCYTYDRNKCYTAVVPLVYGGETKMVETALTPDACYPD

SEQ ID NO: 205 - protein, 463 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSAVALHRPDVCLLPPAREQLNLRESATITCLVKGFSPADVFVQWMQRGQPLSPEKYVTSAPMPEPQAPGRYFAHSILTVSEEEWNTGETYTCVVAHEALPNRVTERTVDKSTEPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 206 - protein, 463 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSAVALHRPDVYLLPPAREQLNLRESATITCLVTGFSPADVFVQWMQRGQPLSPEKYVTSAPMPEPQAPGRYCAHSILTVSEEEWNTGETYTCVVAHEALPNRVTERTVDKSTEPESADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 207 - protein, 463 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSAVALHRPDVCLLPPAREQLNLRESATITCLVTGFSPADVFVQWMQRGQPLSPEKYVTSAPMPEPQAPGRYFAHSILTVSEEEWNTGETYTCVVAHEALPNRVTERTVDKSTEPESADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 208 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPKAPSSKSTSGGTAALGCLVKDYFPEPVTVSWNSGALTSGVHTFPAVLQSSGLYSLSSSVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 209 - protein, 463 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSAVALHRPDVCLLPPAREQLNLRESATITCLVEGFSPADVFVQWMQRGQPLSPEKYVTSAPMPEPQAPGRYFAHSILTVSEEEWNTGETYTCVVAHEALPNRVTERTVDKSTEPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 210 - protein, 463 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSAVALHRPDVYLLPPAREQLNLRESATITCLVEGFSPADVFVQWMQRGQPLSPEKYVTSAPMPEPQAPGRYCAHSILTVSEEEWNTGETYTCVVAHEALPNRVTERTVDKSTEPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 211 - protein, 463 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSAVALHRPDVCLLPPAREQLNLRESATITCLVTGFSPADVFVQWMQRGQPLSPEKYVTSAPMPEPQAPGRYFAHSILTVSEEEWNTGETYTCVVAHEALPNRVTERTVDESTEPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 212 - protein, 463 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSAVALHRPDVYLLPPAREQLNLRESATITCLVTGFSPADVFVQWMQRGQPLSPEKYVTSAPMPEPQAPGRYCAHSILTVSEEEWNTGETYTCVVAHEALPNRVTERTVDESTEPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 213 - protein, 463 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSAVALHRPDVYLLPPAREQLNLRESATITCLVTGFSPADVFVQWMQRGQPLSPEKYVTSAPMPEPQAPGRYCAHSILTVSEEEWNTGETYTCVVAHEALPNRVTERTVDESTEPESADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 214 - protein, 463 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSAVALHRPDVCLLPPAREQLNLRESATITCLVTGFSPADVFVQWMQRGQPLSPEKYVTSAPMPEPQAPGRYFAHSILTVSEEEWNTGETYTCVVAHEALPNRVTERTVDESTEPESADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 215 - protein, 463 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSAVALHRPDVCLLPPAREQLNLRESATITCLVDGFSPADVFVQWMQRGQPLSPEKYVTSAPMPEPQAPGRYFAHSILTVSEEEWNTGETYTCVVAHEALPNRVTERTVDKSTEPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 216 - protein, 463 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSAVALHRPDVYLLPPAREQLNLRESATITCLVDGFSPADVFVQWMQRGQPLSPEKYVTSAPMPEPQAPGRYCAHSILTVSEEEWNTGETYTCVVAHEALPNRVTERTVDKSTEPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 217 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPKAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 218 - protein, 110 aa, synthetic construct
VALHRPDVCLLPPAREQLNLRESATITCLVKGFSPADVFVQWMQRGQPLSPEKYVTSAPMPEPQAPGRYFAHSILTVSEEEWNTGETYTCVVAHEALPNRVTERTVDKST

SEQ ID NO: 219 - protein, 110 aa, synthetic construct; identical to SEQ ID NO: 75

SEQ ID NO: 220 - protein, 110 aa, synthetic construct; identical to SEQ ID NO: 74

SEQ ID NO: 221 - protein, 98 aa, synthetic construct
ASTKGPSVFPKAPSSKSTSGGTAALGCLVKDYFPEPVTVSWNSGALTSGVHTFPAVLQSSGLYSLSSSVTVPSSSLGTQTYICNVNHKPSNTKVDKKV

SEQ ID NO: 222 - protein, 110 aa, synthetic construct
VALHRPDVCLLPPAREQLNLRESATITCLVEGFSPADVFVQWMQRGQPLSPEKYVTSAPMPEPQAPGRYFAHSILTVSEEEWNTGETYTCVVAHEALPNRVTERTVDKST

SEQ ID NO: 223 - protein, 110 aa, synthetic construct
VALHRPDVYLLPPAREQLNLRESATITCLVEGFSPADVFVQWMQRGQPLSPEKYVTSAPMPEPQAPGRYCAHSILTVSEEEWNTGETYTCVVAHEALPNRVTERTVDKST

SEQ ID NO: 224 - protein, 110 aa, synthetic construct
VALHRPDVCLLPPAREQLNLRESATITCLVTGFSPADVFVQWMQRGQPLSPEKYVTSAPMPEPQAPGRYFAHSILTVSEEEWNTGETYTCVVAHEALPNRVTERTVDEST

SEQ ID NO: 225 - protein, 110 aa, synthetic construct
VALHRPDVYLLPPAREQLNLRESATITCLVTGFSPADVFVQWMQRGQPLSPEKYVTSAPMPEPQAPGRYCAHSILTVSEEEWNTGETYTCVVAHEALPNRVTERTVDEST

SEQ ID NO: 226 - protein, 110 aa, synthetic construct; identical to SEQ ID NO: 225

SEQ ID NO: 227 - protein, 110 aa, synthetic construct; identical to SEQ ID NO: 224

SEQ ID NO: 228 - protein, 110 aa, synthetic construct
VALHRPDVCLLPPAREQLNLRESATITCLVDGFSPADVFVQWMQRGQPLSPEKYVTSAPMPEPQAPGRYFAHSILTVSEEEWNTGETYTCVVAHEALPNRVTERTVDKST

SEQ ID NO: 229 - protein, 110 aa, synthetic construct
VALHRPDVYLLPPAREQLNLRESATITCLVDGFSPADVFVQWMQRGQPLSPEKYVTSAPMPEPQAPGRYCAHSILTVSEEEWNTGETYTCVVAHEALPNRVTERTVDKST

SEQ ID NO: 230 - protein, 98 aa, synthetic construct
ASTKGPSVFPKAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 231 - protein, 15 aa, synthetic construct
EPKSADKTHTCPPCP

SEQ ID NO: 232 - protein, 15 aa, synthetic construct
EPESADKTHTCPPCP

SEQ ID NO: 233 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDECLKSGTAHVVCLLNNFYPREAKVQWKVDNALQSGNSQESVTEQDSKDSTYSLSSTLTLSKADYEKHKVYACEVTHQGLSSPVTKSFNRGEA

SEQ ID NO: 234 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDKQLKSGTASVVCLLNNFYPREAKVQWKVDNALQSGNSCESVTEQDSKDSTYSLSSTLTLSKADYEKHKVYACEVTHQGLSSPVTKSFNRGEA

SEQ ID NO: 235 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDKCLKSGTASVVCLLNNFYPREAKVQWKVDNALQSGNSQESVTEQDSKDSTYSLSSTLTLSKADYEKHKVYACEVTHQGLSSPVTKSFNRGEA

SEQ ID NO: 236 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVSCLLNNFYPREAKVQWKVDNALQSGNSQESVTEQDSKDSTYSLDSTLTLSKADYEKHKVYACEVTHQGLSSPVTKSFNRGEC

SEQ ID NO: 237 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTAHVVCLLNNFYPREAKVQWKVDNALQSGNSCESVTEQDSKDSTYSLSSTLTLSKADYEKHKVYACEVTHQGLSSPVTKSFNRGEA

SEQ ID NO: 238 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSKKCLKSGTASVVCLLNNFYPREAKVQWKVDNALQSGNSQESVTEQDSKDSTYSLSSTLTLSKADYEKHKVYACEVTHQGLSSPVTKSFNRGEA

SEQ ID NO: 239 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPKDKCLKSGTASVVCLLNNFYPREAKVQWKVDNALQSGNSQESVTEQDSKDSTYSLSSTLTLSKADYEKHKVYACEVTHQGLSSPVTKSFNRGEA

SEQ ID NO: 240 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTAKVVCLLNNFYPREAKVQWKVDNALQSGNSCESVTEQDSKDSTYSLSSTLTLSKADYEKHKVYACEVTHQGLSSPVTKSFNRGEA

SEQ ID NO: 241 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTARVVCLLNNFYPREAKVQWKVDNALQSGNSCESVTEQDSKDSTYSLSSTLTLSKADYEKHKVYACEVTHQGLSSPVTKSFNRGEA

SEQ ID NO: 242 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDECLKSGTAKVVCLLNNFYPREAKVQWKVDNALQSGNSQESVTEQDSKDSTYSLSSTLTLSKADYEKHKVYACEVTHQGLSSPVTKSFNRGEA

SEQ ID NO: 243 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDECLKSGTARVVCLLNNFYPREAKVQWKVDNALQSGNSQESVTEQDSKDSTYSLSSTLTLSKADYEKHKVYACEVTHQGLSSPVTKSFNRGEA

SEQ ID NO: 244 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSKKQLKSGTASVVCLLNNFYPREAKVQWKVDNALQSGNSCESVTEQDSKDSTYSLSSTLTLSKADYEKHKVYACEVTHQGLSSPVTKSFNRGEA

SEQ ID NO: 245 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPKDKQLKSGTASVVCLLNNFYPREAKVQWKVDNALQSGNSCESVTEQDSKDSTYSLSSTLTLSKADYEKHKVYACEVTHQGLSSPVTKSFNRGEA

SEQ ID NO: 246 - protein, 107 aa, synthetic construct
RTVAAPSVFIFPPSDECLKSGTAHVVCLLNNFYPREAKVQWKVDNALQSGNSQESVTEQDSKDSTYSLSSTLTLSKADYEKHKVYACEVTHQGLSSPVTKSFNRGEA

SEQ ID NO: 247 - protein, 107 aa, synthetic construct
RTVAAPSVFIFPPSDKQLKSGTASVVCLLNNFYPREAKVQWKVDNALQSGNSCESVTEQDSKDSTYSLSSTLTLSKADYEKHKVYACEVTHQGLSSPVTKSFNRGEA

SEQ ID NO: 248 - protein, 107 aa, synthetic construct
RTVAAPSVFIFPPSDKCLKSGTASVVCLLNNFYPREAKVQWKVDNALQSGNSQESVTEQDSKDSTYSLSSTLTLSKADYEKHKVYACEVTHQGLSSPVTKSFNRGEA

SEQ ID NO: 249 - protein, 107 aa, synthetic construct
RTVAAPSVFIFPPSDEQLKSGTASVSCLLNNFYPREAKVQWKVDNALQSGNSQESVTEQDSKDSTYSLDSTLTLSKADYEKHKVYACEVTHQGLSSPVTKSFNRGEC

SEQ ID NO: 250 - protein, 107 aa, synthetic construct
RTVAAPSVFIFPPSDEQLKSGTAHVVCLLNNFYPREAKVQWKVDNALQSGNSCESVTEQDSKDSTYSLSSTLTLSKADYEKHKVYACEVTHQGLSSPVTKSFNRGEA

SEQ ID NO: 251 - protein, 107 aa, synthetic construct
RTVAAPSVFIFPPSKKCLKSGTASVVCLLNNFYPREAKVQWKVDNALQSGNSQESVTEQDSKDSTYSLSSTLTLSKADYEKHKVYACEVTHQGLSSPVTKSFNRGEA

SEQ ID NO: 252 - protein, 107 aa, synthetic construct
RTVAAPSVFIFPPKDKCLKSGTASVVCLLNNFYPREAKVQWKVDNALQSGNSQESVTEQDSKDSTYSLSSTLTLSKADYEKHKVYACEVTHQGLSSPVTKSFNRGEA

SEQ ID NO: 253 - protein, 107 aa, synthetic construct
RTVAAPSVFIFPPSDEQLKSGTAKVVCLLNNFYPREAKVQWKVDNALQSGNSCESVTEQDSKDSTYSLSSTLTLSKADYEKHKVYACEVTHQGLSSPVTKSFNRGEA

SEQ ID NO: 254 - protein, 107 aa, synthetic construct
RTVAAPSVFIFPPSDEQLKSGTARVVCLLNNFYPREAKVQWKVDNALQSGNSCESVTEQDSKDSTYSLSSTLTLSKADYEKHKVYACEVTHQGLSSPVTKSFNRGEA

SEQ ID NO: 255 - protein, 107 aa, synthetic construct
RTVAAPSVFIFPPSDECLKSGTAKVVCLLNNFYPREAKVQWKVDNALQSGNSQESVTEQDSKDSTYSLSSTLTLSKADYEKHKVYACEVTHQGLSSPVTKSFNRGEA

SEQ ID NO: 256 - protein, 107 aa, synthetic construct
RTVAAPSVFIFPPSDECLKSGTARVVCLLNNFYPREAKVQWKVDNALQSGNSQESVTEQDSKDSTYSLSSTLTLSKADYEKHKVYACEVTHQGLSSPVTKSFNRGEA

SEQ ID NO: 257 - protein, 107 aa, synthetic construct
RTVAAPSVFIFPPSKKQLKSGTASVVCLLNNFYPREAKVQWKVDNALQSGNSCESVTEQDSKDSTYSLSSTLTLSKADYEKHKVYACEVTHQGLSSPVTKSFNRGEA

SEQ ID NO: 258 - protein, 107 aa, synthetic construct
RTVAAPSVFIFPPKDKQLKSGTASVVCLLNNFYPREAKVQWKVDNALQSGNSCESVTEQDSKDSTYSLSSTLTLSKADYEKHKVYACEVTHQGLSSPVTKSFNRGEA

SEQ ID NO: 259 - protein, 463 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSAVALHRPDVCLLPPAREQLNLRRSATITCLVEGFSPADVFVQWMQRGQPLSPEKYVTSAPMPEPRAPGRYFAHSILTVSEAEWNTGETYTCVVAHEALPNRVTERTVDKSTEPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 260 - protein, 463 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSAVALHRPDVYLLPPAREQLNLRRSATITCLVEGFSPADVFVQWMQRGQPLSPEKYVTSAPMPEPRAPGRYCAHSILTVSEAEWNTGETYTCVVAHEALPNRVTERTVDKSTEPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 261 - protein, 463 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSAVALHRPDVCLLPPAREQLNLRRSATITCLVDGFSPADVFVQWMQRGQPLSPEKYVTSAPMPEPRAPGRYFAHSILTVSEAEWNTGETYTCVVAHEALPNRVTERTVDKSTEPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 262 - protein, 463 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSAVALHRPDVYLLPPAREQLNLRRSATITCLVDGFSPADVFVQWMQRGQPLSPEKYVTSAPMPEPRAPGRYCAHSILTVSEAEWNTGETYTCVVAHEALPNRVTERTVDKSTEPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 263 - protein, 463 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSAVALHRPDVYLLPPAREQLNLRRSATITCLVTGFSPADVFVQWMQRGQPLSPEKYVTSAPMPEPRAPGRYCAHSILTVSEAEWNTGETYTCVVAHEALPNRVTERTVDESTEPKSADKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 264 - protein, 450 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSASTKGPSVFPRAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSREEMTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 265 - protein, 111 aa, synthetic construct
AVALHRPDVCLLPPAREQLNLRRSATITCLVEGFSPADVFVQWMQRGQPLSPEKYVTSAPMPEPRAPGRYFAHSILTVSEAEWNTGETYTCVVAHEALPNRVTERTVDKST

SEQ ID NO: 266 - protein, 111 aa, synthetic construct
AVALHRPDVYLLPPAREQLNLRRSATITCLVEGFSPADVFVQWMQRGQPLSPEKYVTSAPMPEPRAPGRYCAHSILTVSEAEWNTGETYTCVVAHEALPNRVTERTVDKST

SEQ ID NO: 267 - protein, 111 aa, synthetic construct
AVALHRPDVCLLPPAREQLNLRRSATITCLVDGFSPADVFVQWMQRGQPLSPEKYVTSAPMPEPRAPGRYFAHSILTVSEAEWNTGETYTCVVAHEALPNRVTERTVDKST

SEQ ID NO: 268 - protein, 111 aa, synthetic construct
AVALHRPDVYLLPPAREQLNLRRSATITCLVDGFSPADVFVQWMQRGQPLSPEKYVTSAPMPEPRAPGRYCAHSILTVSEAEWNTGETYTCVVAHEALPNRVTERTVDKST

SEQ ID NO: 269 - protein, 111 aa, synthetic construct
AVALHRPDVYLLPPAREQLNLRRSATITCLVTGFSPADVFVQWMQRGQPLSPEKYVTSAPMPEPRAPGRYCAHSILTVSEAEWNTGETYTCVVAHEALPNRVTERTVDEST

SEQ ID NO: 270 - protein, 98 aa, synthetic construct
ASTKGPSVFPRAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 271 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTASVSCLLNNFYPREAKVQWKVDNALQSGNSQESVTEQDSKDSTYSLESTLTLSKADYEKHKVYACEVTHQGLSSPVTKSFNRGEC

SEQ ID NO: 272 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTAKVVCLLLNFYPREAKVQWKVDNALQSGNSCESVTEQDSKDSTYSLSSTLTLSKADYEKHKVYACEVTHQGLSSPVTKSFNRGEA

SEQ ID NO: 273 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEELKSGTASVSCLLNNFYPREAKVQWKVDNALQSGNSQESVTEQDSKDSTYSLDSTLTLSKADYEKHKVYACEVTHQGLSSPVTKSFNRGEC

SEQ ID NO: 274 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTAKVVCLLANFYPREAKVQWKVDNALQSGNSCESVTEQDSKDSTYSLSSTLTLSKADYEKHKVYACEVTHQGLSSPVTKSFNRGEA

SEQ ID NO: 275 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTAKVVCLLRNFYPREAKVQWKVDNALQSGNSCESVTEQDSKDSTYSLSSTLTLSKADYEKHKVYACEVTHQGLSSPVTKSFNRGEA

SEQ ID NO: 276 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTAKVVCLLKNFYPREAKVQWKVDNALQSGNSCESVTEQDSKDSTYSLSSTLTLSKADYEKHKVYACEVTHQGLSSPVTKSFNRGEA

SEQ ID NO: 277 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEQLKSGTAKVVCLLQNFYPREAKVQWKVDNALQSGNSCESVTEQDSKDSTYSLSSTLTLSKADYEKHKVYACEVTHQGLSSPVTKSFNRGEA

SEQ ID NO: 278 - protein, 214 aa, synthetic construct
DIQMTQSPSSLSASVGDRVTITCRASQDVNTAVAWYQQKPGKAPKLLIYSASFLYSGVPSRFSGSRSGTDFTLTISSLQPEDFATYYCQQHYTTPPTFGQGTKVEIKRTVAAPSVFIFPPSDEDLKSGTASVSCLLNNFYPREAKVQWKVDNALQSGNSQESVTEQDSKDSTYSLDSTLTLSKADYEKHKVYACEVTHQGLSSPVTKSFNRGEC

SEQ ID NO: 279 - protein, 107 aa, synthetic construct
RTVAAPSVFIFPPSDEQLKSGTASVSCLLNNFYPREAKVQWKVDNALQSGNSQESVTEQDSKDSTYSLESTLTLSKADYEKHKVYACEVTHQGLSSPVTKSFNRGEC

SEQ ID NO: 280 - protein, 107 aa, synthetic construct
RTVAAPSVFIFPPSDEQLKSGTAKVVCLLLNFYPREAKVQWKVDNALQSGNSCESVTEQDSKDSTYSLSSTLTLSKADYEKHKVYACEVTHQGLSSPVTKSFNRGEA

SEQ ID NO: 281 - protein, 107 aa, synthetic construct
RTVAAPSVFIFPPSDEELKSGTASVSCLLNNFYPREAKVQWKVDNALQSGNSQESVTEQDSKDSTYSLDSTLTLSKADYEKHKVYACEVTHQGLSSPVTKSFNRGEC

SEQ ID NO: 282 - protein, 107 aa, synthetic construct
RTVAAPSVFIFPPSDEQLKSGTAKVVCLLANFYPREAKVQWKVDNALQSGNSCESVTEQDSKDSTYSLSSTLTLSKADYEKHKVYACEVTHQGLSSPVTKSFNRGEA

SEQ ID NO: 283 - protein, 107 aa, synthetic construct
RTVAAPSVFIFPPSDEQLKSGTAKVVCLLRNFYPREAKVQWKVDNALQSGNSCESVTEQDSKDSTYSLSSTLTLSKADYEKHKVYACEVTHQGLSSPVTKSFNRGEA

SEQ ID NO: 284 - protein, 107 aa, synthetic construct
RTVAAPSVFIFPPSDEQLKSGTAKVVCLLKNFYPREAKVQWKVDNALQSGNSCESVTEQDSKDSTYSLSSTLTLSKADYEKHKVYACEVTHQGLSSPVTKSFNRGEA

SEQ ID NO: 285 - protein, 107 aa, synthetic construct
RTVAAPSVFIFPPSDEQLKSGTAKVVCLLQNFYPREAKVQWKVDNALQSGNSCESVTEQDSKDSTYSLSSTLTLSKADYEKHKVYACEVTHQGLSSPVTKSFNRGEA

SEQ ID NO: 286 - protein, 107 aa, synthetic construct
RTVAAPSVFIFPPSDEDLKSGTASVSCLLNNFYPREAKVQWKVDNALQSGNSQESVTEQDSKDSTYSLDSTLTLSKADYEKHKVYACEVTHQGLSSPVTKSFNRGEC

SEQ ID NO: 287 - protein, 247 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSAVALHRPDVCLLPPAREQLNLRRSATITCLVTGFSPADVFVQWMQRGQPLSPEKYVTSAPMPEPRAPGRYFAHSILTVSEAEWNTGETYTCVVAHEALPNRVTERTVDKSTEPESADKTHHHHHHHH

SEQ ID NO: 288 - protein, 127 aa, synthetic construct
AVALHRPDVCLLPPAREQLNLRRSATITCLVTGFSPADVFVQWMQRGQPLSPEKYVTSAPMPEPRAPGRYFAHSILTVSEAEWNTGETYTCVVAHEALPNRVTERTVDKSTEPESADKTHHHHHHHH

SEQ ID NO: 289 - protein, 107 aa, synthetic construct
RTVAAPSVFIFPPSDKCLKSGTASVVCLLSNFYPREAKVQWKVDNALQSGSSQESTTEQDSKDSTYSLSSTLTLSKADYEKHKVYACEVTHQGLSSPVTKSFNRGEA

SEQ ID NO: 290 - protein, 107 aa, synthetic construct
RTVAAPSVFIFPPSDKCLKSGTASVVCLLNGFYPREAKVKWKVDNALQSGNSQESVTEQDSKDSTYSLSSTLTLSKAEYEKHKVYACEVTHQGLSSPVTKSFNRGEA

SEQ ID NO: 291 - protein, 107 aa, synthetic construct
RTVAAPSVFIFPPSDKCLESGTASVVCLLNNFYPREAKVQWKVDNALQSGNSQESVTEQDSKDSTYSLSSTLTLSKADYEKHKVYACEVTHQGLSSPLTKSFSRGEA

SEQ ID NO: 292 - protein, 107 aa, synthetic construct
RTVAAPSVFIFPPSDKCLRSGSASVVCLLNNFYPREAKVQWKVDNALQTGNSQESVTEQDSKDSTYSLSSTLTLSKADYEKHKVYACEVTHQGLSSPVTKSFNRGEA

SEQ ID NO: 293 - protein, 107 aa, synthetic construct
RTVAAPSVFIFPPSDKCLKSGTATVVCLLNNFYPREAKVQWKVDNALQSGSSQESVTEQDSKDSTYSLSSTLTLSKADYEKHKVYTCEVTHQGLSSPVTKSFNRGEA

SEQ ID NO: 294 - protein, 107 aa, synthetic construct
RTVAAPSVFIFPPSDKCLKSGTASVVCLLNNFYPREIKVQWKVDNALQSGNSQESVTEQDSKDSTYSLSSTLTLSKADYEKHKVYTCEVTHQGLSSPVTKSFNRAEA

SEQ ID NO: 295 - protein, 107 aa, synthetic construct
RTVAAPSVFIFPPSDKCLKSGGASVVCLLNNFYPREIKVQWKVDNALQSGNFQESVTEQDSKDSTYSLSSTLTLSKADYEKHKVYACEVTHQGLSSPVTKSFNRGEA

SEQ ID NO: 296 - protein, 107 aa, synthetic construct
RTVAAPSVFIFPPSDKCLKSGTASVVCLVNNFYPREAKVQWKVDNALQSGNSQESSTEQDSKDSTYSLSSTLTLSKAEYEKHKVYACEVTHQGLSSPVTKSFNRGEA

SEQ ID NO: 297 - protein, 107 aa, synthetic construct
RTVAAPSVFIFPPSDKCLKSGTASVVCLLNNFYPREAKVSWKVDNAAQSGNSQESTTEQDSKDSTYSLSSTLTLSKADYEKHKVYACEVTHQGLSSPVTKSFNRGEA

SEQ ID NO: 298 - protein, 107 aa, synthetic construct
RTVAAPSVFIFPPSDKCLKSGSASVVCLLNNFYPREAKVQWKVDNALQSGNSQEGVTEQDSKDSTYSLSSTLTLSKADYDKHKVYACEVTHQGLSSPVTKSFNRGEA

SEQ ID NO: 299 - protein, 107 aa, synthetic construct
RTVAAPSVFIFPPSDKCLKSGGASVVCLLNNFYPREAKVQWKVDNALQSGNIQESVTEQDSKDSTYSLSSTLTLSKAEYEKHKVYACEVTHQGLSSPVTKSFNRGEA

SEQ ID NO: 300 - protein, 107 aa, synthetic construct
RTVAQPSVFIFPPSDKCLKSGTASVVCLLNNFYPREAKVQWKVDNALQSGNSQESFTDQDSKDSTYSLSSTLTLSKADYEKHKVYACEVTHQGLSSPVTKSFNRGEA

SEQ ID NO: 301 - protein, 107 aa, synthetic construct
RTVAAPSVFIFPPSDKCLESGTASVVCLLNNFYPREVKVQWKVDNALQSGNSQESVTEQDSKDSTYSLSSTLTLSKADYEKHKVYACEVTHQGLSSPVTKSFNRGTA

SEQ ID NO: 302 - protein, 107 aa, synthetic construct
RTVAAPSVFIFPPSDKCLKSGTASVVCLLNNFYPREAKVQWKVDNALQSGNSQESSTEQDSKDKTYSLSSTLTLSKADYEKHKTYACEVTHQGLSSPVTKSFNRGEA

SEQ ID NO: 303 - protein, 107 aa, synthetic construct
RTVTAPSVFIFPPSDKCLKSGTASVVCLLNNFYPREAKVKWKVDNALQSGNSQESVTEQDSKDSTYSLSSTLTLSKADYEKHKVYACEVTHQGLSSPVTKSFSRGEA

SEQ ID NO: 304 - protein, 107 aa, synthetic construct
RTVAAPSVFIFPPSDKCLKSGTATVVCLLNNFYPREAKVQWKVDNALQSGNSQESVVDQDSKDSTYSLSSTLTLSKADYEKHKVYACEVTHQGLSSPVTKSFNRGEA

SEQ ID NO: 305 - protein, 107 aa, synthetic construct
RTVAAPTVFIFPPSDKCLKSGTASVVCLLNNFYPREAKVRWKVDNALQSGNSQEGVTEQDSKDSTYSLSSTLTLSKADYEKHKVYACEVTHQGLSSPVTKSFNRGEA

SEQ ID NO: 306 - protein, 107 aa, synthetic construct
RTVAAPSVFIFQPSDKCLKSGTASVVCLLNNFYPREAKVQWKVDNALQSGDSQESVTEQDSKDSTYSLSSTLTLSKADYEKHKVYSCEVTHQGLSSPVTKSFNRGEA

SEQ ID NO: 307 - protein, 107 aa, synthetic construct
RTVAQPSVFIFPPSDKCLKSGTASVVCLLNNFYPREAKVQWKVDNALQSGSSQESVTEQDSKDKTYSLSSTLTLSKADYEKHKVYACEVTHQGLSSPVTKSFNRGEA

SEQ ID NO: 308 - protein, 107 aa, synthetic construct
RTVAAPSVFIFPPSDKCLKSGSASVVCLLNNFYPREAKVSWKVDNALQSGNSQESSTEQDSKDSTYSLSSTLTLSKADYEKHKVYACEVTHQGLSSPVTKSFNRGEA

SEQ ID NO: 309 - protein, 107 aa, synthetic construct
RTVAAPSVFIFPPSEKCLKSGTASVVCLLNNFYPREVKVQWKVDNALQSGNIQESVTEQDSKDSTYSLSSTLTLSKADYEKHKVYACEVTHQGLSSPVTKSFNRGEA

SEQ ID NO: 310 - protein, 107 aa, synthetic construct
RTVAAPSVFIFPPSDKCLKSGGASVVCLLNNFYPREAKVNWKVDNALQSGNSQESVTEQDSKDSTYSLSSTLTLSKADYEKHKVYSCEVTHQGLSSPVTKSFNRGEA

SEQ ID NO: 311 - protein, 107 aa, synthetic construct
RTVAAPSAFIFPPSDKCLKSGTASVVCLLNNFYPREAKVRWKVDNALQSGNSQESVTEQDSKDSTYSLSSTLTLSKADYEKHKTYACEVTHQGLSSPVTKSFNRGEA

SEQ ID NO: 312 - protein, 107 aa, synthetic construct
RTVAAPSVFIFPPSDKCLKSGTASVVCLLNNFYPREAKVRWKVDNAVQSGNSQESVTEQDSKDSTYSLSSTLTLSKADYEKHKVYSCEVTHQGLSSPVTKSFNRGEA

SEQ ID NO: 313 - protein, 107 aa, synthetic construct
RTVAAPTAFLFPPSDKCLKSGTASVVCLLNNFYPREAKVQWKVDNALQSGNSQESVTEQDSKDSTYSLSSTLTLSKADYEKHKVYACEVTHQGLSSPVTKSFNRGEA

SEQ ID NO: 314 - protein, 107 aa, synthetic construct
RTVAAPSVFLFPPSDKCLKSGTASVVCLLNNFYPREAKVQWKVDNALQNGNSQESLTEQDSKDSTYSLSSTLTLSKADYEKHKVYACEVTHQGLSSPVTKSFNRGEA

SEQ ID NO: 315 - protein, 107 aa, synthetic construct
RTVAAPSVFIFPPSDKCLKSGSASVVCLLNNFYPREIKVQWKVDNALQSGNSQESLTEQDSKDSTYSLSSTLTLSKADYEKHKVYACEVTHQGLSSPVTKSFNRGEA

SEQ ID NO: 316 - protein, 107 aa, synthetic construct
RTVAAPSVFIFPPSDKCLKSGTASVVCLLNNFYPREVKVQWKVDNALQSGDSQESVTEQDSKDSTYSLSSTLTLSKADYEKHKVYACEVTHQGLSSPLTKSFNRGEA

SEQ ID NO: 317 - protein, 107 aa, synthetic construct
RTVAAPSAFIFPPSDKCLKSGTASVVCLLNNFYPREAKVQWKVDGALQSGGSQESVTEQDSKDSTYSLSSTLTLSKADYEKHKVYACEVTHQGLSSPVTKSFNRGEA

SEQ ID NO: 318 - protein, 107 aa, synthetic construct
RTVAAPSVFIFPPSQKCLKSGTASVVCMLNNFYPREAKVQWKVDNALQSGNSQESVTEQDSKDGTYSLSSTLTLSKADYEKHKVYACEVTHQGLSSPVTKSFNRGEA

SEQ ID NO: 319 - protein, 107 aa, synthetic construct
RTVAAPSVFIFPPSQKCLKSGTASVVCLLNNFYPREAKVQWKVDNALQSGNVQESVTEQDSKDSTYSLSSTLTLSKAEYEKHKVYACEVTHQGLSSPVTKSFNRGEA

SEQ ID NO: 320 - protein, 107 aa, synthetic construct
RTVAAPSVFIFPPSDKCLKSGTASVVCLLNNFYPREAKVQWKVDNALQSGNSQESVTDQDSKDNTYSLSSTLTLSKADYEKHKTYACEVTHQGLSSPVTKSFNRGEA

SEQ ID NO: 321 - protein, 107 aa, synthetic construct
RTVAAPSVFIFPPSDKCLKSGTASVVCLLNNFYPREAKVNWKVDNAVQSGNSQESITEQDSKDSTYSLSSTLTLSKADYEKHKVYACEVTHQGLSSPVTKSFNRGEA

SEQ ID NO: 322 - protein, 107 aa, synthetic construct
RTVAAPSVFIFPPSDKCLRSGTASVVCLLNGFYPREAKVQWKVDNALQSGNSQESITEQDSKDSTYSLSSTLTLSKADYEKHKVYACEVTHQGLSSPVTKSFNRGEA

SEQ ID NO: 323 - protein, 107 aa, synthetic construct
RTVAAPSVFIFPPSDKCLKSGTASVVCLLNNFYPREAKVQWKVDNAAQSGNSQESVTEQDSKDSTYSLSSTLTLSKADYEKHKVYACEVTHQSLSSPLTKSFNRGEA

SEQ ID NO: 324 - protein, 107 aa, synthetic construct
RTVAAPSVFIFPPSEKCLKSGTASVVCLLNNFYPREAKVQWKVDNALQSGDSQESVTEQDSKDNTYSLSSTLTLSKADYEKHKVYACEVTHQGLSSPVTKSFNRGEA

SEQ ID NO: 325 - protein, 107 aa, synthetic construct
RTVAAPSVFIFPPSEKCLESGTASVVCMLNNFYPREAKVQWKVDNALQSGNSQESVTEQDSKDSTYSLSSTLTLSKADYEKHKVYACEVTHQGLSSPVTKSFNRGEA

SEQ ID NO: 326 - protein, 107 aa, synthetic construct
RTVAAPSVFLFPPSQKCLKSGTASVVCLLNNFYPREAKVQWKVDNALQSGGSQESVTEQDSKDSTYSLSSTLTLSKADYEKHKVYACEVTHQGLSSPVTKSFNRGEA

SEQ ID NO: 327 - protein, 107 aa, synthetic construct
RTVAAPSAFIFPPSDKCLKSGTASVVCLVNNFYPREAKVNWKVDNALQSGNSQESVTEQDSKDSTYSLSSTLTLSKADYEKHKVYACEVTHQGLSSPVTKSFNRGEA

SEQ ID NO: 328 - protein, 107 aa, synthetic construct
RTVAAPSVFIFPPSQKCLRSGTASVVCLLNNFYPREAKVQWKVDNALQSGNSQESVTEQDSKDSTYSLSSTLTLSKADYEKHKVYTCEVTHQGLSSPVTKSFNRGEA

SEQ ID NO: 329 - protein, 107 aa, synthetic construct
RTVAAPSVFIFPPSDKCLRSGTASVVCLLSNFYPREAKVQWKVDNALQSGNSQESLTEQDSKDSTYSLSSTLTLSKADYEKHKVYACEVTHQGLSSPVTKSFNRGEA

SEQ ID NO: 330 - protein, 107 aa, synthetic construct
RTVAAPSVFIFPPSDKCLKSGTATVVCLLNNFYPREAKVQWKVDNALQSGNSQESVTEQDSKDGTYSLSSTLTLSKADYEKHKVYACEVTHQGLSSPVTKSFNRSEA

SEQ ID NO: 331 - protein, 107 aa, synthetic construct
RTVTAPSVFIFPPSDKCLKSGTASVVCLLNNFYPREAKVSWKVDNALQSGNSQESITEQDSKDSTYSLSSTLTLSKADYEKHKVYACEVTHQGLSSPVTKSFNRGEA

SEQ ID NO: 332 - protein, 107 aa, synthetic construct
RTVAAPSVFIFPPSDKCLESGTASVVCLLNNFYPREAKVQWKVDNALQSGNSQESFTEQDSKDNTYSLSSTLTLSKADYEKHKVYACEVTHQGLSSPVTKSFNRGEA

SEQ ID NO: 333 - protein, 107 aa, synthetic construct
RTVAAPSVFIFPPSDKCLKSGTASVVCLLNNFYPREAKVQWKVDNAAQTGNSQESVVEQDSKDSTYSLSSTLTLSKADYEKHKVYACEVTHQGLSSPVTKSFNRGEA

SEQ ID NO: 334 - protein, 107 aa, synthetic construct
RTVAAPSVFIFPPSDKCLKSGTASVVCLLNNFYPREAKVQWKVDNALQSGNVQESLTEQDSKDSTYSLSSTLTLSKADYEKHKVYACEVTHQGLSSPVTKSFSRGEA

SEQ ID NO: 335 - protein, 107 aa, synthetic construct
RTVAAPSVFIFPPSDKCLKSGTASVVCLLNNFYPREIKVQWKVDNALQSGNSQESVTEQDSKDKTYSLSSTLTLSKAEYEKHKVYACEVTHQGLSSPVTKSFNRGEA

SEQ ID NO: 336 - protein, 107 aa, synthetic construct
RTVAAPSVFIFPPSDKCLKSGTASVVCLLSNFYPREAKVSWKVDNALQSGNSQESVTEQDSKDSTYSLSSTLTLSKADYEKHKVYACEVTHQGLSSPVTKSFNRAEA

SEQ ID NO: 337 - protein, 107 aa, synthetic construct
RTVAAPSVFIFPPSDKCLKSGTASVVCLLNNFYPREAKVKWKVDNALQNGNSQESVTEQDSKDSTYSLSSTLTLSKADYEKHKVYACEVTHQGLSSPVTKSFNRGTA

SEQ ID NO: 338 - protein, 107 aa, synthetic construct
RTVAQPSVFIFPPSDKCLKSGTASVVCLLNNFYPREAKVQWKVDNALQNGNFQESVTEQDSKDSTYSLSSTLTLSKADYEKHKVYACEVTHQGLSSPVTKSFNRGEA

SEQ ID NO: 339 - protein, 107 aa, synthetic construct
RTVAAPTVFIFPPSDKCLKSGTASVVCLLNNFYPREAKVKWKVDNAVQSGNSQESVTEQDSKDSTYSLSSTLTLSKADYEKHKVYACEVTHQGLSSPVTKSFNRGEA

SEQ ID NO: 340 - protein, 107 aa, synthetic construct
RTVAAPSVFIFPPSDKCLKSGTATVVCMLNNFYPREAKVQWKVDNALQSGNSQESVTEQDSKDSTYSLSSTLTLSKADYEKHKVYACEVTHQGLSSPVTKSFNRGSA

SEQ ID NO: 341 - protein, 107 aa, synthetic construct
RTVAAPSVFIFPPSDKCLKSGTASVVCLINNFYPREAKVQWKVDNAAQSGSSQESVTEQDSKDSTYSLSSTLTLSKADYEKHKVYACEVTHQGLSSPVTKSFNRGEA

SEQ ID NO: 342 - protein, 107 aa, synthetic construct
RTVAAPSVFIFQPSDKCLKSGTASVVCLLNNFYPKEAKVQWKVDNALQSGNSQESVTEQDSKDSTYSLSSTLTLSKADYDKHKVYACEVTHQGLSSPVTKSFNRGEA

SEQ ID NO: 343 - protein, 107 aa, synthetic construct
RTVAAPSVFIFPPSDKCLKSGTASVVCLLNNFYPREAKVQWKVDNALQTGGSQESVTEQDSKDSTYSLSSTLTLSKADYEKHKVYACEVTHQGLSSPVTKSFNRGSA

SEQ ID NO: 344 - protein, 107 aa, synthetic construct
RTVAAPTVFIFPPSDKCLKSGTASVVCLLNNFYPREAKVNWKVDNALQSGNSQESVTDQDSKDSTYSLSSTLTLSKADYEKHKVYACEVTHQGLSSPVTKSFNRGEA

SEQ ID NO: 345 - protein, 107 aa, synthetic construct
RTVAAPSVFIFPPSDKCLKSGTASVVCLLNNFYPREIKVRWKVDNALQTGNSQESVTEQDSKDSTYSLSSTLTLSKADYEKHKVYACEVTHQGLSSPVTKSFNRGEA

SEQ ID NO: 346 - protein, 107 aa, synthetic construct
RTVAAPSVFIFPPSDKCLKSGTASVVCLLNNFYPKEAKVQWKVDNALQSGNSQESVTEQDSKDKTYSLSSTLTLSKADYEKHKVYSCEVTHQGLSSPVTKSFNRGEA

SEQ ID NO: 347 - protein, 107 aa, synthetic construct
RTVAAPSVFIFPPSDKCLKSGTASVVCLLNGFYPREAKVQWKVDNALQSGSSQESFTEQDSKDSTYSLSSTLTLSKADYEKHKVYACEVTHQGLSSPVTKSFNRGEA

SEQ ID NO: 348 - protein, 107 aa, synthetic construct
RTVAAPSVFIFPPSDKCLKSGTASVVCLLNNFYPREAKVQWKVDNAVQSGNSQESLTEQDSKDSTYSLSSTLTLSKADYDKHKVYACEVTHQGLSSPVTKSFNRGEA

SEQ ID NO: 349 - protein, 107 aa, synthetic construct
RTVAAPSVFIFPPSDKCLKSGTASVVCLLNNFYPREVKVQWKVDGALQSGNSQESVTEQDSKDSTYSLSSTLTLSKADYEKHKVYTCEVTHQGLSSPVTKSFNRGEA

SEQ ID NO: 350 - protein, 107 aa, synthetic construct
RTVAAPSVFIFQPSEKCLKSGTASVVCLLNNFYPREAKVQWKVDNALQSGNSQESSTEQDSKDSTYSLSSTLTLSKADYEKHKVYACEVTHQGLSSPVTKSFNRGEA

SEQ ID NO: 351 - protein, 107 aa, synthetic construct
RTVAAPSVFIFPPSDKCLKSGTASVVCLLNNFYPREAKVQWKVDNALQSGDSQESVTEQDSKDSTYSLSSTLTLSKADYDKHKVYACEVTHQGLSSPVTKSFNRAEA

SEQ ID NO: 352 - protein, 107 aa, synthetic construct
RTVAAPSVFIFPPSQKCLKSGGASVVCLLNNFYPREAKVQWKVDNALQSGNSQESVTEQDSKDNTYSLSSTLTLSKADYEKHKVYACEVTHQGLSSPVTKSFNRGEA

SEQ ID NO: 353 - protein, 107 aa, synthetic construct
RTVAAPSVFLFPPSDKCLKSGTASVVCLVNNFYPREAKVQWKVDNALQSGNSQESVTEQDSKDSTYSLSSTLTLSKADYEKHKVYACEVTHQGLSSPLTKSFNRGEA

SEQ ID NO: 354 - protein, 107 aa, synthetic construct
RTVAAPSAFIFPPSDKCLKSGTATVVCLLNNFYPREAKVQWKVDNALQSGNSQESITEQDSKDSTYSLSSTLTLSKADYEKHKVYACEVTHQGLSSPVTKSFNRGEA

SEQ ID NO: 355 - protein, 107 aa, synthetic construct
RTVAAPSVFIFPPSDKCLESGTASVVCLLNNFYPREAKVKWKVDNALQSGNSQESTTEQDSKDSTYSLSSTLTLSKADYEKHKVYACEVTHQGLSSPVTKSFNRGEA

SEQ ID NO: 356 - protein, 107 aa, synthetic construct
RTVAAPSVFIFPPSDKCLKSGTASVVCLVNNFYPKEAKVQWKVDNALQSGNFQESVTEQDSKDSTYSLSSTLTLSKADYEKHKVYACEVTHQGLSSPVTKSFNRGEA

SEQ ID NO: 357 - protein, 107 aa, synthetic construct
RTVAAPSVFIFPPSDKCLKSGTASVVCLLNNFYPREAKVQWKVDNALQSGNIQESVTEQDSKDGTYSLSSTLTLSKADYEKHKVYSCEVTHQGLSSPVTKSFNRGEA

SEQ ID NO: 358 - protein, 107 aa, synthetic construct
RTVAAPSVFIFPPSDKCLKSGTASVVCLLNNFYPREAKVQWKVDNALQSGNFQESVTEQDSKDKTYSLSSTLTLSKADYEKHKVYACEVTHQGLSSPVTKSFNRSEA

SEQ ID NO: 359 - protein, 107 aa, synthetic construct
RTVTAPSVFIFPPSDKCLKSGTASVVCLLNNFYPREAKVQWKVDNALQSGNSQESVVEQDSKDSTYSLSSTLTLSKADYEKHKVYTCEVTHQGLSSPVTKSFNRGEA

SEQ ID NO: 360 - protein, 107 aa, synthetic construct
RTVTAPSVFIFPPSDKCLKSGTASVVCLLNNFYPREAKVQWKVDNALQNGNSQESVTEQDSKDSTYSLSSTLTLSKADYDKHKVYACEVTHQGLSSPVTKSFNRGEA

SEQ ID NO: 361 - protein, 107 aa, synthetic construct
RTVAAPSVFIFPPSDKCLKSGTASVVCLINNFYPREAKVQWKVDNALQSGNSQESTVEQDSKDSTYSLSSTLTLSKADYEKHKVYACEVTHQGLSSPVTKSFNRGEA

SEQ ID NO: 362 - protein, 107 aa, synthetic construct
RTVAAPSVFIFPPSEKCLKSGTASVVCLLNNFYPKEAKVQWKVDNALQSGNVQESVTEQDSKDSTYSLSSTLTLSKADYEKHKVYACEVTHQGLSSPVTKSFNRGEA

SEQ ID NO: 363 - protein, 107 aa, synthetic construct
RTVAAPSVFIFPPSDKCLKSGTASVVCLINNFYPKEAKVQWKVDNALQSGNIQESVTEQDSKDSTYSLSSTLTLSKADYEKHKVYACEVTHQGLSSPVTKSFNRGEA

SEQ ID NO: 364 - protein, 107 aa, synthetic construct
RTVAAPSVFIFPPSDKCLRSGGASVVCLLNNFYPREAKVQWKVDNALQSGNVQESVTEQDSKDSTYSLSSTLTLSKADYEKHKVYACEVTHQGLSSPVTKSFNRGEA

SEQ ID NO: 365 - protein, 107 aa, synthetic construct
RTVAQPSVFIFPPSDKCLKSGTASVVCLLNNFYPREAKVQWKVDNALQSGNSQESITEQDSKDSTYSLSSTLTLSKADYEKHKVYACEVTHQGLSSPVTKSFNRGSA

SEQ ID NO: 366 - protein, 107 aa, synthetic construct
RTVAAPSVFIFPPSDKCLKSGTASVVCLLSNFYPREAKVQWKVDNALQNGNSQESVTEQDSKDSTYSLSSTLTLSKADYEKHKVYACEVTHQGLSSPVTKSFSRGEA

SEQ ID NO: 367 - protein, 107 aa, synthetic construct
RTVTAPSVFIFPPSDKCLKSGTASVVCLLNNFYPREAKVQWKVDNALQSGDSQESTTEQDSKDSTYSLSSTLTLSKADYEKHKVYACEVTHQGLSSPVTKSFNRGEA

SEQ ID NO: 368 - protein, 107 aa, synthetic construct
RTVAAPSVFIFPPSDKCLKSGSASVVCLINNFYPREAKVNWKVDNALQSGNSQESVTEQDSKDSTYSLSSTLTLSKADYEKHKVYACEVTHQGLSSPVTKSFNRGEA

SEQ ID NO: 369 - protein, 107 aa, synthetic construct
RTVAAPSVFIFPPSDKCLKSGTASVVCLLNNFYPREAKVQWKVDNALQSGNIQEGVTEQDSKDSTYSLSSTLTLSKADYEKHKVYACEVTHQGLSSPVTKSFNRGTA

SEQ ID NO: 370 - protein, 107 aa, synthetic construct
RTVAAPSVFIFPPSDKCLKSGTASVVCMLNNFYPREAKVQWKVDNALQTGNSQESVTDQDSKDSTYSLSSTLTLSKADYEKHKVYACEVTHQGLSSPVTKSFNRGEA

SEQ ID NO: 371 - protein, 107 aa, synthetic construct
RTVAAPSVFIFPPSDKCLKSGTASVVCLVNNFYPREAKVQWKVDNAVQSGNSQESVTEQDSKDSTYSLSSTLTLSKADYEKHKTYACEVTHQGLSSPVTKSFNRGEA

SEQ ID NO: 372 - protein, 107 aa, synthetic construct
RTVAAPSVFIFPPSDKCLKSGTASVVCLLNNFYPREAKVQWKVDNALQSGNSQEGVTEQDSKDSTYSLSSTLTLSKADYEKHKTYACEVTHQSLSSPVTKSFNRGEA

SEQ ID NO: 373 - protein, 107 aa, synthetic construct
RTVAQPSVFIFPPSDKCLKSGTASVVCLLNNFYPREAKVSWKVDNALQSGNSQESVTEQDSKDSTYSLSSTLTLSKADYEKHKVYACEVTHQGLSSPVTKSFSRGEA

SEQ ID NO: 374 - protein, 107 aa, synthetic construct
RTVAAPSVFIFPPSDKCLKSGTASVVCMLNNFYPREAKVQWKVDNALQSGNVQESVTEQDSKDSTYSLSSTLTLSKADYEKHKVYACEVTHQGLSSPVTKSFNRGEA

SEQ ID NO: 375 - protein, 107 aa, synthetic construct
RTVAAPSVFIFPPSDKCLKSGTASVVCLLNNFYPREAKVQWKVDNALQSGNSQEGVTEQDSKDNTYSLSSTLTLSKADYEKHKVYACEVTHQGLSSPVTKSFNRGEA

SEQ ID NO: 376 - protein, 107 aa, synthetic construct
RTVAAPSVFIFPPSDKCLKSGTASVVCLLNNFYPREAKVQWKVDNALQSGNSQESVTEQDSKDSTYSLSSTLTLSKADYEKHKVYACEVTHQSLSSPVTKSFNRSEA

SEQ ID NO: 377 - protein, 107 aa, synthetic construct
RTVAAPSVFIFPPSDKCLKSGTASVVCLLNNFYPREAKVQWKVDNALQSGNSQESVVEQDSKDSTYSLSSTLTLSKADYEKHKVYACEVTHQGLSSPLTKSFNRGEA

SEQ ID NO: 378 - protein, 107 aa, synthetic construct
RTVAAPSVFIFPPSDKCLKSGTASVVCLINNFYPREAKVQWKVDGALQSGNSQESVTEQDSKDSTYSLSSTLTLSKADYEKHKVYACEVTHQGLSSPVTKSFNRGEA

SEQ ID NO: 379 - protein, 107 aa, synthetic construct
RTVAAPSVFIFPPSDKCLKSGTASVVCLLNNFYPREAKVQWKVDNALQSGNFQESSTEQDSKDSTYSLSSTLTLSKADYEKHKVYACEVTHQGLSSPVTKSFNRGEA

SEQ ID NO: 380 - protein, 107 aa, synthetic construct
RTVAAPSVFLFPPSDKCLKSGTASVVCLLNNFYPREVKVQWKVDNALQSGNSQESVTEQDSKDSTYSLSSTLTLSKADYEKHKVYACEVTHQGLSSPVTKSFNRGEA

SEQ ID NO: 381 - protein, 107 aa, synthetic construct
RTVAAPTVFIFPPSDKCLKSGTASVVCLLNNFYPREAKVQWKVDNAAQSGNSQESVTEQDSKDSTYSLSSTLTLSKADYEKHKVYACEVTHQGLSSPVTKSFNRGEA

SEQ ID NO: 382 - protein, 107 aa, synthetic construct
RTVAAPSVFIFPPSDKCLKSGTASVVCLLSNFYPREAKVRWKVDNALQSGNSQESVTEQDSKDSTYSLSSTLTLSKADYEKHKVYACEVTHQGLSSPVTKSFNRGEA

SEQ ID NO: 383 - protein, 247 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSAVALHRPDVCLLPPAREQLNLRRSATITCLVDGFSPADVFVQWMQRGQPLSPEKYVTSAPMPEPRAPGRYFAHSILTVSEAEWNTGETYTCVVAHEALPNRVTERTVDKSTEPKSADKTHHHHHHHH

SEQ ID NO: 384 - protein, 247 aa, synthetic construct
EVQLVESGGGLVQPGGSLRLSCAASGFNIKDTYIHWVRQAPGKGLEWVARIYPTNGYTRYADSVKGRFTISADTSKNTAYLQMNSLRAEDTAVYYCSRWGGDGFYAMDYWGQGTLVTVSSAVALHRPDVYLLPPAREQLNLRRSATITCLVDGFSPADVFVQWMQRGQPLSPEKYVTSAPMPEPRAPGRYCAHSILTVSEAEWNTGETYTCVVAHEALPNRVTERTVDKSTEPKSADKTHHHHHHHH

SEQ ID NO: 385 - protein, 107 aa, synthetic construct
RTVAAPSVFIFQPSDEQLKSGTAKVVCLLNGFYPREAKVQWKVDNALQSGNSCESVTEQDSKDSTYSLSSTLTLSKADYEKHKVYACEVTHQGLSSPLTKSFNRGEA

SEQ ID NO: 386 - protein, 107 aa, synthetic construct
RTVAAPSVFIFPPSDEQLKSGTASVVCLLNNFYPREAKVQWKVDNALQSGDICESVTEQDSKDSTYSLSSTLTLSKADYEKHKVYACEVTHQGLSSPVTKSFNRGEA

SEQ ID NO: 387 - protein, 107 aa, synthetic construct
RTVAAPSVFIFPPSDEQLKSGTAKVVCLLNNFYPREAKVVWKVDNALQSGNSCESSTEQDSKDSTYSLSSTLTLSKADYEKHKVYACEVTHQGLSSPVTKSFSRGEA

SEQ ID NO: 388 - protein, 107 aa, synthetic construct
RTVAAPSVFIFPPSDQQLKSGTAKVVCLLNNFYPREAKVQWKVDNALQSGNSCESVTEQDSKDSTYSLSSTLTLSKADYEKHKVYACEVTHQGLSSPVTKSFNRSTA

SEQ ID NO: 389 - protein, 107 aa, synthetic construct
RTVAAPSVFIFPPSDEQLKSGTAKVVCLLNNFYPREAKVRWKVDNALQSGNSCESSVEQDSKDSTYSLSSTLTLSKADYEKHKVYACEVTHQGLSSPVTKSFNRGEA

SEQ ID NO: 390 - protein, 107 aa, synthetic construct
RTVAAPSVFIFPPSDEQLKSGTAKVVCLLNNFYPREAKVKWKVDNALQSGGSCESVTEQDSKDSTYSLSSTLTLSKADYDKHKVYACEVTHQGLSSPVTKSFNRGEA

SEQ ID NO: 391 - protein, 107 aa, synthetic construct
RTVAAPSVFIFPPSDEQLKSGSAKVVCLLNNFYPREAKVQWKVDNALQSGGSCESVTEQDSKDSTYSLSSTLTLSKADYEKHKVYACEVTHQGLSSPVTKSFSRGEA

SEQ ID NO: 392 - protein, 107 aa, synthetic construct
RTVTAPSVFIFQPSDEQLKSGTAKVVCLLNNFYPREAKVSWKVDNALQSGNSCESVTEQDSKDSTYSLSSTLTLSKADYEKHKVYACEVTHQGLSSPVTKSFNRGEA

SEQ ID NO: 393 - protein, 107 aa, synthetic construct
RTVAAPSVFIFPPSDEQLKSGTATVVCLLNNFYPREIKVQWKVDNALQSGNSCESVTEQDSKDNTYSLSSTLTLSKADYEKHKVYACEVTHQGLSSPVTKSFNRGEA

SEQ ID NO: 394 - protein, 107 aa, synthetic construct
RTVAAPSVFIFPPSDEQLKSGTAKVVCLINNFYPREAKVNWKVDNALQSGNSCESVTEQDSKDSTYSLSSTLTLSKADYEKHKVYACEVTHQGLSSPVTKSFNRGSA

SEQ ID NO: 395 - protein, 107 aa, synthetic construct
RTVAAPSVFIFPPSEQQLESGTAKVVCLLNNFYPREAKVQWKVDNALQSGNSCESVTEQDSKDSTYSLSSTLTLSKADYEKHKVYACEVTHQGLSSPVTKSFNRGEA

SEQ ID NO: 396 - protein, 107 aa, synthetic construct
RTVAAPSVFIFPPSDEQLKSGTAKVVCLLNNFYPREAKVRWKVDNALQSGSSCESVTEQDSKDSTYSLSSTLTLSKADYEKHKVYACEVTHQGLSSPVTKSFNRGSA

SEQ ID NO: 397 - protein, 107 aa, synthetic construct
RTVAAPSVFIFPPSEEQLKSGTAKVVCLLNNFYPREAKVQWKVDNALQSGNSCESLTDQDSKDSTYSLSSTLTLSKADYEKHKVYACEVTHQGLSSPVTKSFNRGEA

SEQ ID NO: 398 - protein, 107 aa, synthetic construct
RTVAAPSVFIFPPSDEQLKSGTAKVVCLLNNFYPREAKVTWKVDNALQNGNSCESITEQDSKDSTYSLSSTLTLSKADYEKHKVYACEVTHQGLSSPVTKSFNRGEA

SEQ ID NO: 399 - protein, 107 aa, synthetic construct
RTVAAPSVFIFPPSDEQLKSGTAKVVCLVNNFYPREAKVQWKVDNALQSGNSCESTTEQDSKDSTYSLSSTLTLSKADYEKHKVYACEVTHQGLSSPVTKSFNRSEA

SEQ ID NO: 400 - protein, 107 aa, synthetic construct
RTVAAPSVFIFPPSDEQLKSGGAKVVCLLNNFYPREIKVQWKVDNAVQSGNSCESVTEQDSKDSTYSLSSTLTLSKADYEKHKVYACEVTHQGLSSPVTKSFNRGEA

SEQ ID NO: 401 - protein, 107 aa, synthetic construct
RTVAAPSVFLFPPSDEQLKSGTAKVVCLLNNFYPREAKVQWKVDNAAQSGNSCESTTEQDSKDSTYSLSSTLTLSKADYEKHKVYACEVTHQGLSSPVTKSFNRGEA

SEQ ID NO: 402 - protein, 107 aa, synthetic construct
RTVAAPSVFIFPPSDEQLRSGTAKVVCLLNNFYPREAKVNWKVDNAVQSGNSCESVTEQDSKDSTYSLSSTLTLSKADYEKHKVYACEVTHQGLSSPVTKSFNRGEA

SEQ ID NO: 403 - protein, 107 aa, synthetic construct
RTVAAPSVFIFPPSDEQLKSGTAKVVCLLNNFYPREAKVSWKVDNALQSGNSCESSTEQDSKDSTYSLSSTLTLSKADYEKHKVYTCEVTHQGLSSPVTKSFNRGEA

SEQ ID NO: 404 - protein, 107 aa, synthetic construct
RTVAQPSVFIFPPSQEQLKSGTATVVCLLNNFYPREAKVQWKVDNALQSGNSCESVTEQDSKDSTYSLSSTLTLSKADYEKHKVYACEVTHQGLSSPVTKSFNRGEA

SEQ ID NO: 405 - protein, 107 aa, synthetic construct
RTVAAPSVFIFPPSDEQLKSGSAKVVCLLNNFYPREAKVQWKVDNALQTGNICESVTEQDSKDSTYSLSSTLTLSKADYEKHKVYACEVTHQGLSSPVTKSFNRGEA

SEQ ID NO: 406 - protein, 107 aa, synthetic construct
RTVAAPSVFIFPPSDEQLKSGTAKVVCLLNNFYPREAKVTWKVDNALQSGNSCESLTEQDSKDSTYSLSSTLTLSKAEYEKHKVYACEVTHQGLSSPVTKSFNRGEA

SEQ ID NO: 407 - protein, 107 aa, synthetic construct
RTVAAPSVFLFPPSDEQLKSGTASVVCLLNNFYPREAKVQWKVDNALQSGNSCESVTEQDSKDSTYSLSSTLTLSKADYDKHKVYACEVTHQGLSSPVTKSFNRGEA

SEQ ID NO: 408 - protein, 107 aa, synthetic construct
RTVAAPSVFIFPPSDQQLKSGTAKVVCLLNNFYPREAKVSWKVDNALQSGNSCESVTEQDSKDSTYSLSSTLTLSKAEYEKHKVYACEVTHQGLSSPVTKSFNRGEA

SEQ ID NO: 409 - protein, 107 aa, synthetic construct
RTVAAPTVFIFPPSDEQLRSGTAKVVCLLNNFYPREAKVQWKVDNALQSGNSCESVTEQDSKDSTYSLSSTLTLSKADYEKHKVYSCEVTHQGLSSPVTKSFNRGEA

SEQ ID NO: 410 - protein, 107 aa, synthetic construct
RTVAAPSVFIFPPSDEQLKSGTAKVVCLLNNFYPREIKVTWKVDNALQSGNSCESVTEQDSKDSTYSLSSTLTLSKADYEKHKVYACEVTHQGLSSPVTKSFNRGTA

SEQ ID NO: 411 - protein, 107 aa, synthetic construct
RTVAAPSVFIFPPSDEQLKSGTAKVVCLVNNFYPREAKVSWKVDNALQSGNVCESVTEQDSKDSTYSLSSTLTLSKADYEKHKVYACEVTHQGLSSPVTKSFNRGEA

SEQ ID NO: 412 - protein, 107 aa, synthetic construct
RTVAAPSVFIFPPSDEQLKSGTAKVVCLLNNFYPREVKVQWKVDNALQSGNSCESVTEQDSKDSTYSLSSTLTLSKADYDKHKVYTCEVTHQGLSSPVTKSFNRGEA

SEQ ID NO: 413 - protein, 107 aa, synthetic construct
RTVAAPSVFIFPPSDEQLESGTAKVVCLLNNFYPREAKVIWKVDNALQSGNVCESVTEQDSKDSTYSLSSTLTLSKADYEKHKVYACEVTHQGLSSPVTKSFNRGEA

SEQ ID NO: 414 - protein, 107 aa, synthetic construct
RTVAAPSVFIFPPSQEQLKSGTAKVVCLLNNFYPREAKVQWKVDGALQTGNSCESVTEQDSKDSTYSLSSTLTLSKADYEKHKVYACEVTHQGLSSPVTKSFNRGEA

SEQ ID NO: 415 - protein, 107 aa, synthetic construct
RTVAAPSVFIFPPSDEQLKSGTAKVVCLLNNFYPREAKVRWKVDGALQSGNSCESVTEQDSKDSTYSLSSTLTLSKADYEKHKVYACEVTHQGLSSPVTKSFNRSEA

SEQ ID NO: 416 - protein, 107 aa, synthetic construct
RTVAAPSVFIFPPSEEQLKSGTAKVVCLLNNFYPREAKVQWKVDNALQSGNSCESITEQDSKDSTYSLSSTLTLSKADYEKHKVYACEVTHQGLSSPLTKSFNRGEA

SEQ ID NO: 417 - protein, 107 aa, synthetic construct
RTVAQPSVFIFPPSDEQLKSGTAKVVCLLNNFYPREAKVNWKVDNALQSGNSCESVTEQDSKDSTYSLSSTLTLSKADYEKHKVYSCEVTHQGLSSPVTKSFNRGEA

SEQ ID NO: 418 - protein, 107 aa, synthetic construct
RTVAAPSVFIFPPSDEQLKSGTAKVVCLVNNFYPREAKVQWKVDNALQSGNICESVVEQDSKDSTYSLSSTLTLSKADYEKHKVYACEVTHQGLSSPVTKSFNRGEA

SEQ ID NO: 419 - protein, 107 aa, synthetic construct
RTVAAPSVFIFPPSDEQLKSGTATVVCLLNNFYPREAKVIWKVDNALQSGNSCESVTEQDSKDGTYSLSSTLTLSKADYEKHKVYACEVTHQGLSSPVTKSFNRGEA

SEQ ID NO: 420 - protein, 107 aa, synthetic construct
RTVAAPSVFIFPPSDDQLKSGTAKVVCLLNNFYPREAKVVWKVDNALQSGNSCESVTEQDSKDSTYSLSSTLTLSKADYEKHKVYACEVTHQGLSSPVTKSFNRGTA

SEQ ID NO: 421 - protein, 107 aa, synthetic construct
RTVAAPSVFIFQPSDEQLKSGTAKVVCLLNNFYPREAKVQWKVDNALQSGNSCESVTEQDSKDSTYSLSSTLTLSKADYEKHKVYACEVTHQGLSSPVTKSFSRGSA

SEQ ID NO: 422 - protein, 107 aa, synthetic construct
RTVAAPSVFIFPPSEEQLRSGSAKVVCLLNNFYPREAKVQWKVDNALQSGNSCESVTEQDSKDSTYSLSSTLTLSKADYEKHKVYACEVTHQGLSSPVTKSFNRGEA

SEQ ID NO: 423 - protein, 107 aa, synthetic construct
RTVAQPSVFIFPPSDEQLKSGTAKVVCLLNGFYPREAKVQWKVDNALQSGNSCESVTEQDSKDNTYSLSSTLTLSKADYEKHKVYACEVTHQGLSSPVTKSFNRGEA

SEQ ID NO: 424 - protein, 107 aa, synthetic construct
RTVAAPSVFIFPPSQQQLKSGTAKVVCLLNNFYPREAKVQWKVDNALQSGNICESVTEQDSKDSTYSLSSTLTLSKADYEKHKVYACEVTHQGLSSPVTKSFNRGEA

SEQ ID NO: 425 - protein, 107 aa, synthetic construct
RTVAAPSVFIFPPSDEQLKSGTAKVVCLLNNFYPREAKVQWKVDNALQSGNVCESTTEQDSKDNTYSLSSTLTLSKADYEKHKVYACEVTHQGLSSPVTKSFNRGEA

SEQ ID NO: 426 - protein, 107 aa, synthetic construct
RTVAAPTVFIFQPSDEQLKSGTAKVVCLLNNFYPREAKVQWKVDNALQSGNSCESVTEQDSKDKTYSLSSTLTLSKADYEKHKVYACEVTHQGLSSPVTKSFNRGEA

SEQ ID NO: 427 - protein, 107 aa, synthetic construct
RTVAAPSVFIFPPSDEQLKSGTAKVVCLLNNFYPREAKVQWKVDNALQTGNSCESVTEQDSKDSTYSLSSTLTLSKAEYEKHKVYACEVTHQGLSSPVTKSFNRGSA

SEQ ID NO: 428 - protein, 107 aa, synthetic construct
RTVAAPSVFIFPPSDEQLKSGTASVVCLLNNFYPREAKVQWKVDNALQSGNSCESVTEQDSKDSTYSLSSTLTLSKADYEKHKTYACEVTHQGLSSPVTKSFNRGTA

SEQ ID NO: 429 - protein, 107 aa, synthetic construct
RTVAAPTVFIFPPSDEQLKSGSAKVVCLLNNFYPREAKVQWKVDNALQSGNSCESVTEQDSKDSTYSLSSTLTLSKADYEKHKTYACEVTHQGLSSPVTKSFNRGEA

SEQ ID NO: 430 - protein, 107 aa, synthetic construct
RTVAAPSVFIFPPSDEQLKSGTAKVVCLLNNFYPREAKVQWKVDNALQNGNSCESVTEQDSKDGTYSLSSTLTLSKADYDKHKVYACEVTHQGLSSPVTKSFNRGEA

SEQ ID NO: 431 - protein, 107 aa, synthetic construct
RTVAAPSVFIFPPSDEQLKSGTAKVVCLLNNFYPREAKVKWKVDNAVQSGNSCESVTEQDSKDSTYSLSSTLTLSKADYEKHKTYACEVTHQGLSSPVTKSFNRGEA

SEQ ID NO: 432 - protein, 107 aa, synthetic construct
RTVAAPSVFIFPPSDEQLKSGTAKVVCLLNNFYPREAKVQWKVDGALQSGNSCESVTEQDSKDKTYSLSSTLTLSKADYEKHKVYTCEVTHQGLSSPVTKSFNRGEA

SEQ ID NO: 433 - protein, 107 aa, synthetic construct
RTVAAPSVFIFPPSQEQLKSGTAKVVCLLNNFYPREAKVQWKVDNALQSGDSCESVTEQDSKDSTYSLSSTLTLSKADYEKHKTYACEVTHQGLSSPVTKSFNRGEA

SEQ ID NO: 434 - protein, 107 aa, synthetic construct
RTVAAPSVFLFPPSDEQLKSGTAKVVCLLNNFYPREVKVQWKVDNALQNGNSCESVTEQDSKDSTYSLSSTLTLSKADYEKHKVYACEVTHQGLSSPVTKSFNRGEA

SEQ ID NO: 435 - protein, 107 aa, synthetic construct
RTVAAPSVFIFPPSDEQLESGTAKVVCLLNNFYPREAKVQWKVDNALQSGDSCESVTEQDSKDSTYSLSSTLTLSKADYEKHKVYACEVTHQGLSSPVTKSFNRSEA

SEQ ID NO: 436 - protein, 107 aa, synthetic construct
RTVAAPSVFIFPPSDEQLKSGTAKVVCLLNNFYPREAKVQWKVDNALQSGSSCESITEQDSKDSTYSLSSTLTLSKADYEKHKVYACEVTHQGLSSPVTKSFNRGTA

SEQ ID NO: 437 - protein, 107 aa, synthetic construct
RTVAAPSVFIFPPSDEQLKSGTAKVVCLLNGFYPREVKVTWKVDNALQSGNSCESVTEQDSKDSTYSLSSTLTLSKADYEKHKVYACEVTHQGLSSPVTKSFNRGEA

SEQ ID NO: 438 - protein, 107 aa, synthetic construct
RTVAAPSVFIFPPSDEQLKSGTAKVVCLLNNFYPREAKVTWKVDNAAQSGNSCESVTEQDSKDSTYSLSSTLTLSKADYEKHKVYSCEVTHQGLSSPVTKSFNRGEA

SEQ ID NO: 439 - protein, 107 aa, synthetic construct
RTVAAPSVFIFPPSDEQLKSGTAKVVCLLNNFYPREAKVSWKVDNALQSGNSCESVTEQDSKDKTYSLSSTLTLSKADYEKHKVYACEVTHQGLSSPLTKSFNRGEA

SEQ ID NO: 440 - protein, 107 aa, synthetic construct
RTVAAPSVFIFPPSDEQLKSGTAKVVCLLNNFYPREAKVQWKVDNAAQSGNSCESLTEQDSKDNTYSLSSTLTLSKADYEKHKVYACEVTHQGLSSPVTKSFNRGEA

SEQ ID NO: 441 - protein, 107 aa, synthetic construct
RTVAAPSVFIFPPSDEQLKSGTAKVVCLLNNFYPREAKVVWKVDNALQSGGSCESVTEQDSKDKTYSLSSTLTLSKADYEKHKVYACEVTHQGLSSPVTKSFNRGEA

SEQ ID NO: 442 - protein, 107 aa, synthetic construct
RTVAAPSVFIFPPSDQQLKSGTAKVVCLLNNFYPREAKVKWKVDNAAQSGNSCESVTEQDSKDSTYSLSSTLTLSKADYEKHKVYACEVTHQGLSSPVTKSFNRGEA

SEQ ID NO: 443 - protein, 107 aa, synthetic construct
RTVAAPSVFIFPPSEDQLKSGTAKVVCLINNFYPREAKVQWKVDNALQSGNSCESVTEQDSKDSTYSLSSTLTLSKADYEKHKVYACEVTHQGLSSPVTKSFNRGEA

SEQ ID NO: 444 - protein, 107 aa, synthetic construct
RTVAAPSVFIFPPSDDQLKSGTAKVVCLLNNFYPREAKVNWKVDNALQSGNICESVTEQDSKDSTYSLSSTLTLSKADYEKHKVYACEVTHQGLSSPVTKSFNRGEA

SEQ ID NO: 445 - protein, 107 aa, synthetic construct
RTVAAPSVFIFPPSDEQLESGTAKVVCLLNNFYPREAKVVWKVDNALQSGNSCESLTEQDSKDSTYSLSSTLTLSKADYEKHKVYACEVTHQGLSSPVTKSFNRGEA

SEQ ID NO: 446 - protein, 107 aa, synthetic construct
RTVAAPSVFIFPPSDEQLKSGGATVVCLLNNFYPREAKVQWKVDNALQSGDSCESVTEQDSKDSTYSLSSTLTLSKADYEKHKVYACEVTHQGLSSPVTKSFNRGEA

SEQ ID NO: 447 - protein, 107 aa, synthetic construct
RTVAAPSVFIFPPSDEQLKSGGAKVVCLLNNFYPREAKVQWKVDNALQSGNSCESVTEQDSKDGTYSLSSTLTLSKADYEKHKVYACEVTHQGLSSPLTKSFNRGEA

SEQ ID NO: 448 - protein, 107 aa, synthetic construct
RTVAQPSVFIFPPSDEQLKSGTAKVVCLLNNFYPREAKVQWKVDNALQSGNSCESSTEQDSKDSTYSLSSTLTLSKADYEKHKVYACEVTHQGLSSPVTKSFNRSEA

SEQ ID NO: 449 - protein, 107 aa, synthetic construct
RTVAAPSVFLFPPSDEQLKSGGAKVVCLLNNFYPREAKVQWKVDGALQSGNSCESVTEQDSKDSTYSLSSTLTLSKADYEKHKVYACEVTHQGLSSPVTKSFNRGEA

SEQ ID NO: 450 - protein, 107 aa, synthetic construct
RTVAAPSVFIFPPSDEQLRSGTAKVVCLLNNFYPREAKVKWKVDNALQTGNSCESVTEQDSKDSTYSLSSTLTLSKADYEKHKVYACEVTHQGLSSPVTKSFNRGEA

SEQ ID NO: 451 - protein, 107 aa, synthetic construct
RTVAAPSVFIFPPSDEQLKSGTAKVVCLLNNFYPREAKVQWKVDNALQSGSSCESVTEQDSKDSTYSLSSTLTLSKADYDKHKVYSCEVTHQGLSSPVTKSFNRGEA

SEQ ID NO: 452 - protein, 107 aa, synthetic construct
RTVAAPSVFIFPPSDEQLKSGTAKVVCLLNNFYPREAKVVWKVDNALQSGNVCESVTEQDSKDSTYSLSSTLTLSKAEYEKHKVYACEVTHQGLSSPVTKSFNRGEA

SEQ ID NO: 453 - protein, 107 aa, synthetic construct
RTVAAPSVFIFPPSDDQLKSGTAKVVCLLNNFYPREAKVQWKVDNALQNGDSCESVTEQDSKDSTYSLSSTLTLSKADYEKHKVYACEVTHQGLSSPVTKSFNRGEA

SEQ ID NO: 454 - protein, 107 aa, synthetic construct
RTVAAPTVFIFPPSDEQLKSGTAKVVCLLNGFYPREAKVQWKVDNALQSGNSCESLTEQDSKDSTYSLSSTLTLSKADYEKHKVYACEVTHQGLSSPVTKSFNRGEA

SEQ ID NO: 455 - protein, 107 aa, synthetic construct
RTVTAPSVFIFPPSDEQLKSGTAKVVCLLNNFYPREAKVQWKVDGAVQSGNSCESVTEQDSKDSTYSLSSTLTLSKADYEKHKVYACEVTHQGLSSPVTKSFNRGEA

SEQ ID NO: 456 - protein, 107 aa, synthetic construct
RTVAAPSVFIFPPSDEQLKSGTAKVVCLINNFYPREAKVIWKVDNALQNGNSCESVTEQDSKDSTYSLSSTLTLSKADYEKHKVYACEVTHQGLSSPVTKSFNRGEA

SEQ ID NO: 457 - protein, 107 aa, synthetic construct
RTVTAPTVFIFPPSDEQLKSGTAKVVCLLNNFYPREAKVQWKVDNALQSGNSCESVTEQDSKDSTYSLSSTLTLSKADYEKHKVYACEVTHQGLSSPLTKSFNRGEA

SEQ ID NO: 458 - protein, 107 aa, synthetic construct
RTVAAPSVFIFPPSQEQLKSGTAKVVCLLNNFYPREAKVQWKVDNALQSGNSCESTTEQDSKDSTYSLSSTLTLSKADYEKHKVYSCEVTHQGLSSPVTKSFNRGEA

SEQ ID NO: 459 - protein, 107 aa, synthetic construct
RTVAAPSVFIFPPSDEQLKSGTAKVVCLLNNFYPREAKVKWKVDNALQSGNSCESVTEQDSKDSTYSLSSTLTLSKAEYEKHKVYACEVTHQGLSSPVTKSFSRGEA

SEQ ID NO: 460 - protein, 107 aa, synthetic construct
RTVAAPSVFIFPPSDEQLKSGTAKVVCLLNNFYPREIKVQWKVDNALQSGNVCESVTDQDSKDSTYSLSSTLTLSKADYEKHKVYACEVTHQGLSSPVTKSFNRGEA

SEQ ID NO: 461 - protein, 107 aa, synthetic construct
RTVAAPSVFIFPPSDEQLKSGTAKVVCLINGFYPREAKVQWKVDNALQSGNSCESSTEQDSKDSTYSLSSTLTLSKADYEKHKVYACEVTHQGLSSPVTKSFNRGEA

SEQ ID NO: 462 - protein, 107 aa, synthetic construct
RTVAAPSVFIFPPSDEQLKSGTAKVVCLLNNFYPREAKVQWKVDNALQSGGSCESVTEQDSKDGTYSLSSTLTLSKADYEKHKTYACEVTHQGLSSPVTKSFNRGEA

SEQ ID NO: 463 - protein, 107 aa, synthetic construct
RTVAQPSVFIFPPSDEQLKSGTAKVVCLVNNFYPREAKVQWKVDNALQSGNSCESITEQDSKDSTYSLSSTLTLSKADYEKHKVYACEVTHQGLSSPVTKSFNRGEA

SEQ ID NO: 464 - protein, 107 aa, synthetic construct
RTVAAPSVFIFPPSDEQLKSGTATVVCLLNNFYPREAKVQWKVDNALQSGNSCESVVEQDSKDSTYSLSSTLTLSKADYEKHKVYTCEVTHQGLSSPVTKSFNRGEA

SEQ ID NO: 465 - protein, 107 aa, synthetic construct
RTVAAPSVFLFPPSDEQLKSGSAKVVCLLNNFYPREAKVQWKVDNAVQSGNSCESVTEQDSKDSTYSLSSTLTLSKADYEKHKVYACEVTHQGLSSPVTKSFNRGEA

SEQ ID NO: 466 - protein, 107 aa, synthetic construct
RTVAAPSVFIFPPSDEQLKSGTASVVCLLNNFYPREIKVQWKVDNALQSGNSCESTTEQDSKDSTYSLSSTLTLSKADYEKHKVYACEVTHQGLSSPVTKSFNRGEA

SEQ ID NO: 467 - protein, 107 aa, synthetic construct
RTVTAPSVFIFPPSDEQLKSGTASVVCLLNNFYPREAKVQWKVDNALQSGNSCESVVEQDSKDSTYSLSSTLTLSKADYEKHKVYACEVTHQGLSSPVTKSFNRGEA

SEQ ID NO: 468 - protein, 107 aa, synthetic construct
RTVAAPSVFIFPPSDEQLKSGTAKVVCLLNNFYPREVKVQWKVDNALQSGGSCESVVEQDSKDSTYSLSSTLTLSKADYEKHKVYACEVTHQGLSSPVTKSFNRGEA

SEQ ID NO: 469 - protein, 107 aa, synthetic construct
RTVAAPSVFIFQPSDEQLKSGTAKVVCLVNNFYPREAKVQWKVDNALQSGNSCESVTEQDSKDNTYSLSSTLTLSKADYEKHKVYACEVTHQGLSSPVTKSFNRGEA

SEQ ID NO: 470 - protein, 107 aa, synthetic construct
RTVAAPSVFIFPPSDEQLKSGGAKVVCLLNNFYPREAKVRWKVDNALQSGNSCESVTEQDSKDKTYSLSSTLTLSKADYEKHKVYACEVTHQGLSSPVTKSFNRGEA

SEQ ID NO: 471 - protein, 107 aa, synthetic construct
RTVTAPSVFIFPPSDEQLKSGTAKVVCLLNNFYPREAKVQWKVDNALQSGSSCESVTEQDSKDSTYSLSSTLTLSKADYEKHKVYACEVTHQGLSSPVTKSFSRGEA

SEQ ID NO: 472 - protein, 107 aa, synthetic construct
RTVAAPSVFIFPPSDDQLKSGTAKVVCLLNNFYPREVKVRWKVDNALQSGNSCESVTEQDSKDSTYSLSSTLTLSKADYEKHKVYACEVTHQGLSSPVTKSFNRGEA

SEQ ID NO: 473 - protein, 107 aa, synthetic construct
RTVAAPSVFIFPPSDEQLKSGTAKVVCLLNNFYPREAKVQWKVDNAAQSGNSCESVTDQDSKDSTYSLSSTLTLSKADYEKHKVYACEVTHQGLSSPVTKSFNRGSA

SEQ ID NO: 474 - protein, 107 aa, synthetic construct
RTVAAPSVFIFPPSDEQLKSGTAKVVCLLNNFYPREAKVIWKVDNALQTGNSCESITEQDSKDSTYSLSSTLTLSKADYEKHKVYACEVTHQGLSSPVTKSFNRGEA

SEQ ID NO: 475 - protein, 107 aa, synthetic construct
RTVAAPSVFIFPPSDEQLKSGTAKVVCLLNNFYPREAKVIWKVDNALQSGSSCESVTDQDSKDSTYSLSSTLTLSKADYEKHKVYACEVTHQGLSSPVTKSFNRGEA

SEQ ID NO: 476 - protein, 107 aa, synthetic construct
RTVAAPSVFIFPPSDEQLRSGTAKVVCLINNFYPREAKVQWKVDNALQSGNSCESVTDQDSKDSTYSLSSTLTLSKADYEKHKVYACEVTHQGLSSPVTKSFNRGEA

SEQ ID NO: 477 - protein, 107 aa, synthetic construct
RTVAAPSVFIFPPSDEQLKSGTAKVVCLLNNFYPREAKVQWKVDNALQSGNSCESVTEQDSKDGTYSLSSTLTLSKADYEKHKVYTCEVTHQGLSSPVTKSFNRGEA

SEQ ID NO: 478 - protein, 107 aa, synthetic construct
RTVAAPSVFIFPPSDEQLESGTAKVVCLLNNFYPREAKVNWKVDNALQSGNSCESVTEQDSKDSTYSLSSTLTLSKADYEKHKVYACEVTHQGLSSPVTKSFNRGEA

SEQ ID NO: 479 - protein, 111 aa, synthetic construct
AVALHRPDVCLLPPAREQLNLRRSATITCLVKGFSPADVFVQWMQRGQPLSPEKYVTSAPMPEPRAPGRYFAHSILTVSEAEWNTGETYTCVVAHEALPNRVTERTVDKST

SEQ ID NO: 480 - protein, 107 aa, synthetic construct
RTVAAPSVFIFPPSDECLKSGTAEVVCLLLNFYPREAKVQWKVDNALQSGNSQESVTEQDSKDSTYSLSSTLTLSKADYEKHKVYACEVTHQGLSSPVTKSFNRGEA

SEQ ID NO: 481 - protein, 107 aa, synthetic construct
RTVAAPSVFIFPPSDECLKSGTADVVCLLLNFYPREAKVQWKVDNALQSGNSQESVTEQDSKDSTYSLSSTLTLSKADYEKHKVYACEVTHQGLSSPVTKSFNRGEA

SEQ ID NO: 482 - protein, 107 aa, synthetic construct
RTVAAPSVFIFPPSDEQLKSGTAKVVCLLLNFYPREAKVVWKVDNALQSGNVCESVTEQDSKDGTYSLSSTLTLSKAEYEKHKVYACEVTHQGLSSPVTKSFNRGEA

SEQ ID NO: 483 - protein, 107 aa, synthetic construct
RTVAAPSVFIFPPSDEQLKSGTAKVVCLLLNFYPREAKVVWKVDNALQSGNVCESVTEQDSKDSTYSLSSTLTLSKAEYEKHKVYACEVTHQGLSSPVTKSFNRGEA

SEQ ID NO: 484 - protein, 107 aa, synthetic construct
RTVAAPSVFIFPPSDECLKSGTAKVVCLLNNFYPREVKVQWKVDGALQSGNIKESVTEQDSKDGTYSLSSTLTLSKADYEKHKVYACEVTHQGLSSPVTKSFNRGEA

SEQ ID NO: 485 - protein, 121 aa, synthetic construct
QVQLVQSGAEVKKPGSSVKVSCKASGGTFSNYWMHWVRQAPGQGLEWMGATYRGHSDTYYNQKFKGRVTITADKSTSTAYMELSSLRSEDTAVYYCARGAIYDGYDVLDNWGQGTLVTVSS

SEQ ID NO: 486 - protein, 107 aa, synthetic construct
DIQMTQSPSSLSASVGDRVTITCSASQDISNYLNWYQQKPGKAPKLLIYYTSNLHSGVPSRFSGSGSGTDFTLTISSLQPEDFATYYCQQYRKLPWTFGQGTKLEIK

SEQ ID NO: 487 - protein, 122 aa, synthetic construct [sequence omitted]

SEQ ID NO: 488 - protein, 107 aa, synthetic construct [sequence omitted]

SEQ ID NO: 489 - protein, 116 aa, synthetic construct
EVQLVESGGGLVQPGGSLRLSCAASGFTFSSYMMSWVRQAPGKGLEWVATISGGGANTYYPDSVKGRFTISRDNAKNSLYLQMNSLRAEDTAVYYCARQLYYFDYWGQGTTVTVSS

SEQ ID NO: 490 - protein, 107 aa, synthetic construct
DIQMTQSPSSLSASVGDRVTITCLASQTIGTWLTWYQQKPGKAPKLLIYTATSLADGVPSRFSGSGSGTDFTLTISSLQPEDFATYYCQQVYSIPWTFGGGTKVEIK

SEQ ID NO: 491 - protein, 115 aa, synthetic construct
EVQLLESGGGLVQPGGSLRLSCAASGFTFSSYPMSWVRQAPGKGLEWVSAIGGSGGSLPYADIVKGRFTISRDNSKNTLYLQMNSLRAEDTAVYYCARYWPMDIWGQGTLVTVSS

SEQ ID NO: 492 - protein, 108 aa, synthetic construct
EIVLTQSPGTLSLSPGERATLSCRASQSVSSSYLAWYQQKPGQAPRLLMYDASIRATGIPDRFSGSGSGTDFTLTISRLEPEDFAVYYCQQYQSWPLTFGQGTKVEIK

SEQ ID NO: 493 - protein, 116 aa, synthetic construct [sequence omitted]

SEQ ID NO: 494 - protein, 111 aa, synthetic construct [sequence omitted]

SEQ ID NO: 495 - protein, 120 aa, synthetic construct
QVQLVQPGAEVVKPGASVKLSCKTSGYTFTSNWMHWVKQAPGQGLEWIGEIDPSDSYTNYNQNFQGKAKLTVDKSTSTAYMEVSSLRSDDTAVYYCARGSNPYYYAMDYWGQGTSVTVSS

SEQ ID NO: 496 - protein, 104 aa, synthetic construct
EIVLTQSPAIMSASPGERVTMTCSASSGVNYMHWYQQKPGTSPRRWIYDTSKLASGVPARFSGSGSGTSYSLTISSMEPEDAATYYCHQRGSYTFGGGTKLEIK

SEQ ID NO: 497 - protein, 121 aa, synthetic construct
EVQLVESGGGLVKPGGSLKLSCAASGYTFTSYVMHWVRQAPGKGLEWIGYINPYNDGTKYNEKFQGRVTISSDKSISTAYMELSSLRSEDTAMYYCARGTYYYGTRVFDYWGQGTLVTVSS

SEQ ID NO: 498 - protein, 112 aa, synthetic construct
DIVMTQSPATLSLSPGERATLSCRSSKSLQNVNGNTYLYWFQQKPGQSPQLLIYRMSNLNSGVPDRFSGSGSGTEFTLTISSLEPEDFAVYYCMQHLEYPITFGAGTKLEIK

SEQ ID NO: 499 - protein, 107 aa, synthetic construct; identical to SEQ ID NO: 280

SEQ ID NO: 500 - protein, 107 aa, synthetic construct
RTVAAPSVFIFPPSDEKLKSGTAKVVCLLNNFYPREAKVQWKVDNALQSGNSCESVTEQDSKDSTYSLSSTLTLSKADYEKHKVYACEVTHQGLSSPVTKSFNRGEA

SEQ ID NO: 501 - protein, 107 aa, synthetic construct
RTVAAPSVFIFPPSDEQLKSGTAKVVCLLNNFYPREAKVQWKVDNALQSGNSCESVTEQDSKDSTYSLKSTLTLSKADYEKHKVYACEVTHQGLSSPVTKSFNRGEA

SEQ ID NO: 502 - protein, 107 aa, synthetic construct
RTVAAPSVFIFPPSDEKLKSGTAKVVCLLNNFYPREAKVQWKVDNALQSGNSCESVTEQDSKDSTYSLKSTLTLSKADYEKHKVYACEVTHQGLSSPVTKSFNRGEA

SEQ ID NO: 503 - protein, 107 aa, synthetic construct
RTVAAPSVFIFPPSDEKLKSGTAKVVCLLINFYPREAKVQWKVDNALQSGNSCESVTEQDSKDSTYSLSSTLTLSKADYEKHKVYACEVTHQGLSSPVTKSFNRGEA

SEQ ID NO: 504 - protein, 111 aa, synthetic construct
AVALHRPDVELLPPAREQLNLRRSATITCLVDGFSPADVFVQWMQRGQPLSPEKYVTSAPMPEPRAPGRYCAHSILTVSEAEWNTGETYTCVVAHEALPNRVTERTVDKST

SEQ ID NO: 505 - protein, 111 aa, synthetic construct
AVALHRPDVDLLPPAREQLNLRRSATITCLVDGFSPADVFVQWMQRGQPLSPEKYVTSAPMPEPRAPGRYCAHSILTVSEAEWNTGETYTCVVAHEALPNRVTERTVDKST

SEQ ID NO: 506 - protein, 107 aa, synthetic construct
RTVAAPSVFIFPPSDECLKSGTAKVHCLLLNFYPREVKVQWKVDGALQSGNIKESVTEQDSKDGTYSLKSTLTLSKADYEKHKVYACEVTHQGLSSPVTKSFNRGEA

SEQ ID NO: 507 - protein, 111 aa, synthetic construct
AVALHRPDVCLLPPAREQLNLRRSATITCLVDGFSPADVFVQWMQRGQPLSPEKYVTSAPMPEPRAPGRYFAVSILTVSEAEWNTGETYTCVVAHEALPNRVTERTVDKST

SEQ ID NO: 508 - protein, 111 aa, synthetic construct
AVALHRPDVCLLPPAREQLNLRRSATITCLVDGFSPADVFVQWMQRGQPLSPEKYVTSAPMPEPRAPGRYFADSILTVSEAEWNTGETYTCVVAHEALPNRVTERTVDKST

SEQ ID NO: 509 - protein, 111 aa, synthetic construct
AVALHRPDVCLLPPAREQLNLRRSATITCLVDGFSPADVFVQWMQRGQPLSPEKYVTSAPMPEPRAPGEYFAHSILTVSEAEWNTGETYTCVVAHEALPNRVTERTVDKST

SEQ ID NO: 510 - protein, 107 aa, synthetic construct
RTVAAPSVFIFPPSDECLKSGTAKVHCLLNNFYPREVKVQWKVDGALQSGNIKESVTEQDSKDGTYSLSSTLTLSKADYEKHKVYACEVTHQGLSSPVTKSFNRGEA

SEQ ID NO: 511 - protein, 98 aa, synthetic construct
ASTKGPSVRPKAPSSKSTSGGTAALGCLVKDYFPEPVTVSWNSGALTSGVHTFPAVLQSSGLYSLSSVVTVPSSSLGTQTYICNVNHKPSNTKVDKKV

SEQ ID NO: 512 - protein, 98 aa, synthetic construct
ASTKGPSVFPKAPSSKSTSGGTAALGCKVKDYFPEPVTVSWNSGALTSGVHTFPAVLQSSGLYSLSSVVTVPSSSLGTQTYICNVNHKPSNTKVDKKV

SEQ ID NO: 513 - protein, 98 aa, synthetic construct
ASTKGPSVFPKAPSSKSTSGGTAALGCLVKDYFPEPVTVSWNSGALTSGVHTFPAVLQSSGLYSLKSVVTVPSSSLGTQTYICNVNHKPSNTKVDKKV

SEQ ID NO: 514 - protein, 111 aa, synthetic construct
AVALHRPDVYLLPPAREQLNLRRSATITCLVDGFSPADVFVQWMQRGQPLSPEKYVTSAPMPEPRAPGRYCADSILTVSEAEWNTGETYTCVVAHEALPNRVTERTVDKST

SEQ ID NO: 515 - protein, 103 aa, synthetic construct
ASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSC

SEQ ID NO: 516 - protein, 11 aa, synthetic construct
CDKTHTCPPCP

SEQ ID NO: 517 - protein, 8 aa, synthetic construct
CVECPPCP

SEQ ID NO: 518 - protein, 22 aa, synthetic construct
TKVDKKVEPKSCDKTHTCPPCP

SEQ ID NO: 519 - protein, 22 aa, synthetic construct
RTVDKSTEPKSCDKTHTCPPCP

SEQ ID NO: 520 - protein, 22 aa, synthetic construct
SPVTKSFNRGECDKTHTCPPCP

SEQ ID NO: 521 - protein, 22 aa, synthetic construct
TKVDKKVEPKSADKTHTCPPCP

SEQ ID NO: 522 - protein, 22 aa, synthetic construct
RTVDKSTEPKSADKTHTCPPCP

SEQ ID NO: 523 - protein, 22 aa, synthetic construct
SPVTKSFNRGEADKTHTCPPCP

(19) United States
(12) Patent Application Publication
(10) Pub. No.: US 2026/0098094 A1
(43) Pub. Date: Apr. 9, 2026

(54) Cµ4 REGION FOR PAIRING HEAVY AND LIGHT CHAINS IN MULTI-SPECIFIC ANTIBODIES

(71) Applicant: DNA Twopointo, Inc., Newark, CA (US)

(72) Inventors: Jeremy Minshull, Los Altos, CA (US); Sridhar Govindarajan, Los Altos, CA (US)

(73) Assignee: DNA Twopointo, Inc., Newark, CA (US)

(21) Appl. No.: 19/396,664

(22) Filed: Nov. 21, 2025

Related U.S. Application Data
(63) Continuation-in-part of application No. PCT/US25/31661, filed on May 30, 2025.
(60) Provisional application No. 63/654,845, filed on May 31, 2024.

(51) Int. Cl. C07K 16/28; C07K 16/22

(52) U.S. Cl. CPC C07K 16/2827; C07K 16/22; C07K 2317/31; C07K 2317/33; C07K 2317/522; C07K 2317/524; C07K 2317/526; C07K 2317/53; C07K 2317/72; C07K 2317/94

(57) ABSTRACT

The invention provides multi-specific, for example bispecific, antibodies which include two different pairs of heavy and light chains, in which directed correct pairing of heavy and light chains is promoted by inclusion of an IgM Cμ4 region pairing domain, or a modified IgM Cμ4 region pairing domain. The IgM Cμ4 region pairs with a light chain kappa region or a CH1 region in one of the pairings. The other pairing is optionally a conventional CH1 and light chain constant region, or can be another pair of engineered binding domains. The IgM Cμ4 region and kappa or CH1 region preferentially associate with one another in the first pairing, and optionally, CH1 and light chain constant regions preferentially associate with each other in the other pairing thereby promoting correct combinations of heavy and light chain and disfavoring incorrect combinations.

### CROSS-REFERENCE TO RELATED APPLICATIONS

This application is a continuation-in-part of International Application No. PCT/US2025/031661, filed on May 30, 2025, which claims the benefit of U.S. Provisional Patent Application No. 63/654,845, filed on May 31, 2024. Each of these applications is incorporated by reference herein in its entirety for all purposes.

### SEQUENCE LISTING

A Sequence Listing is submitted concurrently herewith electronically in XML format and is hereby incorporated by reference in its entirety. The XML copy, created on Nov. 20, 2025, is named Bispecifics-03-BypassCIP_ST26.xml and is 582,969 bytes in size.

### TECHNICAL FIELD

The present disclosure relates to compositions and methods for the generation of engineered antibodies, specifically, for the generation of engineered orthogonal antibody chains that can be programmed to pair with a preferred designated partner chain. The antibodies thus generated can be multispecific, for example bispecific antibodies, where the two or more antibody arms can be programmed with different epitope binding specificities.

### BACKGROUND

Production of bispecific antibodies requires correct pairing both between heavy and light chains to form a binding site and in pairing of heavy chains to form a heterodimer of binding sites. One approach has been to produce bispecific antibodies in which one or both binding sites are antibody fragments, for example IgG-single chain variable fragment (scFv), Fab-scFv, and scFv-scFv fusion proteins (Coloma et al., Nat Biotechnol 15:125-6, 1997; Lu et al., J Immunol Methods 267:213-26, 2002; Mallender, J Biol Chem 269:199-206, 1994), dual variable domain antibodies (DVD-Ig; Wu et al., Nat Biotechnol 25:1290-7, 2007), and diabodies (Holliger et al., Proc Natl Acad Sci USA 90:6444-8, 1993). Use of fragments allows expression of heavy and light chains as a single contiguous molecule. Bispecific F(ab′)2 antibody fragments have also been produced by chemical coupling (Brennan et al., Science 229:81, 1985) or by using leucine zippers (Kostelny et al., J Immunol 148:1547-53, 1992). Bispecific antibodies have also been made by chemically cross-linking the two heavy chain-light chain pairs produced separately (Karpovsky et al., J Exp Med 160:1686-701, 1984).

More naturally shaped bispecific antibodies can be produced by expressing both required heavy chains and light chains in a single cell. However, mispairing between chains results in up to ten different antibody-like compounds are made by such a cell (see Schaefer et al., Proc Natl Acad Sci USA 108:11187-92, 2011) so that it may be time consuming to purify a desired bispecific antibody out of this mixture. Mispairing of heavy chains with each other can be reduced by inserting an amino acid “knob” into the CH3 region of one of the two heavy chains and a corresponding “hole” into the CH3 region of the other chain so that the different heavy chains can more readily form heterodimers than homodimers, thus reducing formation of a non-bispecific antibody in which both heavy chains are the same, a strategy termed knob-into hole or knob-and-hole (Ridgway et al., Protein Eng 9:617-21, 1996; Atwell et al., J Mol Biol 270:26-35, 1997; and U.S. Pat. No. 7,695,936). However, there are still four different pairings of the two light chains with the two heavy chains, of which only one combination is correct.

### SUMMARY OF THE CLAIMED INVENTION

The present disclosure provides a number of embodiments for the production of antibodies (which includes antibody fragments), where in some embodiments, the antibodies are bispecific or multispecific.

The invention provides a multi-specific antibody comprising:
a first chain comprising a first variable region, a first pairing region and first IgG or IgA CH2 and CH3 regions;
a second chain comprising a second variable region, and a second pairing region;
wherein the first and second variable regions are heavy and light chain variable regions or vice versa;
wherein the first and second pairing regions are (a) an IgM Cμ4 region and (b) a kappa light chain constant region or a first IgG or IgA CH1 region; or vice versa;
wherein the first chain and second chain are paired via association of the first and second pairing regions forming a binding site for a first target antigen epitope;
a third chain comprising a third variable region, a third pairing region, and second IgG or IgA CH2 and CH3 regions;
a fourth chain comprising a fourth variable region and a fourth pairing region;
wherein the third and fourth variable regions are heavy and light chain variable regions or vice versa;
wherein the third and fourth pairing regions are a second IgG or IgA CH1 constant region and a second light chain constant region or vice versa;
wherein the third and fourth chains are paired via association of the third and fourth pairing regions forming a binding site for a second target antigen epitope; and
wherein the paired first and second chains and the paired third and fourth chains are associated via the first and second IgG or IgA CH3 regions thereby forming a tetramer.

Optionally, the first chain further comprises a first at least a portion of an IgG hinge region between the first pairing region and first IgG CH2 and CH3 regions, and the third chain further comprises a second at least a portion of an IgG hinge region between the third pairing region and second IgG CH2 and CH3 regions, wherein disulfide bonding between the first and second at least a portion of a hinge region promotes association of the paired first and second chains and the paired third and fourth chains.

Optionally, the first and second pairing regions each includes an engineered cysteine residue, which form a disulfide bond with one another, promoting pairing of the first and second chains.

Optionally, the first and second pairing regions are (a) the IgM Cμ4 region and (b) the kappa light chain constant region respectively. Optionally, the multi-specific antibody includes an engineered cysteine at position 455 of the IgM Cμ4 region and at position 121, 124 or 131 of the kappa light chain constant region, or an engineered cysteine at position 516 of the IgM Cμ4 region and position 160 of the kappa light chain constant region, or an engineered cysteine at position 471 of the IgM Cμ4 region and position 116 of the kappa light chain constant region, positions being numbered by Kabat numbering, or an engineered cysteine at position 463 of the IgM Cμ4 region and position 116 of the kappa light chain constant region.

Optionally, the first and second pairing regions are (a) the kappa light chain constant region and (b) the IgM Cμ4 region respectively. Optionally, the IgM Cμ4 region includes an engineered cysteine at position 455, and the kappa light chain constant region includes an engineered cysteine at position 131, or the IgM Cμ4 region includes an engineered cysteine at position 516, and the kappa light chain constant region includes an engineered cysteine at position 159, or the IgM Cμ4 region includes an engineered cysteine at position 463 and the kappa light chain constant region includes an engineered cysteine at position 116, positions being numbered by Kabat numbering.

Optionally, the first and second pairing regions are (a) the IgM Cμ4 region and (b) the first IgG or IgA CH1 region respectively. Optionally, the IgM Cμ4 region includes an engineered cysteine at position 455 of the IgM Cμ4 region and position 141 of the first CH1 region, position 516 of the IgM Cμ4 region and position 168 of the first CH1 region, position 463 of the IgM Cμ4 region and position 126 of the first CH1 region or position 457 of the IgM Cμ4 region and position 128 or 143 of the first CH1 region, positions in Cμ4 being numbered by Kabat numbering and positions in CH1 by EU numbering.

Optionally, the first and second pairing regions are (a) the first IgG or IgA CH1 region and (b) the IgM Cμ4 region respectively. Optionally, the IgM Cμ4 region includes an engineered cysteine at position 455 of the IgM Cμ4 region and position 141 of the first CH1 region, position 516 of the IgM Cμ4 region and position 168 of the first CH1 region, position 463 of the IgM Cμ4 region and position 126 of the first CH1 region positions in Cμ4 being numbered by Kabat numbering and positions in CH1 by EU numbering.

Optionally, the second pairing region has a naturally present cysteine substituted or deleted to prevent disulfide bonding of that pairing region to the third or fourth chains. Optionally, the second pairing region is the kappa light chain region and the naturally present cysteine is at the C-terminal position. Optionally, the second pairing region is IgM Cμ4 and the naturally occurring cysteine is at or before position 556 by Kabat numbering. Optionally, the second pairing region is the CH1 region and the C-terminus of the CH1 region is linked to an N-terminal hinge segment, and the naturally occurring cysteine is at position 220 by EU numbering of the N-terminal hinge segment is deleted or mutated if the CH1 region is of human IgG1 isotype or the cysteine at EU position 131 of the CH1 region is deleted or mutated if the CH1 region is of human isotype IgG2, IgG3 or IgG4.

Optionally, the third and fourth pairing regions are the second IgG or IgA CH1 constant region and the second light chain constant region, and the first chain comprises a CH1 region, at least a portion of a hinge and CH2 and CH3 regions each of human IgG1 isotype, and a cysteine residue at EU position 220 of the at least a portion of a hinge of the first chain is mutated is or deleted to prevent disulfide bonding with the second light chain constant region.

Optionally, the third and fourth pairing regions are the second IgG or IgA CH1 constant region, and the second light chain constant region, and the first chain comprises a CH1 region, at least a portion of a hinge and CH2 and CH3 regions, each of human IgG2, 3, or 4 isotype, and a cysteine residue at EU position 131 of the CH1 region of the first pairing region is mutated or deleted to prevent disulfide bonding with the second light chain constant region.

Optionally, the IgM Cμ4 region, when pairing with a CH1 pairing region, can include proline at position 482, tyrosine at position 477, valine at position 456, isoleucine at position 476, alanine or isoleucine at position 556, glutamine at position 549, isoleucine at position 523, valine at position 495, valine at position 475, phenylalanine at position 457, or histidine at position 546 by Kabat numbering.

Optionally, the kappa light chain constant region or first CH1 region and the second light chain constant region are not both linked to the heavy chain variable regions of the first and second binding sites, nor both to the light chain variable regions of the first and second binding sites.

Optionally, the following configuration can be used:
the first variable region is the light chain variable region of the first binding site and the first pairing region is the IgM Cμ4 region,
the second variable region is the heavy chain variable region of the first binding site and the second pairing region is the light chain kappa constant region or the first CH1 region,
the third variable region is the light chain variable region of the second binding site and the third pairing region is the second light chain constant region, and
the fourth variable region is the heavy chain variable region of the second binding site and the fourth pairing region is an IgG or IgA CH1 constant region.

Optionally, the following configuration can be used:
the first variable region is the heavy chain variable region of the first binding site and the first pairing region is the IgM Cμ4 region,
the second variable region is the light chain variable region of the first binding site and the second pairing region is the light chain kappa constant region or the first CH1 region,
the third variable region is the heavy chain variable region of the second binding site and the third pairing region is the second light chain constant region, and
the fourth variable region is the light chain variable region of the second binding site and the fourth pairing region is an IgG or IgA CH1 constant region.

Optionally the second light chain constant region is a second kappa light chain constant region. Optionally, the second light chain constant region is a lambda light chain constant region.

Optionally, the Cμ4 region has a sequence comprising any of SEQ ID NOS:23-25, 53, 54, 56-59, 74-78, 86-92 or 119-190, 218-220, 222-229 and 265-269 and the at least portions of hinge regions, each has a sequence selected independently from sequences comprising CDKTHTCPPCP (SEQ ID NO: 516) or CVECPPCP (SEQ ID NO: 517) or any of SEQ ID NOs: 2, 6, 10, 14, 117, 196-199, 231 and 232.

Optionally, the multi-specific antibody is bispecific.

Optionally, the first and second IgG or IgA CH2 and CH3 regions have complementary knob and hole mutations to promote their association.

Optionally, the first least a portion of a hinge region and the first CH2 and CH3 regions can be of any isotype and any subclass, for example, can be human IgG1, IgG2, IgG3 or IgG4. Optionally, the second at least a portion of a hinge region and the second CH2 and CH3 regions can be all of the same isotype and same subclass, for example, human IgG1, IgG2, IgG3 or IgG4.

Optionally, the first binding site specifically binds to a first target antigen epitope on a target cell and the second binding site specifically binds to a second target antigen epitope on the target cell. Optionally, the first binding site specifically binds to a target antigen epitope on a target cell and the second binding site specifically binds to a target antigen epitope on an effector cell, or vice versa. Optionally, the first binding site specifically binds to a target antigen epitope on a target cell, and the second binding site specifically binds to a checkpoint target. Optionally, the target cell is any of a cancer cell, a cell of a pathogen, or immune cell resulting in autoimmune disease.

Optionally, the first or second at least a portion of a hinge and or the first or second CH2 or CH3 regions include a mutation modulating effector function. Optionally, the first or second CH2 or CH3 regions include a mutation increasing FcRn binding and half-life. Optionally, any or all of the first, second, third and fourth chains are humanized, chimeric, veneered, or human heavy and light chains.

The invention further provides a bispecific antibody comprising:
a first chain comprising a first heavy chain variable region, a first pairing region, a first at least a portion of a hinge region, and first IgG CH2 and CH3 regions, each of human IgG1 isotype;
a second chain comprising a first light chain variable region, and a second pairing region;
wherein the first and second pairing regions are the first IgG and the IgM Cμ4 region respectively;
wherein the first chain and second chain are paired via association of the first and second pairing regions forming a binding site for a first target antigen epitope;
a third chain comprising a third variable region, a third pairing region, a second at least a portion of a hinge region and second IgG CH2 and CH3 regions, each of human IgG1 isotype;
a fourth chain comprising a fourth variable region and a fourth pairing region;
wherein the third and fourth variable regions are heavy and light chain variable regions or vice versa;
wherein the third and fourth pairing regions are the second IgG constant region and the second light chain constant region or vice versa;
wherein the third and fourth chains are paired via association of the third and fourth pairing regions forming a binding site for a second target antigen epitope; and
wherein the paired first and second chains and the paired third and fourth chains are associated via the first and second IgG CH3 regions thereby forming a tetramer and by at least one disulfide bond between the first and second at least partial hinge regions;
where the IgM Cμ4 region includes an engineered cysteine at position 455 of the IgM Cμ4 region and position 141 of the first CH1 region, position 516 of the IgM Cμ4 region and position 168 of the first CH1 region, position 463 of the IgM Cμ4 region and position 126 of the first CH1 region or position 457 of the IgM Cμ4 region and position 128 or 143 of the first CH1 region, positions in Cμ4 being numbered by Kabat numbering and positions in CH1 by EU numbering; and
the C-terminus of the CH1 region is linked to an N-terminal hinge segment, and the naturally occurring cysteine is at position 220 by EU numbering of the N-terminal hinge segment.

The invention further provides a bispecific antibody comprising:
a first chain comprising a first heavy chain variable region, a first pairing region, a first at least a portion of a hinge region, and first IgG CH2 and CH3 regions, each of human IgG1 isotype;
a second chain comprising a first light chain variable region, and a second pairing region;
wherein the first and second pairing regions are an IgM Cμ4 region respectively and a kappa light chain constant region;
wherein the first chain and second chain are paired via association of the first and second pairing regions forming a binding site for a first target antigen epitope;
a third chain comprising a third variable region, a third pairing region, a second at least a portion of a hinge region and second IgG CH2 and CH3 regions, each of human IgG1 isotype;
a fourth chain comprising a fourth variable region and a fourth pairing region;
wherein the third and fourth variable regions are heavy and light chain variable regions or vice versa;
wherein the third and fourth pairing regions are the second IgG CH1 constant region and the second light chain constant region or vice versa;
wherein the third and fourth chains are paired via association of the third and fourth pairing regions forming a binding site for a second target antigen epitope; and
wherein the paired first and second chains and the paired third and fourth chains are associated via the first and second IgG CH3 regions thereby forming a tetramer and by at least one disulfide bond between the first and second at least partial hinge regions;
wherein the multi-specific antibody includes an engineered cysteine at position 455 of the IgM Cμ4 region and at position 121, 124 or 131 of the kappa light chain constant region, or an engineered cysteine at position 516 of the IgM Cμ4 region and position 160 of the kappa light chain constant region, or an engineered cysteine at position 471 of the IgM Cμ4 region and position 116 of the kappa light chain constant region, positions being numbered by Kabat numbering, or an engineered cysteine at position 463 of the IgM Cμ4 region and position 116 of the kappa light chain constant region;
wherein the kappa light chain has a C-terminal cysteine deleted to prevent disulfide bonding of the second pairing region to the third or fourth chain.

The invention further provides a method of preparing a multi-specific antibody as described above, comprising expressing in host cells, the first, second, third and fourth chains, wherein the first and second chains are expressed at higher level than the third and fourth chains; and performing CH1-affinity separation to purify the multi-specific antibody from homodimers comprising pairs of the first and second chains.

In other aspects, one objective of the present disclosure is to create an HC/LC pair or a pair fragment (e.g., Fab) that is orthogonal to normal HC/LC pairing. In one aspect, for bispecific formats that have two heavy chains and two light chains, or fragments thereof (for example, F(ab)₂), using the normal pairing for one half antibody, and the orthogonal pairing for the other, where the orthogonal pairing prevents undesired LC/HC mispairing. Alternatively, engineered components can be used to construct a second orthogonal HC/LC pairing, different from the first HC/LC pairing, thereby constructing a complete antibody (e.g., an antibody comprising a total of two heavy chains and two light chains), or an antibody fragment, where that antibody is specifically engineered to have a bispecific epitope binding.

In one aspect, the disclosure provides an antibody or antibody fragment (e.g., Fab) comprising:
(a) a first chain comprising a first variable region and a first pairing region; and
(b) a second chain comprising a second variable region and a second pairing region; wherein the first and second variable regions are heavy and light chain variable regions, or vice versa; wherein the first and second chains pair to each other via association of the first and second pairing regions, thereby forming a first binding site that specifically binds a first target epitope; wherein the first pairing region is a first IgM Cμ4 region, and the second pairing region is selected from a first kappa light chain constant region and a first CH1 region. In this aspect, the second pairing region can optionally be a kappa light chain constant region. In some aspects, the first CH1 region is selected from an IgA CH1 and an IgG CH1, for example, an IgG1 CH1.

The antibody of the invention can comprise at least one amino acid insertion, deletion or substitution in the first or the second pairing region, or both the first and second pairing regions. In some aspects, the first and second pairing regions each includes an engineered cysteine residue, which form a disulfide bond linking the first and second chains. In some aspects, the at least one amino acid addition, deletion or substitution enhances electrostatic attraction between the first and second pairing regions. For example, the IgM Cμ4 region an antibody of the invention can comprise at least one mutation selected from:
(i) glutamate 468 deleted or mutated to arginine,
(ii) deletion of glutamate 525,
(iii) deletion of glutamate 527,
(iv) glutamate 526 deleted or mutated to alanine, and
(v) glutamine 510 mutated to arginine.

In some aspects, the antibody or antibody fragment such as described above can further comprise a second arm to form a complete HC/LC antibody (e.g., a four chain antibody) or a F(ab)₂ fragment of a four chain antibody. The two arms of the F(ab)₂ fragment can be joined by any suitable coupling, for example, by a disulfide bond as in F(ab′)₂, or by any suitable chemical linkage. In this aspect, the four chain antibody further comprises:
(a) a third chain comprising a third variable region and a third pairing region; and
(b) a fourth chain comprising a fourth variable region and a fourth pairing region; wherein the third and fourth variable regions are heavy and light chain variable regions, or vice versa; wherein the first and second chains are preferentially paired to each other via association of the first and second pairing regions, and wherein the third and fourth chains are preferentially paired to each other via association of the third and fourth pairing regions, thereby forming a second binding site that specifically binds a second target epitope that is different from the first target epitope; further wherein:
   (i) the third pairing region is selected from:
      (A) a second IgM Cμ4 region,
      (B) a second light chain constant region, and
      (C) a first modified CH3 region; and
   (ii) where the third pairing region is (A), (B) or (C), the fourth pairing region is, correspondingly, selected from:
      (A′) a second kappa light chain constant region or a second-CH1 region;
      (B′) a third CH1 region, and
      (C′) a second modified CH3 region, wherein the first and second modified CH3 regions pair preferentially with each other.

In some aspects, the first, second, third and fourth pairing regions collectively comprise a plurality of amino acid deletions, insertions or substitutions such that the first and second pairing regions preferentially pair with each other relative to their pairing with either the third or fourth pairing regions, and the third and fourth pairing regions preferentially pair with each other relative to their pairing with either the first or second pairing regions.

These antibodies of the disclosure can be a multi-specific antibodies, for example, bispecific or trispecific antibodies. In some aspects, the third and fourth chains are covalently coupled to the first and second chains. In some aspects, the first or second chain are part of a contiguous polypeptide that further comprises the third or fourth chain.

In some aspects, the four chain antibody further comprises at least one amino acid addition, deletion or substitution in the first or the second pairing region, or both the first and second pairing regions, wherein the addition, deletion or substitution (a) promotes the pairing of the first and second pairing regions, or (b) disfavors the pairing of the first or second pairing regions with the third or fourth pairing regions. Similarly, in some aspects, the four chain antibody comprises at least one amino acid addition, deletion or amino acid substitution in the third or fourth pairing region, or both the third and fourth pairing regions, wherein the addition, deletion or substitution (a) promotes the pairing of the third and fourth pairing regions, or (b) disfavors the pairing of the third or fourth pairing regions with the first or second pairing regions.

In some aspects, the at least one amino acid addition, deletion or substitution results in the formation of a disulfide bond, thereby covalently linking the first and second pairing regions or the third and fourth pairing regions. In some aspects, the at least one amino acid addition, deletion or substitution prevents the formation of a disulfide bond, thereby preventing covalent linkage between the first or second pairing regions with the third or fourth pairing regions. In some aspects, the at least one amino acid addition, deletion or substitution increases the electrostatic attraction between the first and second pairing regions or the third and fourth pairing regions, thereby promoting pairing between the first and second pairing regions or the third and fourth pairing regions. In some aspects, the at least one amino acid addition, deletion or substitution results in electrostatic repulsion between the first or second pairing regions and the third or fourth pairing regions, thereby suppressing pairing between the first or second pairing regions and the third or fourth pairing regions.

In some aspects, (a) the first pairing region is a first IgM Cμ4 region comprising a substitution of threonine at position 477 (Kabat numbering) to an amino acid selected from histidine, lysine and arginine; and the second pairing region is a first kappa light chain constant region comprising a substitution of serine at position 131 (EU numbering) to an amino acid selected from aspartate and glutamate; or (b) the first pairing region is a first IgM Cμ4 region comprising a substitution of threonine at position 477 (Kabat numbering) to an amino acid selected from aspartate and glutamate; and the second pairing region is a first kappa light chain constant region comprising a substitution of serine at position 131 (EU numbering) to an amino acid selected from histidine, lysine and arginine.

In some aspects of the four chain antibody, the third pairing region is (B) a kappa light chain constant region, comprising one or more substitutions selected from (i) serine 176 to aspartate or glutamate, (ii) valine 133 to serine and (iii) glutamine 124 to aspartate or glutamate; and the fourth pairing region (B′) is an IgG1 CH1 constant region comprising a substitution selected from leucine 128 to lysine or arginine (all EU numbering). In other aspects, where the second pairing region is a first kappa light chain constant region comprising a substitution of asparagine 137 to an amino acid selected from leucine, isoleucine, valine and methionine, and the fourth pairing region (B′) is an IgG1 CH1 constant region.

In some aspects of the four chain antibody, wherein the first pairing region is an IgM Cμ4 region and the second pairing region is a CH1 region, wherein:
(a) the IgM Cμ4 region comprises an engineered cysteine at amino acid position 455 by Kabat numbering, and the CH1 region comprises an engineered cysteine residue at amino acid position 141 by EU numbering; or
(b) the IgM Cμ4 region comprises an engineered cysteine at amino acid position 516 by Kabat numbering, and the CH1 region comprises an engineered cysteine at amino acid position 168 by EU numbering; or
(c) the IgM Cμ4 region comprises an engineered cysteine at amino acid position 463 by Kabat numbering, and the CH1 region comprises an engineered cysteine residue at amino acid position 126 by EU numbering; or
(d) the IgM Cμ4 region comprises an engineered cysteine at amino acid position 457 by Kabat numbering, and the CH1 region comprises an engineered cysteine residue at amino acid position 128 or 143 by EU numbering.

In some aspects of the invention, the first or second pairing region has a naturally present cysteine substituted or deleted to prevent disulfide bonding of the second pairing region to the third or fourth chains. In some aspects, the second pairing region is the kappa light chain constant region and the naturally present cysteine is substituted or deleted at the C-terminal position. In some aspects, the first pairing region is the IgM Cμ4 region, and the naturally present cysteine is substituted or deleted at or before position 556 by Kabat numbering. In some aspects, the second pairing region is the CH1 region and the C-terminus of the CH1 region is linked to an N-terminal IgG1 hinge segment, and the naturally present cysteine is substituted or deleted at position 220 by EU numbering of the N-terminal hinge segment.

In some aspects of the four chain antibody of the invention, the IgM Cμ4 region includes one or more of:
(a) proline at position 482,
(b) tyrosine at position 477,
(c) valine at position 456,
(d) isoleucine at position 476,
(e) alanine or isoleucine at position 556,
(f) glutamine at position 549,
(g) isoleucine at position 523,
(h) valine at position 495,
(i) valine at position 475,
(j) phenylalanine at position 457, and
(k) histidine at position 546, all by Kabat numbering.

In other aspects of a four chain antibody of the invention, the kappa light chain constant region or first CH1 region and the second light chain constant region are not both linked to the heavy chain variable regions of the first and second binding sites, nor both to the light chain variable regions of the first and second binding sites. In some aspects, the second light chain constant region is a second kappa light chain constant region or a lambda light chain constant region.

In some aspects of a four chain antibody of the invention, where the first variable region is the heavy chain variable region of the first binding site and the first pairing region is the kappa light chain constant region or the first CH1 region, the second variable region is the light chain variable region of the first binding site and the second pairing region is the IgM Cμ4 region, the third variable region is the heavy chain variable region of the second binding site and the third pairing region is an IgG or IgA CH1 constant region and the fourth variable region is the light chain variable region of the second binding site and the fourth pairing region is the second light chain constant region.

In other aspects of a four chain antibody of the invention, where the first variable region is the light chain variable region of the first binding site and the first pairing region is the kappa light chain constant region or the first CH1 region, the second variable region is the heavy chain variable region of the first binding site and the second pairing region is the IgM Cμ4 region, the third variable region is the light chain variable region of the second binding site and the third pairing region is an IgG or IgA CH1 constant region and the fourth variable region is the heavy chain variable region of the second binding site and the fourth pairing region is the second light chain constant region.

Variants of an antibody of the invention are no particularly limited. For example, the first, second, third or fourth chains, or any subset thereof, can be humanized, chimeric, veneered, or human heavy and light chains.

In some aspects of the disclosure, the nature of the antibody binding site and the target epitope are considered. In some aspects, the first binding site or the second binding site specifically binds to a first or second target epitope on a target cell, wherein the target cell is any of a cancer cell, a cell of a pathogen, or an immune cell resulting in autoimmune disease.

The nature of the target epitope of an antibody of the invention is not particularly limited. A target epitope can be a soluble antigen epitope. Alternatively, a target epitope can be a cell surface epitope. In some aspects, the first binding site of a bispecific antibody of the invention specifically binds to a first target epitope on a target cell and the second binding site specifically binds to a second target epitope on the same target cell. In other aspects, the first binding site specifically binds to a first target epitope on a first target cell and the second binding site specifically binds to a second target epitope on a second target cell. In this aspect, either the first or second target epitope can be CD3, CD2, CD28, CD44, C69, A13 or G1, but is not limited in this regard. In some aspects, the first or the second target cell is an immune effector cell. In other aspects, either the first or second target epitope is an Fc gamma receptor epitope, for example but not limited to, 3G8, B73.1, LEUL1, VEP13, and AT10.

In other aspects, an antibody of the invention can target a signaling protein. The signaling protein targeted by an antibody of the invention is not particularly limited. As used herein, the term “signaling protein” or related terms refer to any protein involved in cellular communication or signal transduction of any type, for example, signaling proteins that influence various cellular behaviors. Signaling proteins can have a variety of different forms and functions. Many signaling proteins are involved in processes of human health and disease, and as such, are attractive targets for antibody binding for activation or neutralization when the antibodies are used as large molecule therapeutics. Signaling proteins can be cell surface signaling proteins, for example, on immune effector cells, or can be soluble signaling proteins.

In some aspects of the multispecific, e.g., bispecific, antibodies of the invention, the first binding site specifically binds to a first target epitope on a first target cell and the second binding site specifically binds to a signaling protein. Signaling protein targets include, but are not limited to, for example, VEGF, PD-L1 and PD-L2.

In some aspects, the first binding site on an antibody of the invention specifically binds to a first target epitope on a first target cell and the second binding site specifically binds to a checkpoint target on an immune effector cell. The checkpoint target on an immune effector cell can be, for example but not limited to, PD-1, PD-2, CTLA-40, CD47, OX40, B7.1, B7He, LAG3, CD137, KIR, CCR5, CD27 and CD40.

In some aspects, the first or second target epitope is an Fc gamma receptor epitope, such as 3G8, B73.1, LEUL1, VEP13, or AT10.

In some aspects, a bispecific antibody of the invention is characterized where either the first or the second binding site specifically binds a target epitope selected from CD3, CD20, CD22, CD30, CD34, CD40, CD44, CD47, CD52 CD70, CD79a, DR4 DR5, EGFR, CA-125/Muc-16, MC1 receptor, PEM antigen, gp72, EpCAM, Her-2, VEGF or VEGFR, ganglioside GD3, CEA, AFP, CTLA-4, alpha v beta 3, HLA-DR 10 beta, SK-1, PD-1, PD-2, PD-L1, PD-L2, CTLA-40, CD47, OX40, B7.1, B7He, LAG3, CD137, KIR, CCR5, CD27 and CD40.

In other aspects, the present disclosure also provides methods for preparing an antibody of the invention, where the antibody comprises at least one IgM Cμ4 pairing region, where the method has the steps of (a) expressing in host cells, the first, second, third and fourth chains, wherein the first and second chains are expressed at higher level than the third and fourth chains; and (b) performing CH1-affinity separation to purify the multi-specific antibody from homodimers comprising pairs of the first and second chains.

In other specific aspects of the four chain bispecific antibody, where the first pairing region is an IgM Cμ4 region and the second pairing region is a kappa light chain constant region, the antibody is further characterized by: (a) the IgM Cμ4 region comprises an engineered cysteine at amino acid position 455 by Kabat numbering, and the kappa light chain constant region comprises an engineered cysteine residue at amino acid position 121, 124 or 131 by EU numbering; or (b) the IgM Cμ4 region comprises an engineered cysteine at amino acid position 516 by Kabat numbering, and the kappa light chain constant region comprises a cysteine at amino acid position 160 by EU numbering. In this aspect, the kappa light chain constant regions of (a) or (b) further optionally comprise removing or substituting the cysteine at position C214 by EU numbering with an amino acid incapable of forming a disulfide bond.

In other specific aspects of the four chain antibody of the invention, the antibody is modified where:
(a) the first pairing region is a first IgM Cμ4 region comprising an engineered cysteine at amino acid position 455 by Kabat numbering,
(b) the second pairing region is a first kappa light chain constant region comprising an engineered cysteine residue at amino acid position 121, 124 or 131 by EU numbering,
(c) the third pairing region is (A) a second IgM Cμ4 region comprising an engineered cysteine at amino acid position 516 by Kabat numbering, and
(d) the fourth pairing region is (A′) a second kappa light chain constant region comprising a cysteine at amino acid position 160 by EU numbering.

In still other aspects of the four chain antibodies of the invention, the antibodies are modified to comprise at least a portion of a hinge region and CH2 and CH3 constant domains to form a tetramer antibody with two heavy chains and two light chains. More specifically, the antibodies are modified where:
(a) either the first or second chain further comprises:
   (i) a first at least a portion of a hinge region,
   (ii) a first CH2 region, and
   (iii) a first CH3 region, and
(b) either the third or fourth chain further comprises:
   (i′) a second at least a portion of a hinge region,
(ii′) a second CH2 region, and
(iii′) a second CH3 region,
   wherein the first at least a portion of a hinge region is positioned between the first or second pairing region and the first CH2 and CH3 regions, wherein the second at least a portion of a hinge region is positioned between the third or fourth pairing region and the second CH2 and CH3 regions, and wherein the paired first and second chains and the paired third and fourth chains are associated with each other via at least the first and second CH3 regions, thereby forming a tetramer.

In some aspects of the tetramer antibodies, the first CH2 region, the first CH3 region, the second CH2 region, and the second CH3 region are, independently, an IgG isotype or an IgA isotype. Optionally, the first and second hinge or hinge portion regions, the first and second CH2 regions, and the first and second CH3 regions are all of the same isotype and subclass. In some aspects, the first and second hinge or hinge portion regions, the first and second CH2 regions, and the first and second CH3 regions are all of the IgG1 isotype and subclass, and optionally where the second or fourth pairing region are a CH1 region, and said CH1 region is of the IgG1 isotype and subclass.

In some aspects of the tetrameric antibody, the chains are modified to improve pairing properties between the chains. In some aspects, the associated first and second chains and the third and fourth chains are associated with each other, at least in part, by at least one disulfide bond.

In some aspects of the tetramer antibodies of the invention, the second pairing region is a kappa light chain constant region, wherein the kappa light chain constant region has a naturally present cysteine substituted or deleted to prevent disulfide bonding of the second pairing region to the third or fourth chains. In some aspects, the first or second hinge regions or portions thereof comprise removing or substituting the cysteine at the position analogous to C220 of the IgG1 hinge amino acid sequence by EU numbering with an amino acid incapable of forming a disulfide bond. In other aspects, the third and fourth pairing regions are the second CH1 constant region and the second light chain constant region, and the second chain comprises a CH1 pairing region, at least a portion of a hinge and CH2 and CH3 regions of human IgG1 isotype, wherein a cysteine residue at EU position 220 of the at least a portion of a hinge of the second chain is mutated is or deleted to prevent disulfide bonding with the second light chain constant region.

In other aspects, the third and fourth pairing regions are the second CH1 constant region and the second light chain constant region, and the second chain comprises a CH1 region, at least a portion of a hinge and CH2 and CH3 regions of human IgG2, 3, or 4 isotype, wherein a cysteine residue at EU position 131 of the CH1 region of the second pairing region is mutated or deleted to prevent disulfide bonding with the second light chain constant region.

In some aspects, the antibodies are defined where the first or second IgM Cμ4 region comprises an amino acid sequence selected from SEQ ID NOS: 23-25, 53, 54, 56-59, 74-78, 86-92, 119-190, 218-220, 222-229, 265-269, 479, 504-505, and 507-509.

In some aspects, the antibodies are defined where the first or second at least a portion of a hinge region each comprises, independently, a sequence selected from CDKTHTCPPCP (SEQ ID NO: 516), CVECPPCP (SEQ ID NO: 517) and SEQ ID NOs: 2, 6, 10, 14, 117, 196-199, 231 and 232.

In some aspects, the chains comprising the first and second CH2 and CH3 regions comprise at least one pair of complementary knob and hole mutations to promote their association, for example, where the knob and hole mutations are selected from:
(a) Y407T in one chain and T366Y in the other chain,
(b) Y407A in one chain and T366W in the other chain,
(c) F405A in one chain and T394W in the other chain,
(d) F405W in one chain and T394S in the other chain,
(e) Y407T in one chain and T366Y in the other chain,
(f) T366Y and F405A in one chain and T394W and Y407T in the other chain,
(g) T366W and F405W in one chain and T394S and Y407A in the other chain,
(h) F405W and Y407A in one chain and T366W and T394S in the other chain, and
(i) T366W in one chain and T366S, L368A, and Y407V in the other chain;
all by EU numbering.

In some aspects, the chains are further modified where the chains comprising the first and second CH2 and CH3 regions comprise at least one pair of complementary mutations that create interchain disulfide bonds and thereby promote heterodimer formation, for example, where the pair of complementary mutations that create interchain disulfide bonds is selected from:
(a) Y349C in one chain and S354C in the other chain,
(b) Y349C in one chain and E356C in the other chain,
(c) Y349C in one chain and E357C in the other chain,
(d) L351C in one chain and S354C in the other chain,
(e) T394C in one chain and E397C in the other chain, and
(f) D399C in one chain and K392C in the other chain, by EU numbering.

In some aspects of antibodies of the invention, the chains comprising the first and second CH2 and CH3 regions comprise at least one pair of charge pair substitutions and thereby promote heterodimer formation. These pairs of charge pair substitutions can optionally be, for example,
(a) K409D or K409E in one chain and D399K or D399R in the other chain;
(b) K392D or K392E in one chain and D399K or D399R in the other chain;
(c) K439D or K439E in one chain and E356K or E356R in the other chain;
(d) K370D or K370E in one chain and E357K or E357R in the other chain;
(e) K409D and K360D in one chain plus D399K and E356K in the other chain;
(f) K409D and K370D in one chain plus D399K and E357K in the other chain;
(g) K409D and K392D in one chain plusD399K, E356K, and E357K in the other chain;
(h) K409D and K392D in one chain and D399K in the other chain;
(i) K409D and K392D in one chain and D399K and E356K in the other chain;
(j) K409D and K392D in one chain and D399K and D357K in the other chain,
(k) K409D and K370D in one chain and D399K and D357K in the other chain
(l) D399K in one chain and K409D and K360D in the other chain, and
(m) K409D and K439D in one chain and D399K and E356K on the other chain;
all by EU numbering.

In some aspects, the chains comprising the first and second CH2 and CH3 regions comprise at least one set of charge pair substitutions and at least one set of knob-into-hole substitutions, thereby promoting heterodimer formation with a preferred chain.

In some antibodies of the invention, the first at least a portion of a hinge region and the first CH2 and CH3 regions are all any one of human IgG1, IgG2, IgG3 or IgG4. Optionally, the second at least a portion of a hinge region and the second CH2 and CH3 regions are all any of human IgG1, IgG2, IgG3 or IgG4. Optionally, the first or the second at least a portion of a hinge region or the first or the second CH2 or CH3 regions include a mutation modulating effector function, for example, where the first or the second CH2 or CH3 regions include a mutation increasing FcRn binding or increases half-life of the antibody.

As discussed above, in some aspects, the antibody or antibody fragment can further comprise a second arm to form a complete HC/LC antibody (e.g., a four chain antibody) or a F(ab)₂ fragment of a four chain antibody. The two arms of the F(ab)₂ fragment can be joined by any suitable coupling, for example, by a disulfide bond as in F(ab′)₂, or by any suitable chemical linkage. In this aspect, the four chain antibody comprises first and second chains as described above, and further comprises:
(a) a third chain comprising a third variable region and a third pairing region; and
(b) a fourth chain comprising a fourth variable region and a fourth pairing region; wherein the first and second chains are preferentially paired to each other via association of the first and second pairing regions, and wherein the third and fourth chains are preferentially paired to each other via association of the third and fourth pairing regions, further wherein:
   (i) the third pairing region is selected from:
      (A) a second IgM Cμ4 region,
      (B) a second light chain constant region, and
      (C) a first modified CH3 region; and
   (ii) where the third pairing region is (A), (B) or (C), the fourth pairing region is, correspondingly, selected from:
      (A′) a second kappa light chain constant region or a second-CH1 region;
      (B′) a third CH1 region, and
      (C′) a second modified CH3 region, wherein the first and second modified CH3 regions pair preferentially with each other.

In one particular aspect, further in reference to the description above, an example of an antibody of the invention is described as follows:
(a) the fourth pairing region (B′) is an IgG1 CH1 region, and the fourth pairing region comprises substitutions L128K or L128R;
(b) the third pairing region (B) is a second kappa light chain constant region comprising substitutions
   (i) V133S and S176D, or
   (ii) V133S and S176E, or
   (iii) V133S, S176D and Q124E, or
   (iv) V133S, S176D and Q124D;
(c) the first pairing region is an IgM Cμ4 region, wherein said first pairing region comprises mutations:
   (i′) F516C, and/or
   (ii′) E468R or E468del, and/or
   (iii′) E526A or E526del, and/or
   (iv′) Q510R,
(d) the second pairing region is a first kappa light chain constant region, comprising mutations:
   (i″) Q160C and/or
   (ii″) Q124K, and/or
   (iii″) C214 is deleted or substituted to an amino acid that is not capable of forming a disulfide bond,
(e) the first at least portion of a hinge region is deleted or substituted at the naturally present cysteine C220 by EU numbering to an amino acid that is not capable of forming a disulfide bond;
(f) and further wherein, alternatively,
   (A) the first pairing region that is the IgM Cμ4 region comprises T477D or T477E, and the second pairing region that is the first kappa light chain constant region comprises S131K/H/R; or
   (B) the first pairing region that is an IgM Cμ4 region comprises K554E or K554D, and the second pairing region that is the first kappa light chain constant region comprises E123K;
   wherein the second kappa light chain constant region pairs preferably with the first IgG1 CH1 region, and the first kappa light chain constant region pairs preferably with the IgM Cμ4 region.

In another particular aspect, further in reference to the description above, an example of an antibody of the invention is described as follows:
(a) the first pairing region is an IgM Cμ4 region, and the first pairing region comprises substitutions
   (i) T477D or T477E, and
   (ii) Y455C;
(b) the second pairing region is a kappa light chain constant region comprising substitutions:
   (i) Q124C, and
   (ii) S131K or S131H or S131R;
(c) the third pairing region is (B) a second light chain constant region, wherein said third pairing region is a kappa light chain comprising mutations:
   (i) V133S and S176D, or
   (ii) V133S and S176E, or
   (iii) V133S, S176D and Q124E, or
   (iv) V133S, S176D and Q124D;
(d) the fourth pairing region is a (B′) CH1 constant region, comprising mutations:
   (i) L128K or L128R;
(e) the first at least portion of a hinge region is deleted or substituted at the naturally present cysteine C220 by EU numbering to an amino acid that is not capable of forming a disulfide bond; and
(f) the C-terminal cysteine of the second pairing region is deleted or substituted at the naturally present cysteine C214 by EU numbering to an amino acid that is not capable of forming a disulfide bond;
wherein the first pairing region pairs preferably with the second pairing region, and the third pairing region pairs preferably with the fourth pairing region.

In reference to the antibody described above where the first pairing region that is the IgM Cμ4 region, can further optionally comprise one or more of the following substitutions:
(i) E468R or E468del, and
(ii) E526A or E526del, and
(iii) Q510R.

Optionally, also in reference to this antibody, where the second pairing region that is the kappa light chain constant region, the kappa constant region further comprises one or more of the following substitutions:
(i) V133H, and
(ii) A144V, and
(iii) N152G, and
(iv) S159V or S159I, and
(v) Q160K, and
(vi) S171G,

In another particular aspect, further in reference to the description above, an example of an antibody of the invention is described as follows:
(a) the first pairing region is an IgM Cμ4 region comprising substitutions:
   (i) T477D or T477E, and
   (ii) F516C;
(b) the second pairing region is a kappa light chain constant region comprising substitutions:
   (i) Q160C, and
   (ii) S131K or S131H or S131R;
(c) the third pairing region is (B) a second light chain constant region, wherein said third pairing region is a kappa light chain comprising mutations:
   (i) V133S and S176D, or
   (ii) V133S and S176E, or
   (iii) V133S, S176D and Q124E, or
   (iv) V133S, S176D and Q124D;
(d) the fourth pairing region is a (B′) CH1 constant region, comprising mutations:
   (i) L128K or L128R;
(e) the first at least portion of a hinge region is deleted or substituted at the native cysteine C220 by EU numbering to an amino acid that is not capable of forming a disulfide bond; and
(f) the C-terminal cysteine of the second pairing region is deleted or substituted at the native cysteine C214 by EU numbering to an amino acid that is not capable of forming a disulfide bond;
wherein the first pairing region pairs preferably with the second pairing region, and the third pairing region pairs preferably with the fourth pairing region.

In reference to this antibody described above where the first pairing region that is the IgM Cμ4 region, can further optionally comprise one or more of the following substitutions:
(i) E468R or E468del, and
(ii) E526A or E526del, and
(iii) Q510R.

Optionally, also in reference to this antibody, where the second pairing region that is the kappa light chain constant region, the kappa constant region further comprises one or more of the following substitutions:
(i) Q124K, and
(ii) A144V, and
(iii) N152G, and
(iv) S159V or S159I, and
(v) Q160K, and
(vi) S171G.

In another particular aspect, further in reference to the description above, an example of an antibody of the invention is described as follows:
(a) the first pairing region is an IgM Cμ4 region comprising substitutions:
   (i) T477D or T477E, and
   (ii) F516C;
(b) the second pairing region is a kappa light chain constant region comprising substitutions:
   (i) Q160C, and
   (ii) S131K or S131H or S131R;
(c) the third pairing region is (A) a second IgM Cμ4 constant region comprising mutations T477K or T477R;
(d) the fourth pairing region is (A′) a second kappa light chain constant region comprising mutations:
   (i) S131D or S131E, and optionally
   (ii) Q124C;
(e) the first at least portion of a hinge region is deleted or substituted at the naturally present cysteine C220 by EU numbering to an amino acid that is not capable of forming a disulfide bond; and
(f) the C-terminal cysteine of the second pairing region is deleted or substituted at the naturally present cysteine C214 by EU numbering to an amino acid that is not capable of forming a disulfide bond;
wherein the first pairing region pairs preferentially with the second pairing region, and
the third pairing region pairs preferentially with the fourth pairing region.

In reference to this antibody described above where the first pairing region that is the IgM Cμ4 region, the IgM Cμ4 constant region optionally further comprises Y455CThe antibody of claim Error! Reference source not found., wherein the second kappa light chain constant region further comprises substitution Q124C. Optionally, the second at least portion of a hinge region native cysteine C220 by EU numbering is deleted or substituted with an amino acid that is not capable of forming a disulfide bond. Optionally, the C-terminal cysteine of the fourth pairing region is deleted or substituted at the native cysteine C214 by EU numbering to an amino acid that is not capable of forming a disulfide bond. Optionally, the antibody first and third pairing regions that are the IgM Cμ4 pairing regions each independently further comprise one or more of the following:
(i) E468R or E468del, and/or
(ii) E526A or E526del, and/or
(iii) Q510R.

Where the second pairing region that is the kappa light chain constant region, that domain optionally further comprises one or more of the following substitutions:
(i) Q124K, and
(ii) A144V, and
(iii) Q147V, and
(iv) N152G, and
(v) S159V or S159I, and
(vi) S171G, and
(vii) D185E.

Where the fourth pairing region that is the kappa light chain constant region, that domain further optionally comprises one or more of the following substitutions:
(i) A144V, and
(ii) Q147V, and
(iii) N152G, and
(iv) S159V or I, and
(v) Q160D or E, and
(vi) S171G, and
(vii) D185E.

In other aspects, the present disclosure also describes expression systems for producing an antibody of the invention in a host cell, where the system includes:
(a) a first polynucleotide encoding first polypeptide, said first polypeptide comprising a first variable region and a first pairing region;
(b) a second polynucleotide encoding second polypeptide, said second polypeptide comprising a second variable region and a second pairing region;
(c) a host cell suitable for expressing the first and second polypeptides following delivery of said first and second polynucleotides into said host cell;
   wherein the first and second variable regions are heavy and light chain variable regions, orvice versa; wherein the first and second polypeptides pair to each other via association of the first and second pairing regions, thereby forming a first binding site that specifically binds a first target epitope; and wherein the first pairing region is a first IgM Cμ4 region, and the second pairing region is selected from a first kappa light chain constant region and a first CH1 region.

In still other aspects, the present disclosure also describes methods for producing an antibody of the invention in a host cell, where the method includes the steps:
(a) providing:
   (i) a first polynucleotide encoding a first polypeptide, said first polypeptide comprising a first variable region and a first pairing region;
   (ii) a second polynucleotide encoding second polypeptide, said second polypeptide comprising a second variable region and a second pairing region;
   wherein (A) the first and second variable regions are heavy and light chain variable regions, or vice versa, (B) the first and second polypeptides pair to each other via association of the first and second pairing regions, thereby forming a first binding site that specifically binds a first target epitope, and (C) the first pairing region is a first IgM Cμ4 region, and the second pairing region is selected from a first kappa light chain constant region and a first CH1 region;
   (iii) a host cell suitable for expressing the first and second polypeptides following delivery of said first and second polynucleotides into said host cell;
(b) delivering said first and second polynucleotides in the host cell; and
(c) culturing said host cell under conditions for the expression of said first and second polypeptides, thereby producing said antibody.

The method can further include the step of purifying the antibody.

The specification also provides recombinant immunoglobulin polypeptides produced as described in the present disclosure. In various aspects, a polypeptide of the invention includes, minimally,
(a) a variable region selected from a heavy chain variable region and a light chain variable region, and
(b) a pairing region that is a modified IgM Cμ4 region, where the pairing region N-terminus is coupled to the variable region C-terminus,
   wherein the modified IgM Cμ4 region comprises at least one amino acid substitution, addition or deletion, said modified IgM Cμ4 region is capable of preferential binding to either a kappa light chain constant region or a CH1 region, where the preferential binding is assessed relative to the binding between a corresponding native IgM Cμ4 region and the kappa light chain constant region or the CH1 region. Optionally, the pairing region N-terminus is coupled directly to the variable region C-terminus. In other aspects, the pairing region N-terminus is coupled indirectly to the variable region C-terminus by a non-native coupling sequence. As used herein, the expression “non-native coupling sequence” refers to any single amino acid or chain of amino acids that is not a natural, i.e. native, sequence of amino acids that would couple the pairing region N-terminus to the variable region C-terminus.

A polypeptide of the invention can further comprise a modified IgM Cμ4 region comprising at least one amino acid substitution or addition resulting in the creation of a disulfide bond between the modified IgM Cμ4 region and either the kappa light chain constant region or a CH1 region.

The invention also includes any polynucleotide encoding any antibody chain of the invention, namely a polypeptide, as described above, for example, comprising, (a) a variable region selected from a heavy chain variable region and a light chain variable region, and (b) a pairing region that is an unmodified or a modified IgM Cμ4 region, where the pairing region N-terminus is coupled to the variable region C-terminus, which can be a direct coupling or an indirect coupling.

In its broadest sense, the disclosure describes minimally an antibody fragment or portion comprising two chains that are able to specifically pair, and when paired, form a binding site for an antigen or epitope associated with an antigen. These molecules are analogous to monovalent Fab-type molecules with a single “arm.”

In some aspects, these antibodies can comprise:
(a) a first chain comprising a first variable region and a first pairing region; and
(b) a second chain comprising a second variable region and a second pairing region;
wherein the first and second variable regions are heavy and light chain variable regions, or vice versa;
wherein the first and second chains pair to each other via association of the first and second pairing regions, thereby forming a first binding site that specifically binds a first target epitope;
wherein the first pairing region is a first IgM Cμ4 region, and the second pairing region is selected from a first kappa light chain constant region and a first CH1 region.

In further aspects, the antibody comprises at least one amino acid insertion, deletion or substitution in the first or the second pairing region, or both the first and second pairing regions, wherein the at least one amino acid insertion, deletion or substitution results in enhanced affinity of the first and second pairing regions. Where the second pairing region is the first kappa light chain constant region, the first kappa light chain constant region can optionally comprise at least one amino acid substitution selected from S171G, S159I, A144V, N152G, S159V, S159I, S159V, A1441, N138G, L136V, D185E, V1631, Q147V, Q147T, D122E, S171N, S156T, V205L, A111T, D122Q, L154V, E123D, Q147I, N210S and A193S.

The first and second pairing regions each can include an engineered cysteine residue, which form a disulfide bond linking the first chain and second chain.

In some aspects, the second pairing region is a kappa light chain constant region, wherein, alternatively:
(a) the IgM Cμ4 region comprises an engineered cysteine at amino acid position 455 (by Kabat numbering), and the first kappa light chain constant region comprises an engineered cysteine residue at amino acid position 121, 124 or 131 (by EU numbering); or
(b) the IgM Cμ4 region comprises an engineered cysteine at amino acid position 516 (by Kabat numbering), and the first kappa light chain constant region comprises a cysteine at amino acid position 160 by EU numbering.

In some aspects, the kappa light chain constant regions of (a) or (b) further comprise removing or substituting the cysteine at position C214 by EU numbering with an amino acid incapable of forming a disulfide bond.

In some aspects, the at least one amino acid addition, deletion or substitution enhances electrostatic attraction between the first and second pairing regions. For example, where the second pairing region is the first kappa light chain constant region, wherein alternatively:
(a) the first IgM Cμ4 pairing region comprises T477H, T477K or T477R (Kabat numbering); and the second pairing region is a first kappa light chain constant region comprising S131D or S131E (EU numbering), optionally where the first kappa light chain constant region further comprises at least one amino acid substitution selected from V133H and Q160K; or
(b) the first IgM Cμ4 pairing region comprises T477D orT477E (Kabat numbering); and the first kappa light chain constant region comprises S131H or S131K or S131R (EU numbering), where optionally the first kappa light chain constant region further comprises Q124K.

In some aspects where the second pairing region is a CH1 region, the antibody is characterized by:
(a) the IgM Cμ4 region comprises Y455C by Kabat numbering, and the CH1 region comprises A141C by EU numbering; or
(b) the IgM Cμ4 region comprises F516C by Kabat numbering, and the CH1 region comprises H168C by EU numbering; or
(c) the IgM Cμ4 region comprises Q463C by Kabat numbering, and the CH1 region comprises F126C by EU numbering; or
(d) the IgM Cμ4 region comprises L457C by Kabat numbering, and the CH1 region comprises L128C or G143C by EU numbering.

Optionally where the second pairing region is the first CH1 constant region, the IgM Cμ4 region includes one or more of:
(a) A482P,
(b) T477Y,
(c) L456V,
(d) V476I,
(e) T556I or T556A,
(f) E549Q,
(g) V523I,
(h) L495V,
(i) L475V,
(j) L457F, and
(k) R546H,
all by Kabat numbering.

In some aspects, the IgM Cμ4 region comprises at least one mutation elected from:
(i) E468del or E468R,
(ii) E525del,
(iii) E527del,
(iv) E526del or E526A, and
(v) Q510R.

In some aspects, the first pairing region N-terminus can be coupled either directly or indirectly to the first variable region C-terminus.

In other aspects, the disclosure also encompasses at least one polynucleotide encoding the first chain and the second chain of the antibody. In some aspects, the at least one polynucleotide can be a single polynucleotide that encodes both the first polypeptide chain and second polypeptide.

In still other aspects, the disclosure builds on the Fab molecule by further adding two additional chains (a third chain and a fourth chain) that are able to specifically and preferably pair with each other over pairing with either the first or second chain. When paired, the third and fourth chains form a second antibody “arm” that includes a second binding site for an antigen or epitope associated with an antigen. In the context of bispecific molecules, the binding site formed by the third and fourth chains is different from the binding site formed from the first and second chains. In one aspect, these four-chain molecules are analogous to F(ab)₂ and F(ab′)₂ type molecules, and optionally, can be further modified with the addition of Fc sequences to build a full antibody molecule, such as an antibody having a structure similar to the structure of an IgG isotype molecule, such as an IgG1 or IgG4 subclass molecules.

In some general aspects, this antibody of the disclosure can be described as a two chain Fab molecule, as described above, that further comprises:
(a) a third chain comprising a third variable region and a third pairing region; and
(b) a fourth chain comprising a fourth variable region and a fourth pairing region;
wherein the third and fourth variable regions are heavy and light chain variable regions, or vice versa;
wherein the first and second chains are preferentially paired to each other via association of the first and second pairing regions, and
wherein the third and fourth chains are preferentially paired to each other via association of the third and fourth pairing regions, thereby forming a second binding site that specifically binds a second target epitope that is different from the first target epitope;
wherein
   (i) the third pairing region is selected from:
      (A) a second IgM Cμ4 region,
      (B) a second light chain constant region, and
      (C) a first modified CH3 region; and
   (ii) where the third pairing region is (A), (B) or (C), the fourth pairing region is, correspondingly, selected from:
      (A′) a second kappa light chain constant region or a second-CH1 region;
      (B′) a third CH1 region, and
      (C′) a second modified CH3 region, wherein the first modified CH3 region pairs preferentially with the second modified CH3 region.

In some aspects, the first, second, third and fourth pairing regions collectively comprise at least one or preferably a plurality of amino acid deletions, insertions, substitutions or truncations such that the first and second pairing regions preferentially pair, or show improved preferential pairing, with each other relative to their pairing with either the third or fourth pairing regions, and the third and fourth pairing regions preferentially pair, or show improved preferential pairing, with each other relative to their pairing with either the first or second pairing regions. In some aspects, the third and fourth chains are covalently coupled to the first and second chains.

In some aspects, the antibody comprises at least one amino acid addition, deletion or substitution in the first pairing region, second pairing region, third pairing region, fourth pairing region, or any plurality combination thereof, wherein the addition, deletion or substitution:
(a) promotes the pairing of the first and second pairing regions; or
(b) disfavors the pairing of either the first or second pairing regions with either the third or fourth pairing regions.
(c) promotes the pairing of the third and fourth pairing regions; or
(d) disfavors the pairing of either the third or fourth pairing regions with either the first or second pairing regions.

In some aspects, the at least one amino acid addition, deletion or substitution results in the formation of a disulfide bond, i.e., a non-native disulfide bond, thereby covalently linking the first and second pairing regions or the third and fourth pairing regions. In some aspects, the at least one amino acid addition, deletion, substitution or truncation prevents the formation of a disulfide bond, thereby preventing covalent linkage between either the first or second pairing regions with either the third or fourth pairing regions.

In some aspects, the second pairing region is the first CH1 region and the C-terminus of the first CH1 region is linked to an N-terminal IgG1 hinge segment, and the naturally present cysteine at position 220 of the N-terminal hinge segment (by EU numbering) is substituted with an amino acid incapable to forming a disulfide bond or is deleted.

In some aspects of this antibody, the at least one amino acid addition, deletion or substitution increases the electrostatic attraction between (i) the first and second pairing regions, or (ii) the third and fourth pairing regions, thereby promoting pairing between the first and second pairing regions or the third and fourth pairing regions. In other aspects, the at least one amino acid addition, deletion or substitution results in electrostatic repulsion between (i) either the first or second pairing region, and (ii) either the third or fourth pairing region, thereby suppressing pairing between either the first or second pairing regions and either the third or fourth pairing regions.

In some aspects where the third pairing region is (B) a kappa light chain constant region, said region can comprise one or more substitutions selected from:
(i) S176D or S176E,
(ii) V133S and
(iii) Q124D or Q124E; and the fourth pairing region (B′) is an IgG1 CH1 constant region comprising a substitution selected from L128K or L128R (all EU numbering).

In some aspects, the first or second pairing region has a native cysteine substituted with an amino acid incapable of forming a disulfide bond or deleted to prevent disulfide bonding of that pairing region to the third or fourth chains.

In some aspects, the second pairing region is the kappa light chain constant region and the native cysteine at the C-terminal portion of the kappa light chain constant region is substituted with an amino acid incapable of forming a disulfide bond or is deleted, to prevent disulfide bonding of the second pairing region to the third or fourth chains. In some aspects, the first pairing region is the IgM Cμ4 region, wherein said IgM Cμ4 region contains a substitution to remove a native cysteine, where the native cysteine position is (i) substituted with an amino acid that is incapable of forming a disulfide bond, (ii) is deleted, or where the IgM Cμ4 region is truncated to exclude the native cysteine.

In some aspects, the antibody is characterized by:
(a) the first variable region is the light chain variable region of the first binding site and the first pairing region is the IgM Cμ4 region,
(b) the second variable region is the heavy chain variable region of the first binding site and the second pairing region is the first kappa light chain constant region or the first CH1 region,
(c) the third variable region is the light chain variable region of the second binding site and the third pairing region is the second light chain constant region, and
(d) the fourth variable region is the heavy chain variable region of the second binding site and the fourth pairing region is a CH1 constant region selected from an IgG CH1 constant region and an IgA CH1 constant region.

In some aspects, the antibody is characterized by:
(a) the first variable region is the heavy chain variable region of the first binding site and the first pairing region is the IgM Cμ4 region,
(b) the second variable region is the light chain variable region of the first binding site and the second pairing region is the kappa light chain constant region or the first CH1 region,
(c) the third variable region is the heavy chain variable region of the second binding site and the third pairing region is the second light chain constant region, and
(d) the fourth variable region is the light chain variable region of the second binding site and the fourth pairing region is a CH1 constant region selected from an IgG CH1 constant region and an IgA CH1 constant region.

In some aspects, the antibody is characterized by:
(a) the first pairing region is a first IgM Cμ4 region comprising Y455C by Kabat numbering,
(b) the second pairing region is a first kappa light chain constant region comprising S121C, Q124C or K131C, all by EU numbering,
(c) the third pairing region is (A) a second IgM Cμ4 region comprising F516C by Kabat numbering, and
(d) the fourth pairing region is (A′) a second kappa light chain constant region comprising a cysteine at amino acid position Q160C by EU numbering.

In some aspects, the antibody is characterized by:
(a) a first chain comprising a first IgM Cμ4 pairing region comprising amino acid substitutions Y455C and T477K,
(b) a second chain that pairs with the first chain, the second chain comprising a first kappa pairing region comprising amino acid substitutions Q124C, C214A, and either (i) S131D, or (ii) S131E,
(c) a third chain comprising a second IgM C04 pairing region comprising amino acid substitutions F516C and T477D, and
(d) a fourth chain that pairs with the third chain, the fourth chain comprising a second kappa pairing region comprising mutations Q160C, C214A and S131K.

In other aspects, the disclosure also encompasses at least one polynucleotide encoding the first chain, second chain, third chain and fourth chain of any antibody as described above. In some aspects, the at least one polynucleotide is a plurality of polynucleotides, for example, four polynucleotides.

In still other aspects, the disclosure builds on the F(ab)₂ type molecules by further modifying the chains by adding Fc sequences, e.g., including hinge, CH2 and CH3 sequences, to build a full antibody molecule comprising heavy and light chains, such as, for example, IgG type molecules. The isotype and subclass of the Fc sequences added is not limited, and can be any suitable sequences, for example without limitation, derived from IgG1, IgG2, IgG3, IgG4, IgA1 and IgA2 molecules. Such molecules can be constructed using Fc sequences derived from all the same isotype and subclass, or can be chimeric molecules that combine Fc sequences derived from different isotypes and subclasses.

The full antibody molecules, i.e., comprising both Fab and Fc portions, can be described generally, referencing the F(ab)₂ class molecules already described above, wherein:
(a) either the first or second chain further comprises:
   (i) a first at least a portion of a hinge region,
   (ii) a first CH2 region, and
   (iii) a first CH3 region, and
(b) either the third or fourth chain further comprises:
   (i′) a second at least a portion of a hinge region,
   (ii′) a second CH2 region, and
   (iii′) a second CH3 region,
wherein the first at least a portion of a hinge region is positioned between the first or second pairing region and the first CH2 and CH3 regions,
wherein the second at least a portion of a hinge region is positioned between the third or fourth pairing region and the second CH2 and CH3 regions,
wherein the paired first and second chains and the paired third and fourth chains are associated with each other via at least the first and second CH3 regions, thereby forming a tetramer.

In some aspects, the first CH2 region, the first CH3 region, the second CH2 region, and the second CH3 region are, independently, an IgG isotype or an IgA isotype.

In some aspects, the associated first and second chains and the associated third and fourth chains are associated with each other, at least in part, by at least one disulfide bond. In some aspects, the first or second hinge regions or portions thereof comprise removing or substituting the cysteine at the position analogous to C220 of the IgG1 hinge amino acid sequence by EU numbering with an amino acid incapable of forming a disulfide bond. In some aspects, the antibody comprises a chain comprising an IgG1 CH1 pairing region or a IgM Cμ4 pairing region, said chain further comprising at least a portion of a hinge and CH2 and CH3 regions of human IgG1 isotype, wherein a cysteine residue at EU position 220 of the at least a portion of a hinge of that chain is substituted with an amino acid that can not form a disulfide bond or is deleted to prevent disulfide bonding with the C-terminal cysteine of a light chain constant region.

In other aspects, the antibody is described, wherein:
(a) the third pairing region is the second CH1 constant region,
(b) the fourth pairing region is the second light chain constant region, and
(c) the first chain comprises a IgM Cμ4 pairing region, at least a portion of a hinge and CH2 and CH3 regions of human IgG1 isotype, wherein a cysteine residue at EU position 220 of the at least a portion of a hinge of the first chain is substituted with an amino acid that can not form a disulfide bond or deleted to prevent disulfide bonding with the second light chain constant region.

In other aspects, the antibody is described, wherein:
(a) the third pairing region is the second CH1 constant region,
(b) the fourth pairing region is the second light chain constant region, and
(c) the second chain comprises a CH1 pairing region, at least a portion of a hinge and CH2 and CH3 regions of human IgG1 isotype, wherein a cysteine residue at EU position 220 of the at least a portion of a hinge of the second chain is substituted with an amino acid that can not form a disulfide bond is or deleted to prevent disulfide bonding with the second light chain constant region.

In other aspects, the antibody comprises a chain comprising a CH1 region that is an IgG2, IgG3 or IgG4 isotype and subclass, said chain further comprising at least a portion of a hinge and CH2 and CH3 regions of human IgG2, IgG3 or IgG4 isotype, and wherein the CH1 region comprises a cysteine residue at EU position 131 that is substituted with an amino acid that can not form a disulfide bond or is deleted to prevent disulfide bonding with the C-terminal cysteine of a light chain constant region.

In other aspects, the antibody is described, wherein:
(a) the third pairing region is the second CH1 constant region,
(b) the fourth pairing region is the second light chain constant region, and
(c) the second chain comprises a CH1 pairing region, at least a portion of a hinge and CH2 and CH3 regions of human IgG2, IgG3 or IgG4 isotype, wherein the CH1 pairing region comprises a cysteine residue at EU position 131 that is substituted with an amino acid that can not form a disulfide bond or is deleted to prevent disulfide bonding with the second light chain constant region.

In some aspects, the first or second IgM Cμ4 pairing region comprises an amino acid sequence selected from SEQ ID NOS: 23-25, 53, 54, 56-59, 74-78, 86-92, 119-190, 218-220, 222-229, 265-269, 479, 504-505, and 507-509.

In some aspects, the first and second at least a portion of a hinge region each comprises, independently, a sequence selected from CDKTHTCPPCP (SEQ ID NO: 516), CVECPPCP (SEQ ID NO: 517) and SEQ ID NOs: 2, 6, 10, 14, 117, 196-199, 231 and 232.

In some aspects, the chains comprising the first and second CH2 and CH3 regions comprise at least one pair of complementary knob and hole amino acid substitutions to promote their association. In some aspects, the complementary knob and hole amino acid substitutions are selected from:
(a) Y407T in one chain and T366Y in the other chain,
(b) Y407A in one chain and T366W in the other chain,
(c) F405A in one chain and T394W in the other chain,
(d) F405W in one chain and T394S in the other chain,
(e) Y407T in one chain and T366Y in the other chain,
(f) T366Y and F405A in one chain and T394W and Y407T in the other chain,
(g) T366W and F405W in one chain and T394S and Y407A in the other chain,
(h) F405W and Y407A in one chain and T366W and T394S in the other chain, and
(i) T366W in one chain and T366S, L368A, and Y407V in the other chain;
all by EU numbering.

In some aspects, the chains comprising the first and second CH2 and CH3 regions comprise at least one pair of complementary amino acid substitutions that create interchain disulfide bonds and thereby promote heterodimer formation. In some aspects, the pair of complementary amino acid substitutions that create interchain disulfide bonds is selected from:
(a) Y349C in one chain and S354C in the other chain,
(b) Y349C in one chain and E356C in the other chain,
(c) Y349C in one chain and E357C in the other chain,
(d) L351C in one chain and S354C in the other chain,
(e) T394C in one chain and E397C in the other chain, and
(f) D399C in one chain and K392C in the other chain,
by EU numbering.

In some aspects, the first or the second at least a portion of a hinge region or the first or the second CH2 or CH3 regions include at least one mutation modulating effector function. The modulation of effector function can be upregulating effector function or downregulating effector function. In some aspects, the first or the second CH2 or CH3 regions include at least one mutation increasing FcRn binding or that increases half-life of the antibody.

In some aspects, the first or the second CH2 or CH3 regions comprise at least one mutation that eliminates or reduces binding to protein A.

In various aspects, the disclosure also provides pharmaceutical compositions, for example, a composition comprising an antibody as described herein, and further comprising a pharmaceutically acceptable excipient.

In other aspects, the disclosure provides at least one polynucleotide which encodes the antibody first chain, second chain, third chain and fourth chain. In various aspects, the at least one polynucleotide can be a plurality of polynucleotides, comprising:
(a) a first polynucleotide encoding the first chain,
(b) a second polynucleotide encoding the second chain,
(c) a third polynucleotide encoding the third chain, and
(d) a fourth polynucleotide encoding the fourth chain.

In still other aspects, the disclosure also provides expression systems for producing an antibody, for example, in a host cell. The Examples herein provide detailed methodologies and description of the components for an expression systems, including suitable host cells, polynucleotides suitable for delivery into the host cells, and methodologies for the delivery of the polynucleotides into the host cells. Such polynucleotides comprise open reading frames that encode the antibody polypeptide chains described herein and also comprise other nucleotide sequences (such as promoters) to drive the transcription of the open reading frames and expression of the antibody polypeptides, e.g., heavy and light chain polypeptides, in the host cells. In some aspects, an expression system as used herein comprises:
(a) a first polynucleotide encoding first polypeptide, said first polypeptide comprising
   a first variable region and a first pairing region;
(b) a second polynucleotide encoding second polypeptide, said second polypeptide comprising a second variable region and a second pairing region;
(c) a host cell suitable for expressing the first and second polypeptides following delivery of said first and second polynucleotides into said host cell;
wherein the first and second variable regions are heavy and light chain variable regions, or vice versa;
wherein the first and second polypeptides pair to each other via association of the first and second pairing regions, thereby forming a first binding site that specifically binds a first target epitope;
wherein the first pairing region is a first IgM Cμ4 region, and the second pairing region is selected from a first kappa light chain constant region and a first CH1 region.

The expression system above can be used, for example, for the manufacture of F(ab)₂ type antibodies, and also adapted for the manufacture of full antibody molecules also comprising Fc domains.

In still other aspects, the disclosure also provides methods for the production of antibodies described herein. The Examples herein provide detailed methodologies for producing antibodies, including descriptions of polynucleotides, host cells, methodologies for delivery of polynucleotides into host cells, culture of the host cells, and multiple methods for collection and purification of the antibodies. In various aspects, a method for producing an antibody as described in the present disclosure comprises:
(a) providing:
   (i) a first polynucleotide encoding a first polypeptide, said first polypeptide comprising a first variable region and a first pairing region;
   (ii) a second polynucleotide encoding a second polypeptide, said second polypeptide comprising a second variable region and a second pairing region; wherein
      (A) the first and second variable regions are heavy and light chain variable regions, or vice versa,
      (B) the first and second polypeptides pair to each other via association of the first and second pairing regions, thereby forming a first binding site that specifically binds a first target epitope, and
      (C) the first pairing region is a first IgM Cμ4 region, and the second pairing region is selected from a first kappa light chain constant region and a first CH1 region;
   (iii) a host cell suitable for expressing the first and second polypeptides following delivery of said first and second polynucleotides into said host cell;
(b) delivering said first and second polynucleotides in said host cell;
(c) culturing said host cell under conditions for the expression of said first and second polypeptides, thereby producing said antibody.

The methods above can be used, for example, for the manufacture of F(ab)₂ type antibodies and also adapted for the manufacture of full antibody molecules also comprising Fc domains.

In other aspects, the disclosure provides examples of detailed antibody structures for the purpose of illustrating the antibodies that are encompassed by the disclosure. These particular antibodies are intended merely to serve as examples and are not indicative of the full scope of the antibodies of the disclosure. These antibodies are intended only to help the reader understand the antibodies of the disclosure and are not intended to be limiting with regard to the scope of antibodies recited in the description and claims herein.

As one example antibody of the disclosure, an antibody is provided, comprising:
(a) a first chain comprising:
   a first heavy chain variable region,
   a first pairing region that is a first IgG or IgA CH1 region,
   a first at least a portion of a hinge region, and
   first IgG or IgA CH2 and CH3 regions;
(b) a second chain comprising:
   a first light chain variable region, and
   a second pairing region that is an IgM Cμ4 region;
   wherein the first chain and second chain preferentially pair with each other via association of the first and second pairing regions, thereby forming a first binding site that specifically binds a first target epitope;
(c) a third chain comprising:
   a third variable region,
   a third pairing region,
   a second at least a portion of a hinge region, and
   second IgG or IgA CH2 and CH3 regions;
(d) a fourth chain comprising
   a fourth variable region, and
   a fourth pairing region;
      wherein (i) the third and fourth variable regions are heavy and light chain variable regions, or vice versa; (ii) the third and fourth pairing regions are a second IgG or IgA CH1 constant region and a second light chain constant region or vice versa; and (iii) the third and fourth chains are preferentially paired with each other via association of the third and fourth pairing regions, thereby forming a second binding site that specifically binds a second target epitope; further wherein the paired first and second chains and the paired third and fourth chains are associated via the first and second IgG or IgA CH3 regions, thereby forming a tetramer, and further associated by at least one disulfide bond between the first and second at least partial hinge regions; wherein the IgM Cμ4 region and CH1 region comprise engineered cysteine residues, alternatively, at the following positions:
(A) IgM Cμ4 at position 455 and the first CH1 at position 141,
(B) IgM Cμ4 at position 516 and the first CH1 at position 168,
(C) IgM Cμ4 at position 463 and the first CH1 at position 126,
(D) IgM Cμ4 at position 457 and the first CH1 at position 128 or 143,
positions in Cμ4 being numbered by Kabat numbering and positions in CH1 by EU numbering; and
   wherein the C-terminus of the CH1 region is linked to an N-terminal at least hinge portion, and
   the naturally occurring cysteine at position 220 by EU numbering of the N-terminal hinge segment is mutated or deleted.

In another illustrating example, the disclosure provides an antibody comprising:
(a) a first chain comprising:
   a first heavy chain variable region,
   a first pairing region that is an IgM Cμ4 region,
   a first at least a portion of a hinge region, and
   first IgG or IgA CH2 and CH3 regions;
(b) a second chain comprising:
   a first light chain variable region, and
   a second pairing region that is a kappa light chain constant region;
   wherein the first chain and second chain preferentially pair with each other via association of the first and second pairing regions, thereby forming a first binding site that specifically binds a first target epitope;
(c) a third chain comprising:
   a third variable region,
   a third pairing region,
   a second at least a portion of a hinge region, and
   second IgG or IgA CH2 and CH3 regions;
(d) a fourth chain comprising
   a fourth variable region, and
   a fourth pairing region;
   wherein (i) the third and fourth variable regions are heavy and light chain variable regions, or vice versa, (ii) the third and fourth pairing regions are a second IgG or IgA CH1 constant region and a second light chain constant region, or vice versa, (iii) the third and fourth chains are preferentially paired with each other via association of the third and fourth pairing regions, thereby forming a second binding site that specifically binds a second target epitope;
      further wherein the paired first and second chains and the paired third and fourth chains are associated via the first and second IgG or IgA CH3 regions, thereby forming a tetramer, and further associated by at least one disulfide bond between the first and second at least partial hinge regions; wherein the IgM Cμ4 region and kappa region of the first and second pairing regions comprise engineered cysteine residues, alternatively, at the following positions:
(A) IgM Cμ4 at position 455 and the kappa light chain at position 121, 124 or 131,
(B) IgM Cμ4 at position 516 and the kappa light chain at position 160,
positions in IgM Cμ4 being numbered by Kabat numbering and positions in kappa by EU numbering; and
   wherein the kappa light chain has a C-terminal cysteine deleted to prevent disulfide bonding of the second pairing region to the hinge region of the third chain, and the hinge region of the first chain has a cysteine deleted to prevent disulfide bonding of the fourth pairing region to the hinge region of the first chain.

### BRIEF DESCRIPTION OF THE FIGURES

FIGS. 1A-1F represent one format for a bispecific antibody. In FIG. 1A, the CH1 region of a first half-antibody is replaced by an IgM Cμ4 region. Pairing is maintained between the variable regions (V1a and V1b) because of pairing between the kappa light chain constant region and the IgM Cμ4 region. In FIG. 1B a second half-antibody has the natural organization with a heavy chain pairing to a light chain by interactions between the CH1 region and the light chain constant region (CL). FIG. 1C shows heterodimers are formed between the two half-antibodies. In FIG. 1D, the IgM Cμ4 region and kappa constant region of the first half-antibody are switched between heavy and light chains. In FIG. 1E the same switch is made between the pairing regions of the second half-antibody. FIG. 1F shows heterodimers are formed between these two half-antibodies.

FIG. 2A shows the bispecific format resulting from combining the chains shown in FIGS. 1D and 1E. FIG. 2B shows the bispecific format resulting from combining the chains shown in FIGS. 1A and 1E. The bispecific antibodies shown in both FIGS. 2A and 2B have only a single CH1 region which is on a light chain to facilitate a two-step affinity purification process. FIG. 2C shows the bispecific format resulting from combining the chains shown in FIGS. 1D and 1B and FIG. 2D shows the bispecific format resulting from combining the chains shown in FIGS. 1A and 1B, except that in both cases the heavy chain comprising the CH1 constant region further comprises mutations in the CH2 and CH3 regions that reduce or eliminate protein A binding.

FIGS. 3A-3F represent one format for a bispecific antibody. In FIG. 1A, the kappa region of a first half-antibody is replaced by an IgM Cμ4 region. Pairing is maintained between the variable regions (V1a and V1b) because of pairing between the CH1 constant region and the IgM Cμ4 region. In FIG. 1B a second half-antibody has the natural organization with a heavy chain pairing to a light chain by interactions between the CH1 region and the light chain constant region (CL). FIG. 1C shows heterodimers are formed between the two half-antibodies. In FIG. 1D, the IgM Cμ4 region and CH1 constant region of the first half-antibody are switched between heavy and light chains. In FIG. 1E the same switch is made between the pairing regions of the second half-antibody. FIG. 1F shows heterodimers are formed between these two half-antibodies. FIG. 4A shows a reduced gel and FIG. 4B shows a non-reduced gel of the polypeptides purified by binding and elution from protein A resin. The sequence details of the co-expressed polypeptides in each lane are shown in FIG. 16 Table 4.

FIGS. 5A and 5B compare (A) a conventional cross-over antibody in which both half antibodies comprise a CH1 region (b) with an exemplary antibody of the invention in which one half-antibody has IgM Cμ4 and the other half antibody has CH1.

FIG. 6A shows a reduced gel and FIG. 6B shows a non-reduced gel of the polypeptides purified by binding and elution from protein A resin. The sequence details of the co-expressed polypeptides in each lane are shown in FIG. 17 Table 5.

FIGS. 7A-7H show exemplary human immunoglobulin sequences. FIG. 7A shows IgG1 and IgG2 sequences. FIG. 7B shows IgG4 and IgG4 sequences. FIG. 7C shows IgA1 and IgA2 sequences. FIG. 7D shows IgM sequences. FIG. 7E on three sheets shows cysteine modifications for kappa (light chain) and Cμ4 (heavy chain) sequences. FIG. 7F on two sheets shows cysteine modifications for Cμ4 (light chain) and kappa (heavy chain) sequences. FIG. 7G on three sheets shows cysteine modifications for CH1 (light chain) and Cμ4 (heavy chain) sequences. FIG. 7H on two sheets shows cysteine modifications for Cμ4 (light chain) and CH1 (heavy chain) sequences.

FIG. 8A (upper) shows CH1-IgG1 hinge region junction. The bolded cysteine can form a disulfide bond with a C-terminal cysteine of a kappa light chain region. The middle part of the figure shows a Cμ4-IgG1 hinge region. Again the bolded cysteine can form a disulfide bond with a C-terminal cysteine of a kappa light chain. The lower portion of the figure shows a kappa-IgG1 hinge junction. Part of the hinge region is deleted so the C-terminal cysteine of the kappa light chain aligns with the bolded cysteine in the full IgG1 hinge region. FIG. 8B shows the same three hinge regions with the cysteine capable of forming a disulfide bond with a light chain mutated to alanine (represented as a bold “A”). Other amino acid substitutions replacing the cysteine with an amino acid incapable of forming a disulfide bond are also acceptable, for example, the cysteine can be mutated to valine, isoleucine, glycine, threonine, serine, methionine, or any other amino acid that is incapable of forming a disulfide bond.

FIG. 9A shows a reduced gel and FIG. 9B shows a non-reduced gel of the polypeptides purified by binding and elution from protein A resin. The sequence details of the co-expressed polypeptides in each lane are shown in FIG. 18 Table 6.

FIGS. 10A and 10B show non-reduced gels of the polypeptides present in culture supernatant. The sequence details of the co-expressed polypeptides in each lane are shown in FIG. 19 Table 7.

FIG. 11 shows a non-reduced gel of the polypeptides present in culture supernatant. The sequence details of the co-expressed polypeptides in each lane are shown in FIG. 22 Table 10.

FIG. 12 shows a non-reduced gel of the polypeptides present in culture supernatant. The sequence details of the co-expressed polypeptides in each lane are shown in FIG. 23 Table 11.

FIG. 13 provides Table 1 showing conventional definitions of CDR's using Kabat numbering.

FIG. 14 provides Table 2 showing the numbering used for the IgM Cμ4 region, namely, the Kabat Residues numbering convention.

FIG. 15 provides Table 3 which lists examples of commercial antibodies and their targets.

FIG. 16 provides Table 4 showing the different chain combinations constructed and tested, and identifies the SEQ ID NOs corresponding to the amino acid sequences of each mature chain. Table 4 shows the two polypeptide chains co-expressed from polynucleotides as described in Example 1. Column A shows the polypeptide combination name; Column B shows the name of chain 1; Column C shows the chain 1 variable sequence name (VL is the light chain variable region, VH is the heavy chain variable region); Column D shows the chain 1 constant region name; Column E shows the SEQ ID NO corresponding to the amino acid sequence of mature chain 1; Column F shows the name of chain 2; Column G shows the chain 2 variable sequence name (VL is the light chain variable region, VH is the heavy chain variable region); Column H shows the chain 2 constant region name; Column I shows the SEQ ID NO corresponding to the amino acid sequence of mature chain 1; Column J shows the gel lane in FIG. 4A corresponding to the protein A-purified polypeptide combination; Column K shows the gel lane in FIG. 4B corresponding to the protein A-purified polypeptide combination.

FIG. 17 provides Table 5, showing different chain combinations constructed and tested, and identifies the SEQ ID NOs corresponding to the amino acid sequences of each mature chain. Column A shows the polypeptide combination name; Column B shows the SEQ ID NO corresponding to the amino acid sequence of mature chain LC2; Column C shows the SEQ ID NO corresponding to the amino acid sequence of mature chain HC2; Column D shows the SEQ ID NO corresponding to the amino acid sequence of mature chain HCl; Column E shows the SEQ ID NO corresponding to the amino acid sequence of mature chain LC1; Column F shows the name of the LC1 chain; Column G shows the gel lane in FIG. 6A corresponding to the protein A-purified polypeptide combination; Column H shows the gel lane in FIG. 6B corresponding to the protein A-purified polypeptide combination.

FIG. 18 provides Table 6, showing different chain combinations constructed and tested, and identifies the SEQ ID NOs corresponding to the amino acid sequences of each mature chain. Table 6 shows the two polypeptide chains co-expressed from polynucleotides as described in Example 3. Column A shows the polypeptide combination name; Column B shows the name of chain 1; Column C shows the chain 1 variable sequence name (VL is the light chain variable region, VH is the heavy chain variable region); Column D shows the chain 1 constant region name; Column E shows the SEQ ID NO corresponding to the amino acid sequence of mature chain 1; Column F shows the name of chain 2; Column G shows the chain 2 variable sequence name (VL is the light chain variable region, VH is the heavy chain variable region); Column H shows the chain 2 constant region name; Column I shows the SEQ ID NO corresponding to the amino acid sequence of mature chain 1; Column J shows the gel lane in FIG. 9A corresponding to the protein A-purified polypeptide combination; Column K shows the gel lane in FIG. 9B corresponding to the protein A-purified polypeptide combination.

FIG. 19 provides Table 7 summarizing different variant chain combinations constructed and tested, and identifies the SEQ ID NOs corresponding to the amino acid sequences of each mature chain. Because IgM Cμ4 pairs promiscuously with either CH1 or kappa constant regions, it is beneficial to modify both components of the desired pair to reduce non-specific pairing and improve specific pairing. Structural models of interactions between the IgG CH1 and IgM Cμ4 domains were used to identify the locations of residues that might be replaced by cysteines that might be capable of forming a covalent disulfide bond between the two chains. In this way, 11 potential pairs of substitutions were identified, summarized in Table 7.

FIG. 20 provides Table 8, showing antibody expression data of IgM Cμ4 pairing region variants. Column A shows the variant name, column B is the SEQ ID NO corresponding to the amino acid sequence of the IgM Cμ4 pairing region, and column C shows the titer (in mg/L) of antibody produced.

FIG. 21 provides Table 9, showing the contributions of different amino acid substitutions to assembled antibody titer. Mean values for the regression weights were calculated for each substitution. Column A shows the amino acid position, column B shows the amino acid naturally found at this position in an IgM Cμ4 pairing region, column C shows the amino acid substitution at this position and column D shows the average model weight from the expression data shown in FIG. 20 Table 8.

FIG. 22 provides Table 10. Structural models of interactions between kappa and IgM Cμ4 were used to identify the locations of residues within each of these domains that might be replaced by cysteines that might be capable of forming a covalent disulfide bond between the two chains. Three potential pairs of substitutions were identified, which are summarized in Table 10.

FIG. 23 provides Table 11, which shows various chain combinations tested, and identifies the SEQ ID NOs corresponding to the amino acid sequences of each mature chain. Antibody light chains comprised a kappa pairing region (column G), and a human kappa constant region (with amino acid sequence SEQ ID NO:35). The SEQ ID NO corresponding to the mature amino acid sequence of each light chain is shown in column F. The antibody heavy chains each comprised a sequence encoding, from N-terminus to C-terminus: a secretion signal, the heavy chain variable region of the antibody (with amino acid sequence SEQ ID NO:29), a human IgM Cμ4 constant region (with amino acid sequence corresponding to a SEQ ID NO shown in column I), a hinge region (with amino acid sequence corresponding to a SEQ ID NO shown in column J) and an Fc region (human IgG1 CH2 plus CH3 regions) with amino acid sequence SEQ ID NO:118. The SEQ ID NO corresponding to the mature amino acid sequence of each heavy chain is shown in FIG. 23 Table 11, column H.

FIG. 24 provides Table 12, which shows the results of a study to identify IgM Cμ4 constant region mutations that reduce binding to receptors that mediate effector function, namely FcpR, FcapR, and plgR. The mature light and heavy chains of this antibody have amino acid sequences SEQ ID NO:193 and 195 respectively. Additional amino acid changes were incorporated to create a set of variants of this antibody, the additional changes shown column E. Table 12 shows a qualitative measure of the binding response of each antibody to the three receptors FcapR, FcpR, and plgR as seen in columns B, C and D respectively.

FIG. 25 provides Table 13, which examines that ability to increase specificity of binding between light and heavy chains by engineering additional electrostatic interactions. The objective was to introduce mutually attractive changes into corresponding pairing regions that would at the same time result in mutually repulsive changes in non-corresponding pairing regions. Antibody light chains using a kappa pairing region each comprised a sequence encoding, from N-terminus to C-terminus: a secretion signal, the light chain variable region of the antibody (with amino acid sequence SEQ ID NO:28) and a kappa pairing region (with an amino acid sequence corresponding to a SEQ ID NO shown in column F). The SEQ ID NO corresponding to the mature amino acid sequence of each light chain is shown in column E. The position of engineered cysteines is indicated in column B (as well as the amino acid that is replaced), whether the natural cysteine at position 214 was allowed to remain is indicated in column C, other mutations are indicated in column D.

FIG. 26 provides Table 14, which describes the engineering of antibody molecules having various mutations, and examining the ability of these mutations to increase specificity of binding between light and heavy chains. Antibody heavy chains each comprised a sequence encoding, from N-terminus to C-terminus: a secretion signal, the heavy chain variable region of the antibody (with amino acid sequence SEQ ID NO:29), a human IgM Cμ4 constant region or a human IgG CH1 region (with amino acid sequence corresponding to a SEQ ID NO shown in column G if the pairing region was an IgG CH1 region, or in column F if the pairing region was an IgM Cμ4 pairing region), a hinge region (with amino acid sequence corresponding to a SEQ ID NO shown in column H) and an Fc region (human IgG1 CH2 plus CH3 regions) with amino acid sequence SEQ ID NO:118. The SEQ ID NO corresponding to the mature amino acid sequence of each heavy chain is shown in column E. The position of engineered cysteines is indicated in column B (as well as the amino acid that is replaced), whether the natural cysteine at hinge position 220 was allowed to remain is indicated in column C, and other mutations are indicated in column D.

FIG. 27 provides Table 15, which summarizes antibody titers (in mg/L) following expression of various combinations of heavy chain (or modified heavy chain), named in column A according to FIG. 26 Table 14, and light chain (or modified light chain), named in row 1 according to FIG. 25 Table 13. (nd=not done)

FIG. 28 provides Table 16, which summarizes antibody titers (in mg/L) following expression of various combinations of heavy chain (or modified heavy chain), named in column A according to FIG. 26 Table 14, and light chain (or modified light chain), named in row 1 according to FIG. 25 Table 13. (nd=not done)

FIG. 29 provides Table 17, which summarizes antibody titers (in mg/L) following expression of various combinations of heavy chain (or modified heavy chain), named in column A according to FIG. 26 Table 14, and light chain (or modified light chain), named in row 1 according to FIG. 25 Table 13. (nd=not done)

FIG. 30 provides Table 18, which summarizes antibody titers (in mg/L) following expression of various combinations of heavy chain (or modified heavy chain), named in column A according to FIG. 26 Table 14, and light chain (or modified light chain), named in row 1 according to FIG. 25 Table 13. (nd=not done)

FIG. 31 provides Table 19, which measures the percentage of correctly assembled antibody tetramer of the different chain combinations tested (the light chain used is shown in column A, the heavy chain used is shown in column B) and the resulting antibody titers in mg/L (column C).

FIG. 32 provides Table 20, which provides Tm testing data for various chain pairings. Polynucleotides encoding HC21-Fab and one light chain variant were co-transfected into HEK 293 cells. Expressed proteins were purified and Tm measured by DSF. Columns provide the SEQ ID NO corresponding to the amino acid sequence of the modified kappa region and for melting temperature in degrees Celsius (Tm). In some cases, the sequence modifications resulted in an antibody that was not expressed. In those cases, no Tm measurement was made, indicated as n/d (“not done”).

FIG. 33 provides Table 21, which provides data addressing the contributions of different amino acid substitutions to Fab melting temperature. Mean values and standard deviations for the regression weights were calculated for each substitution. The columns show the position in the kappa pairing region by EU numbering (“Position”), the amino acid being changed (“From”), the amino acid change being made (“To”), the mean regression weight (“Mean RW”) and the regression weight standard deviation (“RW SD”).

FIG. 34 provides Table 22, which shows the results of testing various combinations of the amino acid substitutions predicted to have the most positive effect on Fab antibody Tm. The protocol used Fab antibodies comprising heavy chain HC21-Fab and light chain LC10, wherein LC10 further comprised one or more substitutions selected from S171G, S159V, S159I, A144V and N152G. This table shows a column for each of these substitutions, a 1 indicates that the substitution was present in a variant, a 0 indicates its absence. The Tm column indicates the measured Tm in degrees Celsius.

FIG. 35 provides Table 23, which shows the results of testing various combinations of the amino acid substitutions predicted to have the most positive effect on Fab antibody Tm. The protocol used Fab antibodies comprising heavy chain HC46 and light chain LC35, where the columns show the name of each variant (“Name”) and the presence (indicated by a 1) or absence (indicated by a 0) of Q160K, S171G, S159I, N152G or A144V in their respective columns. The melting temperature measured by DSF is shown in the Tm column.

FIG. 36 provides Table 24, which provides Tm testing data for various chain pairings. Polynucleotides encoding HC47-Fab and one light chain variant were co-transfected into HEK 293 cells. Expressed proteins were purified and Tm measured by DSF. Columns provide the SEQ ID NO corresponding to the amino acid sequence of the modified kappa region and for melting temperature in degrees Celsius (Tm).

FIG. 37 provides Table 25, which shows data addressing the contributions of different amino acid substitutions to Fab melting temperature. Mean values and standard deviations for the regression weights were calculated for each substitution. The columns show the position in the kappa pairing region by EU numbering (“Position”), the amino acid being changed (“From”), the amino acid change being made (“To”), the mean regression weight (“Mean RW”) and the regression weight standard deviation (“RW SD”).

FIG. 38 provides Table 26, which shows the effects of various mutations in the pairing regions on the titers and percentage of correctly assembled Fab molecules. Mutations addressed include the introduction of engineered disulfide bonds. Kappa and IgM Cμ4 pairing regions were tested. The position of engineered cysteines is indicated (as well as the amino acid that is replaced), the substitution at serine 131 in kappa domain, mutations in the IgM Cμ4 domain, and other mutations are shown in the various columns. Polynucleotides encoding one heavy chain (or modified heavy chain) and one light chain (or modified light chain) were co-transfected into HEK 293 cells and cultured. Protein was purified by affinity chromatography on protein A resin, and the antibody yield was used to calculate the titer in the original culture. The table shows the different chain combinations tested and the resulting antibody titers in mg/L (column K). Protein was also run over an SEC column, and the % of product running at the correct size for a properly assembled antibody is shown in column M.

FIG. 39 provides Table 27, which describes the construction of variant pairing regions to be tested for the optimization of Fab pairings. New CH1 pairing regions comprising L128K were constructed, comprising one of the following additional substitutions: F126R, L145K or S183K. These positively charged substitutions were selected to introduce additional electrostatic repulsions against the positively charged IgM Cμ4-engineered kappas comprising a positively charged substitution at S131. The substitutions in these pairing regions are shown in the table. New substitutions were also introduced into IgM Cμ4-engineered kappas. New IgM Cμ4 pairing regions comprising T477D were constructed. These pairing regions either comprised an additional negatively charged substitution (H518D, R514E, Y455D, Y455E) designed to increase binding with the positively charged IgM Cμ4-engineered kappas, or H518V. These substitutions in these pairing regions are shown in the table.

FIG. 40 provides Table 28, which describes the construction of variant pairing regions to be tested for the optimization of Fab pairings.

FIG. 41 provides Table 29, which provides testing data showing the effects of various mutations in the pairing regions on the titers and percentage of correctly assembled Fab molecules. The various pairing region combinations as shown in the table were tested. Columns A and B provide the SEQ ID NOs corresponding to the amino acid sequences of the light and heavy pairing regions respectively. Subsequent pairs of columns give, in the left hand column of each pair, the titer of antibody produced (in mg/L), and in the right hand column of each pair, the percentage of the purified antibody that is correctly assembled as measured by size exclusion chromatography. Above each pair of columns is indicated the pairing regions used, first the light chain pairing region, then an underscore, then the heavy chain pairing region. Details about each pairing region, including the substitutions in each and the SEQ ID NO corresponding to the amino acid sequence of the pairing region are shown in FIG. 40 Table 28 for the light chain pairing regions and in FIG. 39 Table 27 for the heavy chain pairing regions.

FIG. 42 provides Table 30, which provides testing data addressing the effects of various substitutions in Cμ4-engineered kappa pairing regions with corresponding Cμ4 pairing regions. The Cμ4-engineered kappa pairing regions comprised an engineered cysteine at position 124, and the Cμ4 pairing regions comprised an engineered cysteine at position 455. Columns A and B provide the SEQ ID NOs corresponding to the amino acid sequences of the light and heavy pairing regions respectively. Subsequent pairs of columns give, in the left hand column of each pair, the titer of antibody produced (in mg/L), and in the right hand column of each pair, the percentage of the purified antibody that is correctly assembled as measured by size exclusion chromatography. Above each pair of columns is indicated the pairing regions used, first the light chain pairing region, then an underscore, then the heavy chain pairing region. Details about each pairing region, including the substitutions in each and the SEQ ID NO corresponding to the amino acid sequence of the pairing region are shown in FIG. 40 Table 28 for the light chain pairing regions and in FIG. 39 Table 27 for the heavy chain pairing regions. In this Table 30, rows 1-8 and 10-17 give titers or percentage of correctly assembled antibody for each combination, and rows 9 and 18 give average values for a single pairing region combination with eight different antibody variable regions.

FIG. 43 provides Table 31, which provides testing data for various combinations of kappa pairing regions with corresponding CH1 pairing regions, wherein the kappa pairing regions comprised substitutions Q124E, V133S and S176E, and the CH1 pairing region comprised substitution L128K, and optionally a further substitution selected from F126R, L145K or S183K. Columns A and B provide the SEQ ID NOs corresponding the amino acid sequences of the light and heavy pairing regions, respectively.

FIG. 44 provides Table 32, which provides testing data measuring mispairing propensity for various chain pairings. The pairing of LP51, a kappa pairing region comprising substitutions Q124E, V133S and S176E, with two high performing IgM Cμ4 pairing regions: HP46 comprising electrostatic substitution T477D and engineered cysteine Y455C, and HP122 comprising electrostatic substitution T477D and engineered cysteine F516C and further comprising electrostatic substitution Y455D. Columns A and B provide the SEQ ID NOs corresponding to the amino acid sequences of the light and heavy pairing regions respectively. Subsequent columns give the titer of antibody produced (in mg/L).

FIG. 45 provides Table 33, which shows testing data measuring propensity for mispairing for various chain pairings. Mispairing propensity between Cμ4-engineered kappas and CH1 pairing regions were previously observed, so a test set of variable regions was designed to include combinations that were particularly prone to unwanted association. This analysis data is shown in Table 33. Columns A and B provide the SEQ ID NOs corresponding to the amino acid sequences of the light and heavy pairing regions respectively. Subsequent columns give the titer of antibody produced (in mg/L) Above each of columns C-F is indicated the pairing regions used, first the light chain pairing region, then an underscore, then the heavy chain pairing region. Details about each pairing region, including the substitutions in each and the SEQ ID NO corresponding to the amino acid sequence of the pairing region are shown in FIG. 40 Table 28 for the light chain pairing regions and in FIG. 39 Table 27 for the heavy chain pairing regions. Rows 1-8 give titers for each combination, and row 9 gives average values for a single pairing region combination.

### DEFINITIONS

Multi-specific antibodies of the invention are typically provided in isolated form. This means that a multi-specific antibody is typically at least 50% w/w pure of interfering proteins and other contaminants arising from its production or purification including mis-paired complexes of heavy and/or light chains but does not exclude the possibility that the multi-specific antibody is combined with an excess of pharmaceutical acceptable carrier(s) or other vehicle intended to facilitate its use. Sometimes multi-specific antibodies are at least 60, 70, 80, 90, 95 or 99% w/w pure of interfering proteins and contaminants from production or purification. Often a multi-specific antibody is the predominant macromolecular species remaining after its purification.

Specific binding of multi-specific antibody to its target antigen epitope means an affinity of at least 10⁶, 10⁷, 10⁸, 10⁹, or 10¹⁰ M⁻¹. Affinities can be different for the different targets. Specific binding is detectably higher in magnitude and distinguishable from non-specific binding occurring to at least one unrelated target. Specific binding can be the result of formation of bonds between particular functional groups or particular spatial fit (e.g., lock and key type) whereas nonspecific binding is usually the result of van der Waals forces. Specific binding does not however necessarily imply that a multi-specific antibody with two different binding sites binds only against targets for these two binding sites.

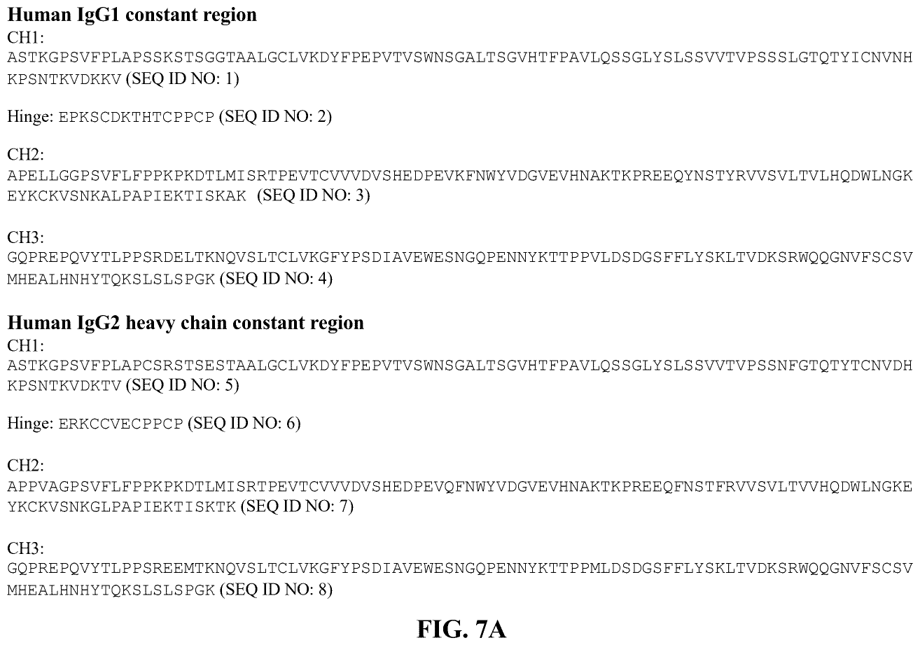

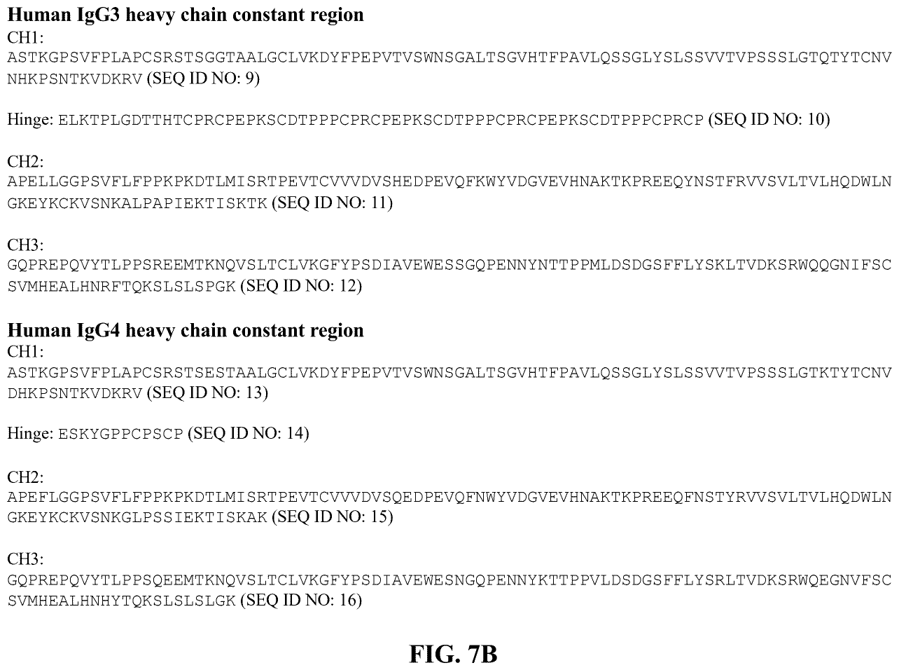

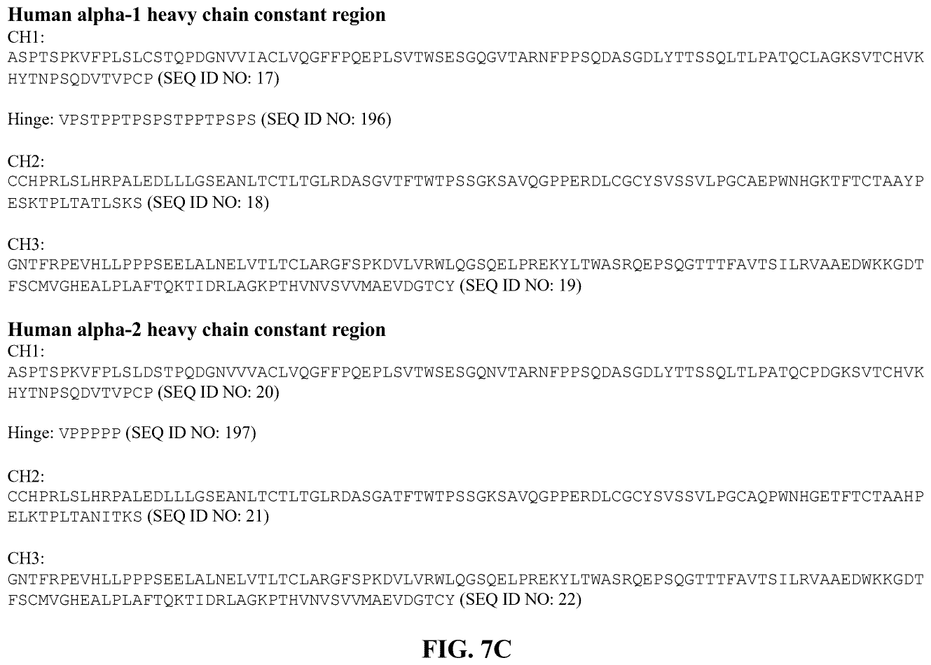

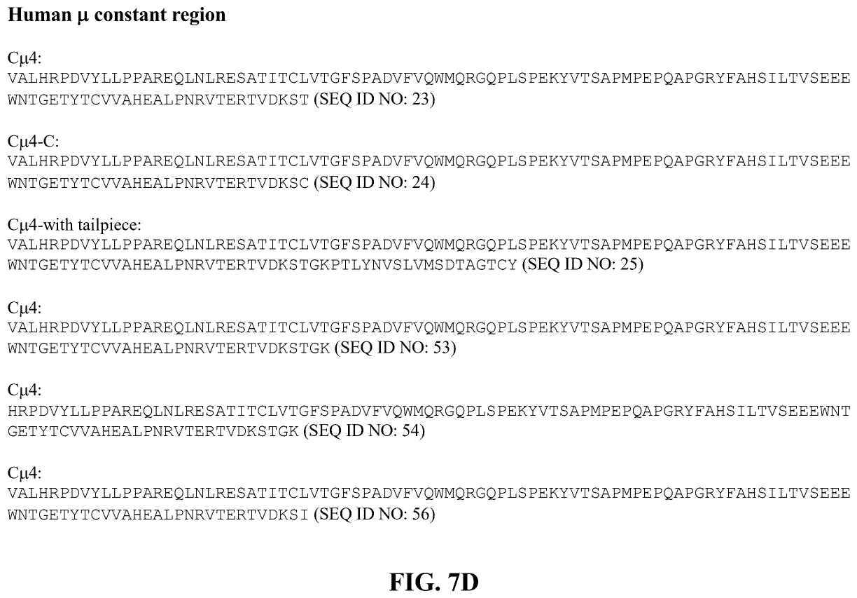

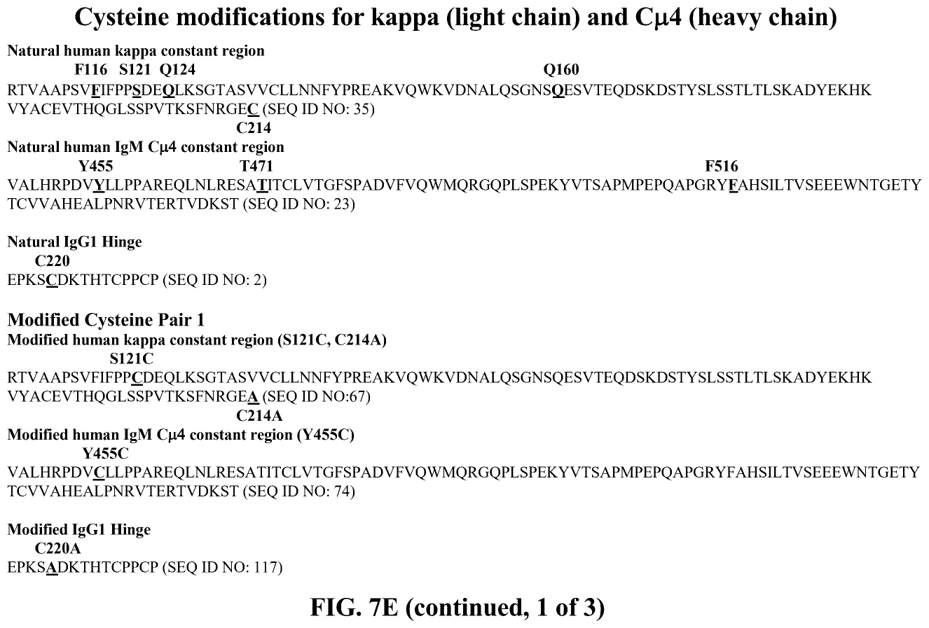

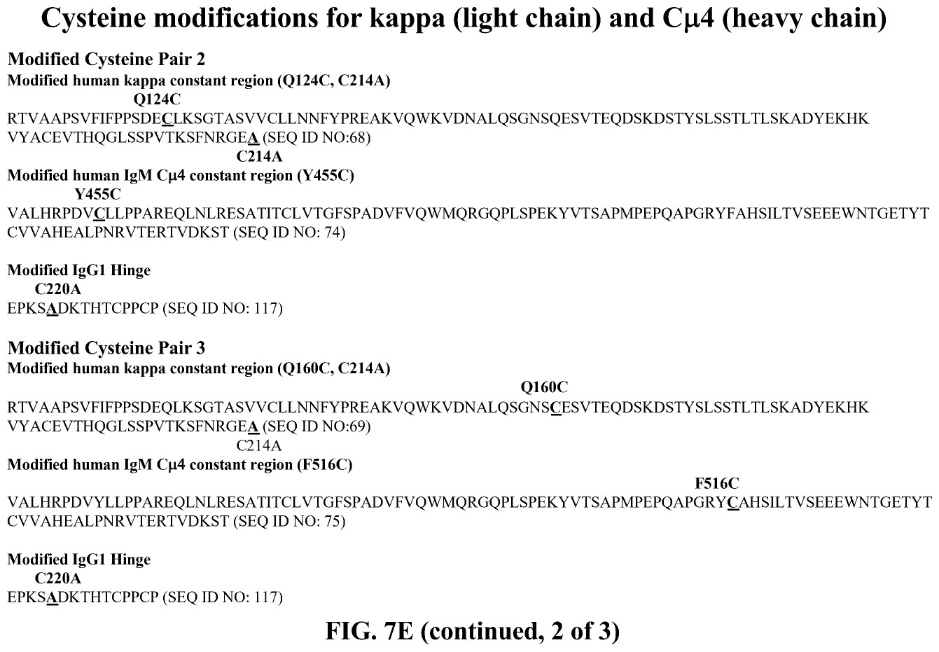

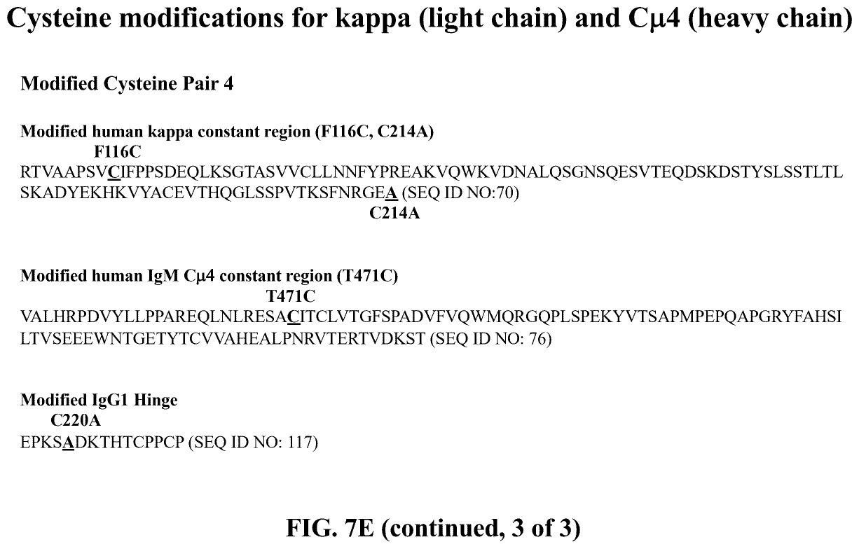

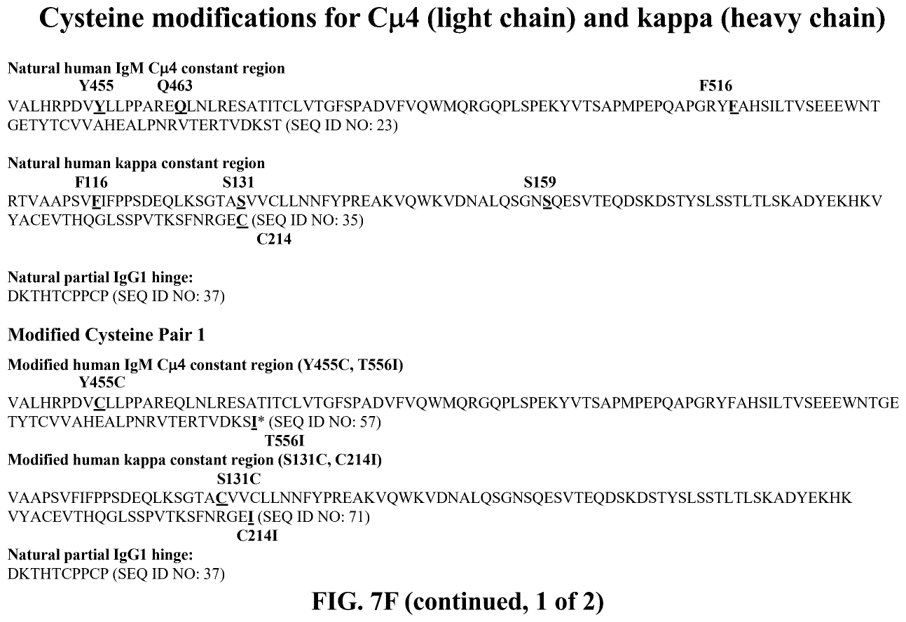

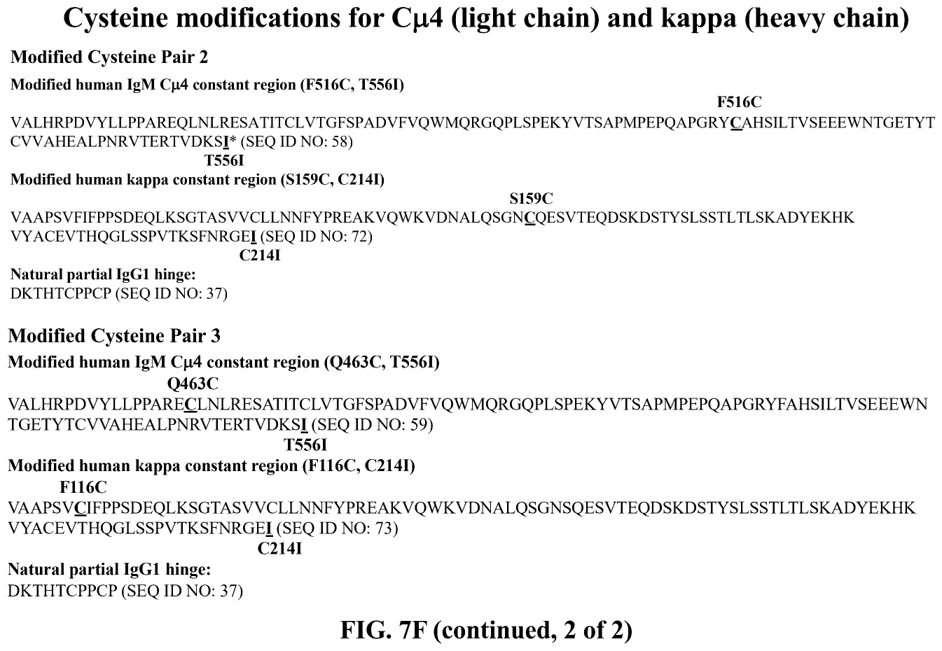

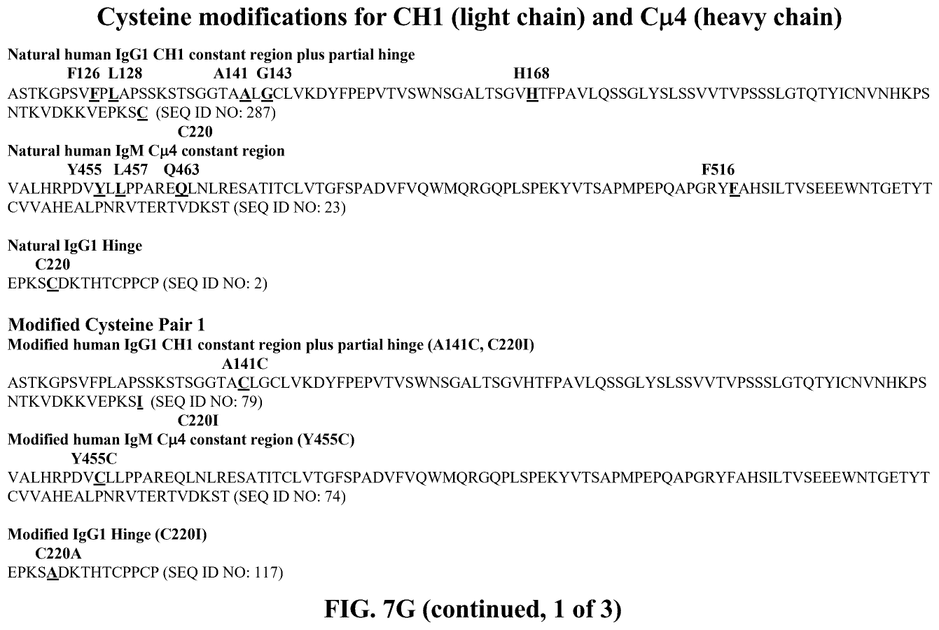

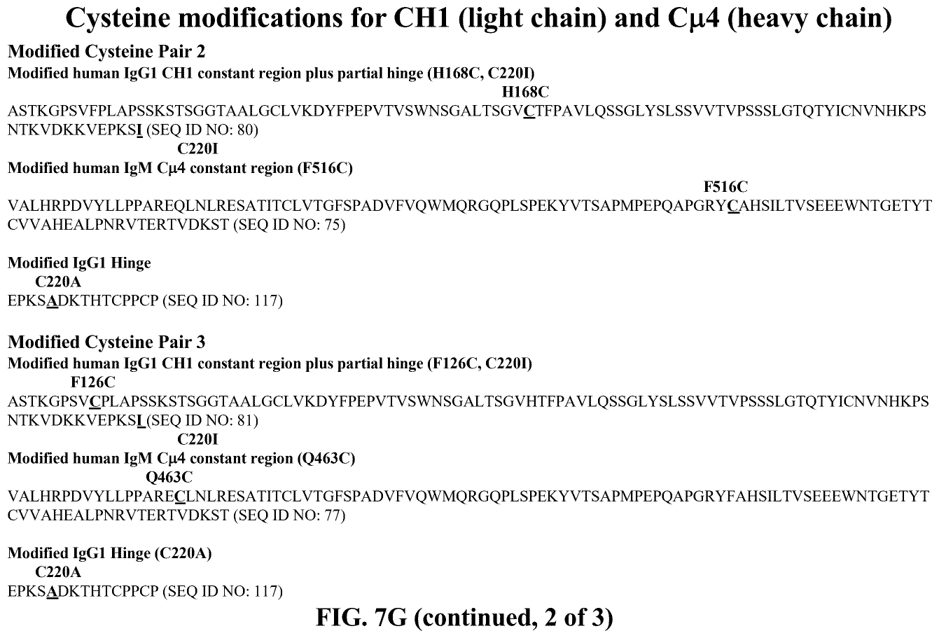

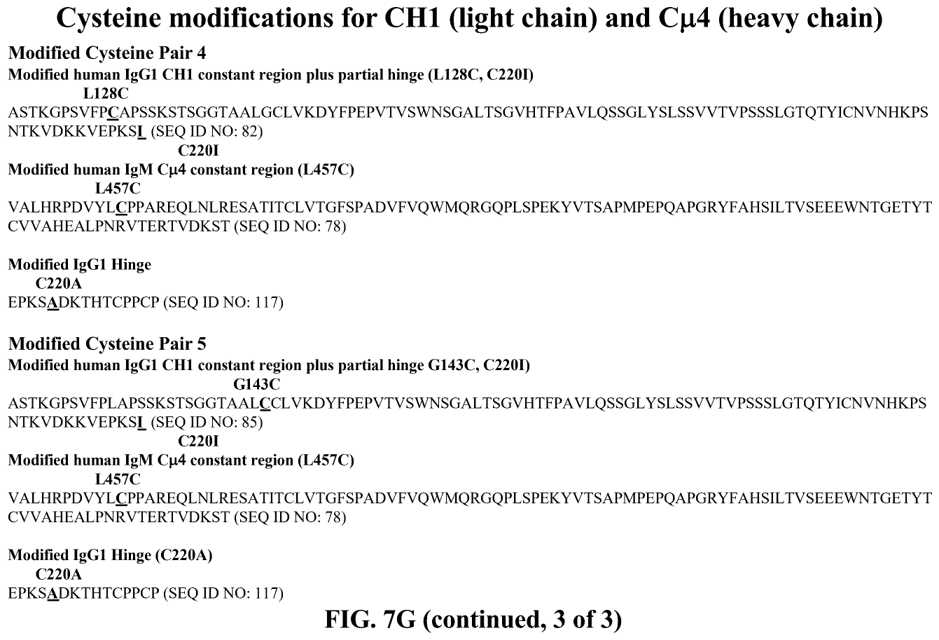

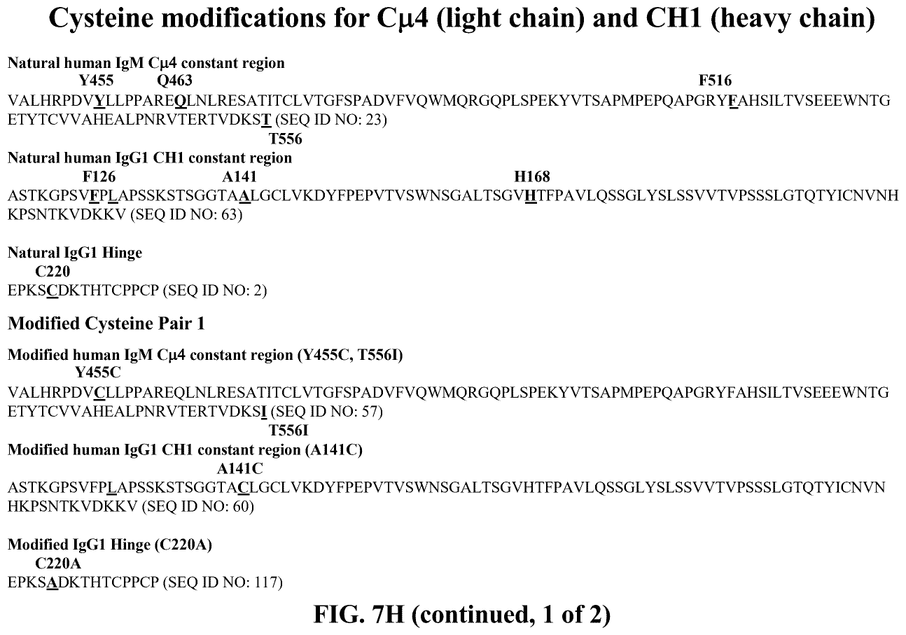

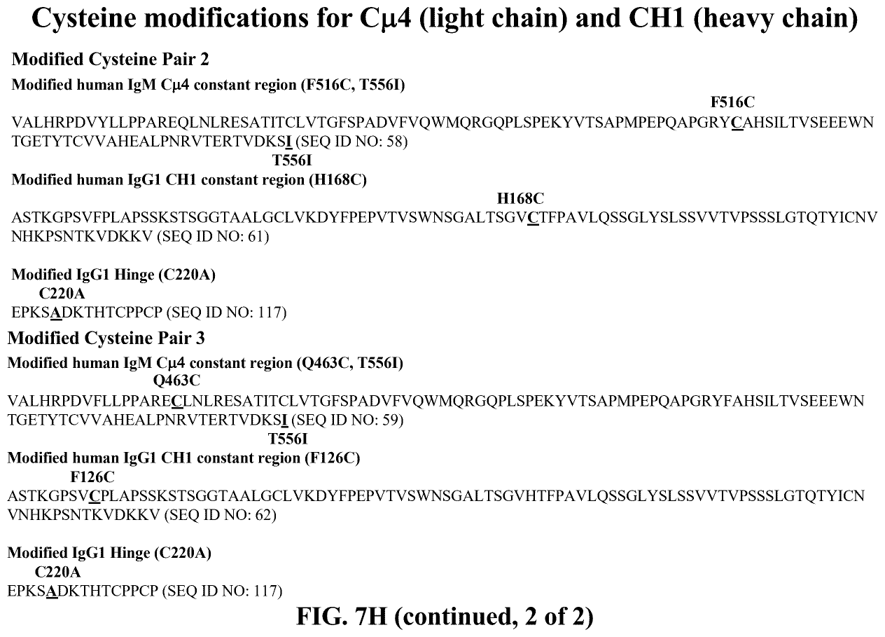

A basic antibody structural unit is a tetramer of subunits. Each tetramer includes two identical pairs of polypeptide chains, each pair having one “light” (about 25 kDa) and one “heavy” chain (about 50-70 kDa). The amino-terminal portion of each chain includes a variable region of about 100 to 110 or more amino acids primarily responsible for antigen recognition. This variable region is initially expressed linked to a cleavable signal peptide. The variable region without the signal peptide is sometimes referred to as a mature variable region. Thus, for example, a light chain mature variable region means a light chain variable region without the light chain signal peptide. However, reference to a variable region does not mean that a signal sequence is necessarily present; and in fact signal sequences are cleaved once the multi-specific antibodies of the invention have been expressed and secreted. A pair of heavy and light chain variable regions defines a binding region of an antibody. The carboxy-terminal portion of the light and heavy chains respectively defines light and heavy chain constant regions. The heavy chain constant region is primarily responsible for effector function. In IgG antibodies, the heavy chain constant region is divided into CH1, hinge, CH2, and CH3 regions. For IgA, a demarcation between CH1, hinge, CH2 and CH3 is shown in FIG. 7C. The CH1 region pairs with the light chain constant region by disulfide and noncovalent bonding. The hinge region provides flexibility between the binding and effector regions of an antibody and the upper part of the CH2 region provides sites for intermolecular disulfide bonding between the two heavy chain constant regions in a tetramer subunit. The CH2 and CH3 regions are the primary site of effector functions and FcRn binding.

Light chains are classified as either kappa or lambda. Heavy chains are classified as gamma, mu, alpha, delta, or epsilon, and define the antibody's isotype as IgG, IgM, IgA, IgD and IgE, respectively. Within light and heavy chains, the variable and constant regions are joined by a “J” segment of about 12 or more amino acids, with the heavy chain also including a “D” segment of about 10 or more amino acids. (See generally, Fundamental Immunology (Paul, W., ed., 2nd ed. Raven Press, N.Y., 1989), Ch. 7) (incorporated by reference in its entirety for all purposes).

The mature variable regions of each light/heavy chain pair form the antibody binding site. Thus, an intact antibody has two binding sites, i.e., is divalent. In natural antibodies, the binding sites are the same. However, in multi-specific antibodies, these binding sites can be the same or different depending on the format (see, e.g., Songsivilai and Lachmann, Clin. Exp. Immunol., 79:315-321 (1990); Kostelny et al., J. Immunol., 148:1547-53 (1992)). The variable regions all exhibit the same general structure of relatively conserved framework regions (FR) joined by three hypervariable regions, also called complementarity determining regions or CDRs. The CDRs from the two chains of each pair are aligned by the framework regions, enabling binding to a specific epitope. From N-terminal to C-terminal, both light and heavy chains comprise the regions FR1, CDR1, FR2, CDR2, FR3, CDR3 and FR4. The assignment of amino acids to each region is in accordance with the definitions of Kabat, Sequences of Proteins of Immunological Interest (National Institutes of Health, Bethesda, Md., 1987 and 1991), or Chothia & Lesk, J. Mol. Biol. 196:901-917 (1987); Chothia et al., Nature 342:878-883 (1989), composite Kabat Chothia, Abm, Contact or IMGT.

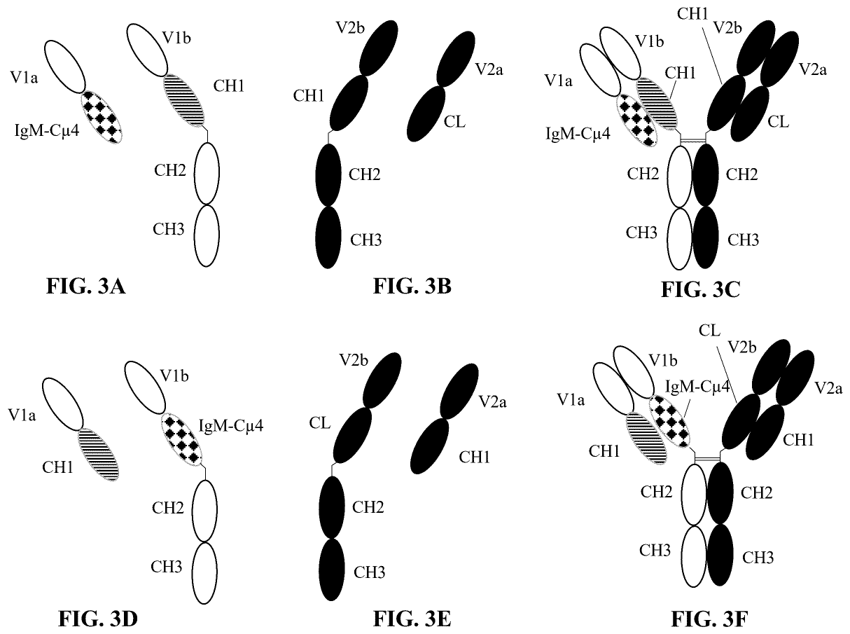

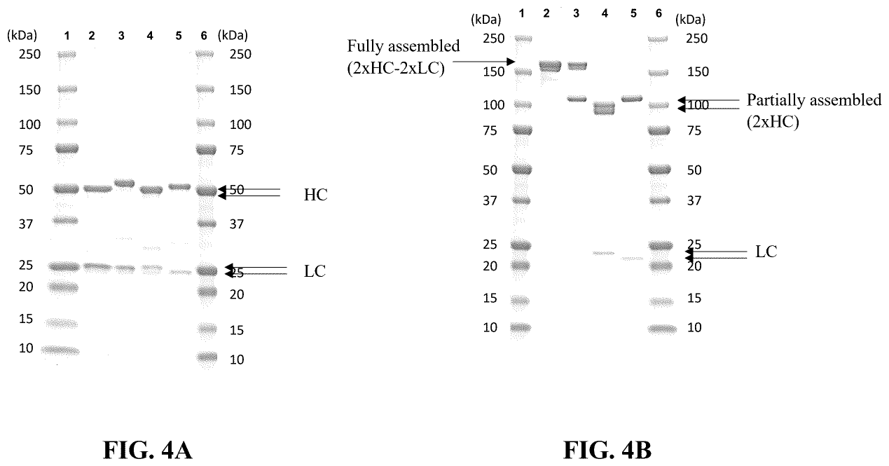

Kabat also provides a widely used numbering convention (Kabat numbering) in which corresponding residues between different heavy chain variable regions or between different light chain variable regions are assigned the same number. Kabat numbering can also be used for antibody constant regions. The EU index (also called EU numbering) is more commonly used for heavy chain CH1, hinge, CH2 and CH3 regions. In this application, EU numbering is used for CH1, hinge, CH2 and CH3 regions and Kabat numbering for variable regions, light chain constant regions and Cμ4 unless indicated otherwise. The numbering used for the IgM Cμ4 region is taken from Keyt et aL. (2020) “Structure, Function and Therapeutic Use of IgM Antibodies”, Antibodies 9: 53, shown in FIG. 3 in that publication and shown in FIG. 14 Table 2 and is that of Kabat Residues in an antibody chain can also be numbered based on alignment with an antibody sequence provided in the present application with the aligned residues being assigned the same number. For example, on maximal alignment of respective sequences being compared, the residue in an antibody sequence aligning with the position Y455 in Cμ4 as shown in FIG. 7E is also assigned position 455. Similarly, the residue in an antibody sequence aligning with residue C220 in the IgG1 hinge as shown in FIG. 7E is also assigned as position 220.

A multi-specific antibody has at least two different binding sites. A bispecific antibody has two different binding sites. Any reference to a multi-specific antibody should be understood as including reference to a bispecific antibody.

As used herein, the term “antibody” is used in the broadest sense to include antibodies comprising full heavy and light chain configurations, but also include antigen-binding fragments, immunospecific fragments, variants, or derivatives thereof, which are all encompassed within the teaching and spirit of the present disclosure. In this spirit, and as used herein and well known to one of ordinary skill in the art, the term “antibody” encompasses, but is not limited to, single chain antibodies, epitope-binding fragments, e.g., Fab, Fab′ and F(ab′)2, Fd, Fvs, single-chain Fvs (scFv), single-chain antibodies, disulfide-linked Fvs (sdFv), fragments comprising either a VL or VH domain. Antibody fragments, including single-chain antibodies, may comprise the variable region(s) alone or in combination with the entirety or a portion of the following: hinge region, CH1, CH2, and CH3 domains. Any antibody as described herein can be adapted to any of these antibody formats listed above. Bispecific single chain antibodies are also known in the art.

Single chain antibody formats in particular, including single chain bispecific antibody formats, find use with the antibodies described herein. As one of skill will recognize, existing methods for the manufacture of single chain antibodies can be readily adapted to manufacture the antibodies described herein as single chain antibodies. Common linkers for the manufacture of single-chain antibodies are known, and are generally flexible, often based on repetitions of the pentapeptide linker (Gly4-Ser), for example with lengths between 15 and 20 amino acids being typical. Other examples of suitable linkers include non-repetitive, modified, or functionalized linkers tailored to specific antibody applications, where the linkers chosen can be selected and optimized to improve antibody stability, solubility, and/or biological activity depending on the intended use of the antibody.

In one aspect of the manufacture of single chain antibodies as described in the present disclosure, an antibody of the invention can be a Fab-type antibody comprising (i) a first chain comprising a first variable region and a first pairing region, and (ii) a second chain comprising a second variable region and a second pairing region, where the first chain and second chain pair to each other via association of the first and second pairing regions. In this example, the first chain and second chain can be produced as a single contiguous polypeptide with suitable linkers, as known in the art. Two compatible Fab molecules can both be manufactured as single chain antibodies, and can be used to produce a divalent bispecific antibody.

The term “epitope” refers to a site on an antigen to which an arm of a multi-specific antibody binds. An epitope can be formed from contiguous amino acids or noncontiguous amino acids juxtaposed by tertiary folding of one or more proteins. Epitopes formed from contiguous amino acids (also known as linear epitopes) are typically retained on exposure to denaturing solvents whereas epitopes formed by tertiary folding (also known as conformational epitopes) are typically lost on treatment with denaturing solvents. Some antibodies bind to an end-specific epitope, meaning an antibody binds preferentially to a polypeptide with a free end relative to the same polypeptide fused to another polypeptide resulting in loss of the free end. An epitope typically includes at least 3, and more usually, at least 5 or 8-10 amino acids in a unique spatial conformation. Methods of determining spatial conformation of epitopes include, for example, x-ray crystallography and 2-dimensional nuclear magnetic resonance. See, e.g., Epitope Mapping Protocols, in Methods in Molecular Biology, Vol. 66, Glenn E. Morris, Ed. (1996).

The term “antigen” or “target antigen” indicates a target molecule bound by one binding site of a multi-specific antibody. An antigen may be a protein of any length (natural, synthetic or recombinantly expressed), a nucleic acid or carbohydrate among other molecules. Antigens include receptors, ligands, counter receptors, and coat proteins. Antigens can possess a plurality of epitope sites which are recognized by different antibody binding sites.

Antibodies that recognize the same or overlapping epitopes can be identified in a simple immunoassay showing the ability of one antibody to compete with the binding of another antibody to a target antigen. The epitope of an antibody can also be defined by X-ray crystallography of the antibody bound to its antigen to identify contact residues. Alternatively, two antibodies have the same epitope if all amino acid mutations in the antigen that reduce or eliminate binding of one antibody reduce or eliminate binding of the other. Two antibodies have overlapping epitopes if some amino acid mutations that reduce or eliminate binding of one antibody reduce or eliminate binding of the other.

Competition between antibodies is determined by an assay in which an antibody under test inhibits specific binding of a reference antibody to a common antigen (see, e.g., Junghans et al., Cancer Res. 50:1495, 1990). A test antibody competes with a reference antibody if an excess of a test antibody (e.g., at least 2 times, 5 times, 10 times, 20 times or 100 times) inhibits binding of the reference antibody by at least 50% but preferably 75%, 90% or 99% as measured in a competitive binding assay. Antibodies identified by competition assay (competing antibodies) include antibodies binding to the same epitope as the reference antibody and antibodies binding to an adjacent epitope sufficiently proximal to the epitope bound by the reference antibody for steric hindrance to occur.

The term “subject” includes human and other mammalian subjects that receive either prophylactic or therapeutic treatment. Other mammalian subjects include animal models of a human condition (e.g., rodent, non-human primate) and veterinary subjects.

For purposes of classifying amino acids substitutions as conservative or nonconservative, amino acids are grouped as follows:

| | |
|---|---|
| Group I (hydrophobic side chains) | Met (M), Ala (A), Val (V), |
| | Leu (L), Ile (I) |
| Group II (neutral hydrophilic side chains) | Cys (C), Ser (S), Thr (T) |
| Group III (acidic side chains) | Asp (D), Glu (E) |
| Group IV (basic side chains) | Asn (N), Gln (Q), His (H), |
| | Lys (K), Arg (R) |
| Group V (residues influencing | Gly (G), Pro (P) |
| chain orientation) | |
| Group VI (aromatic side chains) | Trp (W), Tyr (Y), Phe (F) |

Conservative substitutions involve substitutions between amino acids in the same class. Non-conservative substitutions constitute exchanging a member of one of these classes for a member of another.

Percentage sequence identities are determined with antibody sequences maximally aligned by the EU numbering for CH1, hinge, CH2 and CH3 region and Kabat numbering for variable regions, light chain constant regions and Cμ4. For sequences that cannot be aligned by Kabat or EU numbering conventions, EMBL-EBI EMBOSS Needle pairwise alignment can be used with default parameters. After alignment, if a subject antibody region (e.g., the entire mature variable region of a heavy or light chain) is being compared with the same region of a reference antibody, the percentage sequence identity between the subject and reference antibody regions is the number of positions occupied by the same amino acid in both the subject and reference antibody region divided by the total number of aligned positions of the two regions, with gaps not counted, multiplied by 100 to convert to percentage.

Compositions or methods “comprising” one or more recited elements may include other elements not specifically recited. For example, a composition that comprises antibody may contain the antibody alone or in combination with other ingredients.

The term “antibody-dependent cellular cytotoxicity,” or ADCC, is a mechanism for inducing cell death that depends upon the interaction of antibody-coated target cells (i.e., cells with bound antibody) with immune cells possessing lytic activity (also referred to as effector cells, e.g., immune effector cells). Such effector cells include natural killer cells, monocytes/macrophages and neutrophils. ADCC is triggered by interactions between the Fc region of an antibody bound to a cell and Fcγ receptors, particularly FcγRI and FcγRIII, on immune effector cells such as neutrophils, macrophages and natural killer cells. The target cell is eliminated by phagocytosis or lysis, depending on the type of mediating effector cell. Death of the antibody-coated target cell occurs as a result of effector cell activity.

The term opsonization also known as “antibody-dependent cellular phagocytosis”, or ADCP, refers to the process by which antibody-coated cells are internalized, either in whole or in part, by phagocytic immune cells (e.g., macrophages, neutrophils and dendritic cells) that bind to an immunoglobulin Fc region.

The term “complement-dependent cytotoxicity” or CDC (also called CMC) refers to a mechanism for inducing cell death in which an Fc effector region(s) of a target-bound antibody activates a series of enzymatic reactions culminating in the formation of holes in the target cell membrane. Typically, antigen-antibody complexes such as those on antibody-coated target cells bind and activate complement component C1q which in turn activates the complement cascade leading to target cell death. Activation of complement may also result in deposition of complement components on the target cell surface that facilitate ADCC by binding complement receptors (e.g., CR3) on leukocytes.

The multi-specific antibodies are formed from pairs of heavy and light chain variable regions from component antibodies. The component antibodies can be rodent, chimeric, veneered, humanized, primatized, primate or human among others. The component antibodies can be of the same or different types; for example, one can be humanized and the other human.

The production of other non-human monoclonal antibodies, e.g., murine, guinea pig, primate, rabbit or rat, against an antigen can be accomplished by, for example, immunizing the animal with the antigen or a fragment thereof, or cells bearing the antigen. See Harlow & Lane, Antibodies, A Laboratory Manual (CSHP NY, 1988) (incorporated by reference for all purposes). Such an antigen can be obtained from a natural source, by peptide synthesis or by recombinant expression. Optionally, the antigen can be administered fused or otherwise complexed with a carrier protein. Optionally, the antigen can be administered with an adjuvant. Several types of adjuvant can be used as described below. Complete Freund's adjuvant followed by incomplete adjuvant is preferred for immunization of laboratory animals.

A humanized antibody is a genetically engineered antibody in which the CDRs from a non-human “donor” antibody are grafted into human “acceptor” antibody sequences (see, e.g., Queen, U.S. Pat. Nos. 5,530,101 and 5,585,089; Winter, U.S. Pat. No. 5,225,539, Carter, U.S. Pat. No. 6,407,213, Adair, U.S. Pat. Nos. 5,859,205 and 6,881,557, Foote, U.S. Pat. No. 6,881,557). The acceptor antibody sequences can be, for example, a mature human antibody sequence, a composite of such sequences, a consensus sequence of human antibody sequences, or a germline region sequence. Thus, a humanized antibody is an antibody having some or all CDRs entirely or substantially from a donor antibody and variable region framework sequences and constant regions, if present, entirely or substantially from human antibody sequences. Similarly, a humanized heavy chain has at least one, two and usually all three CDRs entirely or substantially from a donor antibody heavy chain, and a heavy chain variable region framework sequence and heavy chain constant region, if present, substantially from human heavy chain variable region framework and constant region sequences. Similarly, a humanized light chain has at least one, two and usually all three CDRs entirely or substantially from a donor antibody light chain, and a light chain variable region framework sequence and light chain constant region, if present, substantially from human light chain variable region framework and constant region sequences. Other than nanobodies and dAbs, a humanized antibody comprises a humanized heavy chain and a humanized light chain. A CDR in a humanized antibody is substantially from a corresponding CDR in a non-human antibody when at least 85%, 90%, 95% or 100% of corresponding residues (as defined by Kabat) are identical between the respective CDRs. The variable region framework sequences of an antibody chain or the constant region of an antibody chain are substantially from a human variable region framework sequence or human constant region respectively when at least 85%, 90%, 95% or 100% of corresponding residues defined by Kabat are identical.

Although humanized antibodies often incorporate all six CDRs (preferably as defined by Kabat) from a mouse antibody, they can also be made with less than all CDRs (e.g., at least 3, 4, or 5 CDRs from a mouse antibody) (e.g., Pascalis et al., J. Immunol. 169:3076, 2002; Vajdos et al., Journal of Molecular Biology, 320: 415-428, 2002; Iwahashi et al., Mol. Immunol. 36:1079-1091, 1999; Tamura et al, Journal of Immunology, 164:1432-1441, 2000).

A chimeric antibody is an antibody in which the mature variable regions of light and heavy chains of a non-human antibody (e.g., a mouse) are combined with human light and heavy chain constant regions. Such antibodies substantially or entirely retain the binding specificity of the mouse antibody and are about two-thirds human sequence.

A veneered antibody is a type of humanized antibody that retains some and usually all of the CDRs and some of the non-human variable region framework residues of a non-human antibody but replaces other variable region framework residues that may contribute to B- or T-cell epitopes, for example exposed residues (Padlan, Mol. Immunol. 28:489, 1991) with residues from the corresponding positions of a human antibody sequence. The result is an antibody in which the CDRs are entirely or substantially from a non-human antibody and the variable region frameworks of the non-human antibody are made more human-like by the substitutions.

A human antibody can be isolated from a human, or otherwise result from expression of human immunoglobulin genes (e.g., in a transgenic mouse, in vitro or by phage display). Methods for producing human antibodies include the trioma method of Oestberg et al., Hybridoma 2:361-367 (1983); Oestberg, U.S. Pat. No. 4,634,664; and Engleman et al., U.S. Pat. No. 4,634,666, use of transgenic mice including human immunoglobulin genes (see, e.g., Lonberg et al., WO93/12227 (1993); U.S. Pat. Nos. 5,877,397, 5,874,299, 5,814,318, 5,789,650, 5,770,429, 5,661,016, 5,633,425, 5,625,126, 5,569,825, 5,545,806, Nature 148, 1547-1553 (1994), Nature Biotechnology 14, 826 (1996), Kucherlapati, WO 91/10741 (1991)) and phage display methods (see, e.g. Dower et al., WO 91/17271 and McCafferty et al., WO 92/01047, U.S. Pat. Nos. 5,877,218, 5,871,907, 5,858,657, 5,837,242, 5,733,743 and 5,565,332).

Antibodies can be screened for specific binding to the antigen. Antibodies may be further screened for binding to a specific region of the antigen, competition with a reference antibody, agonism or antagonism of cells bearing the antigen. Non-human antibodies can be converted to chimeric, veneered or humanized forms as described above.

As used herein, the term “orthogonal” or “orthogonal system” or “orthogonal component” or the like refers generally to two or more biomolecules that are able to function independently of each other, without interfering with or being affected by each other's operations or substrates, and without causing unintended cross-talk or interference with one another. In some aspects, a multispecific antibody of the present disclosure comprises first, second, third and fourth chains such that the first and second chains preferentially associate with each other, and the third and fourth chains associate preferentially with each other: the pairing of the first and second chains is orthogonal to the pairing of the third and fourth chains, with each orthogonal pair of chains producing a monovalent binding site with defined specificity. The antibody comprising orthogonal pairing regions is produced in a host cell using the host cell transcription and translation machinery for its manufacture. In one aspect, a core feature of the multispecific antibody is the design and engineering of multiple orthogonal pairing regions that have the ability to specifically self assemble. Through this programmed interaction, in some aspects, preferred heavy chain and light chain orthogonal pairings are made, resulting in, for example, multispecific, for example, bispecific, antibody molecules. These programmed interactions allow preferred heavy/light chain pairings to create desired bispecific antibody molecules, and deter unwanted heavy/light mispairings.

Before describing the invention in detail, it is to be understood that this invention is not limited to particular biological systems or reagents, for example, particular host cells, which can, of course, vary. It is also to be understood that the terminology used herein is for the purpose of describing particular aspects only, and is not intended to be limiting. As used in this specification and the appended claims, the singular forms “a”, “an” and “the” include plural referents unless the content clearly dictates otherwise. Thus, for example, reference to “an antibody” includes, as a practical matter, many copies of that antibody.

Unless defined herein and below in the reminder of the specification, all technical and scientific terms used herein have the same meaning as commonly understood by one of ordinary skill in the art to which the invention pertains.

### DETAILED DESCRIPTION

### 1. General

The disclosure provides multi-specific antibodies including two different pairs of heavy and light chains, in which pairing of heavy and light chains is promoted by inclusion of an IgM Cμ4 region.

The present disclosure provides discussion of mechanistic theories explaining in vivo phenomena, including the assembly of multispecific, for example, bispecific antibodies. However, it is not intended that the invention be limited in any regard to a proposed molecular mechanism of action, and knowledge of such mechanisms is not required to make or use any invention described herein.

In a first aspect of the invention, the IgM Cμ4 region replaces a CH1 constant region in one of the pairings, where it pairs with a kappa light chain constant region. The other pairing can have conventional CH1 and light chain constant regions. The IgM Cμ4 region associates with the kappa light chain constant region in the first pairing and CH1 and light chain constant regions associate with each other in the other pairing.

In a second aspect of the invention, the IgM Cμ4 region replaces a light chain region in one of the pairings, where it pairs with a CH1 constant region. The other pairing can have conventional CH1 and light chain constant regions. The IgM Cμ4 region associates with the CH1 constant region in the first pairing and CH1 and light chain constant regions associate with each other in the other pairing.

Because the IgM Cμ4 region can pair with either a kappa light chain or with a CH1 constant region, the sequences of the IgM Cμ4 region and the either kappa light chain constant region or CH1 constant region to which it pairs can be modified to promote specific pairing between desired pairs of partners and inhibit non-specific binding that can lead to mispairing. This can be done in the first instance by mutation of the cysteine residues that normally covalently link the IgM Cμ4 region to the kappa or to the CH1, and then by introduction of new cysteine residues into the IgM Cμ4 region and into the kappa or the CH1 to which the IgM Cμ4 region should pair. Additional mutations may optionally be introduced into one or both of the pairing regions to improve expression and/or packing of the pairing regions with each other.

In some instances, inclusion of the IgM Cμ4 region facilitates separation of desired heterodimers from undesired homodimers following expression and assembly of the two pairings. Incorporation of IgM Cμ4 is compatible with various cross-over formats, such as exchange of heavy and light chain variable regions in one of the pairings, which promote correct pairing of heavy and light chains variable regions over incorrect pairing. The resulting multi-specific antibodies can assemble from expression of four chains in the same cell. Such antibodies can have a similar tetrameric shape as natural antibodies including two paired heavy and light chain variable regions forming two binding sites.

Thus, the disclosure provides a multi-specific antibody including first and second binding sites. The first binding site includes the following components. A first chain comprising a first variable region, a first pairing region and first IgG or IgA CH2 and CH3 region. A second chain comprising a second variable region, and a second pairing region. The first and second variable regions are heavy and light chain variable regions or vice versa. The first and second pairing regions are (a) an IgM Cμ4 region and (b) a kappa light chain constant region or a first IgG or IgA CH1 region; or vice versa. The first chain and second chain are paired via association of the first and second pairing regions forming a binding site for a first target antigen.

The second binding site includes the following components. A third chain comprising a third variable region, a third pairing region, and second IgG or IgA CH2 and CH3 regions. A fourth chain comprising a fourth variable region and a fourth pairing region. The third and fourth variable regions are heavy and light chain variable regions or vice versa. The third and fourth pairing regions are a second IgG or IgA CH1 constant region and a second light chain constant region or vice versa. The third and fourth chains are paired via association of the third and fourth pairing regions forming a binding site for a second target antigen. The paired first and second chains and the paired third and fourth chains are associated via the first and second IgG or IgA CH3 regions thereby forming a tetramer.

The first and second pairing regions can each include an engineered cysteine residue, which form a disulfide bond with one another, promoting pairing of the first and second chains. When the first and second pairing regions are (a) the IgM Cμ4 region and (b) the kappa light chain constant region respectively, combinations of engineered cysteines include an engineered cysteine at position 455 of the IgM Cμ4 region and at position 121, 124 or 131 of the kappa light chain constant region, or an engineered cysteine at position 516 of the IgM Cμ4 region and position 160 of the kappa light chain constant region, or an engineered cysteine at position 471 of the IgM Cμ4 region and position 116 of the kappa light chain constant region, or an engineered cysteine at position 463 of the IgM Cμ4 region and position 116 of the kappa light chain constant region, positions being numbered by Kabat numbering.

When the first and second pairing regions are the kappa light chain constant region and the IgM Cμ4 region respectively, combinations of engineered cysteines include an engineered cysteine at position 455 in the IgM Cμ4 region, and the kappa light chain constant region includes an engineered cysteine at position 131, or the IgM Cμ4 region includes an engineered cysteine at position 516, and the kappa light chain constant region includes an engineered cysteine at position 159, or the IgM Cμ4 region includes an engineered cysteine at position 463 and the kappa light chain constant region includes an engineered cysteine at position 116, positions being numbered by Kabat numbering.

When the first and second pairing regions are the IgM Cμ4 region and the first IgG or IgA CH1 region respectively, combinations of engineered cysteines include an engineered cysteine at position 455 of the IgM Cμ4 region and position 141 of the first CH1 region, or position 516 of the IgM Cμ4 region and position 168 of the first CH1 region, or position 463 of the IgM Cμ4 region and position 126 of the first CH1 region, or position 457 of the IgM Cμ4 region and position 128 or 143 of the first CH1 region, positions in Cμ4 being numbered by Kabat numbering and positions in CH1 by EU numbering.

When the first and second pairing regions are (a) the first IgG or IgA CH1 region and (b) the IgM Cμ4 region respectively, combinations of engineered cysteines include an engineered cysteine at position 455 of the IgM Cμ4 region and position 141 of the first CH1 region, or position 516 of the IgM Cμ4 region and position 168 of the first CH1 region, or position 463 of the IgM Cμ4 region and position 126 of the first CH1 region, positions in Cμ4 being numbered by Kabat numbering and positions in CH1 by EU numbering.

The second pairing region can have a naturally present cysteine substituted or deleted to prevent disulfide bonding of the second pairing region to the third or fourth chains. When the second pairing region is the kappa light chain, the naturally present cysteine can be at the C-terminal position (Kabat position 214). When the second pairing region is IgM Cμ4, the naturally occurring cysteine can be at or before 556 by Kabat numbering. When the second pairing region is the IgG1 CH1 region and the C-terminus of the CH1 region is linked to an N-terminal hinge segment, the naturally occurring cysteine can be at position 220 by EU numbering of the N-terminal hinge segment. When the second pairing region is the CH1 region of human IgG2, IgG3 or IgG4, the naturally occurring cysteine can be at position 131 by EU numbering of the CH1 region.

When the third and fourth pairing regions are the second IgG or IgA CH1 constant region and the second light chain constant region, and the first chain comprises a CH1 region, at least a portion of a hinge and CH2 and CH3 regions, each of human IgG1 isotype, a cysteine residue at EU position 220 of the at least a portion of a hinge of the first chain is mutated or deleted to prevent disulfide bonding with the second light chain constant region. When the third and fourth pairing regions are the second IgG or IgA CH1 constant region, and the second light chain constant region, and the first chain comprises a CH1 region, at least a portion of a hinge and CH2 and CH3 regions, each of human IgG2, 3, or 4 isotype, a cysteine residue at EU position 131 of the CH1 region of the first pairing region can be mutated or deleted to prevent disulfide bonding with the second light chain constant region.

II. Multi-Specific Antibody Components with IgM Cμ4—Kappa Pairing

The multi-specific antibodies include two pairs of chains. Each pair includes a pair of heavy and light chain variable regions forming a binding site. The chains of the first pair can be referred to as first and second chains. The chains of the second pair can be referred to as third and fourth chains. The first and second pairs are sometimes each referred to as being a half-antibody because they each contribute one binding site to a bispecific antibody with two binding sites. The first chain comprises a first variable region, a first pairing region an optional hinge region, or portion thereof, and IgG or IgA CH2 and CH3 regions. The second chain comprises a second variable region, and a second pairing region. The first and second variable regions are heavy and light chain variable regions, which together form a first binding site. The first chain can include the heavy chain variable region or the light chain variable region. The second chain includes whichever of the heavy chain and light chain variable region is not included in the first chain. The pairing regions are so named because they mediate pairwise association between the first and second chains. The first and second pairing regions are an IgM Cμ4 region and a kappa light chain constant region or vice versa. In other words, if the first pairing region is the IgM Cμ4 region, the second pairing region is the kappa light chain constant region and if the first pairing region is the kappa light chain constant region, the second pairing region is the IgM Cμ4 region. The first chain and second chain are paired via association of the IgM Cμ4 region and kappa light chain constant region forming a binding site for a first target antigen.

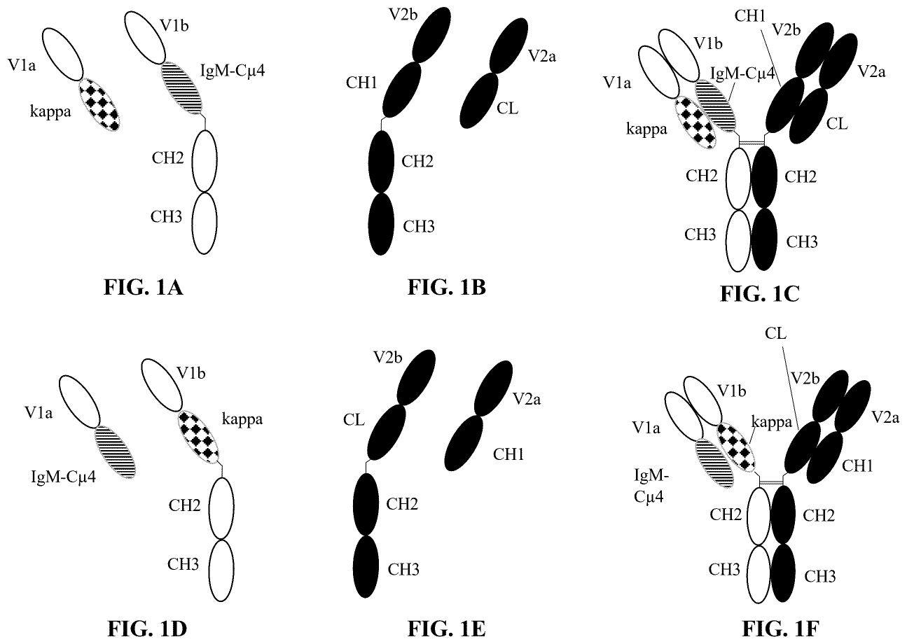

FIGS. 1A-1F represent this format for a bispecific antibody. In FIG. 1A the CH1 region of the first chain of a first half-antibody is replaced by an IgM Cμ4 region. Pairing is maintained between the variable regions (V1a and V1b) because of pairing between these variable regions and between the kappa light chain constant region and the IgM Cμ4 region. In FIG. 1B a second half-antibody has the natural organization with a heavy chain pairing to a light chain by interactions between the CH1 region and the light chain constant region (CL). Heterodimers are formed between the two half-antibodies (FIG. 1C). The kappa light chain constant region of the first half pair may be in the second chain (FIG. 1A), or it may be in the first chain that further comprises an optional hinge region, or portion thereof, and IgG or IgA CH2 and CH3 regions with the CH1 constant region being in the light chain (FIG. 1D). Similarly, the light chain constant region of the second half pair may be in the fourth chain (FIG. 1B), or it may be in the third chain that further comprises an optional hinge region, or portion thereof, and IgG or IgA CH2 and CH3 regions with the CH1 constant region being in the light chain (FIG. 1E). The configuration for the first pair shown in either FIG. 1A or FIG. 1D may be combined with that shown for the second pair in either FIG. 1B or FIG. 1E. Combinations shown in FIG. 1C and FIG. 1F are shown as examples only, and not as limitations. The variable regions are labelled V1a, V1b, V2a and V2b because the variable regions that were originally associated with a heavy or a light chain may be switched. Thus, the variable region originally associated with a light chain constant region in a natural antibody format may be placed at either V1a or V1b. Similarly, the variable region originally associated with a heavy chain constant region in a natural antibody format may be placed at either V1a or V1b, so long as variable regions V1a and V1b are complementary. The same is true of variable regions V2a and V2b.

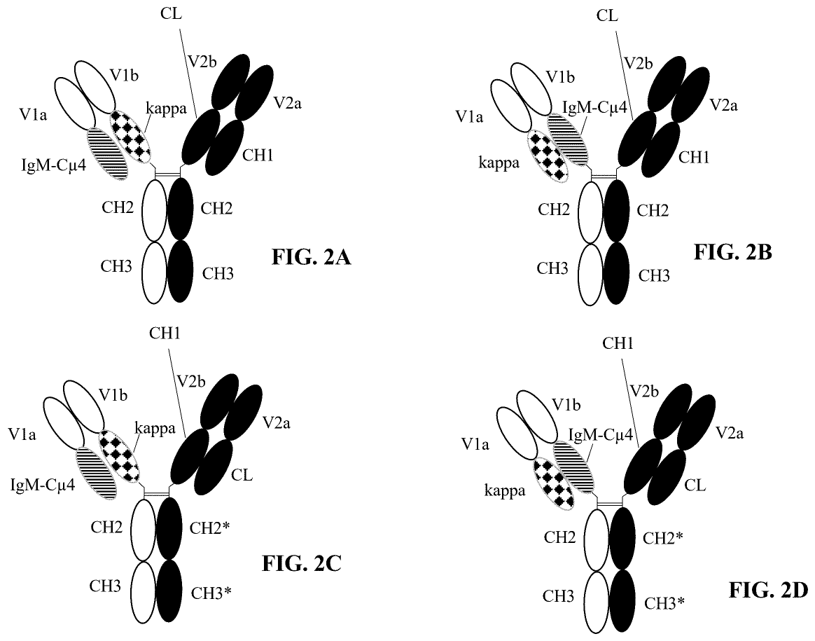

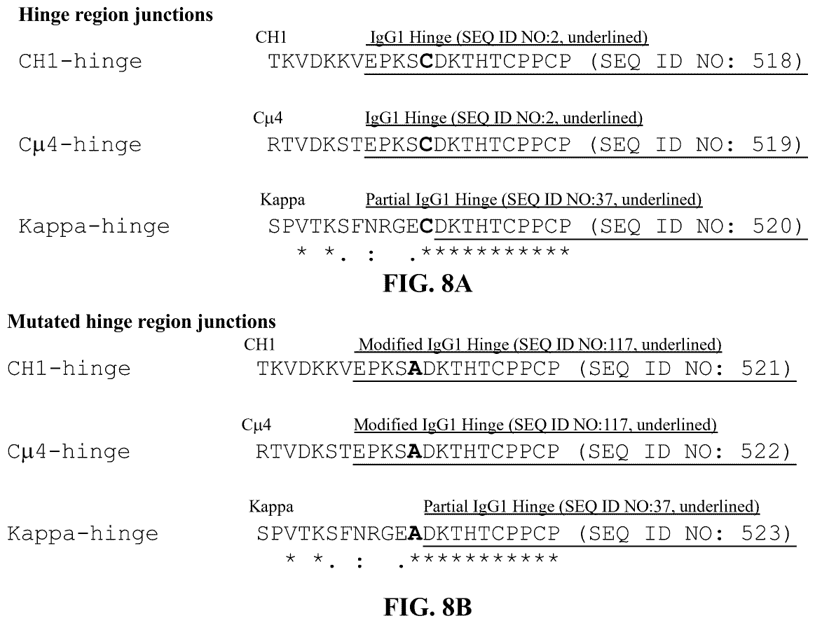

The use of an IgM Cμ4 region to replace a CH1 region in one half antibody offers an advantage over a conventional IgG1 CH1 region in facilitating purification of heterodimers including the first chain paired with the second chain and third chain with paired with the fourth chain, from homodimers. If the remaining CH1 constant region in the second half antibody is on a chain which cannot be purified by protein A (either because it is on the light chain that does not comprise a CH2 or CH3 domain, or because the CH2/CH3 domains have been mutated to reduce or eliminate protein A binding) then a two-step affinity purification can be used. FIGS. 2A and 2B show a cross-over antibody in which the first half-antibody comprises chains shown in FIG. 1D or FIG. 1A respectively, and the second half antibody is configured as in FIG. 1E. A first purification step on protein A resin will capture homodimers and heterodimers, but any excess CH1-containing light chain will be removed. A second purification step with a CH1-binding resin will capture only heterodimers and homodimers of the CH1-containing half-antibody, homodimers of the half antibody in which the CH1 has been replaced by IgM Cμ4 will be removed. By biasing expression to favor production of the half antibody in which the CH1 has been replaced by IgM Cμ4, simple affinity purification will be facilitated. Alternatively, FIGS. 2C and 2D show a cross-over antibody in which the first half-antibody comprises chains shown in FIG. 1D or FIG. 1A respectively, and the second half antibody is configured as in FIG. 1B but further comprises mutations in the Fc that reduce or eliminate protein A binding. A first purification step on protein A resin will then capture heterodimers and homodimers of the first but not the second half-antibody. A second purification step with a CH1-binding resin will capture only heterodimers, homodimers of the half antibody in which the CH1 has been replaced by IgM Cμ4 will be removed. If, in the first pair of chains, the IgM Cμ4 region is part of the first chain that further comprises an optional hinge region, or portion thereof (as represented in FIG. 1A), then the C-terminal cysteine of a kappa light chain, or the cysteine that is the penultimate residue of a lambda light chain (both at position 214 by Kabat numbering) may normally form a covalent disulfide bond with the cysteine in the hinge region corresponding to the first cysteine in the IgG1 hinge region or portion thereof (shown as the fifth residue from left of IgG1 hinge region shown in FIG. 8A in bold following the amino acid sequence EPKS, EU position 220). To prevent the formation of a disulfide bond between a light chain from the second pair of chains and the hinge region of the first pair, this hinge cysteine (equivalent to IgG1 hinge EU position 220) can be mutated to another amino acid incapable of forming a disulfide bond, for example to an isoleucine or a threonine or an alanine (an exemplary hinge region with an alanine substitution has amino acid sequence SEQ ID NO:117, and is shown in FIG. 8B and as the modified IgG1 hinges in FIG. 7E). Similarly, to prevent the formation of a disulfide bond between the kappa light chain from the first pair of chains and the hinge region of the second pair of chains, the C-terminal cysteine (EU position 214) of the kappa light chain of the first pair can be mutated to another amino acid incapable of forming a disulfide bond, for example to an isoleucine, threonine or alanine (as shown in the modified kappa chains in FIG. 7E). To produce a first new and specific disulfide bond between the first and second chain, a cysteine may be introduced into the IgM Cμ4 region of the first pair by replacing the tyrosine at position 455 in the IgM Cμ4 region (for example to give a modified IgM Cμ4 region comprising polypeptide sequence SEQ ID NO:74), with a corresponding second cysteine introduced into the kappa light chain constant region of the first pair by replacing the serine at EU position 121 (for example to give a modified kappa light chain constant region comprising polypeptide sequence SEQ ID NO:67). This is shown as Modified Cysteine Pair 1 in FIG. 7E. Alternatively, a second new and specific disulfide bond between the first and second chain may be produced by introducing a cysteine into the IgM Cμ4 region of the first pair by replacing the tyrosine at position 455 in the IgM Cμ4 region (for example to give a modified IgM Cμ4 region comprising polypeptide sequence SEQ ID NO:74), with a corresponding second cysteine introduced into the kappa light chain constant region of the first pair by replacing the glutamine at Kabat position 124 (for example to give a modified kappa light chain constant region comprising polypeptide sequence SEQ ID NO:68). This is shown as Modified Cysteine Pair 2 in FIG. 7E. Alternatively, a third new and specific disulfide bond between the first and second chain may be produced by introducing a first cysteine into the IgM Cμ4 region of the first pair by replacing the phenylalanine at position 516 in the IgM Cμ4 region (for example to give a modified IgM Cμ4 region comprising polypeptide sequence SEQ ID NO:75), with a corresponding second cysteine introduced into the kappa light chain constant region of the first pair by replacing the glutamine at Kabat position 160 (for example to give a modified kappa light chain constant region comprising polypeptide sequence SEQ ID NO:69). This is shown as Modified Cysteine Pair 3 in FIG. 7E. Alternatively, a fourth new and specific disulfide bond between the first and second chain may be produced by introducing a first cysteine into the IgM Cμ4 region of the first pair by replacing the threonine at position 471 in the IgM Cμ4 region (for example to give a modified IgM Cμ4 region comprising polypeptide sequence SEQ ID NO:76), with a corresponding second cysteine introduced into the kappa light chain constant region of the first pair by replacing the phenylalanine at Kabat position 116 (for example to give a modified kappa light chain constant region comprising polypeptide sequence SEQ ID NO:70). This is shown as Modified Cysteine Pair 4 in FIG. 7E.

If, in the first pair of chains, the kappa constant region is part of the first chain that further comprises an optional hinge region, or portion thereof (as represented in FIG. 1D), then it may be fused to the portion of the hinge region such that the first cysteine of the hinge is preserved, shown for example as the Kappa-hinge in FIG. 8A. If this cysteine is preserved, then the C-terminal cysteine of a human IgG1 CH1 constant region or cysteine at position 131 of a human IgG2, 3 or 4 CH1 constant region used as a light chain in a cross-mab format, or the C-terminal cysteine of a kappa light chain (as kappa light chains can form heterodimers) may form a covalent disulfide bond with the cysteine in the hinge region corresponding to the first cysteine in the IgG1 hinge region or portion thereof (EU position 220 shown as the cysteine immediately preceding the partial IgG1 hinge region shown in FIG. 8A in bold following the amino acid sequence NRGE). To prevent the formation of a disulfide bond between the fourth chain from the second pair of chains and the hinge region of the first chain, this cysteine of the first chain hinge region or portion thereof can be mutated to another amino acid incapable of forming a disulfide bond, for example to an isoleucine, threonine or alanine as shown for the Kappa-hinge in FIG. 8B. In this case the IgM Cμ4 chain is the second chain that does not further comprise an optional hinge region, or portion thereof, so to prevent the formation of a disulfide bond between the IgM Cμ4-containing second chain and the hinge region of the third chain, the IgM Cμ4 chain of the first pair should lack a cysteine near the C-terminus that is capable of forming such a disulfide bond. A natural IgM Cμ4 chain (for example a polypeptide with amino acid sequence SEQ ID NO:25, as shown in FIG. 7D) has a “tailpiece” which has a cysteine as the penultimate residue. To prevent inappropriate disulfide bond formation between the IgM Cμ4 chain from the first pair of chains and a chain from the second pair, this cysteine should be removed. This can be accomplished in a number of ways: the cysteine may be mutated to another amino acid incapable of forming a disulfide bond, for example to an isoleucine; the cysteine may be removed by truncating the IgM Cμ4 chain, exemplary sequences are shown in FIG. 7D as a polypeptide with amino acid sequence SEQ ID NO:23 or 53; the cysteine may be removed by truncating the IgM Cμ4 chain and mutating the C-terminal residue for example to an alanine or an isoleucine, an exemplary sequence is shown in FIG. 7D as a polypeptide with amino acid sequence SEQ ID NO:56. To produce a first new and specific disulfide bond between the first and second chain, a cysteine may be introduced into the IgM Cμ4 region of the first pair by replacing the tyrosine at position 455 in the IgM Cμ4 region (for example to give a modified IgM Cμ4 region comprising polypeptide sequence SEQ ID NO:57), with a corresponding second cysteine introduced into the kappa light chain constant region of the first pair by replacing the serine at Kabat position 131 (for example to give a modified kappa light chain constant region comprising polypeptide sequence SEQ ID NO:71). This is shown as Modified Cysteine Pair 1 in FIG. 7F. Alternatively, a second new and specific disulfide bond between the first and second chain may be produced by introducing a first cysteine into the IgM Cμ4 region of the first pair by replacing the phenylalanine at position 516 in the IgM Cμ4 region (for example to give a modified IgM Cμ4 region comprising polypeptide sequence SEQ ID NO:58), with a corresponding second cysteine introduced into the kappa light chain constant region of the first pair by replacing the serine at Kabat position 159 (for example to give a modified kappa light chain constant region comprising polypeptide sequence SEQ ID NO:72). This is shown as Modified Cysteine Pair 2 in FIG. 7F. Alternatively, a third new and specific disulfide bond between the first and second chain may be produced by introducing a first cysteine into the IgM Cμ4 region of the first pair by replacing the glutamine at position 463 in the IgM Cμ4 region (for example to give a modified IgM Cμ4 region comprising polypeptide sequence SEQ ID NO:59), with a corresponding second cysteine introduced into the kappa light chain constant region of the first pair by replacing the phenylalanine at Kabat position 116 (for example to give a modified kappa light chain constant region comprising polypeptide sequence SEQ ID NO:73). This is shown as Modified Cysteine Pair 3 in FIG. 7F.

III. Multi-specific antibody components with IgM Cμ4—IgG CH1 pairing

The multi-specific antibodies include two pairs of chains. Each pair includes a pair of heavy and light chain variable regions forming a binding site. The chains of the first pair can be referred to as first and second chains. The chains of the second pair can be referred to as third and fourth chains. The first and second pairs are sometimes each referred to as being a half-antibody because they each contribute one binding site to a bispecific antibody with two binding sites. The first chain comprises a first variable region, a first pairing region, an optional hinge region, or portion thereof, and IgG or IgA CH2 and CH3 regions; the second chain comprises a second variable region and a second pairing region. The first and second variable regions are heavy and light chain variable regions, which together form a first binding site. The first chain can include the heavy chain variable region or the light chain variable region. The second chain includes whichever of the heavy chain and light chain variable region is not included in the first chain. The pairing regions are so named because they mediate pairwise association between the first and second chains. The first and second pairing regions are an IgM Cμ4 region and an IgG CH1 constant region or vice versa. In other words, if the first pairing region is the IgM Cμ4 region the second pairing region is the IgG CH1 constant region and if the first pairing region is the IgG CH1 constant region, the second pairing region is the IgM Cμ4 region. In cross-over formats in which the IgG CH1 region is included adjacent a light chain variable region, the CH1 region can also include part of an adjacent hinge region up to and including EU residue 220 to permit disulfide bonding with the other chain of a half antibody. The first chain and second chain are paired via association of the IgM Cμ4 region and IgG CH1 constant region forming a binding site for a first target antigen.

FIGS. 3A-3F represent this format for a bispecific antibody. In FIG. 3A the light chain constant region of a first half-antibody is replaced by an IgM Cμ4 region. Pairing is maintained between the variable regions (V1a and V1b) because of pairing between these variable regions and between the IgG CH1 constant region and the IgM Cμ4 region. In FIG. 3B a second half-antibody has the natural organization with a heavy chain pairing to a light chain by interactions between the CH1 region and the light chain constant region (CL). Heterodimers are formed between the two half-antibodies (FIG. 3C). The IgM Cμ4 constant region of the first half pair may be in the second (light) chain (FIG. 3A), or it may be in the first chain that further comprises an optional hinge region, or portion thereof, and IgG or IgA CH2 and CH3 regions with the CH1 constant region being in the light chain (FIG. 3D). Similarly, the light chain constant region of the second half pair may be in the fourth (light) chain (FIG. 3B), or it may be in the third chain that further comprises an optional hinge region, or portion thereof, and IgG or IgA CH2 and CH3 regions with the CH1 constant region being in the light chain (FIG. 3E). The configuration for the first pair shown in either FIG. 3A or FIG. 3D may be combined with that shown for the second pair in either FIG. 3B or FIG. 3E. Combinations shown in FIG. 3C and FIG. 3F are shown as examples only, and not as limitations. The variable regions are labelled V1a, V1b, V2a and V2b because the variable regions that were originally associated with a heavy or a light chain may be switched. Thus the variable region originally associated with a light chain constant region in a natural antibody format may be placed at either V1a or V1b. Similarly the variable region originally associated with a heavy chain constant region in a natural antibody format may be placed at either V1a or V1b, so long as variable regions V1a and V1b are complementary. The same is true of variable regions V2a and V2b.

If the IgM Cμ4 region is part of the first chain further comprising a hinge region, or portion thereof, CH2 and CH3 region of each of human IgG1 isotype, then a C-terminal cysteine of a kappa light chain, or the cysteine that is the penultimate residue of a lambda light chain in the third chain may normally form a covalent disulfide bond with the cysteine in the hinge region corresponding to the first cysteine in the hinge region or portion thereof (shown as the fifth residue from left of IgG1 hinge region shown in FIG. 8A, in bold following the amino acid sequence EPKS, EU position 220). To prevent the formation of a disulfide bond between a light chain of the third chain and the hinge region of the first chain, hinge region cysteine at EU positions 220 can be mutated to another amino acid incapable of forming a disulfide bond, for example to an isoleucine or alanine or threonine. Similarly, to prevent the formation of a disulfide bond between an IgG1 CH1 constant region of the second chain, which typically comprises at its C-terminus an N-terminal segment of the hinge region up to and including C220 by EU numbering (e.g., with amino acid sequence EPKSC) in a cross-mAb format where CH1 is part of the light chain, and the fourth (light) chain of the second pair of chains, the C-terminal cysteine of the IgG1 CH1 of the second chain (which actually occurs in the partial hinge region at EU position 220) can be mutated to another amino acid incapable of forming a disulfide bond, for example to an isoleucine, threonine or alanine or simply by deleting this C-terminal cysteine entirely). This mutation in the second chain will also prevent disulfide bond formation between the second chain and the hinge region of the third chain if the third chain is part of a cross-mab format as shown in FIG. 3E. If the second chain is a CH1 of human IgG2, IgG3 or IgG4 isotypes, then the cysteine at EU position 131 may be mutated to another amino acid incapable of forming a disulfide bond, for example to an isoleucine, threonine or alanine to prevent the formation of a disulfide bond between the second chain and the fourth (light) chain of the second pair of chains. To produce a first new and specific disulfide bond between the first and second chain a first cysteine may be introduced into the IgM Cμ4 region of the first pair by replacing the tyrosine at position 455 in the IgM Cμ4 region (for example to give a modified IgM Cμ4 region comprising polypeptide sequence SEQ ID NO:74), with a corresponding second cysteine introduced into the IgG CH1 constant region of the first pair by replacing the alanine at EU position 141 (for example to give a modified IgG CH1 constant region comprising polypeptide sequence SEQ ID NO:60 or 79). This is shown as Modified Cysteine Pair 1 in FIG. 7G. Alternatively, a second new and specific disulfide bond between the first and second chain may be produced by introducing a first cysteine into the IgM Cμ4 region of the first pair by replacing the phenylalanine at position 516 in the IgM Cμ4 region (for example to give a modified IgM Cμ4 region comprising polypeptide sequence SEQ ID NO:75), with a corresponding second cysteine introduced into the IgG CH1 constant region of the first pair by replacing the histidine at EU position 168 (for example to give a modified IgG CH1 constant region comprising polypeptide sequence SEQ ID NO:61 or 80). This is shown as Modified Cysteine Pair 2 in FIG. 7G. Alternatively, a third new and specific disulfide bond between the first and second chain may be produced by introducing a first cysteine into the IgM Cμ4 region of the first pair by replacing the glutamine at position 463 in the IgM Cμ4 region (for example to give a modified IgM Cμ4 region comprising polypeptide sequence SEQ ID NO:77), with a corresponding second cysteine introduced into the IgG CH1 constant region of the first pair by replacing the phenylalanine at EU position 126 (for example to give a modified IgG CH1 constant region comprising a polypeptide with amino acid sequence SEQ ID NO:62 or 81). This is shown as Modified Cysteine Pair 3 in FIG. 7G. Alternatively, a fourth new and specific disulfide bond between the first and second chain may be produced by introducing a first cysteine into the IgM Cμ4 region of the first pair by replacing the leucine at position 457 in the IgM Cμ4 region (for example to give a modified IgM Cμ4 region comprising polypeptide sequence SEQ ID NO:78), with a corresponding second cysteine introduced into the IgG CH1 constant region of the first pair by replacing the leucine at EU position 128 (for example to give a modified IgG CH1 constant region comprising polypeptide sequence SEQ ID NO:82 or 83). This is shown as Modified Cysteine Pair 4 in FIG. 7G. Alternatively, a fifth new and specific disulfide bond between the first and second chain may be produced by introducing a first cysteine into the IgM Cμ4 region of the first pair by replacing the leucine at position 457 in the IgM Cμ4 region (for example to give a modified IgM Cμ4 region comprising polypeptide sequence SEQ ID NO:78), with a corresponding second cysteine introduced into the IgG CH1 constant region of the first pair by replacing the glycine at EU position 143 (for example to give a modified IgG CH1 constant region comprising polypeptide sequence SEQ ID NO:84 or 85). This is shown as Modified Cysteine Pair 5 in FIG. 7G.

If the CH1 region is part of the first chain that further comprises a hinge region, or portion thereof, CH2 and CH3 regions, then if the IgG CH1 constant region, the a hinge region, or portion thereof, CH2 and CH3 regions, are each of human IgG1 isotype, then the C-terminal cysteine of a kappa light chain constant region, or the cysteine that is the penultimate residue of a lambda light chain of the fourth chain, may form a covalent disulfide bond with the cysteine in the hinge region corresponding to the first cysteine in the IgG1 hinge region or portion thereof (shown as the cysteine of the IgG1 hinge region shown in FIG. 8A in bold following the amino acid sequence EPKS). To prevent such mis-pairing, the IgG1 hinge region cysteine in EU position 220 may be mutated to a residue incapable of forming a disulfide bond such as an alanine, a threonine or an isoleucine. If the first chain comprises an IgG CH1 constant region, an optional hinge region, or portion thereof, CH2 and CH3 regions, each of human IgG2, 3 or 4 isotype, then the C-terminal cysteine of a kappa light chain constant region, or the cysteine that is the penultimate residue of a lambda light chain of the fourth chain may form a covalent disulfide bond with the cysteine at EU position 131 of the CH1 region of the first chain. To prevent the formation of a disulfide bond between the fourth chain and the IgG2, IgG3 or IgG4 CH1, this cysteine at EU position 131 can be mutated to another amino acid incapable of forming a disulfide bond, for example to an isoleucine, a threonine or an alanine. In this case the IgM Cμ4 chain is not part of the chain that further comprises an optional hinge region, or portion thereof, so to prevent the formation of a disulfide bond between the IgM Cμ4 chain from the first pair of chains and the hinge region of the second pair of chains, the IgM Cμ4 chain of the first pair should lack a cysteine near the C-terminus that is capable of forming such a disulfide bond. A natural IgM Cμ4 chain (for example a polypeptide with amino acid sequence SEQ ID NO:25, as shown in FIG. 7D) has a “tailpiece” which has a cysteine as the penultimate residue (Kabat position 575). To prevent inappropriate disulfide bond formation between the IgM Cμ4 chain from the first pair of chains and a chain from the second pair, this cysteine should be removed. This can be accomplished in a number of ways: the cysteine may be mutated to another amino acid incapable of forming a disulfide bond, for example to an isoleucine (as shown in FIG. 8B), or a threonine or an alanine; the cysteine may be removed by truncating the IgM Cμ4 chain, exemplary sequences are shown in FIG. 7D as a polypeptide with amino acid sequence SEQ ID NO:23 or 53; the cysteine may be removed by truncating the IgM Cμ4 chain and mutating the C-terminal residue for example to an alanine or an isoleucine, an exemplary sequence is shown in FIG. 7D as a polypeptide with amino acid sequence SEQ ID NO:56. To produce a new and specific disulfide bond between the first and second chain a first cysteine may be introduced into the IgM Cμ4 region of the first pair by replacing the tyrosine at position 455 in the IgM Cμ4 region (for example to give a modified IgM Cμ4 region comprising polypeptide sequence SEQ ID NO:57), with a corresponding second cysteine introduced into the IgG CH1 constant region of the first pair by replacing the alanine at EU position 141 (for example to give a modified IgG CH1 constant region comprising polypeptide sequence SEQ ID NO:60). This is shown as Modified Cysteine Pair 1 in FIG. 7H. Alternatively, a second new and specific disulfide bond between the first and second chain may be produced by introducing a first cysteine into the IgM Cμ4 region of the first pair by replacing the phenylalanine at position 516 in the IgM Cμ4 region (for example to give a modified IgM Cμ4 region comprising polypeptide sequence SEQ ID NO:58), with a corresponding second cysteine introduced into the IgG CH1 constant region of the first pair by replacing the histidine at EU position 168 (for example to give a modified IgG CH1 constant region comprising polypeptide sequence SEQ ID NO:61). This is shown as Modified Cysteine Pair 2 in FIG. 7H. Alternatively, a third new and specific disulfide bond between the first and second chain may be produced by introducing a first cysteine into the IgM Cμ4 region of the first pair by replacing the glutamine at position 463 in the IgM Cμ4 region (for example to give a modified IgM Cμ4 region comprising polypeptide sequence SEQ ID NO:59), with a corresponding second cysteine introduced into the IgG CH1 constant region of the first pair by replacing the phenylalanine at EU position 126 (for example to give a modified IgG CH1 constant region comprising polypeptide sequence SEQ ID NO:62). This is shown as Modified Cysteine Pair 3 in FIG. 7H.

IV. Multi-Specific Antibody Components with IgM Cμ4—IgG CH1 or IgM Cμ4—Kappa Pairing

The third chain comprises a third variable region, a third pairing region an optional hinge region, or portion thereof, and IgG or IgA CH2 and CH3 regions. The optional hinge region, or portion thereof, and IgG or IgA CH2 and CH3 regions can be referred to as a second hinge region or portion thereof and second IgG or IgA CH2 and CH3 regions to distinguish them from the at least a portion of a hinge region and IgG or IgA CH2 and CH3 regions of the first chain described above. The fourth chain comprises a fourth variable region and a fourth pairing region. The third and fourth variable regions are heavy and light chain variable regions or vice versa, which can pair to form a second binding site. If the third chain includes a heavy chain variable region, the fourth chain includes a light chain variable region and versa. The third and fourth pairing regions are an IgG or IgA CH1 constant region and a light chain constant region or vice versa. That is, if the third pairing region is an IgG or IgA CH1 constant region, the fourth pairing region is the light chain constant region. If the third pairing region is the light chain constant region, the fourth pairing region is the IgG or IgA CH1 constant region. The light chain region can be kappa or lambda. The third and fourth chains are paired via association of the third and fourth pairing regions, which may be augmented by mutual affinity of the third and fourth variable regions for each other, forming a binding site for a second target antigen.

The first and second pairs of heavy and light chain are associated via the IgG or IgA CH3 regions forming a tetramer. Association can be strengthened by disulfide bonding between IgG hinge regions or portions thereof (e.g., one or two disulfide bonds).

The designation of chains as first, second, third and fourth is for ease of reference only. Thus, the description for the first and second chains could be transposed with one another as can the descriptions for the third and fourth chains. Likewise, descriptions for first and second chains can be transposed with those of third and fourth chains.

Correct combination of heavy and light chain variable region pairs can be promoted by use of a cross-over format in which pairing regions are transposed in one but not both of the binding sites. In other words, if the IgG or IgA at least a portion of a hinge, CH2 and CH3 in the first binding site is linked to the light chain variable region (i.e., cross-over format) then the IgG or IgA at least a portion of a hinge, CH2 and CH3 region in the second binding site is linked to the heavy chain variable region (i.e., non-cross-over format). Conversely if the IgG or IgA at least a portion of a hinge, CH2 and CH3 in the first binding site is linked to a heavy chain variable region (i.e., non-cross-over format) then the IgG or IgA at least a portion of a hinge, CH2 and CH3 in the second binding site is linked to the light chain variable region. The pairing regions can be transposed with or without transposition of heavy chain constant region components naturally linked to CH1 (i.e., hinge, CH2 and CH3). Thus, CH1-hinge-CH2-CH3 can be transposed with light chain constant region kappa or lambda, such that CH1-hinge-CH2-CH3 is linked to a light chain variable region and the light chain constant region linked to a heavy chain variable region. Likewise, Cμ4-hinge-CH2-CH3 can be transposed with a kappa light chain constant region so Cμ4-hinge-CH2-CH3 is linked to a light chain variable region and the kappa light chain constant region to a heavy chain variable region. Likewise, Cμ4-hinge-CH2-CH3 can be transposed with a CH1 region so Cμ4-hinge-CH2-CH3 is linked to a light chain variable region and the CH1 region to a heavy chain variable region. Transposition of pairing regions with linked heavy chain constant region components with respect to the variable regions is equivalent to transposing heavy and light chain variable regions with respect to the other components. Cross-over formats promote correct combinations of heavy and light chain variable regions because the heavy and light chain variable regions intended to be paired have pairing regions with pairwise affinity for one another, whereas byproduct heavy and light chain combinations have pairing regions without pairwise affinity (e.g., CH1 and Cμ4, or two light chain constant regions).

In a preferred format, the first variable region is the heavy chain variable region of the first binding site and the first pairing region is the light chain kappa constant region, the second variable region is the light chain variable region of the first binding site and the second pairing region is the IgM Cμ4 region, the third variable region is the heavy chain variable region of the second binding site and the third pairing region is an IgG or IgA CH1 constant region and the fourth variable region is the light chain variable region of the second binding site and the fourth pairing region is the second light chain constant region.

In another preferred format, the first variable region is the heavy chain variable region of the first binding site and the first pairing region is the IgM Cμ4 region, the second variable region is the light chain variable region of the first binding site and the second pairing region is the light chain kappa constant region, the third variable region is the light chain variable region of the second binding site and the third pairing region is an IgG or IgA CH1 constant region and the fourth variable region is the heavy chain variable region of the second binding site and the fourth pairing region is the second light chain constant region.

In a preferred format, the first variable region is the heavy chain variable region of the first binding site and the first pairing region is a CH1 region, the second variable region is the light chain variable region of the first binding site and the second pairing region is the IgM Cμ4 region, the third variable region is the heavy chain variable region of the second binding site and the third pairing region is an IgG or IgA CH1 constant region and the fourth variable region is the light chain variable region of the second binding site and the fourth pairing region is the second light chain constant region.

In another preferred format, the first variable region is the light chain variable region of the first binding site and the first pairing region is a light chain kappa constant region, the second variable region is the heavy chain variable region of the first binding site and the second pairing region is the IgM Cμ4 region, the third variable region is the light chain variable region of the second binding site and the third pairing region is an IgG or IgA CH1 constant region and the fourth variable region is the heavy chain variable region of the second binding site and the fourth pairing region is the second light chain constant region.

The tables below summarize combinations of the components in the four chains. Any of the eight combinations of first and second chains can be used with any of the four combinations of third and fourth chains for 32 combinations.

| | | | | | | | | |
|---|---|---|---|---|---|---|---|---|
| 1^{st} | VH, Cμ4, | VH, Cμ4, | VH, CH1, | VH, CL1, | VL, Cμ4, | VL, Cμ4, | VL, CH1, | VL, CL, |
| chain | hinge, | hinge, | hinge, | hinge, | hinge, | hinge, | hinge, | hinge, |
| | CH2, | CH2, | CH2, | CH2, | CH2, | CH2, | CH2, | CH2, |
| | CH3 | CH3 | CH3 | CH3 | CH3 | CH3 | CH3 | CH3 |
| 2^{nd} | VL, CH1 | VL, CL | VL, Cμ4 | VL, Cμ4 | VH, CH1 | VH, CL | VH, Cμ4 | VH, Cμ4 |
| chain | | | | | | | | |

| | | | | |
|---|---|---|---|---|
| 3^{rd} | VH, CH1, | VH, CL, | VL, CH1, | VL, CL, |
| chain | hinge, | hinge | hinge, | hinge, |
| | CH2, CH3 | CH2, CH3 | CH2, CH3 | CH2, CH3 |
| 4^{th} | VL, CL | VL, CH1 | VH, CL | VH, CH1 |
| chain | | | | |

In another purification scheme, a bispecific antibody can be purified from homodimers by successive purifications on CH1-binding resin and Cμ4 binding resin, in either order. Heterodimers but not homodimers of either component antibody bind to both resins. Thus, by retaining material binding to the column at each step substantial enrichment for bispecific antibodies can be obtained.

Enrichment of a bispecific antibody over homodimers can also be obtained using a CH1-binding resin under conditions that distinguish binding of a single CH1, two CH1's and no CH1, such that antibodies with no CH1 regions pass over such a resin, antibodies with a single CH1 and two CH1's both bind to the resin, but antibodies with the dual CH1 are eluted in a later fraction than antibodies with a single CH1 due to avidity effects. Analogous results can be obtained with a Cμ4-binding resin.

Typically, the two heavy chain variable regions of a bispecific antibody are different from one another as are the two light chains, and consequently the two combinations of heavy and light chains. Each combination of heavy and light chain includes a binding site for a target antigen epitope. Typically, the binding sites and target antigens are different from one another, although a multi-specific antibody can have two binding sites for epitopes at different sites in the same target antigen.

Typically all components of a heavy chain constant region, except for the IgM Cμ4 region and sometimes the hinge region are of the same IgG or IgA isotype and subclass. Preferably the isotype is human IgG1, IgG2, IgG3 or IgG4, or human IgA1 or IgA2. If the hinge region is present, it preferably has the same isotype as other components, e.g. human IgG1, IgG2, IgG3 or IgG4, but can be a different isotype or a hybrid of isotypes.

Components of heavy chain constant regions in two half-antibodies (i.e., first and third chains) are also all preferably of the same isotype and subclass except for IgM Cμ4 region and sometimes the hinge region. Preferably all components of both heavy chain constant regions except for IgM Cμ4 region and sometimes the hinge region are of the same IgG isotype and subclass. Preferably hinge region(s) or a portion(s) or segment(s) thereof are of also of the same IgG isotype and subclass as other IgG components.

Correct pairing of first and second heavy chains with each as a heterodimers via the IgG or IgA CH2 and CH3 regions as distinct from undesired formation of homodimers can be promoted by inserting knobs and holes into the CH3 regions of the respective heavy chains (Ridgway et al., Protein Eng 9:617-21, 1996; Atwell et al., J Mol Biol 270:26-35, 1997; and U.S. Pat. No. 7,695,936). In accordance with previous usage in the art, knobs and holes refer to mutations relative to the corresponding amino acid(s) of natural immunoglobulin sequences (e.g., as provided in the Swiss Prot database) that allow a knob (i.e., protrusion) to couple with a corresponding hole (i.e., an indentation) thereby promoting association of immunoglobulin chains bearing the knob and hole. A knob is created by substituting a native amino acid with a larger amino acid by molecular weight and a hole is created by substituting a native amino acid with a smaller amino acid by molecular weight.

The following corresponding knobs and holes substitutions in the individual polypeptide chains of an Fc-region of an IgG antibody of subclass IgG1 have been found to increase heterodimer formation (see, for example, US 20170342168) (EU numbering):
1) Y407T in one chain and T366Y in the other chain;
2) Y407A in one chain and T366W in the other chain;
3) F405A in one chain and T394W in the other chain;
4) F405W in one chain and T394S in the other chain;
5) Y407T in one chain and T366Y in the other chain;
6) T366Y and F405A in one chain and T394W and Y407T in the other chain;
7) T366W and F405W in one chain and T394S and Y407A in the other chain;
8) F405W and Y407A in one chain and T366W and T394S in the other chain;
9) T366W in one chain and T366S, L368A, and Y407V in the other chain; and
10) S354C, T366W in one chain and Y349C, T366S, L368A, Y407V in the other.

In addition, changes creating new disulfide bonds between the two Fc-region polypeptide chains facilitate heterodimer formation (see, e.g., US 2003/0078385). The following substitutions resulting in appropriately spaced apart cysteine residues for the formation of new interchain disulfide bonds in the individual polypeptide chains of an Fc-region of an IgG antibody of subclass IgG1 have been found to increase heterodimer formation: Y349C in one chain and S354C in the other; Y349C in one chain and E356C in the other; Y349C in one chain and E357C in the other; L351C in one chain and S354C in the other; T394C in one chain and E397C in the other; or D399C in one chain and K392C in the other. Further examples of heterodimerization-facilitating amino acid changes are the so-called “charge pair substitutions,” also termed “electrostatic steering substitutions” (see, e.g., WO 2009/089004). The following charge pair substitutions in the individual polypeptide chains of an Fc-region of an IgG antibody of subclass IgG1 have been found to increase heterodimer formation:
1) K409D or K409E in one chain and D399K or D399R in the other chain;
2) K392D or K392E in one chain and D399K or D399R in the other chain;
3) K439D or K439E in one chain and E356K or E356R in the other chain;
4) K370D or K370E in one chain and E357K or E357R in the other chain;
5) K409D and K360D in one chain plus D399K and E356K in the other chain;
6) K409D and K370D in one chain plus D399K and E357K in the other chain;
7) K409D and K392D in one chain plusD399K, E356K, and E357K in the other chain;
8) K409D and K392D in one chain and D399K in the other chain;
9) K409D and K392D in one chain and D399K and E356K in the other chain;
10) K409D and K392D in one chain and D399K and D357K in the other chain;
11) K409D and K370D in one chain and D399K and D357K in the other chain;
12) D399K in one chain and K409D and K360D in the other chain; and
13) K409D and K439D in one chain and D399K and E356K on the other.

Knob-into-hole and charge pair (i.e., electrostatic) substitutions can also be combined. The following corresponding electrostatic substitutions combined with knobs and holes substitutions in the individual polypeptide chains of an Fc-region of an IgG antibody of subclass IgG1 have been found to increase heterodimer formation (see, e.g., International Appl. WO2024/206820, and Klein et al. “Progress in overcoming the chain association issue in bispecific heterodimeric IgG antibodies” mAbs 4:6, 653-663 (November/December 2012)): Y407T in one chain and T366Y in the other chain combined with 1) D356K or D356R or D356H in one chain and K439D or K439E in the other chain; or combined with 2) (D356K or D356R or D356H) and (K439R or K439H) in one chain and D356E and (K439D or K439E) in the other. The above substitutions are in EU numbering with the original amino acid first and replacement amino acid second. Although exemplary substitutions are provided for human IgG1 isotype, the same or corresponding substitutions (substitution at same position with same replacement residue or same type of replacement, e.g., small to large residue to make a knob or large to small residue to make a hole) can be made for other isotypes and sub-isotypes. Heavy chain pairing can also be promoted by fusing the C-terminus of the heavy chains to leucine zippers or other molecules with pairwise affinity for one another (see, e.g., International Application WO2018/237192). Heterodimerization between chains comprising knob and hole mutations can be improved by incorporating the substitution P374A into the IgG1 CH3 region.

The isotypes of the two heavy chains (other than the presence of IgM Cμ4 in one of the chains) can be the same or different from each other.

As previously mentioned, pairwise affinity of an IgM Cμ4 region and kappa light chain constant region in one heavy light chain pair and of CH1 and light chain constant region in the other promotes pairing of heavy and light chains. Co-association of IgG or IgA CH3 regions between the two heavy chains promotes pairing of the two pairs of heavy and light chains in the form of a tetramer in similar manner to a natural antibody. Such antibodies have two binding sites. Depending on isotype and subtype, and presence of mutations, presence of IgG or IgA CH2 and CH3 regions can confer effector functions, such as ADCC, CDC and opsonization, FcRn binding, protein A and G binding.

The hinge region provides flexibility between the binding region and effector region of an antibody and contributes to efficient effector functions, such as ADCC, opsonization and CDC. The hinge region is also the site of disulfide bonds that link a pair of IgG heavy chains together. IgA does not have a hinge region according to the Kabat delineation of regions. However, the residues in CH1 and CH2 flanking the border between these regions in IgA provide flexibility effectively serving the role of a hinge region. Formation of disulfide bonds between cysteines in the hinge region of the first and third chains for IgGs, or between cysteines in the CH2 region for IgAs promotes association of the chains and formation of a tetrameric structure. IgG2 and IgG4 have multiple isomeric forms differing in whether cysteine residues at EU positions 219 and 220 for human IgG2 and EU 226 and 229 for human IgG4 in one half antibody form interchain disulfides at the corresponding position of a hinge region in the other half-antibody, or instead disulfide bond with cysteines in the same half antibody (Vidarsson et al., Frontiers in Immunol. 550, 520 (2014)). The different isomeric forms can undergo reversible conversions to one another. A preparation of an IgG2 or IgG4 antibody can thus include multiple isomeric forms. Loss of disulfide bonds between half-antibodies in IgG4 results in Fab arm exchange. Such can be inhibited by inclusion of an S228P mutation (EU numbering) (J Biol Chem. 2015 Feb. 27; 290(9): 5462-5469).

Reference to a human IgG, IgA or IgM region (i.e., CH1, hinge, CH2, CH3, Cμ4) refers to the exemplified sequences or allotypes or isoallotypes thereof or other variant sequence having at least 90, 95, 98 or 99% sequence identity with an exemplified sequence and/or differing from the exemplified sequence by up to 1, 2, 3, 4, 5, 10, or 15 deletions, substitutions or internal insertions in the case of CH1, CH2, CH3, up to 1, 2, 3, 4, 5, 1, 15, 20 or 25 deletions, substitutions or internal insertions for Cμ4 and one, two, or three deletions, substitutions or internal insertions for IgG1, 2 or 4 hinge regions and up to 1, 2, 3, 4, 5, or 6 deletions, substitutions or internal insertions for an IgG3 hinge region.

Residues in any variant of an exemplified SEQ ID NO. are numbered as the corresponding residues in the exemplified SEQ ID NO. after maximal alignment of the respective sequences.

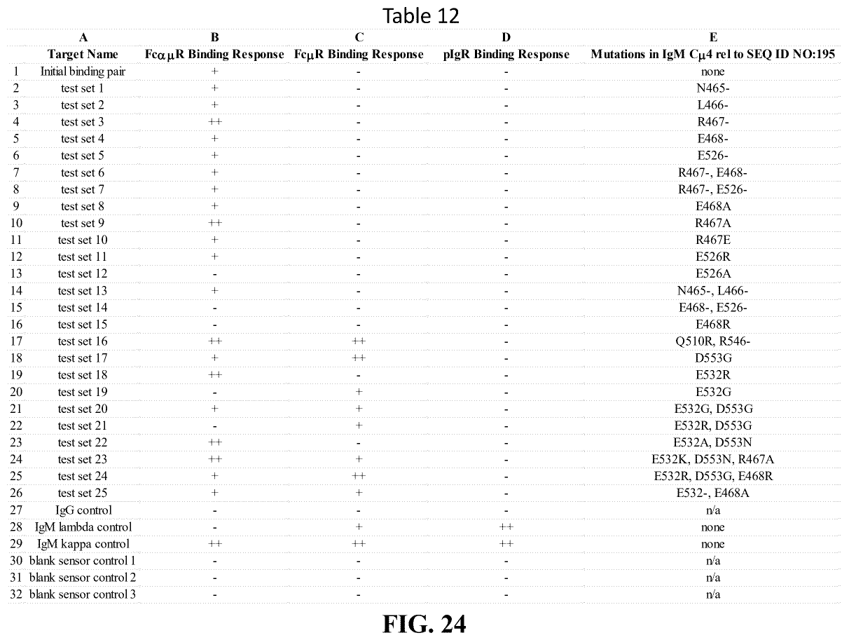

Some variations from a natural human Cμ4 region occur in the C-terminal 20 amino acids, some or all of which may be truncated. Optionally, the C-terminal residue of a Cμ4 region is mutated to a cysteine to facilitate disulfide bonding between the Cμ4 region and a c-terminal cysteine of a kappa constant region. FIG. 7D shows five exemplary Cμ4 regions. SEQ ID NO:23 has a two amino acid C-terminal deletion of a human Cμ4 region. SEQ ID NO:24: has a two amino acid C-terminal deletion of a human Cμ4 regions and substitution of a T to a C at the C-terminal amino acid after the deletion. SEQ ID NO:53 is a full length human Cμ4 region. SEQ ID NO:25 is a full length human Cμ4 region including an 18 amino acid “tailpiece” at the C-terminus. A truncated form of a full length human Cμ4 region can also referred to a portion of a Cμ4 region. Mutations can also occur at Kabat position Q510, E468 or E526 to reduce effector functions (see Example 8 and FIG. 24 Table 12), and addition of engineered cysteines at any of Kabat positions 455, 457, 463, 471, and 516. One or more internal loops can be deleted.

FIG. 8A shows an IgG1 hinge fused to CH1 and Cμ4 regions, and an IgG1 hinge truncated by five N-terminal amino acids linked to a kappa constant region. Reference to a portion of a hinge region means a contiguous sequence of at least five amino acids of an exemplified hinge region sufficient to promote formation of at least one and optionally two or more interchain disulfide bonds between half-antibodies and optionally a further disulfide bond between a hinge region and a Cμ4 region. Truncations are preferably at the N-terminus of a natural hinge region, such as loss of 5 amino acids at the N-terminus, as shown in FIG. 8A or variant maximally aligned with the hinge portion shown in FIG. 8A.

Substitutions, if present, in any of the above-mentioned regions can be conservative. Some substitutions serve to remove a cysteine residue not required for interchain pairing, a glycosylation site or proteolytic cleavage site. Some substitutions introduce a cysteine residue for disulfide bonding between hinge regions, between a kappa constant region and Cμ4, or between a hinge and Cμ4.

Human constant regions show allotypic variation and isoallotypic variation between different individuals, that is, the constant regions can differ in different individuals at one or more polymorphic positions. Isoallotypes differ from allotypes in that sera recognizing an isoallotype bind to a non-polymorphic region of a one or more other isotypes. Reference to a human constant region includes a constant region with any natural allotype (including isoallotypes) or any permutation of residues occupying polymorphic positions in natural allotypes. Sequences of non-human constant regions are provided by e.g., the Swiss-Prot or Genbank databases. Reference to a non-human constant region likewise includes allotypic or isoallotypic variants, and permutations of the same, or other variants sequences differing from natural sequences. The scope of variations is defined by sequence identity and/or number of substitutions with respect to natural sequences of non-human constant regions in analogous fashion to the above description of variants with respect to human constant regions.

If a hinge region is used, part of the hinge can be replaced by a synthetic linker molecule typically formed of any of gly, ala, ser, and leu and combinations thereof. The hinge region can also be replaced in its entirety by a synthetic linker or omitted without replacement.

With the possible exception of a synthetic linker replacing part or all of a hinge region and one or a few amino acid substitutions to enhance or suppress effector functions or FcRn binding as discussed further below and IgM Cμ4 regions, it is preferred that heavy and light chains contain no sequences other than those mentioned above. Nevertheless, other sequences, such as for example, a hexa-histidine tag, can be added but are not necessary.

Additional binding sites in the form of scFvs or other antibody fragments can also be incorporated at the N-terminus or C-terminus of any or all of the heavy and light chains. Additional binding sites can have specificity for additional target antigens or the one or both of the same target antigens as the basic tetrameric antibody structure.

### V. Selection of Constant Region

The multi-specific antibody just described includes at least a portion of a constant region, i.e., CH2 and CH3 regions of IgG or IgA isotype. The constant region can be rodent, e.g., mouse or rat, primate, or human among others. The choice of constant region depends, in part, whether antibody-dependent cell-mediated cytotoxicity, antibody dependent cellular phagocytosis and/or complement dependent cytotoxicity are desired. For example, human isotypes IgG1 and IgG3 have complement-dependent cytotoxicity and human isotypes IgG2 and IgG4 do not. Light chain constant regions can be lambda or kappa. Human IgG1 and IgG3 also induce stronger cell mediated effector functions than human IgG2 and IgG4. ADCC, ADCP and CDC may be useful in providing an additional mechanism of action against cancer or infected cells bound by one arm of the multi-specific antibodies, it is not useful for agonizing effector cells.

One or several amino acids at the amino or carboxy terminus of the light and/or heavy chain, such as the C-terminal lysine of the heavy chain, may be missing or derivatized in a proportion or all of the molecules. Amino acid substitutions can be made in the constant regions to reduce or increase effector functions such as complement-mediated cytotoxicity or ADCC (see, e.g., Winter et al., U.S. Pat. No. 5,624,821; Tso et al., U.S. Pat. No. 5,834,597; and Lazar et al., Proc. Natl. Acad. Sci. USA 103:4005, 2006). Still other mutations can be made to either the light and/or heavy chain(s) for the purpose of prolonging the half-life of an antibody in humans (see, e.g., Hinton et al., J. Biol. Chem. 279:6213, 2004).

For example, there are many known mutations in IgG Fc that increase FcRn binding. FcRn refers to the neonatal Fc receptor. Exemplary substitutions include Gln at position 250 and/or Leu at position 428, Ser or Asn at position 434, Tyr at position 252, Thr at position 254, Glu at position 256, and Ala at position 434 (EU numbering). Increased FcRn binding is advantageous in making the hybrid proteins of the present invention compete more strongly with endogenous IgG for binding to FcRn. Also numerous mutations are known for reducing any of ADCC, ADCP or CDC. (see, e.g., Winter et al., U.S. Pat. No. 5,624,821; Tso et al., U.S. Pat. No. 5,834,597; and Lazar et al., Proc. Natl. Acad. Sci. USA 103:4005, 2006). Optionally the IgM Cμ4 region comprises a mutation or a deletion of Q510, E468 or E526 (Kabat numbering) to reduce or eliminate binding to FcpR or FCapR, thereby reducing any IgM effector functions; see Example 8 and FIG. 24 Table 12. Substitution of any of amino acid residues at positions 234, 235, 236 and/or 237 reduce affinity for Fcγ receptors, particularly FcγRI receptor (see, e.g., U.S. Pat. No. 6,624,821). Optionally, amino acid residues at positions 234, 236 and/or 237 in human IgG2 are substituted with Ala and at position 235 with Gln or Glu (See, e.g., U.S. Pat. No. 5,624,821). Other substitutions reducing effector functions include Ala at position 268, Gly or Ala at position 297, Leu at position 309, Ala at position 322, Gly at position 327, Ser at position 330, Ser at position 331, Ser at position 238, Ala at position 268, Leu at position 309 (EU numbering). Other substitutions that can be included to reduce protein A binding in one of the heavy chains include (T307P, L309Q, and Q311R or “TLQ”), or (H435R/Y436F) in the Fc region of human IgG1 (EU numbering).

### VI. Target Antigens

The diversity of antigens/epitopes of interest that find use as targets for the Fab, bispecific or multispecific antibodies described herein is not limited in any regard, and further, no attempt is made herein to recite the full spectrum of antigens that might beneficially be targeted by antibodies described herein.

A multi-specific antibody has at least two binding sites for two different target antigens. The target antigens can both be present on the same target cell, such as a cancer cell, virus, pathogen-infected cell or other pathological cell. Such an antibody can have greater specificity for the target cell than an antibody directed against a single target antigen on the target cell. Alternatively, one binding site can be for a target antigen on such a target cell and the other on an effector cell to be recruited to induce an immune response against the target cell. Some multi-specific antibodies include one binding site against a target antigen on a target cell and another binding site for a checkpoint inhibitor antigen. Other multi-specific antibodies include binding regions for both a receptor and its ligand or counter-receptor. Such antibodies can exert greater inhibition than antibodies binding receptor or ligand/counterreceptor alone.

Target antigens of interest include receptors on cancer cells and their ligands or counter-receptors (e.g., CD3, CD20, CD22, CD30, CD34, CD40, CD44, CD47, CD52 CD70, CD79a, DR4 DR5, EGFR, CA-125/Muc-16, MC1 receptor, PEM antigen, gp72, EpCAM, Her-2, VEGF or VEGFR, ganglioside GD3, CEA, AFP, CTLA-4, alpha v beta 3, HLA-DR 10 beta, SK-1). Other targets of interest are autoantibodies or T-cell subsets mediating autoimmune disease. Other targets of interest include any CD antigens from CD1a to CD371. Other targets of interest are growth factor receptors (e.g., FGFR, HGFR, PDGFR, EFGR, NGFR, and VEGFR) and their ligands. Other targets are G-protein receptors and include substance K receptor, the angiotensin receptor, the a and 3 adrenergic receptors, the serotonin receptors, and PAF receptor. See, e.g., Gilman, Ann. Rev. Biochem. 56:625 649 (1987). Other targets include ion channels (e.g., calcium, sodium, potassium channels), muscarinic receptors, acetylcholine receptors, GABA receptors, glutamate receptors, and dopamine receptors (see Harpold, U.S. Pat. Nos. 5,401,629, 5,436,128). Other targets are adhesion proteins such as integrins, selectins, and immunoglobulin superfamily members (see Springer, Nature 346:425 433 (1990). Osborn, Cell 62:3 (1990); Hynes, Cell 69:11 (1992)). Other targets are cytokines, such as interleukins IL-1 through about IL-37 to-date, tumor necrosis factors, interferon, tumor growth factor beta, colony stimulating factor (CSF) and granulocyte monocyte colony stimulating factor (GM-CSF). See Human Cytokines: Handbookfor Basic and Clinical Research (Aggrawal et al. eds., Blackwell Scientific, Boston, Mass. 1991). Other targets are amyloidogenic peptides, such as Abeta, alpha-synuclein or prion peptide. Other targets are hormones, enzymes, and intracellular and intercellular messengers, such as, adenyl cyclase, guanyl cyclase, and phospholipase C. Target molecules can be human, mammalian or bacterial. Other targets are antigens, such as proteins, glycoproteins and carbohydrates from microbial pathogens, both viral and bacterial, and tumors.

Checkpoint inhibitors block the immune system from attacking cancer cells. Some examples of target antigens that are checkpoint inhibitors include PD-1, PD-2, PD-L1, PD-L2, CTLA-40, CD47, OX40, B7.1, B7He, LAG3, CD137, KIR, CCR5, CD27, or CD40.

Other target antigens are on the surface of T-cells or NK cells. Human T-cell antigens likely to be suitable include CD3, CD2, CD28, CD44, C69, A13 and G1. Suitable antigens on natural killer cells include FC Gamma receptors (3G8, B73.1, LEUL1, VEP13, and AT10).

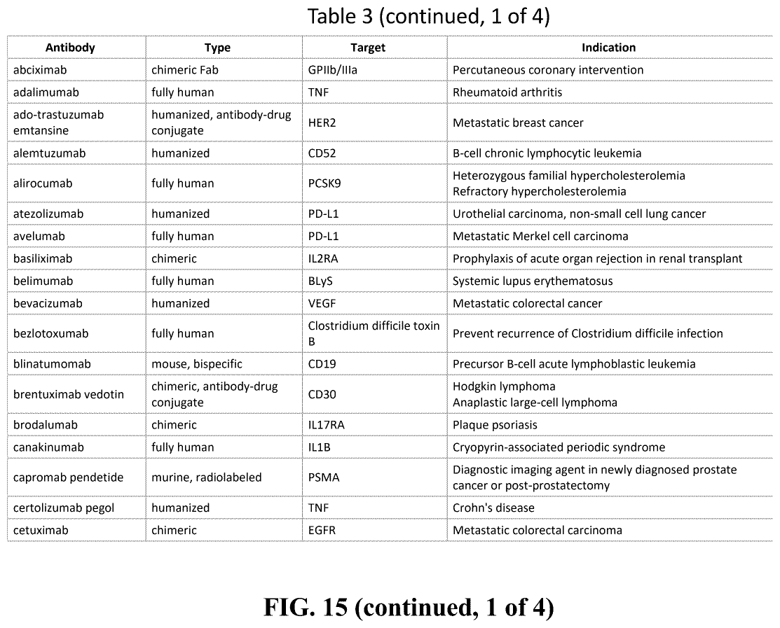

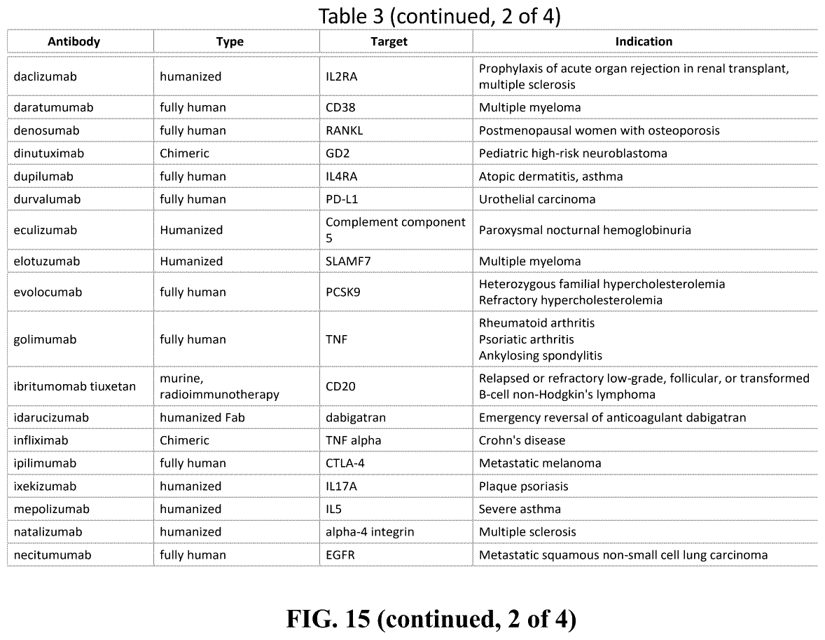

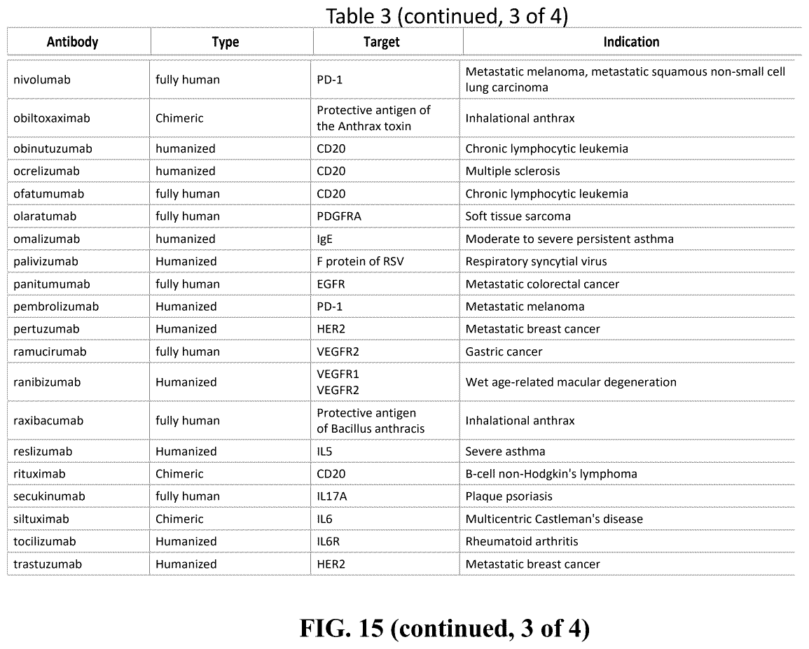

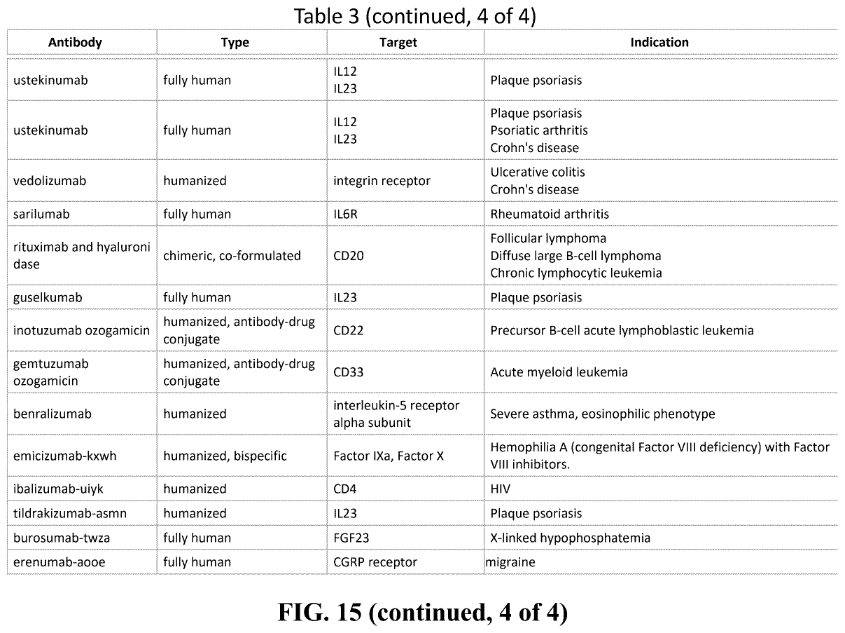

Some examples of commercial antibodies and their targets are shown in the FIG. 15 Table 3. A binding site of any of these commercial antibodies can be included in a multi-specific antibody of the invention.

### VII. Expression of Recombinant Antibodies

Multi-specific antibodies can be produced by recombinant expression with all chains expressed in the same cells. Chains can be expressed from the same or different vectors. Chains can be expressed from separate or combined transcriptional units with individual chains separated by IRES or viral 2A/CHYSEL sequences. When first and second chains include Cμ4 and kappa light chain constant region as pairing regions and third and four chains include CH1 and a second light chain constant region, it can be advantageous to express the first and second chains at a higher level than the third and four chains. If there is variation in expression levels between first and second chains, and/or between third and fourth chains, the expression level of first and second chain is considered higher than that of the third and four chains based on the mean expression level of the first and second chains and mean expression level of the third and fourth chains. Such expression generates predominantly heterodimers including all four chains, and homodimers of the first and second chain pair. The heterodimers can be purified from these homodimers by C1 affinity chromatography. As mentioned, it can be advantageous to express one Recombinant polynucleotide constructs typically include an expression control sequence operably linked to the coding sequences of antibody chains, including naturally associated or heterologous expression control elements, such as a promoter. The expression control sequences can be promoter systems in vector(s) capable of transforming or transfecting eukaryotic or prokaryotic host cells. Once the vector(s) have been incorporated into the appropriate host, the host is maintained under conditions suitable for high level expression of the nucleotide sequences and the collection and purification of multi-specific antibodies.

Nucleic acids encoding any or all of the light chains and heavy chain of the multispecific antibodies can be integrated into the genome of host cells by incorporating the nucleic acids, and optionally regulatory sequences between inverted repeats of a transposon and using a transposase to transpose the transposon into a cellular genome, among other methods. Exemplary transposons for such purpose are the piggyBac transposon described by e.g., Shi et al., BMC Biotechnol. 2007; 7:5. doi: 10.1186/1472-6750-7-5. and piggyBac like transposases described by U.S. Pat. Nos. 11,162,102, 11,060,109, 11,060,098, 11,060,086, 10,927,384, 10,435,696, 10,344,285, 10, 253,454, 10,041,077, 9,580,697, 9,574,209, 9,534,234, 9,428,767, and US20090042297.

These expression vectors are typically replicable in the host organisms either as episomes or as an integral part of the host chromosomal DNA. Commonly, expression vectors contain selection markers, e.g., ampicillin resistance or kanamycin resistance for propagation in bacterial hosts, or glutamine synthetase, dihydrofolate reductase, puromycin resistance, blasticidin resistance or hygromycin resistance for propagation in mammalian hosts, to permit detection of those cells transformed with the desired DNA sequences.

E. coli is a prokaryotic host useful for expressing antibodies, particularly antibody fragments. Microbes, such as yeast, are also useful for expression. Saccharomyces is a yeast host with suitable vectors having expression control sequences, an origin of replication, termination sequences, and the like as desired. Typical promoters include 3-phosphoglycerate kinase and other glycolytic enzymes. Inducible yeast promoters include, among others, promoters from alcohol dehydrogenase, isocytochrome C, and enzymes responsible for maltose and galactose utilization.

Mammalian cells can be used for expressing nucleotide segments encoding immunoglobulins or fragments thereof. See Winnacker, From Genes to Clones, (VCH Publishers, NY, 1987). A number of suitable host cell lines capable of secreting intact heterologous proteins have been developed, and include CHO cell lines, various COS cell lines, HeLa cells, HEK293 cells, L cells, and non-antibody-producing myelomas including Sp2/0 and NSO. The cells can be nonhuman. Expression vectors for these cells can include expression control sequences, such as an origin of replication, a promoter, an enhancer (Queen et al., Immunol. Rev. 89:49 (1986)), and necessary processing information sites, such as ribosome binding sites, RNA splice sites, polyadenylation sites, and transcriptional terminator sequences. Expression control sequences can include promoters derived from endogenous genes, cytomegalovirus, SV40, adenovirus, bovine papillomavirus, and the like. See Co et al., J. Immunol. 148:1149 (1992).

Alternatively, antibody coding sequences can be incorporated in transgenes for introduction into the genome of a transgenic animal and subsequent expression in the milk of the transgenic animal (see, e.g., U.S. Pat. Nos. 5,741,957; 5,304,489; and 5,849,992). Suitable transgenes include coding sequences for light and/or heavy chains operably linked with a promoter and enhancer from a mammary gland specific gene, such as casein or beta lactoglobulin.

The vectors containing the DNA segments of interest can be transferred into the host cell by methods depending on the type of cellular host. For example, calcium chloride transfection is commonly utilized for prokaryotic cells, whereas calcium phosphate treatment, electroporation, lipofection, biolistics, or viral-based transfection can be used for other cellular hosts. Other methods used to transform mammalian cells include the use of polybrene, protoplast fusion, liposomes, electroporation, and microinjection. For production of transgenic animals, transgenes can be microinjected into fertilized oocytes or can be incorporated into the genome of embryonic stem cells, and the nuclei of such cells transferred into enucleated oocytes.

Having introduced vector(s) encoding antibody heavy and light chains into cell culture, cell pools can be screened for productivity and quality of antibodies in serum-free media. Top-producing cell pools can then be subjected to FACS-based single-cell cloning to generate monoclonal lines. Antibodies produced by single cell clones can also be tested for turbidity, filtration properties, PAGE, IEF, UV scan, HP-SEC, carbohydrate-oligosaccharide mapping, mass spectrometry, and binding assay, such as ELISA or BIACORE™. A selected clone can then be banked in multiple vials and stored frozen for subsequent use.

Once expressed, multi-specific antibodies can be purified according to procedures including CH1 affinity chromatography, protein A capture, HPLC purification, viral inactivation, diafiltration, anion and cation column chromatography, gel electrophoresis and the like (see generally, Scopes, Protein Purification (Springer-Verlag, NY, 1982)). Purification can include separating multi-specific antibodies from mismatched associations of their component chains as well as from host impurities. CAPTURE SELECT® CH1-XL Affinity Matrix from THERMO FISHER SCIENTIFIC recognizes the CH1 region of human IgG antibodies, and can be used for purification of any bispecific antibody including a CH1 region away from homodimeric antibodies having a Cμ4 region replacing CH1.

Methodology for commercial production of antibodies can be employed, including codon optimization, selection of promoters, selection of transcription elements, selection of terminators, serum-free single cell cloning, cell banking, use of selection markers for amplification of copy number, or improvement of protein titers (see, e.g., U.S. Pat. Nos. 5,786,464; 6,114,148; 6,063,598; 7,569,339; WO2004/050884; WO2008/012142; WO2008/012142; WO2005/019442; WO2008/107388; WO2009/027471; and U.S. Pat. No. 5,888,809).

### VIII. Nucleic Acids/Polynucleotides

The invention further provides nucleic acids encoding any of the antibody chains described above. Optionally, such nucleic acids further encode a signal peptide and can be expressed with the signal peptide linked to the constant region coding sequences of nucleic acids can be operably linked with regulatory sequences to ensure expression of the coding sequences, such as a promoter, enhancer, ribosome binding site, transcription termination signal, and the like. The nucleic acids encoding heavy and light chains can occur in isolated form or can be cloned into one or more vectors. The nucleic acids can be synthesized by, for example, solid state synthesis or PCR of overlapping oligonucleotides. Nucleic acids encoding heavy and light chains can be joined as one contiguous nucleic acid, e.g., within an expression vector, or can be separate, e.g., each cloned into its own expression vector.

### IX. Methods of Treatment and Pharmaceutical Compositions

The multi-specific antibodies of the invention can be used for treating cancers in which at least one arm of a multi-specific antibody binds to a target antigen expressed or overexpressed in the cancer. The multi-specific antibodies can be used to treat solid tumors, and hematological malignancies. Hematological malignancies include leukemia (e.g., T cell large granular lymphocyte leukemia), lymphoma (Hodgkin's or Non-Hodgkin's), or multiple myeloma. Solid tumors include skin (e.g., melanoma), ovarian, endometrial, kidney, liver, pancreas, bladder, breast, ovarian, prostate, rectum, colon, gastric, intestinal, pancreatic, lung, thymus, thyroid, kidney and brain.

Multi-specific antibodies of the invention can also be used for treating pathogenic infections when the multi-specific antibody has at least one arm specifically binding to an antigen epitope expressed in infected cells or on a pathogen. Such an antigen can be encoded by the pathogen or can be expressed by the cell in response to infection by the pathogen. Examples of such antigens expressed in infected cells are human immune deficiency virus (HIV) glycoproteins gp41 and gp120, human T-cell leukemia virus type 1 (HTLV-1) Env protein, herpes simplex virus (HSV) glycoproteins gB and gH, influenza hemagglutinin (HA) and neuraminidase (NA), and respiratory syncytial virus (RSV) F protein. Examples of pathogenic infections treatable with multi-specific antibodies include viral, bacterial, protozoan or fungal infection. Some example of viral infections include HIV, hepatitis (A, B, or C), herpes virus (e.g., VZV, HSV-1, HAV-6, HSV-II, CMV, and Epstein Barr virus), adenovirus, XMRV, influenza virus, flaviviruses, echovirus, rhinovirus, coxsackie virus, coronavirus, respiratory syncytial virus, mumps virus, rotavirus, measles virus, rubella virus, parvovirus, vaccinia virus, HTLV virus, dengue virus, MLV-related virus, papillomavirus, molluscum virus, poliovirus, rabies virus, JC virus and arboviral encephalitis virus. Some examples of bacterial infections include chlamydia, rickettsia bacteria, mycobacteria, staphylococci, streptococci, pneumococci, meningococci and gonococci, klebsiella, proteus, serratia, pseudomonas, legionella, diphtheria, salmonella, bacilli, cholera, tetanus, botulism, anthrax, plague, leptospirosis, Lyme's disease bacteria, streptococci, or neisseria. Some examples of pathogenic fungi include Candida, Aspergillus, Cryptococcus, Histoplasma, Pneumocystis and Stachybotrys. Examples of protozoa include Cryptosporidium, Giardia lamblia and plasmodium.

Multi-specific antibodies are administered in an effective regime meaning a dosage, route of administration and frequency of administration that delays the onset, reduces the severity, inhibits further deterioration, and/or ameliorates at least one sign or symptom of a condition. If a subject is already suffering from a disorder, the regime can be referred to as a therapeutically effective regime. If the subject is at elevated risk of the condition relative to the general population but is not yet experiencing symptoms, the regime can be referred to as a prophylactically effective regime. In some instances, therapeutic or prophylactic efficacy can be observed in an individual subject relative to historical controls or past experience in the same subject. In other instances, therapeutic or prophylactic efficacy can be demonstrated in a preclinical or clinical trial in a population of treated subjects relative to a control population of untreated subjects.

Preferably a multi-specific antibody exhibits at least additive and more preferably synergistic activity against a cancer or infected cell compared with its component antibodies individually. Synergy is preferably assessed quantitatively such as discussed by Tallarida, Genes Cancer. 2011 November; 2(11): 1003-1008. Preferably a multi-specific antibody also exhibits increased activity compared with a mixture of its component antibodies, each at equimolar concentration with the multi-specific antibody.

Exemplary dosages for a multi-specific antibody are 0.01-20, or 0.5-5, or 0.01-1, or 0.01-0.5 or 0.05-0.5 mg/kg body weight (e.g., 0.1, 0.5, 1, 2, 3, 4 or 5 mg/kg) or 10-1500 mg as a fixed dosage. The dosage depends on the condition of the patient and response to prior treatment, if any, whether the treatment is prophylactic or therapeutic and whether the disorder is acute or chronic, among other factors.

Administration can be parenteral, intravenous, oral, subcutaneous, intra-arterial, intracranial, intrathecal, intraperitoneal, topical, intranasal or intramuscular. Administration into the systemic circulation by intravenous or subcutaneous administration is preferred. Intravenous administration can be, for example, by infusion over a period such as 30-90 min.

The frequency of administration depends on the half-life of the multi-specific antibody in the circulation, the condition of the subject and the route of administration among other factors. The frequency can be daily, weekly, monthly, quarterly, or at irregular intervals in response to changes in the patient's condition or progression of the disorder being treated. An exemplary frequency for intravenous administration is between weekly and quarterly over a continuous cause of treatment, although more or less frequent dosing is also possible. For subcutaneous administration, an exemplary dosing frequency is daily to monthly, although more or less frequent dosing is also possible.

The number of dosages administered depends on whether the disorder is acute or chronic and the response of the disorder to the treatment. For acute disorders or acute exacerbations of chronic disorders, between 1 and 10 doses are often sufficient. Sometimes a single bolus dose, optionally in divided form, is sufficient for an acute disorder or acute exacerbation of a chronic disorder. Treatment can be repeated for recurrence of an acute disorder or acute exacerbation. For chronic disorders, a multi-specific antibody can be administered at regular intervals, e.g., weekly, fortnightly, monthly, quarterly, every six months for at least 1, 5 or 10 years, or the life of the subject.

Pharmaceutical compositions are preferably suitable for parenteral administration to a human (e.g., according to the standard of the FDA). Pharmaceutical compositions for parenteral administration are preferably sterile and substantially isotonic and manufactured under GMP conditions. Pharmaceutical compositions can be provided in unit dosage form (i.e., the dosage for a single administration). Pharmaceutical compositions can be formulated using one or more pharmaceutically acceptable carriers, diluents, excipients or auxiliaries. Pharmaceutically acceptable means suitable for human administration, e.g., approved or approvable by the FDA. The formulation depends on the route of administration chosen. For injection, antibodies can be formulated in aqueous solutions, preferably in physiologically compatible buffers such as Hank's solution, Ringer's solution, or physiological saline or acetate buffer (to reduce discomfort at the site of injection). The solution can contain formulatory agents such as suspending, stabilizing and/or dispersing agents. Alternatively antibodies can be in lyophilized form for constitution with a suitable vehicle, e.g., sterile pyrogen-free water, before use.

Treatment with the multi-specific antibodies of the invention can be combined with other treatments effective against the disorder being treated. When used in treating cancer, the multi-specific antibodies of the invention can be combined with chemotherapy, radiation, stem cell treatment, surgery or treatment with other biologics such as Herceptin™ (trastuzumab) against the HER2 antigen, Avastin™ (bevacizumab) against VEGF, or antibodies to the EGF receptor, such as (Erbitux™, cetuximab), and Vectibix™ (panitumumab). Chemotherapy agents include chlorambucil, cyclophosphamide or melphalan, carboplatin, daunorubicin, doxorubicin, idarubicin, and mitoxantrone, methotrexate, fludarabine, and cytarabine, etoposide or topotecan, vincristine and vinblastine. For infections, treatment can be in combination with antibiotics, anti-virals, anti-fungal or anti-protozoan agents or the like.

### X. Other Methods

The multi-specific antibodies of the invention also find use in diagnostic, prognostic and laboratory methods. They may be used to measure the level of an antigen expressed by a cancer or in the circulation of a patient with a cancer, to determine if the level is measurable or even elevated, and therefore to follow and guide treatment of the cancer, because cancers associated with measurable or elevated levels of an antigen are most susceptible to treatment with a multi-specific antibody comprising an arm binding to the cancer. The multi-specific antibodies can be used for an ELISA assay, radioimmunoassay or immunohistochemistry among others. The multi-specific antibodies can be labeled with fluorescent molecules, spin-labeled molecules, enzymes or radioisotopes, and may be provided in the form of a kit with all the necessary reagents to perform the assay.

All citations of patent filings, websites, other publications, sequence comparison algorithms, accession numbers and the like cited above or below are incorporated by reference in their entirety for all purposes to the same extent as if each individual item were specifically and individually indicated to be so incorporated by reference. If different versions of an accession number are available at different times, the version in effect at the effective filing date of this application is meant. The effective filing date means the earlier of the actual filing date or filing date of a priority application referring to the accession number if applicable. Likewise if different versions of a publication, algorithm, website or the like are published at different times, the version most recently published at the effective filing date of the application is meant unless otherwise indicated. Any feature, step, element, aspect, or aspect of the invention can be used in combination with any other unless specifically indicated otherwise. Although the present invention has been described in some detail by way of illustration and example for purposes of clarity and understanding, it will be apparent that certain changes and modifications may be practiced within the scope of the appended claims.

### EXAMPLES

The objective as described in the present disclosure was to create an HC/LC pair that is orthogonal to normal HC/LC pairing (which happens between the CH1 of the heavy chain and the kappa or lambda constant region of the light chain). For bispecific formats that have two heavy chains and two light chains, using the normal pairing for one half antibody, and the orthogonal pairing for the other is intended to prevent LC/HC chain mispairing. Alternatively, engineered components can also be used to construct a second orthogonal HC/LC pairing, different from the first HC/LC pairing, thereby constructing a complete antibody (e.g., an antibody comprising a total of two heavy chains and two light chains), where that entire antibody is specifically engineered to have a desired bispecific epitope binding specificity.

### Example 1

### Association Between IgM Cμ4 and Kappa Constant Regions

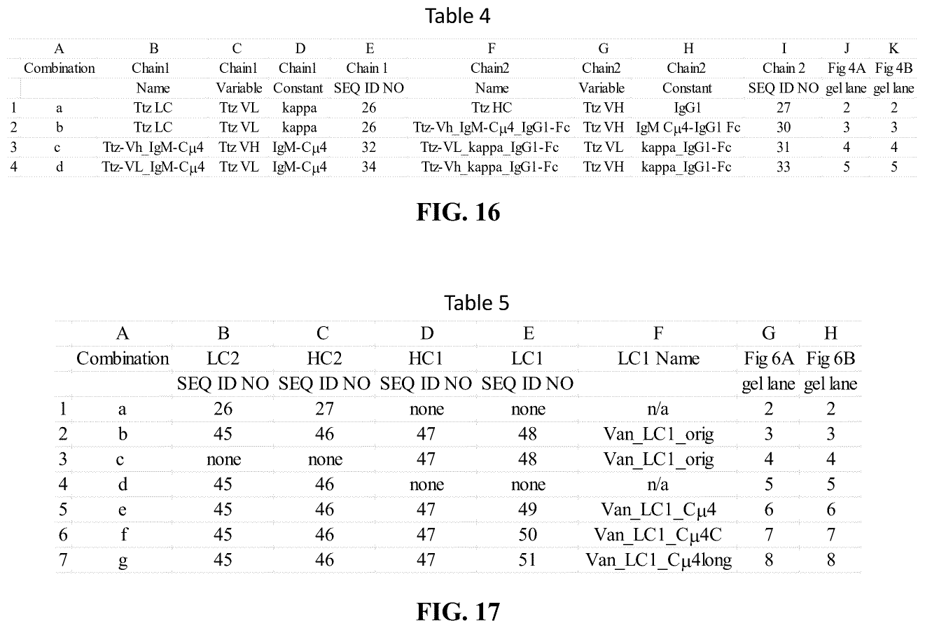

The pairing of heavy and light antibody chains was examined with various regions being replaced by the IgM Cμ4 region. Polynucleotides encoding antibody chains were constructed, each comprised an open reading frame encoding an antibody chain operably linked to a CMV promoter and rabbit globin polyadenylation sequence such that the open reading frame was expressible in a cultured mammalian cell. These polynucleotides are summarized in FIG. 16 Table 4 and were as follows.

A polynucleotide for expression of an antibody light chain (named Ttz LC, with mature amino acid sequence SEQ ID NO:26) comprised a sequence encoding, from N-terminus to C-terminus: a secretion signal, the light chain variable region of the antibody (with amino acid sequence SEQ ID NO:28), a human kappa constant region (with amino acid sequence SEQ ID NO:35).

A polynucleotide for expression of an antibody heavy chain (named Ttz HC, with mature amino acid sequence SEQ ID NO:27) comprised a sequence encoding, from N-terminus to C-terminus: a secretion signal, the heavy chain variable region of the antibody (with amino acid sequence SEQ ID NO:29), and a human IgG1 constant region (with amino acid sequence SEQ ID NO:36).

A polynucleotide for expression of a modified version of the heavy chain (named Ttz-Vh_IgM-Cμ4_IgG1-Fc, with mature amino acid sequence SEQ ID NO:30) comprised a sequence encoding, from N-terminus to C-terminus: a secretion signal, the heavy chain variable region of the antibody (with amino acid sequence SEQ ID NO:29), an alanine residue, a Cμ4 region of human IgM (with amino acid sequence SEQ ID NO:23), a human IgG1 hinge region (with amino acid sequence SEQ ID NO:2), a human IgG1 CH2 region (with amino acid sequence SEQ ID NO:3) and a human IgG1 CH3 region (with amino acid sequence SEQ ID NO:4).

A polynucleotide for expression of a modified version of the heavy chain (named Ttz-VL_kappa_IgG1-Fc, with mature amino acid sequence SEQ ID NO:31) comprised a sequence encoding, from N-terminus to C-terminus: a secretion signal, the light chain variable region of the antibody (with amino acid sequence SEQ ID NO:28), a human kappa region (with amino acid sequence SEQ ID NO:26), a partial human IgG1 hinge region (with amino acid sequence SEQ ID NO:37), a human IgG1 CH2 region (with amino acid sequence SEQ ID NO:3) and a human IgG1 CH3 region (with amino acid sequence SEQ ID NO:4). The partial truncation of the hinge placed the final cysteine of the kappa constant region in the same position in the hinge normally occupied by the first IgG1 hinge cysteine.

A polynucleotide for expression of a modified version of the light chain (named Ttz-Vh_IgM-Cμ4, with mature amino acid sequence SEQ ID NO:32) comprised a sequence encoding, from N-terminus to C-terminus: a secretion signal, the heavy chain variable region of the antibody (with amino acid sequence SEQ ID NO:29), an alanine residue and a truncated human IgM Cμ4 constant region (with amino acid sequence SEQ ID NO:23).

A polynucleotide for expression of a modified version of the heavy chain (named Ttz-Vh_kappa_IgG1-Fc, with mature amino acid sequence SEQ ID NO:33) comprised a sequence encoding, from N-terminus to C-terminus: a secretion signal, the heavy chain variable region of the antibody (with amino acid sequence SEQ ID NO:29), an alanine residue, a human kappa region (with amino acid sequence SEQ ID NO:26), a partial human IgG1 hinge region (with amino acid sequence SEQ ID NO:37), a human IgG1 CH2 region (with amino acid sequence SEQ ID NO:3) and a human IgG1 CH3 region (with amino acid sequence SEQ ID NO:4). The partial truncation of the hinge placed the final cysteine of the kappa constant region in the same position in the hinge normally occupied by the first IgG1 hinge cysteine.

A polynucleotide for expression of a modified version of the light chain (named Ttz-VL_IgM-Cμ4, with mature amino acid sequence SEQ ID NO:34) comprised a sequence encoding, from N-terminus to C-terminus: a secretion signal, the light chain variable region of the antibody (with amino acid sequence SEQ ID NO:28), a glycine residue and a truncated human IgM Cμ4 constant region (with amino acid sequence SEQ ID NO:23).

Polynucleotides encoding one heavy chain (or modified heavy chain) and one light chain (or modified light chain) were co-transfected into HEK 293 cells, and the cells were cultured for 7 days in THERMO FISHER SCIENTIFIC Expi293™ media. Protein was purified from clarified culture supernatant using protein A affinity chromatography and analyzed on SDS polyacrylamide gels with or without reducing agent. FIG. 16 Table 4 shows the different chain combinations tested, and identifies the SEQ ID NOs corresponding to the amino acid sequences of each mature chain.

FIG. 4A shows a reduced gel and FIG. 4B shows a non-reduced gel of the polypeptides purified by binding and elution from protein A resin. The sequence details of the co-expressed polypeptides in each lane are shown in FIG. 16 Table 4. Light chain bands are indicated by LC, heavy chain bands are indicated by HC, fully assembled molecules are also indicated. Lanes 1 and 6 in FIGS. 4A and 4B contain molecular weight markers.

FIG. 16 Table 4 shows the two polypeptide chains co-expressed from polynucleotides as described in Example 1. Column A shows the polypeptide combination name; Column B shows the name of chain 1; Column C shows the chain 1 variable sequence name (VL is the light chain variable region, VH is the heavy chain variable region); Column D shows the chain 1 constant region name; Column E shows the SEQ ID NO corresponding to the amino acid sequence of mature chain 1; Column F shows the name of chain 2; Column G shows the chain 2 variable sequence name (VL is the light chain variable region, VH is the heavy chain variable region); Column H shows the chain 2 constant region name; Column I shows the SEQ ID NO corresponding to the amino acid sequence of mature chain 1; Column J shows the gel lane in FIG. 4A corresponding to the protein A-purified polypeptide combination; Column K shows the gel lane in FIG. 4B corresponding to the protein A-purified polypeptide combination.

FIG. 4A lane 2 shows the behavior of an unmodified antibody (FIG. 16 Table 4 combination a). The heavy and light chains are both purified by a protein A column. Protein A does not bind to the antibody variable region or to the kappa constant region, it binds to the CH2/CH3 region of IgGs, so the light chain co-purifies with the heavy chain by virtue of its association with the heavy chain. The association of light and heavy chains of the unmodified antibody can be seen directly on the non-reduced gel in FIG. 4B lane 2.

FIG. 4A lane 3 shows that for polypeptide combination b, wherein the IgG1 CH1 region of the antibody is replaced by an IgM Cμ4 region with amino acid sequence SEQ ID NO:23, the size of the heavy chain is increased slightly, and the light chain co-purifies with the heavy chain, indicating that heavy and light chains are associated with each other during the protein A purification. The association of a light chain (with a native sequence kappa constant region; i.e., an unmodified kappa constant region) and a heavy chain (with IgG1 CH1 region replaced by a native sequence IgM Cμ4 region, i.e., an unmodified IgM Cμ4 region) can be seen directly on the non-reduced gel in FIG. 4B lane 3. Although most of the modified antibody in FIG. 4B lane 3 remains fully assembled in the non-reduced gel, some appears to dissociate: a partially assembled (2xHC) band is visible at approximately 100 kDa, and some LC at approximately 20 kDa. In an unmodified antibody, there is a covalent disulfide bond formed between the C-terminal cysteine of the kappa constant region (Kabat position 214) (or the cysteine which is the penultimate residue in the lambda constant region, Kabat position 214) and a cysteine in the IgG hinge region of human IgG1 (EU position 220) or EU position 131 in the CH1 of human IgG2, IgG3 or IgG4. In the modified heavy chain with the IgG1 CH1 region replaced by an IgM Cμ4 region, the IgG hinge region is intact with its cysteines in their original position. However, it appears that a minor fraction of the light chain is associated with the heavy chain (because they co-purify) but not covalently linked through a cysteine-cysteine disulfide bond (because they dissociate on an SDS gel).

FIG. 4A lane 5 shows that for polypeptide combination d, wherein the IgG1 CH1 region of the heavy chain is replaced by the kappa light constant region, and the kappa constant region of the light chain is replaced by an IgM Cμ4 region with amino acid sequence SEQ ID NO:23, the size of the heavy chain is increased slightly and the size of the light chain is decreased slightly compared to the unmodified antibody (compare FIG. 4A lane 5 with lane 2). IgM molecules in general, and the IgM Cμ4 region in particular, do not bind to protein A resin. Thus the presence of heavy and light chains in the reduced gel indicates that heavy and light chains were associated with each other during the protein A purification. However, in the non-reduced gel (FIG. 4B, lane 5), no fully assembled molecules are visible, although heavy chain dimer (2xHC) and light chain can be seen to have co-purified. The reason for this lack of fully assembled molecules on the non-reduced gel is that the IgM Cμ4 region used, with amino acid sequence SEQ ID NO:23, lacks a C-terminal cysteine. It is therefore unable to form a covalent cysteine-cysteine disulfide bond with the heavy chain.

FIG. 4A lane 4 shows results for polypeptide combination c, which is similar to combination d except that the heavy variable region is moved to the light chain, and the light variable region is moved to the heavy chain. As for combination d, both heavy and light chains can be seen in the reduced gel, indicating that heavy and light chains were associated with each other during protein A purification. Also, in the non-reduced gel (FIG. 4B, lane 4), no fully assembled molecules are visible, although heavy chain dimer (2xHC) and light chain can be seen to have co-purified. The lack of fully assembled molecules on the non-reduced gel is again attributable to the sequence of the IgM Cμ4 region used. The lack of a C-terminal cysteine prevents formation of a covalent cysteine-cysteine disulfide bond with the heavy chain. From this data, it is concluded that an IgM Cμ4 region and a kappa constant region are compatible antibody-pairing regions.

### Example 2

### Use of IgM Cμ4 to Prevent Light Chain Mispairing in a Four Chain Bispecific Antibody

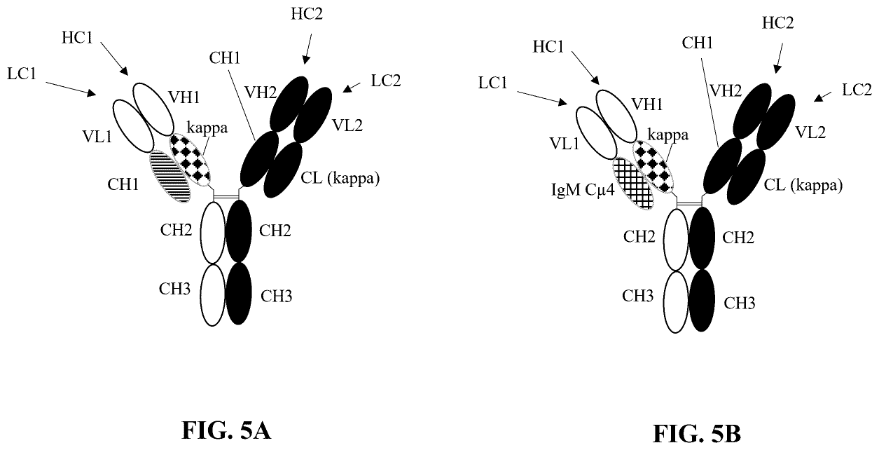

Polynucleotides encoding four chains of a four-chain bispecific antibody were constructed, as well as a modified version of a four-chain bispecific antibody comprising an IgM Cμ4 region. FIG. 5A shows the original molecule, FIG. 5B shows the modified molecule comprising the IgM Cμ4 region.

FIG. 5A represents a four chain bispecific antibody. The second half antibody (LC2 and HC2) comprise normally arranged chains. The light chain, LC2 comprises a light chain variable region and a kappa constant region. The heavy chain, HC2 comprises a heavy chain variable region an IgG1 CH1 region, a hinge region, an IgG1 CH2 region and an IgG1 CH3 region. The first half antibody (LC1 and HCl) comprises modified chains. Light chain LC1 comprises a light chain variable region and an IgG1 CH1 region. Heavy chain HCl comprises a heavy chain variable region, a kappa constant region, an IgG1 hinge region, an IgG1 CH2 region and an IgG1 CH3 region. FIG. 5B represents a modified version of this four chain bispecific antibody. HCl, LC2 and HC2 are all as described for FIG. 5A. LC1 is modified so that it comprises the same heavy chain variable region as the molecule in FIG. 5A, but the IgG1 CH1 region has been replaced by an IgM Cμ4 region.

Polynucleotides encoding chains of the four-chain bispecific antibody were constructed, each comprised an open reading frame encoding an antibody chain operably linked to a CMV promoter and rabbit globin polyadenylation sequence such that the open reading frame was expressible in a cultured mammalian cell. The polynucleotides were as follows.

A polynucleotide for expression of an antibody light chain (named Van_LC2), with mature amino acid sequence SEQ ID NO:45) comprised a sequence encoding, from N-terminus to C-terminus: a secretion signal, the light chain variable region of the antibody (with amino acid sequence SEQ ID NO:38), a human kappa constant region (with amino acid sequence SEQ ID NO:35).

A polynucleotide for expression of a modified antibody heavy chain (named Van_HC2, with mature amino acid sequence SEQ ID NO:46) comprised a sequence encoding, from N-terminus to C-terminus: a secretion signal, the heavy chain variable region of the antibody (with amino acid sequence SEQ ID NO:39), a human IgG1 CH1 region (with amino acid sequence SEQ ID NO:1), a human IgG1 hinge region (with amino acid sequence SEQ ID NO:2), a human IgG1 CH2 region (with amino acid sequence SEQ ID NO:3) and a modified human IgG1 CH3 region (with amino acid sequence SEQ ID NO:43).

A polynucleotide for expression of a modified antibody heavy chain (named Van_HCl, with mature amino acid sequence SEQ ID NO:47) comprised a sequence encoding, from N-terminus to C-terminus: a secretion signal, the heavy chain variable region of the antibody (with amino acid sequence SEQ ID NO:40), a modified human kappa region (with amino acid sequence SEQ ID NO:42), a truncated human IgG1 hinge region (with amino acid sequence SEQ ID NO:37), a human IgG1 CH2 region (with amino acid sequence SEQ ID NO:3) and a modified human IgG1 CH3 region (with amino acid sequence SEQ ID NO:44).

A polynucleotide for expression of a modified antibody light chain (named Van_LC1_orig), with mature amino acid sequence SEQ ID NO:48) comprised a sequence encoding, from N-terminus to C-terminus: a secretion signal, the light chain variable region of the antibody (with amino acid sequence SEQ ID NO:41), a human IgG1 CH1 constant region (with amino acid sequence SEQ ID NO:35) and a truncated upper hinge region (with amino acid sequence SEQ ID NO:52).

A polynucleotide for expression of a modified antibody light chain (named Van_LC1_Cμ4), with mature amino acid sequence SEQ ID NO:49) comprised a sequence encoding, from N-terminus to C-terminus: a secretion signal, the light chain variable region of the antibody (with amino acid sequence SEQ ID NO:41), a glycine residue and a truncated human IgM Cμ4 constant region (with amino acid sequence SEQ ID NO:23).

A polynucleotide for expression of a modified antibody light chain (named Van_LC1_Cμ4 C), with mature amino acid sequence SEQ ID NO:50) comprised a sequence encoding, from N-terminus to C-terminus: a secretion signal, the light chain variable region of the antibody (with amino acid sequence SEQ ID NO:41), a glycine residue and a truncated human IgM Cμ4 constant region in which the final threonine residue has been altered to a cysteine residue (with amino acid sequence SEQ ID NO:24).

A polynucleotide for expression of a modified antibody light chain (named Van_LC1_Cμ4 long), with mature amino acid sequence SEQ ID NO:51) comprised a sequence encoding, from N-terminus to C-terminus: a secretion signal, the light chain variable region of the antibody (with amino acid sequence SEQ ID NO:41), a glycine residue and a human IgM Cμ4 constant region (with amino acid sequence SEQ ID NO:25).

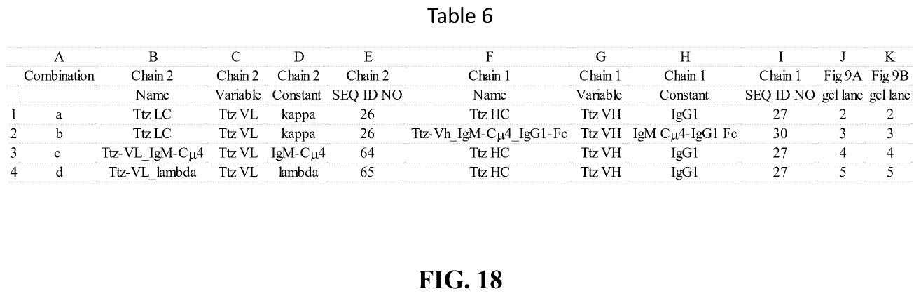

Polynucleotides encoding one or two heavy chain (or modified heavy chains) and one or two light chains (or modified light chains) were co-transfected into HEK 293 cells, and the cells were cultured for seven days in THERMO FISHER SCIENTIFIC Expi293™ media. Protein was purified from clarified culture supernatant using protein A affinity chromatography and analyzed on SDS polyacrylamide gels with or without reducing agent. FIG. 17 Table 5 shows the different chain combinations tested and identifies the SEQ ID NOs corresponding to the amino acid sequences of each mature chain.

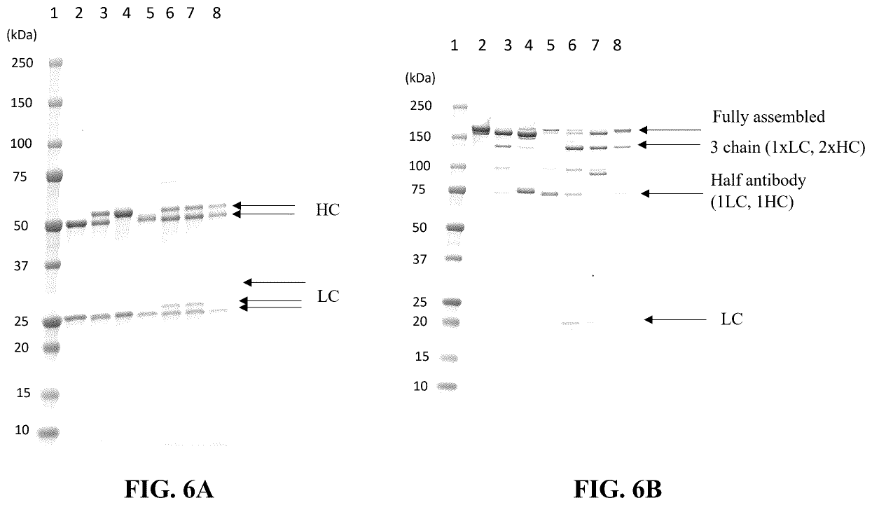

FIG. 6A shows a reduced gel and FIG. 6B shows a non-reduced gel of the polypeptides purified by binding and elution from protein A resin. The details of the co-expressed polypeptides in each lane are shown in FIG. 17 Table 5. Light chain bands are indicated by LC, heavy chain bands are indicated by HC. On the non-reduced gel, fully assembled molecules and some partial assembly products are also indicated. Lane 1 contains molecular weight markers.

FIG. 17 Table 5 shows the two polypeptide chains co-expressed from polynucleotides as described in Example 2. Column A shows the polypeptide combination name; Column B shows the SEQ ID NO corresponding to the amino acid sequence of mature chain LC2; Column C shows the SEQ ID NO corresponding to the amino acid sequence of mature chain HC2; Column D shows the SEQ ID NO corresponding to the amino acid sequence of mature chain HCl; Column E shows the SEQ ID NO corresponding to the amino acid sequence of mature chain LC1; Column F shows the name of the LC1 chain; Column G shows the gel lane in FIG. 6A corresponding to the protein A-purified polypeptide combination; Column H shows the gel lane in FIG. 6B corresponding to the protein A-purified polypeptide combination.

FIG. 6A in lane 2 shows the heavy and light chains of a control antibody (the same antibody shown in FIGS. 4A and 4B lane 2). FIG. 6B lane 2 shows that this antibody runs on a non-reduced gel with an apparent molecular weight of around 150 kDa, consistent with a tetramer comprising two heavy and two light chains of 50 and 25 kDa respectively.

FIG. 6A in lane 3 shows the protein A-purified products when Van_LC2, Van_HC2, Van_HCl and the original Van_LC1_orig were co-expressed. Two distinct heavy chains can be seen in the reduced gel (FIG. 6A), though the light chains cannot be resolved. Multiple bands are visible in the non-reduced gel (FIG. 6B, lane 3). The uppermost band, at approximately the same size as the fully assembled control antibody (approximately 150 kDa) corresponds to fully assembled four-chain product. Other bands have not been fully characterized, but from their sizes they are most likely approximately 75 kDa, half antibody (one heavy and one light chain); approximately 100 kDa, two heavy chains; approximately 125 kDa, three chains, one light and two heavy.

FIG. 6A in lane 4 in shows the expression of only one half of the bispecific antibody: Van_LC1_orig and Van_HCl alone, and lane 5 shows the expression of only the other half Van_LC2 and Van_HC2 alone. Comparison of FIG. 6A lanes 4 and 5 shows the two light chains migrate at the same size, though the heavy chains can be resolved. FIG. 6B lane 4 shows that the Van_LC1_orig/Van_HCl half antibody has a preference to assemble into homodimers: there is significantly more material in the “fully assembled” band at approximately 150 kDa than there is in the half antibody band at approximately 75 kDa. In contrast, there is more of the LC2/HC2 half antibody in FIG. 6B lane 5 in the half-antibody band at approximately 75 kDa than there is in the fully assembled band at approximately 150 kDa. This behavior is consistent with the presence of bulky “knob” mutations in Van_HC2 which disfavor homodimerization, while Van_HCl contains less bulky “hole” mutations which interfere less with the homodimerization of Van_HCl. The Van_LC1_orig/Van_HCl pair is also a cross-mab format, with the kappa constant region present on the heavy chain, and the IgG1 CH1 region plus a part of the upper IgG1 hinge present on the light chain (see FIG. 5A).

FIG. 6A in lanes 6, 7 and 8 show the presence of a second resolvable light chain band. In lanes 6 and 7 this band runs just above the original LC1/LC2 because the IgM Cμ4 region is larger than the IgG1 CH1 region of the original LC1. In FIG. 6A lane 8, the LC1 band is significantly larger as well as more diffuse. This is because the full IgM Cμ4 region includes an N-linked glycosylation site (the amino acid sequence Asn-Val-Ser) near its C-terminus, the heterogeneity of glycosylation makes the chain larger and more heterogeneous in size. This sequence is lacking in the truncated versions of the IgM Cμ4 region used in the proteins analyzed in lanes 6 and 7, but present in the full sequence analyzed in lane 8. Thus FIG. 6A shows that all four chains described in FIG. 17 Table 5 are co-expressed. In FIG. 6B, lane 6 shows the assembly products when a truncated Cμ4 region with amino acid sequence SEQ ID NO:23 is used to replace the IgG1 CH1 region plus partial hinge in Van_LC1_orig. Many of the partial assembly products seen in the original molecule can be seen (compare lane 3 with lane 6). Notably, however, very little full-sized assembly product can be seen, while there appears to be a very significant amount of three-chain product 1xLC 2xHC. This is because the IgM Cμ4 region used (with amino acid sequence SEQ ID NO:23) lacks a cysteine near its C-terminus, so the IgM Cμ4-containing Van_LC1_Cμ4 cannot covalently link to its corresponding heavy chain, as was seen in the comparable case in Example 1. As for the previous example, however, Van_LC1_Cμ4 can be seen to have co-purified with the bispecific antibody in lane 6 on both FIG. 6A and FIG. 6B. When the C-terminal threonine of the truncated IgM Cμ4 region is changed to a cysteine (Van_LC1_Cμ4 C has IgM Cμ4 region with amino acid sequence SEQ ID NO:24), the assembly pattern changes in the non-reduced gel (FIG. 6B lane 7). The intensity of the band containing three-chain product (1xLC 2xHC) is somewhat reduced, while the intensity of fully assembled four-chain bispecific antibody is increased, indicating that the addition of the C-terminal cysteine leads to the formation of covalently-bonded bispecific antibody. Some free LC can still be seen in the non-reduced gel, indicating that disulfide bond formation is not complete and suggesting that further optimization of the C-terminal sequence may lead to more complete disulfide bond formation between LC1 and HCl. The full IgM Cμ4 region (with amino acid sequence SEQ ID NO:25) has a cysteine as its penultimate residue. When this sequence was used to replace the IgG1 CH1 region plus partial hinge of the original molecule, the amount of partial products seen was further reduced, and the assembly of fully assembled four chain bispecific antibody was increased (FIG. 6B lane 8). The presence of all four chains of the bispecific antibody were confirmed as present in the protein A-purified material from combinations e, f and g using mass spectrometry. From this data, it is concluded that a native, i.e., unmodified, IgM Cμ4 region can be used to pair with a kappa constant region in a multi-chain multi-specific antibody.

### Example 3

### Association Between IgM Cμ4 and IgG CH1 Constant Regions

The pairing of heavy and light antibody chains was examined with various regions being replaced by the IgM Cμ4 region. Polynucleotides encoding antibody chains were constructed, each comprised an open reading frame encoding an antibody chain operably linked to a CMV promoter and rabbit globin polyadenylation sequence such that the open reading frame was expressible in a cultured mammalian cell. The polynucleotides constructed were as follows.

A polynucleotide for expression of an antibody light chain (named Ttz LC, with mature amino acid sequence SEQ ID NO:26) comprised a sequence encoding, from N-terminus to C-terminus: a secretion signal, the light chain variable region of the antibody (with amino acid sequence SEQ ID NO:28), a human kappa constant region (with amino acid sequence SEQ ID NO:35).

A polynucleotide for expression of an antibody heavy chain (named Ttz HC, with mature amino acid sequence SEQ ID NO:27) comprised a sequence encoding, from N-terminus to C-terminus: a secretion signal, the heavy chain variable region of the antibody (with amino acid sequence SEQ ID NO:29), a human IgG1 constant region (with amino acid sequence SEQ ID NO:36).

A polynucleotide for expression of a modified version of the heavy chain (named Ttz-Vh_IgM-Cμ4_IgG1-Fc, with mature amino acid sequence SEQ ID NO:30) comprised a sequence encoding, from N-terminus to C-terminus: a secretion signal, the heavy chain variable region of the antibody (with amino acid sequence SEQ ID NO:29), an alanine residue, a Cμ4 region of human IgM (with amino acid sequence SEQ ID NO:23), a human IgG1 hinge region (with amino acid sequence SEQ ID NO:2), a human IgG1 CH2 region (with amino acid sequence SEQ ID NO:3) and a human IgG1 CH3 region (with amino acid sequence SEQ ID NO:4).

A polynucleotide for expression of a modified version of the light chain (named Ttz-VL_IgM-C μ4, with mature amino acid sequence SEQ ID NO:64) comprised a sequence encoding, from N-terminus to C-terminus: a secretion signal, the light chain variable region of the antibody (with amino acid sequence SEQ ID NO:28), a glycine residue and a truncated human IgM Cμ4 constant region with a C-terminal cysteine added (with amino acid sequence SEQ ID NO:24).

A polynucleotide for expression of a modified version of the light chain (named Ttz-VL_lambda, with mature amino acid sequence SEQ ID NO:65) comprised a sequence encoding, from N-terminus to C-terminus: a secretion signal, the light chain variable region of the antibody (with amino acid sequence SEQ ID NO:28) and a human lambda constant region (with amino acid sequence SEQ ID NO:66).

Polynucleotides encoding one heavy chain (or modified heavy chain) and one light chain (or modified light chain) were co-transfected into HEK 293 cells, and the cells were cultured for seven days in THERMO FISHER SCIENTIFIC Expi293™ media. Protein was purified from clarified culture supernatant using protein A affinity chromatography and analyzed on SDS polyacrylamide gels with or without reducing agent. FIG. 18 Table 6 shows the different chain combinations tested, and identifies the SEQ ID NOs corresponding to the amino acid sequences of each mature chain.

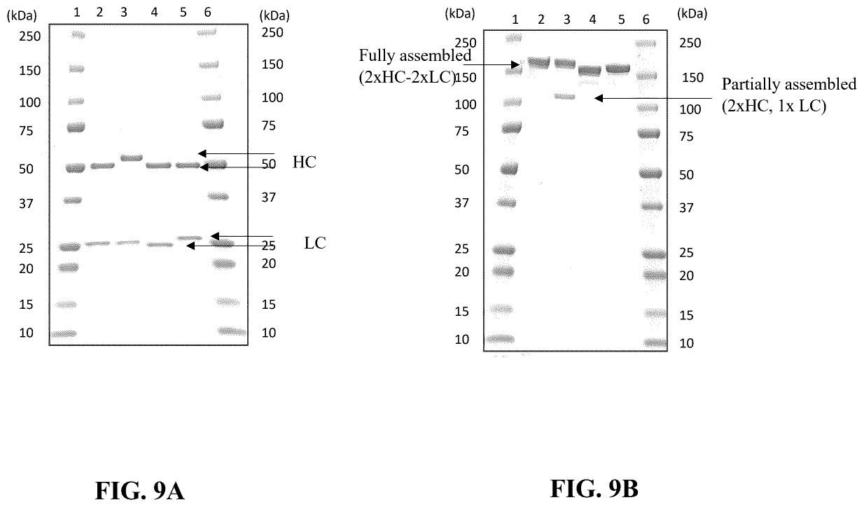

FIG. 9A shows a reduced gel and FIG. 9B shows a non-reduced gel of the polypeptides purified by binding and elution from protein A resin. The sequence details of the co-expressed polypeptides in each lane are shown in FIG. 18 Table 6. Light chain bands are indicated by LC, heavy chain bands are indicated by HC, fully assembled molecules are also indicated. Lanes 1 and 6 contain molecular weight markers.

FIG. 18 Table 6 shows the two polypeptide chains co-expressed from polynucleotides as described in Example 3. Column A shows the polypeptide combination name; Column B shows the name of chain 1; Column C shows the chain 1 variable sequence name (VL is the light chain variable region, VH is the heavy chain variable region); Column D shows the chain 1 constant region name; Column E shows the SEQ ID NO corresponding to the amino acid sequence of mature chain 1; Column F shows the name of chain 2; Column G shows the chain 2 variable sequence name (VL is the light chain variable region, VH is the heavy chain variable region); Column H shows the chain 2 constant region name; Column I shows the SEQ ID NO corresponding to the amino acid sequence of mature chain 1; Column J shows the gel lane in FIG. 9A corresponding to the protein A-purified polypeptide combination; Column K shows the gel lane in FIG. 9B corresponding to the protein A-purified polypeptide combination.

FIG. 9A lane 2 shows the behavior of an unmodified antibody (FIG. 18 Table 6 combination a). The heavy and light chains are both purified by a protein A column. Protein A does not bind to the antibody variable region or to the kappa constant region, it binds to the CH2/CH3 region of IgGs, so the light chain co-purifies with the heavy chain by virtue of its association with the heavy chain. The association of light and heavy chains of the unmodified antibody can be seen directly on the non-reduced gel in FIG. 9B lane 2.

FIG. 9A lane 3 shows that for polypeptide combination b, wherein the IgG1 CH1 region of the antibody is replaced by an IgM Cμ4 region with amino acid sequence SEQ ID NO:23, the size of the heavy chain is increased slightly, and the light chain co-purifies with the heavy chain, indicating that heavy and light chains are associated with each other during the protein A purification.

The association of unmodified light chain (comprising an unmodified, i.e., native, kappa constant region) and heavy chain (with IgG1 CH1 region replaced by IgM Cμ4 region) can be seen directly on the non-reduced gel in FIG. 9B lane 3. Although most of the modified antibody in FIG. 9B lane 3 remains fully assembled in the non-reduced gel, some appears to dissociate: a partially assembled (2xHC) band is visible at approximately 100 kDa, and some LC at approximately 20 kDa. In an unmodified antibody, there is a covalent disulfide bond formed between the C-terminal cysteine of the kappa constant region (or the cysteine which is the penultimate residue in the lambda constant region) and a cysteine in the IgG1 hinge region. In the modified heavy chain with the IgG1 CH1 region replaced by an IgM Cμ4 region, the IgG1 hinge region is intact with its cysteines in their original position. However, it appears that a minor fraction of the light chain is associated with the heavy chain (because they co-purify) but not covalently linked through a cysteine-cysteine disulfide bond (because they dissociate on an SDS gel).

FIG. 9A lane 4 shows that for polypeptide combination c, wherein the kappa region of the light chain is replaced by an IgM Cμ4 region with amino acid sequence SEQ ID NO:24 (which comprises a C-terminal cysteine), the size of the light chain is decreased slightly compared to the unmodified antibody (compare FIG. 9A lane 4 with lane 2). IgM molecules in general, and the IgM Cμ4 region in particular, do not bind to protein A resin. Thus, the presence of heavy and light chains in the reduced gel indicates that heavy and light chains were associated with each other during the protein A purification, showing that the unmodified, i.e., native sequence, IgM Cμ4 region pairs with the unmodified, i.e., native sequence, IgG CH1 region of the heavy chain.

In the non-reduced gel (FIG. 9B, lane 4), fully assembled molecules are visible, with no heavy chain dimer (2xHC) or unbound light chain visible.

FIG. 9A lane 5 shows results for polypeptide combination d, which is similar to combination a, except that the light chain kappa constant region is changed to a lambda constant region. As for combination a, both heavy and light chains can be seen in the reduced gel, indicating that heavy and light chains were associated with each other during protein A purification. Also, in the non-reduced gel (FIG. 9B, lane 5), only fully assembled molecules are visible.

This data shows that an IgM Cμ4 region and an IgG CH1 constant region are compatible antibody-pairing regions.

### Example 4

### Cysteine Modifications Conferring Specificity to IgM Cμ4—IgG CH1 Pairing

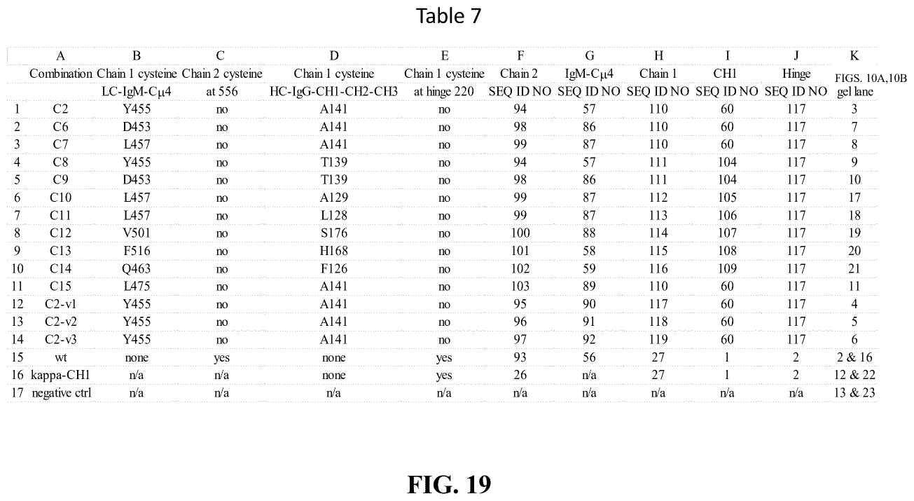

As shown in Example 3, an IgM Cμ4 region and an IgG CH1 constant region are compatible antibody-pairing regions. Because IgM Cμ4 pairs promiscuously with either CH1 or kappa constant regions, it is beneficial to modify both components of the desired pair to reduce non-specific pairing and improve specific pairing. Structural models of interactions between IgG CH1 and IgM Cμ4 were used to identify the locations of residues within each of these domains that might be replaced by cysteines that might be capable of forming a covalent disulfide bond between the two chains. In this way, 11 potential pairs of substitutions were identified, summarized in FIG. 19 Table 7.
(1) pair C2 comprised an IgG CH1 cysteine substitution at EU position 141 (normally an alanine) and a corresponding IgM Cμ4 cysteine substitution at position 455 (normally a tyrosine);
(2) pair C6 comprised an IgG CH1 cysteine substitution at EU position 141 (normally an alanine) and a corresponding IgM Cμ4 cysteine substitution at position 453 (normally an aspartate);
(3) pair C7 comprised an IgG CH1 cysteine substitution at EU position 141 (normally an alanine) and a corresponding IgM Cμ4 cysteine substitution at position 457 (normally a leucine);
(4) pair C8 comprised an IgG CH1 cysteine substitution at EU position 139 (normally a threonine) and a corresponding IgM Cμ4 cysteine substitution at position 455 (normally a tyrosine);
(5) pair C9 comprised an IgG CH1 cysteine substitution at EU position 139 (normally a threonine) and a corresponding IgM Cμ4 cysteine substitution at position 453 (normally an aspartate);
(6) pair C10 comprised an IgG CH1 cysteine substitution at EU position 129 (normally an alanine) and a corresponding IgM Cμ4 cysteine substitution at position 457 (normally a leucine);
(7) pair C11 comprised an IgG CH1 cysteine substitution at EU position 128 (normally a leucine) and a corresponding IgM Cμ4 cysteine substitution at position 457 (normally a leucine);
(8) pair C12 comprised an IgG CH1 cysteine substitution at EU position 176 (normally a serine) and a corresponding IgM Cμ4 cysteine substitution at position 501 (normally a valine);
(9) pair C13 comprised an IgG CH1 cysteine substitution at EU position 168 (normally a histidine) and a corresponding IgM Cμ4 cysteine substitution at position 516 (normally a phenylalanine);
(10) pair C14 comprised an IgG CH1 cysteine substitution at EU position 126 (normally a phenylalanine) and a corresponding IgM Cμ4 cysteine substitution at position 463 (normally a glutamine);
(11) pair C15 comprised an IgG CH1 cysteine substitution at EU position 141 (normally an alanine) and a corresponding IgM Cμ4 cysteine substitution at position 475 (normally a leucine).

Each of these cysteine pairs was tested for expression and disulfide bond formation in the context of an antibody, in which the kappa constant region was replaced by the IgM Cμ4 region. Polynucleotides encoding antibody chains were constructed, each comprised an open reading frame encoding an antibody chain operably linked to a CMV promoter and rabbit globin polyadenylation sequence such that the open reading frame was expressible in a cultured mammalian cell. The polynucleotides constructed were as follows.

Polynucleotides for expression of antibody light chains using an IgM Cμ4 pairing region each comprised a sequence encoding, from N-terminus to C-terminus: a secretion signal, the light chain variable region of the antibody (with amino acid sequence SEQ ID NO:28), a glycine residue and an IgM Cμ4 pairing region (with an amino acid sequence corresponding to a SEQ ID NO shown in FIG. 19 Table 7, column G). A polynucleotide for expression of a control antibody light chain, with mature amino acid sequence SEQ ID NO:26, comprised a sequence encoding, from N-terminus to C-terminus: a secretion signal, the light chain variable region of the antibody (with amino acid sequence SEQ ID NO:28), a human kappa constant region (with amino acid sequence SEQ ID NO:35). The SEQ ID NO corresponding to the mature amino acid sequence of each light chain is shown in FIG. 19 Table 7, column F.

Polynucleotides for expression of antibody heavy chains each comprised a sequence encoding, from N-terminus to C-terminus: a secretion signal, the heavy chain variable region of the antibody (with amino acid sequence SEQ ID NO:29), a human IgG1 CH1 constant region (with amino acid sequence corresponding to a SEQ ID NO shown in FIG. 19 Table 7, column I), a hinge region (with amino acid sequence corresponding to a SEQ ID NO shown in FIG. 19 Table 7, column J) and an Fc region (human IgG1 CH2 plus CH3 regions) with amino acid sequence SEQ ID NO:118. The SEQ ID NO corresponding to the mature amino acid sequence of each heavy chain is shown in FIG. 19 Table 7, column H.

Polynucleotides encoding one heavy chain (or modified heavy chain) and one light chain (or modified light chain) were co-transfected into HEK 293 cells, and the cells were cultured for seven days in THERMO FISHER SCIENTIFIC Expi293™ media. Culture supernatants were analyzed on SDS polyacrylamide gels without reducing agent. FIG. 19 Table 7 shows the different chain combinations tested, and identifies the SEQ ID NOs corresponding to the amino acid sequences of each mature chain.

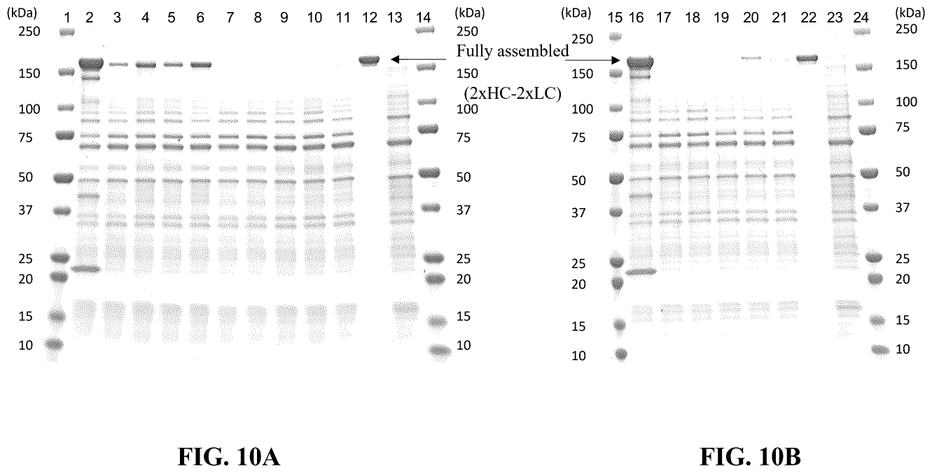

FIG. 10A shows lanes 1-14 and FIG. 10B shows lanes 15-24 of a non-reduced gel of the polypeptides expressed in the culture supernatant. The sequence details of the co-expressed polypeptides in each lane are shown in FIG. 19 Table 7. Fully assembled tetramers, comprising two light and two heavy chains migrate at a little over 150 kDa, and are indicated by arrows. Purified antibody comprising a natural kappa light chain and a natural IgG1 heavy chain is shown in lanes 12 and 22. Lanes 2 and 16 show supernatant from cells transfected with constructs comprising a natural IgG heavy chain and a light chain with an IgM Cμ4 pairing region in which the threonine at position 556 is mutated to a cysteine after which the chain is terminated, as described in Example 3 and shown in FIGS. 9A and 9B, lanes 4. Lanes 3, 7-11 and 17-21 show the assembled antibody produced when the heavy chain IgG hinge cysteine is mutated (as shown in FIG. 8B) and the terminal amino acid (position 556) of the IgM Cμ4 pairing region is mutated to an isoleucine and then alternative cysteine residues are introduced, one into the IgG CH1 region, the other into the IgM Cμ4 pairing region. Of the eleven different cysteine pairs tested, three resulted in detectable fully assembled antibody. These were:
(1) pair C2: CH1 with alanine 141 mutated to cysteine paired with IgM Cμ4 with a tyrosine 455 mutated to cysteine (FIG. 10A, lane 3);
(9) pair C13: CH1 with histidine 168 mutated to cysteine paired with IgM Cμ4 with phenylalanine 516 mutated to cysteine (FIG. 10B, lane 20), and
(10) pair C14: CH1 with a phenylalanine 126 mutated to cysteine paired with IgM Cμ4 with a glutamine 463 mutated to cysteine (FIG. 10B, lane 21).

Although the other eight cysteine pair combinations appeared appropriately positioned to form specific inter-chain disulfide bonds in structural models, the chains failed to express to give fully assembled antibodies.

It was also observed that the overall expression of each fully assembled antibody comprising any of the three successful cysteine pairs was substantially lower than seen for the original CH1-IgM Cμ4 pair (compare FIGS. 10A and 10B, lanes 2 and 16 with lanes 3, 20 and 21). This likely results from some disruption of expression, folding or packing between the chains modified to accommodate the new cysteine residues. Such disruption can potentially be mitigated by introducing additional mutations into one or both of the chains to improve expression and packing. Additional amino acid changes to the IgM Cμ4 chain comprising tyrosine 455 mutated to cysteine were added, the assembly of full antibody was measured when paired with a heavy chain comprising IgG1 CH1 with alanine 141 mutated to cysteine. These assembled antibodies are shown in FIG. 10A lanes 4, 5 and 6 which correspond respectively to the mutation of isoleucine at IgM Cμ4 position 556 to an alanine, the mutation of proline at IgM Cμ4 position 458 to an alanine, and the mutation of threonine at IgM Cμ4 position 477 to a tyrosine. FIG. 10A clearly shows that each of these mutations improves the expression of fully assembled antibody.

From this data, it is concluded that CH1 with alanine 141 mutated to cysteine can pair with IgM Cμ4 with a tyrosine 455 mutated to cysteine; CH1 with histidine 168 mutated to cysteine can pair with IgM Cμ4 with phenylalanine 516 mutated to cysteine; CH1 with a phenylalanine 126 mutated to cysteine can pair with IgM Cμ4 with a glutamine 463 mutated to cysteine. It is further concluded that reductions in the amount of fully assembled antibody that result from introduction of these new cysteine pairs may be reversed, and expression restored, by the introduction of additional mutations into one or both chains.

### Example 5

### Increasing Expression of an Antibody with Cysteine-Modified IgM Cμ4—IgG CH1 Light Chain Pairing

As shown in Example 4, an IgM Cμ4 region and an IgG CH1 constant region are compatible antibody-pairing regions, and these pairing regions can be modified by removing existing cysteine residues and introducing new cysteine residues to create new disulfide bonds that can be used to generate specific light chain pairs, for example in the context of a bi-specific antibody with two or more different light chain-heavy chain pairs.

Pair C2 described in Example 4 comprised an IgG CH1 cysteine substitution at EU position 141 (normally an alanine) and a corresponding IgM Cμ4 cysteine substitution at position 455 (normally a tyrosine). As described in Example 4 and shown in FIGS. 10A and 10B, expression of an antibody with heavy and light chains comprising these mutations is significantly reduced relative to expression of the same antibody comprising a non-mutated CH1 and hinge on the heavy chain and an IgM Cμ4 region terminating at a cysteine at position 556 (compare FIG. 10A lanes 2 and 3. Example 4 also describes how the mutation of isoleucine at IgM Cμ4 position 556 to an alanine, the mutation of proline at IgM Cμ4 position 458 to an alanine, or the mutation of threonine at IgM Cμ4 position 477 to a tyrosine increase the expression of fully assembled antibody. With this information, using structural modeling, a set of variant IgM Cμ4 regions were designed with a cysteine substitution at position 455 (normally a tyrosine). The amino acid sequences of these variants are SEQ ID NOs: 119-187.

Each of these variant light chain pairing regions were tested for expression and antibody assembly in the context of an antibody, in which the kappa constant region was replaced by the IgM Cμ4 region. Polynucleotides encoding antibody chains were constructed, each comprised an open reading frame encoding an antibody chain operably linked to a CMV promoter and rabbit globin polyadenylation sequence such that the open reading frame was expressible in a cultured mammalian cell. The polynucleotides constructed were as follows.

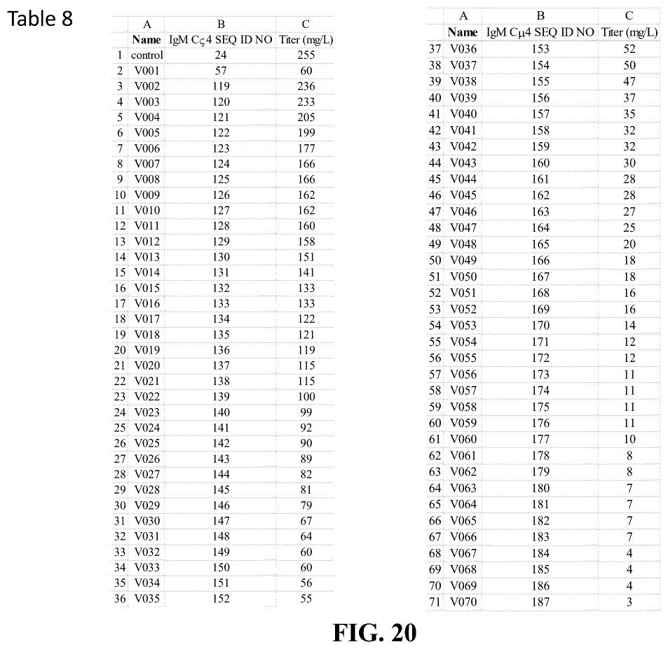

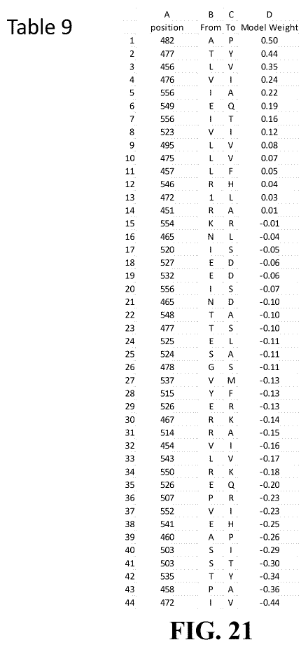

Polynucleotides for expression of antibody light chains using an IgM Cμ4 pairing region each comprised a sequence encoding, from N-terminus to C-terminus: a secretion signal, the light chain variable region of the antibody (with amino acid sequence SEQ ID NO:28), a glycine residue and an IgM Cμ4 pairing region (with an amino acid sequence corresponding to a SEQ ID NO shown in FIG. 20 Table 8, column B).

All but one polynucleotides for expression of antibody heavy chains each comprised a sequence encoding, from N-terminus to C-terminus: a secretion signal, the heavy chain variable region of the antibody (with amino acid sequence SEQ ID NO:29), a human IgG1 CH1 constant region (with amino acid sequence SEQ ID NO:60), a hinge region (with amino acid sequence SEQ ID NO:117) and an Fc region (human IgG1 CH2 plus CH3 regions) with amino acid sequence SEQ ID NO:118. A control polynucleotide for expression of an antibody heavy chain with no CH1 or IgG1 hinge cysteine modifications comprised a sequence encoding, from N-terminus to C-terminus: a secretion signal, the heavy chain variable region of the antibody (with amino acid sequence SEQ ID NO:29), a human IgG1 CH1 constant region (with amino acid sequence SEQ ID NO:1), a hinge region (with amino acid sequence SEQ ID NO:2) and an Fc region (human IgG1 CH2 plus CH3 regions) with amino acid sequence SEQ ID NO:118.

Polynucleotides encoding one heavy chain (or modified heavy chain) and one light chain (or modified light chain) were co-transfected into HEK 293 cells, and the cells were cultured for 7 days in THERMO FISHER SCIENTIFIC Expi293™ media. Antibody protein level was quantified using protein A tips on a Sartorius Octet according to the manufacturer's instructions. Culture supernatants were also analyzed on SDS polyacrylamide gels without reducing agent to verify that fully assembled antibody had been formed (data not shown). FIG. 20 Table 8 shows the variant name in column A, in column B is SEQ ID NO corresponding to the amino acid sequence of the IgM Cμ4 pairing region, column C shows the titer (in mg/L) of antibody produced. All heavy chains comprised the cysteine mutation at CH1 EU position 141 and comprised the IgG1 hinge region with mutated cysteine except for the antibody shown in row 1 which is the control without cysteine modifications.

FIG. 20 Table 8, column C shows that, in comparison to an antibody in which the kappa constant region is replaced by an IgM Cμ4 pairing region with amino acid sequence SEQ ID NO:24 (FIG. 20 Table 8, row 1) which had titer 255 mg/L; cysteine modification of the IgG CH1 by replacing the alanine at EU position 141 with cysteine and replacing the IgG1 hinge region cysteine at EU position 220 with an isoleucine together with cysteine modification of the IgM Cμ4 pairing region by replacing cysteine at position 556 with isoleucine and replacing tyrosine at position 455 with cysteine resulted in a decrease in titer of over 4-fold to 60 mg/L (FIG. 20 Table 8, row 2). Addition of further changes to the IgM Cμ4 pairing region results in a range of assembled antibody titers, as shown in FIG. 20 Table 8, rows 3-71. Significantly some combinations of these changes restore the titer to nearly the original levels: variants V002-V004 (with IgM Cμ4 pairing region with amino acid sequences SEQ ID NOS: 119-121 respectively) have titers in excess of 200 mg/L.

The contributions of different amino acid substitutions to assembled antibody titer was modelled as described previously in U.S. Pat. No. 8,635,029, and mean values for the regression weights were calculated for each substitution. These are shown in FIG. 0.21 Table 9: column A shows the amino acid position, column B shows the amino acid naturally found at this position in an IgM Cμ4 pairing region, column C shows the amino acid substitution at this position and column D shows the average model weight from the expression data shown in FIG. 20 Table 8. Substitutions with positive model weights are those that contribute positively to increasing expression of fully assembled antibody where an IgM Cμ4 pairing region comprises a cysteine at position 556 and a cysteine at position 455. Particularly advantageous substitutions include A482P, T477Y, L456V, V476I, 1556A, 1556T (IgM Cμ4 normally has threonine at position 556, but this was modified to isoleucine in the cysteine-modified version), E549Q, V523I, L495V, L475V, L457F and R546H. When the IgM Cμ4 T556 position is not used to incorporate an engineered cysteine, that position can be advantageously modified to T556I or T556A, for example, when IgM Cμ4 is paired with the CH1 pairing region.

### Example 6

### Cysteine Modifications Conferring Specificity to IgM Cμ4—Kappa Pairing

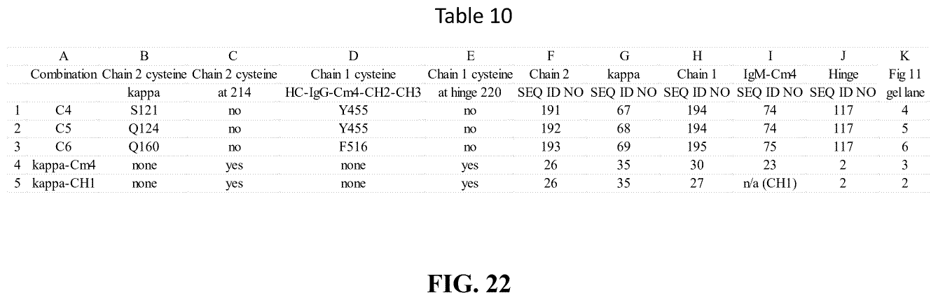

As shown in Example 1, an IgM Cμ4 region and a kappa constant region are compatible antibody-pairing regions. Because IgM Cμ4 pairs promiscuously with either CH1 or kappa constant regions, it is beneficial to modify both components of the desired pair to reduce non-specific pairing and improve specific pairing. Structural models of interactions between kappa and IgM Cμ4 were used to identify the locations of residues within each of these domains that might be replaced by cysteines that might be capable of forming a covalent disulfide bond between the two chains. In this way, three potential pairs of substitutions were identified, which are summarized in FIG. 22 Table 10.
Pair C4 comprised a kappa cysteine substitution at Kabat position 121 (normally a serine) and a corresponding IgM Cμ4 cysteine substitution at position 455 (normally a tyrosine);
Pair C5 comprised a kappa cysteine substitution at Kabat position 124 (normally a glutamine) and a corresponding IgM Cμ4 cysteine substitution at position 455 (normally a tyrosine);
Pair C6 comprised a kappa cysteine substitution at Kabat position 160 (normally a glutamine) and a corresponding IgM Cμ4 cysteine substitution at position 516 (normally a phenylalanine).

Each of these cysteine pairs was tested for expression and disulfide bond formation in the context of an antibody, in which the IgG1 CH1 constant region was replaced by the IgM Cμ4 region. Polynucleotides encoding antibody chains were constructed, each comprised an open reading frame encoding an antibody chain operably linked to a CMV promoter and rabbit globin polyadenylation sequence such that the open reading frame was expressible in a cultured mammalian cell. The polynucleotides constructed were as follows.

Polynucleotides for expression of antibody light chains using a kappa pairing region each comprised a sequence encoding, from N-terminus to C-terminus: a secretion signal, the light chain variable region of the antibody (with amino acid sequence SEQ ID NO:28) and a kappa pairing region (with an amino acid sequence corresponding to a SEQ ID NO shown in FIG. 22 Table 10, column G). A polynucleotide for expression of a control antibody light chain, with mature amino acid sequence SEQ ID NO:26, comprised a sequence encoding, from N-terminus to C-terminus: a secretion signal, the light chain variable region of the antibody (with amino acid sequence SEQ ID NO:28), a human kappa constant region (with amino acid sequence SEQ ID NO:35). The SEQ ID NO corresponding to the mature amino acid sequence of each light chain is shown in FIG. 22 Table 10, column F.

Polynucleotides for expression of antibody heavy chains each comprised a sequence encoding, from N-terminus to C-terminus: a secretion signal, the heavy chain variable region of the antibody (with amino acid sequence SEQ ID NO:29), a human IgM Cμ4 constant region (with amino acid sequence corresponding to a SEQ ID NO shown in FIG. 22 Table 10, column I), a hinge region (with amino acid sequence corresponding to a SEQ ID NO shown in FIG. 22 Table 10 column J) and an Fc region (human IgG1 CH2 plus CH3 regions) with amino acid sequence SEQ ID NO:118. A polynucleotide for expression of a control antibody heavy chain, with mature amino acid sequence SEQ ID NO:27, comprised a sequence encoding, from N-terminus to C-terminus: a secretion signal, the heavy chain variable region of the antibody (with amino acid sequence SEQ ID NO:29), a human IgG1 CH1 constant region (with amino acid sequence SEQ ID NO:1), a human IgG1 hinge region (with amino acid sequence SEQ ID NO:2), a human IgG1 CH2 region (with amino acid sequence SEQ ID NO:3) and a human IgG1 CH3 region (with amino acid sequence SEQ ID NO:4). The SEQ ID NO corresponding to the mature amino acid sequence of each heavy chain is shown in FIG. 22 Table 10, column H.

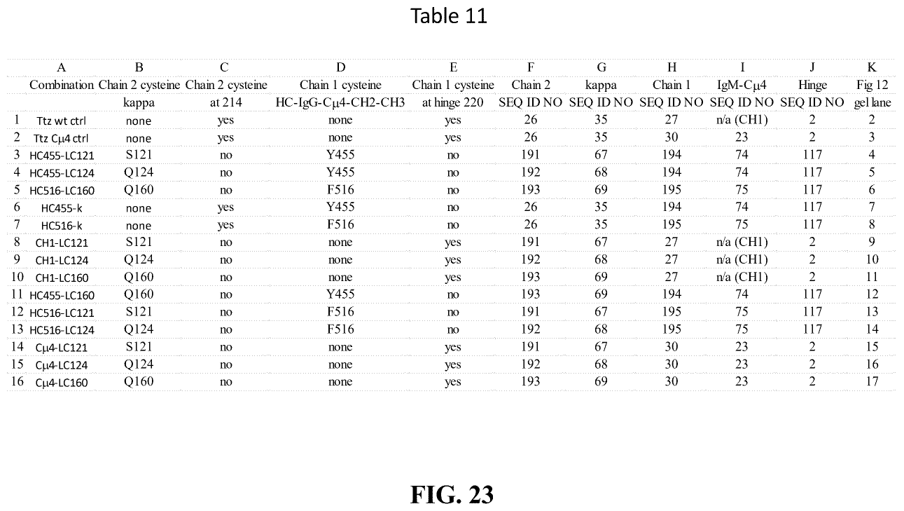

Polynucleotides encoding one heavy chain (or modified heavy chain) and one light chain (or modified light chain) were co-transfected into HEK 293 cells, and the cells were cultured for 7 days in THERMO FISHER SCIENTIFIC Expi293™ media. Culture supernatants were analyzed on SDS polyacrylamide gels without reducing agent. FIG. 23 Table 11 shows the different chain combinations tested and identifies the SEQ ID NOs corresponding to the amino acid sequences of each mature chain.

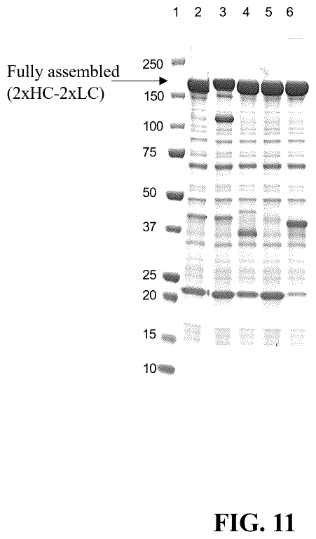

FIG. 11 shows a non-reduced gel of the polypeptides expressed in the culture supernatant. The sequence details of the co-expressed polypeptides in each lane are shown in FIG. 22 Table 10. Fully assembled tetramers, comprising two light and two heavy chains migrate at a little over 150 kDa, and are indicated by arrows. Antibody comprising a natural kappa light chain and a natural IgG1 heavy chain is shown in lane 2. Lane 3 shows supernatant from cells transfected with constructs comprising a natural kappa light chain and a heavy chain in which the IgG1 CH1 region is replaced with an IgM Cμ4 pairing region up to position 556. Lanes 4-6 show the assembled antibody produced when the heavy chain IgG1 hinge cysteine is mutated (as shown in FIG. 8B) and the terminal amino acid (Kabat position 214) of the kappa pairing region is mutated to an alanine and then alternative cysteine residues are introduced, one into the kappa constant region, the other into the IgM Cμ4 pairing region. All three of the different cysteine pairs tested resulted in detectable fully assembled antibody. These were kappa with serine 121 mutated to cysteine paired with IgM Cμ4 with a tyrosine 455 mutated to cysteine (FIG. 11, lane 4); kappa with glutamine 124 mutated to cysteine paired with IgM Cμ4 with tyrosine 455 mutated to cysteine (FIG. 11, lane 5), and kappa with glutamine 160 mutated to cysteine paired with IgM Cμ4 with phenylalanine 516 mutated to cysteine (FIG. 11, lane 6).

It was also observed that the overall expression of each fully assembled antibody comprising any of the three successful cysteine pairs was at least equal to that seen for the original kappa-IgM Cμ4 pair (compare lane 3 with lanes 4-6). Furthermore, the original kappa-IgM Cμ4 pair showed a significant band above 100 kDa which is likely partially assembled antibody: either two heavy chains, or two heavy chains plus a light chain. Thus the engineered cysteines appear to give better expression of more fully assembled antibody than did the simple pairing region substitution.

From this data it is concluded that kappa with serine 121 mutated to cysteine or with glutamine 124 mutated to cysteine can pair with IgM Cμ4 with a tyrosine 455 mutated to cysteine; kappa with glutamine 160 mutated to cysteine can pair with IgM Cμ4 with phenylalanine 516 mutated to cysteine. It is further concluded that these changes improve expression of fully assembled antibody.

### Example 7

### Orthogonality of Pairing Between IgM Cμ4 and Kappa Constant Regions Comprising Different Engineered Cysteines

Example 6 describes three different combinations of engineered cysteines that promote pairing between an IgM Cμ4 region and a kappa constant region: kappa with serine 121 mutated to cysteine paired with IgM Cμ4 with a tyrosine 455 mutated to cysteine, kappa with glutamine 124 mutated to cysteine paired with IgM Cμ4 with tyrosine 455 mutated to cysteine, and kappa with glutamine 160 mutated to cysteine paired with IgM Cμ4 with phenylalanine 516 mutated to cysteine. To test the orthogonality of the pairing resulting from the incorporation of engineered cysteines, the assembly of full-length antibody from different combinations of heavy and light chains with and without engineered cysteines was measured.

Polynucleotides encoding antibody chains were constructed, each comprised an open reading frame encoding an antibody chain operably linked to a CMV promoter and rabbit globin polyadenylation sequence such that the open reading frame was expressible in a cultured mammalian cell. The constructed polynucleotides were as follows.

Polynucleotides for expression of antibody light chains using a kappa pairing region each comprised a sequence encoding, from N-terminus to C-terminus: a secretion signal, the light chain variable region of the antibody (with amino acid sequence SEQ ID NO:28) and a kappa pairing region (with an amino acid sequence corresponding to a SEQ ID NO shown in FIG. 23 Table 11, column G). A polynucleotide for expression of a control antibody light chain, with mature amino acid sequence SEQ ID NO:26, comprised a sequence encoding, from N-terminus to C-terminus: a secretion signal, the light chain variable region of the antibody (with amino acid sequence SEQ ID NO:28), a human kappa constant region (with amino acid sequence SEQ ID NO:35). The SEQ ID NO corresponding to the mature amino acid sequence of each light chain is shown in FIG. 23 Table 11, column F.

Polynucleotides for expression of antibody heavy chains each comprised a sequence encoding, from N-terminus to C-terminus: a secretion signal, the heavy chain variable region of the antibody (with amino acid sequence SEQ ID NO:29), a human IgM Cμ4 constant region (with amino acid sequence corresponding to a SEQ ID NO shown in FIG. 23 Table 11, column I), a hinge region (with amino acid sequence corresponding to a SEQ ID NO shown in FIG. 23 Table 11, column J) and an Fc region (human IgG1 CH2 plus CH3 regions) with amino acid sequence SEQ ID NO:118. A polynucleotide for expression of a control antibody heavy chain, with mature amino acid sequence SEQ ID NO:27, comprised a sequence encoding, from N-terminus to C-terminus: a secretion signal, the heavy chain variable region of the antibody (with amino acid sequence SEQ ID NO:29), a human IgG1 CH1 constant region (with amino acid sequence SEQ ID NO:1), a human IgG1 hinge region (with amino acid sequence SEQ ID NO:2), a human IgG1 CH2 region (with amino acid sequence SEQ ID NO:3) and a human IgG1 CH3 region (with amino acid sequence SEQ ID NO:4). The SEQ ID NO corresponding to the mature amino acid sequence of each heavy chain is shown in FIG. 23 Table 11, column H.

Polynucleotides encoding one heavy chain (or modified heavy chain) and one light chain (or modified light chain) were co-transfected into HEK 293 cells, and the cells were cultured for 7 days in THERMO FISHER SCIENTIFIC Expi293™ media. Culture supernatants were analyzed on SDS polyacrylamide gels without reducing agent. FIG. 23 Table 11 shows the different chain combinations tested and identifies the SEQ ID NOs corresponding to the amino acid sequences of each mature chain.

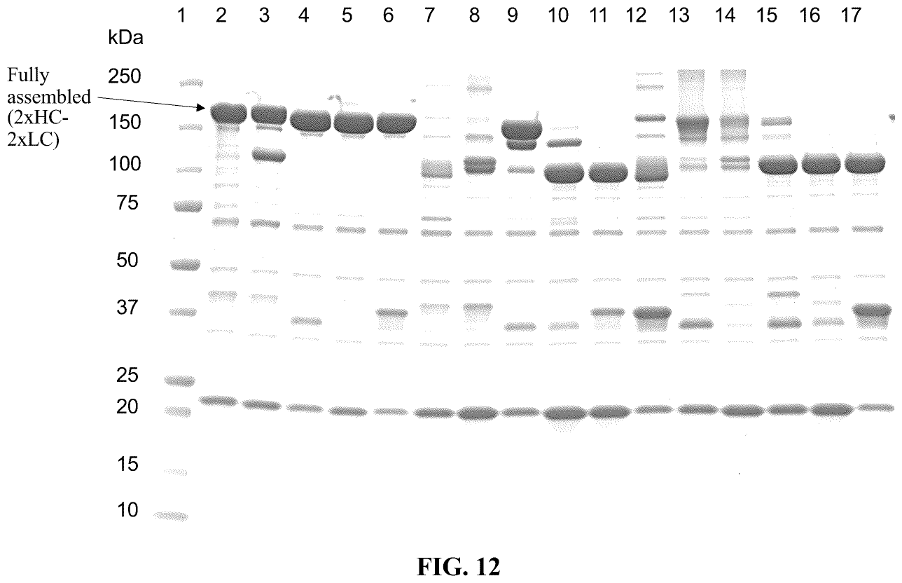

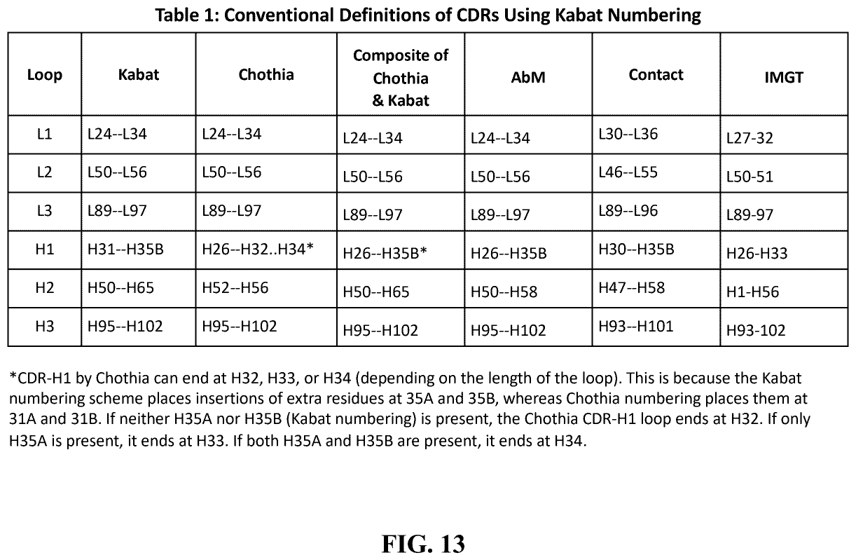

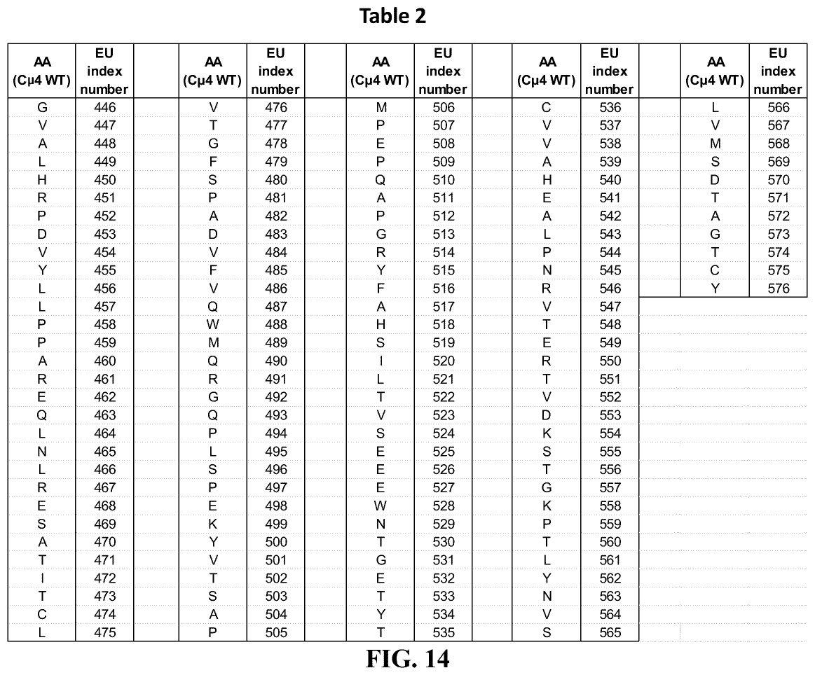

FIG. 12 shows a non-reduced gel of the polypeptides expressed in the culture supernatant. The sequence details of the co-expressed polypeptides in each lane are shown in FIG. 23 Table 11. Fully assembled tetramers, comprising two light and two heavy chains migrate at a little over 150 kDa, and are indicated by arrows. Antibody comprising a natural kappa light chain and a natural IgG1 heavy chain is shown in FIG. 12, lane 2. Lane 3 shows supernatant from cells transfected with constructs comprising a natural kappa light chain and a heavy chain in which the IgG1 CH1 region is replaced with an IgM Cμ4 pairing region up to position 556. Lanes 4-6 show the assembled antibody produced when the heavy chain IgG1 hinge cysteine is mutated (as shown in FIG. 8B) and the terminal amino acid (Kabat position 214) of the kappa pairing region is mutated to an alanine and then alternative cysteine residues are introduced, one into the kappa constant region, the other into the IgM Cμ4 pairing region. These were:
(i) kappa with S121C paired with IgM Cμ4 with Y455C (FIG. 12 lane 4);
(ii) kappa with Q124C paired with IgM Cμ4 with Y455C (FIG. 12, lane 5), and
(iii) kappa with Q160C paired with IgM Cμ4 with F516C (FIG. 12, lane 6).

These three engineered cysteine pairs show good levels of expression and assembly, comparable to the levels seen with a natural IgG1 CH1-kappa containing antibody (FIG. 12, lane 2).

FIG. 12, lanes 7 and 8 show proteins produced when a natural kappa chain without an engineered cysteine is paired with a heavy chain comprising an IgM Cμ4 region replacing the IgG1 CH1, with the hinge region at EU position 220 mutated to an alanine, and the IgM Cμ4 region having either a Y455C mutation (FIG. 12, lane 7) or an F516C mutation (FIG. 12, lane 6). In both cases, no fully assembled antibody was observed, demonstrating that these cysteine-engineered IgM Cμ4 regions combined with IgG1 hinge regions lacking a cysteine at EU position 220 were unable to assemble with an unmodified kappa chain.

FIG. 12, lanes 9-11 show proteins produced when a natural heavy chain with an IgG1 CH1 region and without an engineered cysteine is paired with a light chain comprising a kappa chain with cysteine at position 214 mutated to an alanine, and the kappa chain further comprising either a S121C mutation (FIG. 12 lane 9), or a Q124C mutation (FIG. 12 lane 10), or a Q160C mutation (FIG. 12 lane 11). No fully assembled antibody was observed with the kappa regions comprising either Q124C or Q160C, demonstrating that these cysteine-engineered kappa regions were unable to assemble with an unmodified IgG CH1 region.

FIG. 12, lanes 12-14 show proteins produced when chains with engineered cysteines are paired with chains with which they were not designed to assemble. A heavy chain comprising an IgM Cμ4 region replacing the IgG1 CH1, with the hinge region at EU position 220 mutated to an alanine, and the IgM Cμ4 region having a Y455C mutation was paired with kappa with Q160C and C214A mutations. Very little fully assembled antibody was observed (FIG. 12 lane 12) compared to what was seen when this heavy chain was paired with kappa with S121C and C214A, or Q124 and C214A mutations (FIG. 12, lanes 4 and 5, respectively), demonstrating orthogonality between these cysteine-engineered heavy and light chains. A heavy chain comprising an IgM Cμ4 region replacing the IgG1 CH1, with the hinge region at EU position 220 mutated to an alanine, and the IgM Cμ4 region having a F516C mutation was paired with kappa with S121C and C214A, or Q124 and C214A mutations (FIG. 12, lanes 13 and 14, respectively). Little fully assembled antibody was observed compared to what was seen when this heavy chain was paired with kappa with Q160C and C214A mutations (compare with FIG. 12 lane 5), demonstrating orthogonality between these cysteine-engineered heavy and light chains.

FIG. 12, lanes 15-17 show proteins produced when a heavy chain in which the IgG1 CH1 region is replaced by an IgM Cμ4 region without an engineered cysteine is paired with a light chain comprising a kappa chain with cysteine at position 214 mutated to an alanine, and the kappa chain further comprising either a S121C mutation (FIG. 12 lane 15), or a Q124C mutation (FIG. 12, lane 16), or a Q160C mutation (FIG. 12 lane 17). No fully assembled antibody was observed with the kappa regions comprising either Q124C or Q160C, demonstrating that these cysteine-engineered kappa regions were unable to assemble with an IgM Cμ4 region without an engineered cysteine.

From this example, it is concluded that the engineered cysteines described are useful for producing specific pairing between antibody heavy chains and light chains.

### Example 8

### Binding of Antibodies Comprising IgM Cμ4 Constant Regions to Receptors Mediating IgM Effector Function

Human IgM has been reported to bind to three different receptors that mediate effector function: FcpR, FcapR, and plgR. In some instances it is advantageous to reduce or eliminate IgM effector function in an antibody comprising an IgM Cμ4 constant region. The binding to each of these receptors was test for a set of mutations within an antibody in which the CH11 gG region was replaced with an IgM Cμ4 constant region comprising an F516C mutation, and wherein the kappa light chain comprised Q160C and C214A mutations. The mature light and heavy chains of this antibody have amino acid sequences SEQ ID NO:193 and 195 respectively. Additional amino acid changes were incorporated to create a set of variants of this antibody, the additional changes are shown in FIG. 24 Table 12 in column E using the standard one letter code and with—indicating a deletion. Positions are given using Kabat numbering, as shown in FIG. 14 Table 2.

Kinetic binding of antibodies to three human IgM Fc Receptors (FcpR, FcapR, and plgR) was assessed using a Sierra SPR©-32 Pro (Bruker). Antibodies were immobilized on a High Capacity Amine Sensor (Part No:1862614, Bruker Daltonic) via amine coupling chemistry. The soluble portion of each receptor was purchased (FcpR from R&D Systems, cat #9494-MU had amino acid sequence SEQ ID NO:202; FcapR from R&D Systems, cat #9278-FC had amino acid sequence SEQ ID NO:201; plgR from R&D Systems, cat #2717-PG had amino acid sequence SEQ ID NO:200) and titrated over the captured antibodies in HEPES buffer (Teknova H1030; 10 mM HEPES pH 7.4, 0.15 M NaCl, 500 mM EDTA, 0.05% Tween 20). A minimum of six concentrations for each receptor were titrated over each captured antibody to characterize binding. Binding analysis was performed using Bruker SPR Analyzer 4 software.

FIG. 24 Table 12 shows a qualitative measure of the binding response of each antibody to the three different receptors FcapR, FcpR, and plgR as seen in columns B, C and D respectively. The binding of an antibody comprising two heavy chains (each including an IgM Cμ4 constant region) the with amino acid sequence SEQ ID NO:195 and two light chains with amino acid sequence SEQ ID NO:193 is shown in row 1. An IgG negative control antibody comprising two heavy chains with amino acid sequence SEQ ID NO:27 and two light chains with amino acid sequence SEQ ID NO:26 is shown in row 27. A purchased IgM (lambda light chain) was purchased from Southern Biotech, (cat #0158L-01) and used as a positive control (FIG. 24 Table 12, row 28). An IgM (kappa light chain) comprising a light chain with amino acid sequence SEQ ID NO: 26, a heavy chain with amino acid sequence SEQ ID NO: 203 and a J chain with amino acid sequence SEQ ID NO: 204 was used as a positive control (FIG. 24 Table 12, row 29). Blank sensors were run as negative controls, shown in FIG. 24 Table 12, rows 30-32.

Antibodies comprising an IgM Cμ4 constant region comprising a glutamate to arginine substitution at position 468 (FIG. 24 Table 12, row 16), a glutamate to alanine mutation at position 526 (FIG. 24 Table 12, row 13), or deletions of glutamate at positions 468 and 526 (FIG. 24 Table 12, row 14) all showed binding to any of the three IgM receptors at comparable levels to the IgG negative control (FIG. 24 Table 12, row27). Thus deletion or mutation of glutamate 468 and/or glutamate 526 in the IgM Cμ4 constant region can be used to reduce or eliminate IgM effector functions in antibodies comprising an IgM Cμ4 constant region.

### Example 9

### Orthogonality of Pairing Between IgM Cμ4 and Kappa Constant Regions by Combining Engineered Cysteines with Electrostatic Steering

Examples 6 and 7 describe three different combinations of engineered cysteines that promote pairing between an IgM Cμ4 region and a kappa constant region: kappa with serine 121 mutated to cysteine paired with IgM Cμ4 with a tyrosine 455 mutated to cysteine, kappa with glutamine 124 mutated to cysteine paired with IgM Cμ4 with tyrosine 455 mutated to cysteine, and kappa with glutamine 160 mutated to cysteine paired with IgM Cμ4 with phenylalanine 516 mutated to cysteine. FIG. 12 shows that, although kappa with serine 121 mutated to cysteine paired preferentially with IgM Cμ4 with tyrosine 455 mutated to cysteine (FIG. 12 lane 4), it also paired to a much lesser degree with IgM Cμ4 with phenylalanine 516 mutated to cysteine (FIG. 12 lane 13). Similarly, kappa with glutamine 124 mutated to cysteine paired preferentially with IgM Cμ4 with tyrosine 455 mutated to cysteine (FIG. 12 lane 5), it also paired to a much lesser degree with IgM Cμ4 with phenylalanine 516 mutated to cysteine (FIG. 12 lane 14). Conversely, kappa with glutamine 160 mutated to cysteine paired preferentially with IgM Cμ4 with phenylalanine 516 mutated to cysteine (FIG. 12 lane 6), it also paired to a much lesser degree with IgM Cμ4 with tyrosine 455 mutated to cysteine (FIG. 12 lane 14).

FIG. 12 also shows that kappa with serine 121 mutated to cysteine was able to assemble with a normal CH1-containing IgG heavy chain (FIG. 12 lane 9). Expression of IgG heavy chains containing the CH1 domain normally requires expression of a light chain to interact with the CH1 domain, facilitating its folding and release of the endoplasmic reticulum chaperone BiP. Without this interaction, BiP remains bound to the CH1-containing heavy chain and retains it within the endoplasmic reticulum. FIG. 12 shows that although kappa chains with either glutamine 124 or glutamine 160 mutated to cysteine did not assemble and form disulfide bonds with a normal CH1-containing IgG heavy chain (FIG. 12 lanes 10 and 11 respectively), there is a highly expressed band visible at about 100 kDa. This band corresponds to a pair of heavy chains bound to each other, but with neither bound to a corresponding light chain. However, the presence of so much secreted protein indicates that the light chain must have interacted, at least transiently, with the heavy chain CH1, in order to facilitate the release of BiP and its subsequent exit from the endoplasmic reticulum and secretion from the cell.

To further increase specificity of pairing between light and heavy chains, residues were engineered in both heavy and light chains to create additional electrostatic interactions. The objective was to introduce mutually attractive changes into corresponding pairing regions that would at the same time result in mutually repulsive changes in non-corresponding pairing regions. These changes were combinations of one or more of:
mutation of leucine 128 to lysine and mutation of valine 185 to serine in CH1,
mutation of valine 133 to serine and mutation of serine 176 to aspartate in a kappa chain that retained its original cysteine at position 214,
mutation of glutamate 123 to lysine,
mutation of aspartate 122 to lysine,
mutation of serine 121 to lysine,
mutation of serine 131 to histidine, lysine or arginine in kappa chains where cysteine at 214 was also mutated (for example to alanine, threonine, isoleucine, valine or another non-cysteine bonding residue) and an alternative cysteine was introduced by mutation of glutamine 124 or glutamine 160;
mutation of lysine 554 to glutamate or aspartate,
mutation of threonine 477 to lysine, glutamate or aspartate and upper hinge lysine 218 mutated to glutamate in IgM Cμ4 with tyrosine 455 or phenylalanine 516 mutated to cysteine and mutation of upper hinge cysteine 220 to alanine.

Polynucleotides encoding antibody chains were constructed, each comprised an open reading frame encoding an antibody chain operably linked to a CMV promoter and rabbit globin polyadenylation sequence such that the open reading frame was expressible in a cultured mammalian cell. Polynucleotides were as follows.

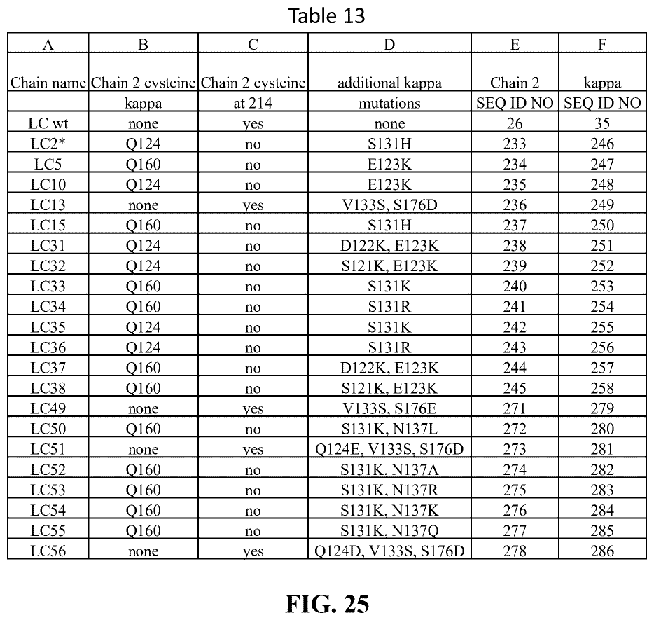

Polynucleotides for expression of antibody light chains using a kappa pairing region each comprised a sequence encoding, from N-terminus to C-terminus: a secretion signal, the light chain variable region of the antibody (with amino acid sequence SEQ ID NO:28) and a kappa pairing region (with an amino acid sequence corresponding to a SEQ ID NO shown in FIG. 25 Table 13, column F). The SEQ ID NO corresponding to the mature amino acid sequence of each light chain is shown in FIG. 25 Table 13, column E. The position of engineered cysteines is indicated in column B (as well as the amino acid that is replaced), whether the natural cysteine at position 214 was allowed to remain is indicated in column C, other mutations are indicated in column D of FIG. 25 Table 13.

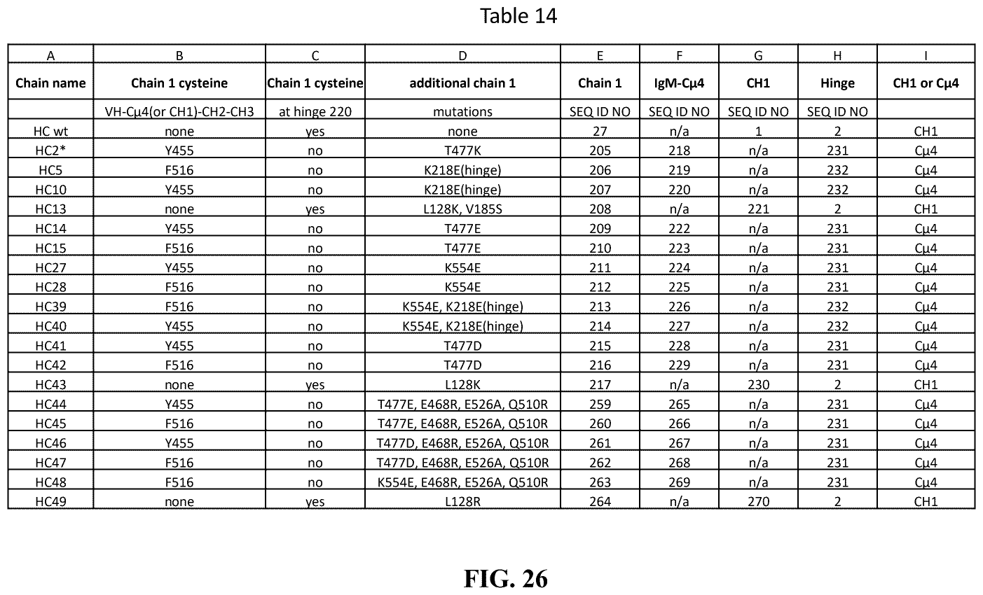

Polynucleotides for expression of antibody heavy chains each comprised a sequence encoding, from N-terminus to C-terminus: a secretion signal, the heavy chain variable region of the antibody (with amino acid sequence SEQ ID NO:29), a human IgM Cμ4 constant region or a human IgG CH1 region (with amino acid sequence corresponding to a SEQ ID NO shown in FIG. 26 Table 14, column G if the pairing region was an IgG CH1 region, or in column F if the pairing region was an IgM Cμ4 pairing region), a hinge region (with amino acid sequence corresponding to a SEQ ID NO shown in FIG. 26 Table 14, column H) and an Fc region (human IgG1 CH2 plus CH3 regions) with amino acid sequence SEQ ID NO:118. The SEQ ID NO corresponding to the mature amino acid sequence of each heavy chain is shown in FIG. 26 Table 14, column E. The position of engineered cysteines is indicated in column B (as well as the amino acid that is replaced), whether the natural cysteine at hinge position 220 was allowed to remain is indicated in column C, other mutations are indicated in column D of FIG. 26 Table 14.

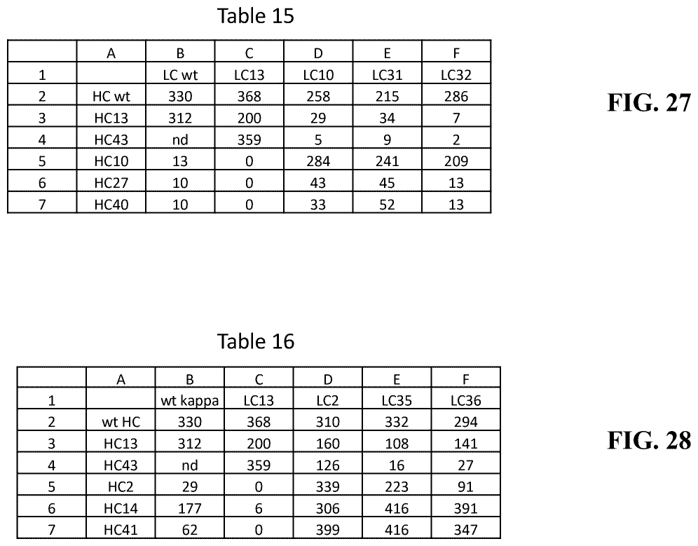

Polynucleotides encoding one heavy chain (or modified heavy chain) and one light chain (or modified light chain) were co-transfected into HEK 293 cells, and the cells were cultured for 7 days in THERMO FISHER SCIENTIFIC Expi293™ media. Protein was purified by affinity chromatography on protein A resin, and the antibody yield was used to calculate the titer in the original culture. Tables 15-18 provided in FIGS. 27-30 show the different chain combinations tested and the resulting antibody titers in mg/L (nd=not done).

FIG. 27 Table 15 shows the titers of antibodies (in mg/L) produced with various combinations of light chains (named in row 1 according to FIG. 25 Table 13) and heavy chains (named in column A according to FIG. 26 Table 14). Of particular interest, HC10 comprised an IgM Cμ4 pairing region comprising engineered cysteine at position 455 (Y455C), an IgG1 hinge region with mutation of hinge cysteine 220 (to alanine: C220A) and a second mutation in the upper hinge, where lysine 218 was mutated to glutamate (K218E), HC10 further comprised an IgG1 Fc (CH2 and CH3) region. This heavy chain resulted in high antibody titers when paired with kappa light chains comprising an engineered cysteine at position 124 (Q124C), a mutation of cysteine 214 (to alanine: C214A) and either:
(i) mutation of glutamate 123 to lysine (E123K) as in LC10 (FIG. 27 Table 15, row 5, column D),
(ii) mutation of glutamate 123 to lysine (E123K) and mutation of aspartate 122 to lysine (D122K) as in LC31 (FIG. 27 Table 15 row 5, column E), or
(iii) mutation of glutamate 123 to lysine (E123K) and mutation of serine 121 to lysine (S121K) as in LC32 (FIG. 27 Table 15, row 5, column F).

Non-reduced gels of the protein product showed a clean single band at the expected size of approximately 150 kDa, showing that light chains LC10, LC31 and LC32 were all able to pair with HC10, facilitate its folding and export from the endoplasmic reticulum and assemble into fully formed antibody. In contrast, heavy chain HC10 produced less than 5% of the levels of antibody seen with LC10, LC31 and LC32 when HC10 was instead paired with wt kappa (FIG. 27 Table 15, row 5, column B) and no measurable antibody when paired with LC13, whose kappa region comprised mutations of serine at position 176 to aspartate (S176D) and valine at position 133 to serine (V133S) (FIG. 27 Table 15, row 5, column C).

However all of LC10, LC31 and LC32 were able to facilitate folding and secretion of an unmodified CH1 HC almost as well as the unmodified kappa and LC13 could (compare FIG. 27 Table 15, row 2, columns D, E and F with columns B and C), although on a non-reduced gel the proteins produced by LC10, LC31 and LC32 in combination with the unmodified CH1 HC were largely improperly assembled (lacking disulfide-bonded light chains), similar to lanes 10 and 11 in FIG. 12. In contrast when the CH1 was modified to comprise an additional mutation of leucine 128 to lysine (L128K) as in HC43, the production of antibody of this heavy chain in combination with LC10, LC31 or LC32 was reduced by a factor of at least 20 (compare FIG. 27 Table 15, columns D, E and F row 2 (unmodified CH1) and row (with the L128K mutation).

Thus an improved orthogonal set of heavy and light chains, in which the light chains are able to interact well with their corresponding heavy chains, enabling their folding, secretion and correct disulfide bonding, but wherein the light chains do not interact with their non-corresponding heavy chains even sufficient to enable efficient secretion of the non-corresponding heavy chain comprises
(i) a first chain comprising an IgM Cμ4 pairing region comprising mutation Y455C within the IgM Cμ4 region (for example a pairing region with amino acid sequence SEQ ID NO:220), joined to an IgG1 hinge region comprising mutations K218E and C220A (for example a hinge region with amino acid sequence SEQ ID NO:232) and joined to an IgG Fc (CH2 and CH3) region,
(ii) a corresponding second chain comprising a kappa pairing region comprising mutations Q124C, C214A and either (a) E123K (for example a pairing region with amino acid sequence SEQ ID NO:248), (b) E123K and D122K (for example a pairing region with amino acid sequence SEQ ID NO:251), or (c) E123K and S121K (for example a pairing region with amino acid sequence SEQ ID NO:252),
(iii) a third chain comprising a CH1 pairing region comprising mutation L128K (for example a pairing region with amino acid sequence SEQ ID NO:230), and
(iv) a fourth chain corresponding to the third chain, the fourth chain comprising a kappa pairing region comprising mutations S176D and V133S (for example a pairing region with amino acid sequence SEQ ID NO:249); wherein the third or fourth chain further comprise an IgG hinge region and IgG Fc (CH2 and CH3) region.

FIG. 28 Table 16 shows the titers of antibodies (in mg/L) produced with various combinations of light chains (named in row 1 according to FIG. 25 Table 13) and heavy chains (named in column A according to FIG. 26 Table 14). Of particular interest, HC14 comprised an IgM Cμ4 pairing region comprising engineered cysteine at position 455 (Y455C) and mutation of threonine at position 477 to glutamate (T477E); HC41 comprised an IgM Cμ4 pairing region comprising engineered cysteine at position 455 (Y455C) and mutation of threonine at position 477 to aspartate (T477D). HC14 and HC41 each further comprised an IgG1 hinge region with mutation of hinge cysteine 220 (to alanine: C220A) and an IgG1 Fc (CH2 and CH3) region. These heavy chains resulted in high antibody titers when paired with kappa light chains comprising an engineered cysteine at position 124 (Q124C), a mutation of cysteine 214 (to alanine: C214A) and mutation of serine at position 131 to either (i) histidine (S131H) as in LC2 (FIG. 28 Table 16, rows 6 and 7, column D), (ii) lysine (S131K) as in LC35 (FIG. 28 Table 16 rows 6 and 7, column E), or (iii) arginine (S131R) as in LC36 (FIG. 28 Table 16, rows 6 and 7, column F). Non-reduced gels of the protein product showed a clean single band at the expected size of approximately 150 kDa, showing that light chains LC2, LC35 and LC36 were all able to pair with HC14 or HC41, facilitate corresponding heavy chain folding and export from the endoplasmic reticulum and assemble into fully formed antibody. In contrast, heavy chains HC14 and 41 produced less than 3% of the levels of antibody seen with LC2, LC35 and LC36 when HC14 or HC41 were instead paired with LC13, whose kappa region comprised mutations of serine at position 176 to aspartate (S176D) and valine at position 133 to serine (V133S) (FIG. 28 Table 16 rows 6 and 7 column C).

However all of LC2, LC35 and LC36 were able to facilitate folding and secretion of an unmodified CH1 HC almost as well as the unmodified kappa and LC13 could (compare FIG. 28 Table 16 row 2, columns D, E and F with columns B and C), although on a non-reduced gel the proteins produced by LC2, LC35 and LC36 in combination with the unmodified CH1 HC were largely improperly assembled (lacking disulfide-bonded light chains), similar to lanes 10 and 11 in FIG. 12. In contrast when the CH1 was modified to comprise an additional mutation of leucine 128 to lysine (L128K) as in HC43, the production of antibody of this heavy chain in combination with LC2 was reduced nearly 3-fold relative to co-expression with unmodified CH1 (compare FIG. 28 Table 16 rows 2 and 4, column D), and when HC43 was expressed in combination with LC35 or LC36, titer was reduced by a factor of at least 10 relative to co-expression with unmodified CH1 (compare FIG. 28 Table 16, columns E and F, row 2 (unmodified CH1) and row 4 (with the L128K mutation).

Thus an improved orthogonal set of heavy and light chains, in which the light chains are able to interact well with their corresponding heavy chains, enabling their folding, secretion and correct disulfide bonding, but wherein the light chains do not interact with their non-corresponding heavy chains even sufficient to enable efficient secretion of the non-corresponding heavy chain comprises:
(i) a first chain comprising an IgM Cμ4 pairing region comprising mutation Y455C and either (a) T477E or (b) T477D within the IgM Cμ4 region (for example a pairing region with an amino acid sequence selected from SEQ ID NOs:222 and 228);
(ii) a corresponding second chain comprising a kappa pairing region comprising mutations Q124C, C214A and either (a) S131H, (b) S131K or (c) S131R (for example a pairing region with amino acid sequence selected from SEQ ID NOs:246, 255 or 256) and wherein the first or second chain further comprise an IgG hinge region comprising a mutation at C220, for example C220A, C2201 or C220T and IgG Fc (CH2 and CH3) region;
(iii) a third chain comprising a CH1 pairing region comprising mutation L128K (for example a pairing region with amino acid sequence SEQ ID NO:230), and
(iv) a fourth chain corresponding to the third chain, the fourth chain comprising a kappa pairing region comprising mutations S176D and V133S (for example a pairing region with amino acid sequence SEQ ID NO:249); wherein the third or fourth chain further comprise an IgG hinge region and IgG Fc (CH2 and CH3) region.

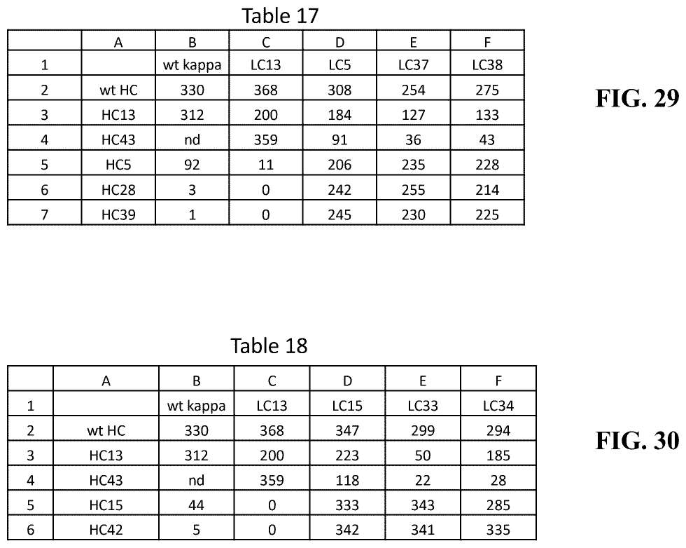

FIG. 29 Table 17 shows the titers of antibodies (in mg/L) produced with various combinations of light chains (named in row 1 according to FIG. 25 Table 13) and heavy chains (named in column A according to FIG. 26 Table 14). Of particular interest: HC5 comprised an IgM Cμ4 pairing region comprising engineered cysteine at position 516 (F516C) a mutation of hinge cysteine 220 (to alanine: C220A) and a second mutation in the upper hinge, where lysine 218 was mutated to glutamate (K218E); HC39 comprised an IgM Cμ4 pairing region comprising engineered cysteine at position 516 (F516C) and a mutation of lysine at position 554 to glutamate, a mutation of hinge cysteine 220 (to alanine: C220A) and a second mutation in the upper hinge, where lysine 218 was mutated to glutamate (K218E); HC28 comprised an IgM Cμ4 pairing region comprising engineered cysteine at position 516 (F516C) and a mutation of lysine at position 554 to glutamate, and a mutation of hinge cysteine 220 (to alanine: C220A). These three heavy chains resulted in high antibody titers with kappa light chains comprising an engineered cysteine at position 160 (Q160C), a mutation of cysteine 214 (to alanine: C214A) and either (i) mutation of glutamate 123 to lysine (E123K) as in LC5 (FIG. 29 Table 17, rows 5-7, column D), (ii) mutation of glutamate 123 to lysine (E123K) and mutation of aspartate 122 to lysine (D122K) as in LC37 (FIG. 29 Table 17, rows 5-7, column E), or (iii) mutation of glutamate 123 to lysine (E123K) and mutation of serine 121 to lysine (S121K) as in LC38 (FIG. 29 Table 17, rows 5-7, column F). Non-reduced gels of the protein product showed a clean single band at the expected size of approximately 150 kDa, showing that light chains LC5, LC37 and LC38 were all able to pair with HC5, HC28 and HC39, facilitate heavy chain folding and export from the endoplasmic reticulum and assemble into fully formed antibody. In contrast, heavy chains HC5 produced less than 50% of the levels of antibody seen with LC5, LC37 and LC38 when HC5 was instead paired with wt kappa (FIG. 29 Table 17, row 5, column B) and less than 5% of the levels of antibody seen with LC5, LC37 and LC38 when HC5 was when paired with LC13, whose kappa region comprised mutations of serine at position 176 to aspartate (S176D) and valine at position 133 to serine (V133S) (FIG. 29 Table 17, row 5, column C). Heavy chains HC28 and HC39 produced less than 3% of the levels of antibody seen with LC5, LC37 and LC38 when HC28 or HC39 were instead paired with wt kappa (FIG. 29 Table 17, rows 6-7, column B) and no detectable antibody was seen when HC28 and HC39 were paired with LC13, whose kappa region comprised mutations of serine at position 176 to aspartate (S176D) and valine at position 133 to serine (V133S) (FIG. 29 Table 17, rows 6-7, column C).

However all of LC5, LC37 and LC38 were able to facilitate folding and secretion of an unmodified CH1 HC almost as well as the unmodified kappa and LC13 could (compare FIG. 29 Table 17, row 2, columns D, E and F with columns B and C), although on a non-reduced gel the proteins produced by LC10, LC31 and LC32 in combination with the unmodified CH1 HC were largely improperly assembled (lacking disulfide-bonded light chains), similar to lanes 10 and 11 in FIG. 12. In contrast when the CH1 was modified to comprise an additional mutation of leucine 128 to lysine (L128K) as in HC43, the production of antibody of this heavy chain in combination with LC5, was reduced by a factor of 3 (compare FIG. 29 Table 17, column D, row 2 (unmodified CH1) and row 4 (with the L128K mutation). When the CH1 was modified to comprise an additional mutation of leucine 128 to lysine (L128K) as in HC43, the production of antibody of this heavy chain in combination with LC37 or LC38 was reduced by a factor of 7 (compare FIG. 29 Table 17, columns E and F, row 2 (unmodified CH1) and row 4 (with the L128K mutation).

Thus an improved orthogonal set of heavy and light chains, in which the light chains are able to interact well with their corresponding heavy chains, enabling their folding, secretion and correct disulfide bonding, but wherein the light chains do not interact with their non-corresponding heavy chains even sufficient to enable efficient secretion of the non-corresponding heavy chain comprises:
(i) a first chain comprising (a) an IgM Cμ4 pairing region comprising mutation F516C within the IgM Cμ4 region (for example a pairing region with amino acid sequence SEQ ID NO:219) joined to an IgG1 hinge region comprising mutations K218E and C220A (for example a hinge region with amino acid sequence SEQ ID NO:232), or (b) an IgM Cμ4 pairing region comprising mutations F516C and K554E within the IgM Cμ4 region (for example a pairing region with amino acid sequence SEQ ID NO:225), joined to an IgG1 hinge region comprising mutations K218E and C220A (for example a hinge region with amino acid sequence SEQ ID NO:232), or (c) an IgM Cμ4 pairing region comprising mutations F516C and K554E within the IgM Cμ4 region (for example a pairing region with amino acid sequence SEQ ID NO:226), joined to an IgG1 hinge region comprising mutation C220A (for example a hinge region with amino acid sequence SEQ ID NO:231) and joined to an IgG Fc (CH2 and CH3) region,
(ii) a corresponding second chain comprising a kappa pairing region comprising mutations Q160C, C214A and either (a) E123K (for example a pairing region with amino acid sequence SEQ ID NO:247), (b) E123K and D122K (for example a pairing region with amino acid sequence SEQ ID NO:257), or (c) E123K and S121K (for example a pairing region with amino acid sequence SEQ ID NO:258),
(iii) a third chain comprising a CH1 pairing region comprising mutation L128K (for example a pairing region with amino acid sequence SEQ ID NO:230), and
(iv) a fourth chain corresponding to the third chain, the fourth chain comprising a kappa pairing region comprising mutations S176D or alternatively S176E, and V133S (for example a pairing region with amino acid sequence SEQ ID NO:249); wherein the third or fourth chain further comprise an IgG hinge region and IgG Fc (CH2 and CH3) region.

FIG. 30 Table 18 shows the titers of antibodies (in mg/L) produced with various combinations of light chains (named in row 1 according to FIG. 25 Table 13) and heavy chains (named in column A according to FIG. 26 Table 14). Of particular interest, HC15 comprised an IgM Cμ4 pairing region comprising engineered cysteine at position 516 (F516C) and mutation of threonine at position 477 to glutamate (T477E); HC42 comprised an IgM Cμ4 pairing region comprising engineered cysteine at position 516 (F516C) and mutation of threonine at position 477 to aspartate (T477D). HC15 and HC42 each further comprised an IgG1 hinge comprising mutation of cysteine 220 (to alanine: C220A) and an IgG1 Fc (CH2 and CH3) region. These heavy chains resulted in high antibody titers when paired with kappa light chains comprising an engineered cysteine at position 160 (Q160C), a mutation of cysteine 214 (to alanine: C214A) and mutation of serine at position 131 to either (i) histidine (S131H) as in LC15 (FIG. 30 Table 18, rows 5 and 6, column D), (ii) lysine (S131K) as in LC33 (FIG. 30 Table 18, rows 5 and 6, column E), or (iii) arginine (S131R) as in LC34 (FIG. 30 Table 18, rows 5 and 6, column F). Non-reduced gels of the protein product showed a clean single band at the expected size of approximately 150 kDa, showing that light chains LC15, LC33 and LC34 were all able to pair with HC15 or HC42, facilitate corresponding heavy chain folding and export from the endoplasmic reticulum and assemble into fully formed antibody. In contrast, heavy chain HC15 produced about 15% of the levels of antibody seen with LC15, LC33 and LC34 when HC15 was instead paired with wt kappa (FIG. 30 Table 18, row 5, column B); heavy chain HC42 produced less than 2% of the levels of antibody seen with LC15, LC33 and LC34 when HC15 was instead paired with wt kappa (FIG. 30 Table 18 row 6 column B); and neither HC15 nor HC42 produced detectable antibody when they were instead paired with LC13, whose kappa region comprised mutations of serine at position 176 to aspartate (S176D) and valine at position 133 to serine (V133S) (FIG. 30 Table 18, rows 5 and 6, column C).

However all of LC15, LC33 and LC34 were able to facilitate folding and secretion of an unmodified CH1 HC almost as well as the unmodified kappa and LC13 could (compare FIG. 30 Table 18, row 2, columns D, E and F with columns B and C), although on a non-reduced gel the proteins produced by LC15, LC33 and LC34 in combination with the unmodified CH1 HC were largely improperly assembled (lacking disulfide-bonded light chains), similar to lanes 10 and 11 in FIG. 12. In contrast, when the CH1 was modified to comprise an additional mutation of leucine 128 to lysine (L128K) as in HC43, the production of antibody of this heavy chain in combination with LC15 was reduced nearly 3-fold relative to co-expression with unmodified CH1 (compare FIG. 30 Table 18, rows 2 and 4, column D), and when HC43 was expressed in combination with LC35 or LC36, titer was reduced by a factor of at least 10 relative to co-expression with unmodified CH1 (compare FIG. 30 Table 18, columns E and F, row 2 (unmodified CH1) and row 4 (with the L128K mutation)).

Thus an improved orthogonal set of heavy and light chains, in which the light chains are able to interact well with their corresponding heavy chains, enabling their folding, secretion and correct disulfide bonding, but wherein the light chains do not interact with their non-corresponding heavy chains even sufficient to enable efficient secretion of the non-corresponding heavy chain comprises:
(i) a first chain comprising an IgM Cμ4 pairing region comprising mutation F516C and either (a) T477E or (b) T477D within the IgM Cμ4 region (for example a pairing region with an amino acid sequence selected from SEQ ID NOs:223 and 229);
(ii) a corresponding second chain comprising a kappa pairing region comprising mutations Q160C, C214A and either (a) S131H, (b) S131K or (c) S131R (for example a pairing region with amino acid sequence selected from SEQ ID NOs:250, 253 or 254) and wherein the first or second chain further comprise an IgG hinge region comprising a mutation at C220, for example C220A, C2201 or C220T and IgG Fc (CH2 and CH3) region;
(iii) a third chain comprising a CH1 pairing region comprising mutation L128K (for example a pairing region with amino acid sequence SEQ ID NO:230), and
(iv) a fourth chain corresponding to the third chain, the fourth chain comprising a kappa pairing region comprising mutations S176D and V133S (for example a pairing region with amino acid sequence SEQ ID NO:249); wherein the third or fourth chain further comprise an IgG hinge region and IgG Fc (CH2 and CH3) region.

### Example 10

### Kappa Mutations to Favor Pairing to Either IgG CH1 or to IgM Cμ4

As described in Example 9, the addition of electrostatic steering mutations into the kappa, CH1 and Cμ4 pairing regions improves the specificity of pairing. However, a low level of residual unwanted pairing was observed between the kappa chains engineered to pair specifically with Cμ4, and the CH1 pairing region. The structure of kappa bound to CH1 was analyzed, and observed that asparagine at position 137 in the kappa chain (N137) appeared to H-bond with histidine at position 168 and threonine at position 187 in CH1 (EU numbering). A variety of substitutions were tested (alanine, leucine, arginine, lysine and glutamine) at N137 in LC33 (LCs 50 and 52-55 as shown in FIG. 25 Table 13) in combination with either the intended pairing partner HC47, or the non-paring partner HC43 (HC mutations are shown in FIG. 26 Table 14).

Kappa chain LC13, whose intended pairing partner is the CH1 pairing region, was observed to have a slightly higher pairing with HC47 (which comprised three mutations that reduce binding to the IgM receptors: E468R, E526A and Q510R) than with HC42 (otherwise identical but lacking these three mutations). Glutamine at position 124 was identified in the kappa chain (Q124) as a potentially mutable position and tested substitutions with either aspartate or glutamate to reduce pairing with Cμ4.

Polynucleotides encoding antibody chains were constructed, each comprised an open reading frame encoding an antibody chain operably linked to a CMV promoter and rabbit globin polyadenylation sequence such that the open reading frame was expressible in a cultured mammalian cell. Polynucleotides were as follows.

Polynucleotides for expression of antibody light chains using a kappa pairing region each comprised a sequence encoding, from N-terminus to C-terminus: a secretion signal, the light chain variable region of the antibody (with amino acid sequence SEQ ID NO:28) and a kappa pairing region (with an amino acid sequence corresponding to a SEQ ID NO shown in FIG. 25 Table 13, column F). The SEQ ID NO corresponding to the mature amino acid sequence of each light chain is shown in FIG. 25 Table 13, column E. The position of engineered cysteines is indicated in column B (as well as the amino acid that is replaced), whether the natural cysteine at position 214 was allowed to remain is indicated in column C, other mutations are indicated in column D of FIG. 25 Table 13.

Polynucleotides for expression of antibody heavy chains each comprised a sequence encoding, from N-terminus to C-terminus: a secretion signal, the heavy chain variable region of the antibody (with amino acid sequence SEQ ID NO:29), a human IgM Cμ4 constant region or a human IgG CH1 region (with amino acid sequence corresponding to a SEQ ID NO shown in FIG. 26 Table 14, column G if the pairing region was an IgG CH1 region, or in column F if the pairing region was an IgM Cμ4 pairing region), a hinge region (with amino acid sequence corresponding to a SEQ ID NO shown in FIG. 26 Table 14, column H) and an Fc region (human IgG1 CH2 plus CH3 regions) with amino acid sequence SEQ ID NO:118. The SEQ ID NO corresponding to the mature amino acid sequence of each heavy chain is shown in FIG. 26 Table 14, column E. The position of engineered cysteines is indicated in column B (as well as the amino acid that is replaced), whether the natural cysteine at hinge position 220 was allowed to remain is indicated in column C, other mutations are indicated in column D of FIG. 26 Table 14.

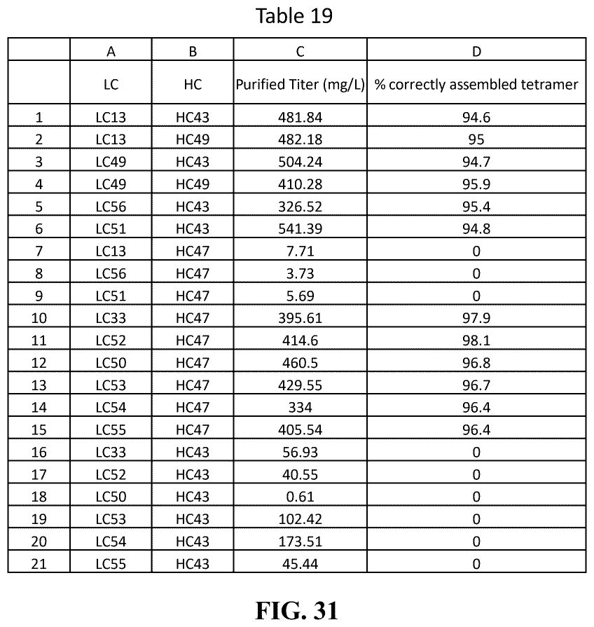

Polynucleotides encoding one heavy chain (or modified heavy chain) and one light chain (or modified light chain) were co-transfected into HEK 293 cells, and the cells were cultured for 7 days in THERMO FISHER SCIENTIFIC Expi293™ media. Protein was purified by affinity chromatography on protein A resin, and the antibody yield was used to calculate the titer in the original culture. FIG. 31 Table 19 shows the different chain combinations tested (the light chain used is shown in column A, the heavy chain used is shown in column B) and the resulting antibody titers in mg/L (column C).

FIG. 31 Table 19, rows 1-4 show the effect of mutating the CH1 leucine at position 128 to either lysine (L128K, as in HC43, FIG. 31 Table 19, rows 1 and 3) or to arginine (L128R, as in HC49, FIG. 31 Table 19, rows 2 and 4), and pairing with a kappa comprising the mutation valine 133 to serine (V133S) and either serine 176 to aspartate (S176D and V133S as in LC13), or serine 176 to glutamate (S176E and V133S as in LC49). All four combinations produced high titers of assembled antibody.

FIG. 31 Table 19, rows 5 and 6 show the effect of mutating kappa glutamine 124 to either aspartate (Q124D) or glutamate (Q124E), in addition to the LC13 mutations S176D and V133S. These kappa light chains were paired with heavy chains comprising the CH1 heavy chain pairing region HC43 (which comprises the L128K mutation). The kappa Q124D mutation added to LC13 led to a slight reduction in titer when paired with HC43, compared to the pairing of HC43 with LC13 (compare FIG. 31 Table 19, rows 1 and 5), but the kappa Q124E mutation added to LC13 led to a slight increase in titer when paired with HC43, compared to the pairing of HC43 with LC13 (compare FIG. 31 Table 19, rows 1 and 6). When these two kappa muteins were paired with what is intended to be the orthogonal Cμ4 HC47 pairing region (HC47 comprises mutations T477D, E468R, E526A, Q510R and F516C), both resulted in even lower titers than the pairing with LC13 (compare FIG. 31 Table 19, row 7 with rows 8 and 9). Thus mutation of kappa glutamine 124 to either aspartate (Q124D) or glutamate (Q124E) can be used to reduce unwanted kappa pairing with an IgM Cμ4 pairing region while maintaining desired pairing with a CH1 pairing region.

FIG. 31 Table 19, rows 10-15 show the effect of mutating kappa asparagine 137 in LC33 (which also comprises mutations Q160C and S131K) on its pairing with HC47. Mutation of asparagine 137 to lysine produced a small reduction in antibody titer (compare FIG. 31 Table 19, rows 10 and 14), but mutation of asparagine 137 to alanine, leucine, arginine or glutamine had little effect (compare FIG. 31 Table 19, row 10 with rows 11-13 and 15). The same kappa pairing regions were tested also in combination with CH1-based heavy chain pairing region HC43. Most kappa asparagine 137 mutations had only small effects on this pairing, but one: asparagine 137 to leucine (N137L) dramatically reduced antibody titer (compare FIG. 31 Table 19, row 18 with row 16). Thus, mutation of kappa asparagine 137 to leucine (N137L) can be used to reduce unwanted kappa pairing with an IgG CH1 pairing region while maintaining desired pairing with a IgM Cμ4 pairing region.

### Example 11

### Kappa Mutations to Increase Thermostability of Pairing with IgM Cμ4

Thermostability is an important attribute of a therapeutic antibody for, amongst other things, efficacy, shelf life and manufacturability. In general, it is desirable for the melting temperature (Tm) of the Fab portion of an antibody to be at least equal to the melting temperature for the Fc region, which typically melts at around 65° C. A Fab region was prepared consisting of:
(a) light chain LC10 with mature amino acid sequence SEQ ID NO:235 (see FIG. 25 Table 13) comprising a light chain variable region with mature amino acid sequence SEQ ID NO:28 fused to a modified kappa pairing region with amino acid sequence SEQ ID NO:248 and
(b) heavy chain HC21-Fab with mature amino acid sequence SEQ ID NO:287 comprising a heavy chain variable region with mature amino acid sequence SEQ ID NO:29 fused to a modified IgM Cμ4 pairing region, upper hinge and polyhistidine tract with amino acid sequence SEQ ID NO:288.

The protein was expressed in HEK cells, purified over nickel NTA resin and Tm of this Fab protein was measured by Differential Scanning Fluorimetry (DSF) and determined to be 54.5° C. Since this value is significantly below the desired Tm for the Fab portion of an antibody, a study was initiated to identify substitutions that could be made in the kappa pairing region to increase the Tm when paired with an IgM Cμ4 pairing region.

Approximately 500 sequence homologs (with amino acid identity >40%) of the human kappa light chain pairing region were identified, and were used to identify a set of 60 candidate amino acid substitutions using methods described previously in U.S. Pat. No. 8,635,029. A set of 94 variant kappa pairing regions were designed comprising one or more of these substitutions. The sequences of the variant kappa regions had amino acid sequences of SEQ ID NOs: 289-382.

A polynucleotide comprising an open reading frame encoding a light chain comprising a secretion signal, a light chain variable region with mature amino acid sequence SEQ ID NO:28 fused to one of the variant kappa pairing regions was prepared for each of these variant light chain pairing regions. An HC21-Fab-encoding polynucleotide comprised an open reading frame encoding a heavy chain comprising a secretion signal and a heavy variable region fused to a modified mature amino acid sequence IgM Cμ4 pairing region, upper hinge and polyhistidine tract, the mature heavy chain Fab chain consisting of amino acid sequence SEQ ID NO:287. The open reading frame in each polynucleotide was operably linked to a CMV promoter and rabbit globin polyadenylation sequence such that the open reading frame was expressible in a cultured mammalian cell.

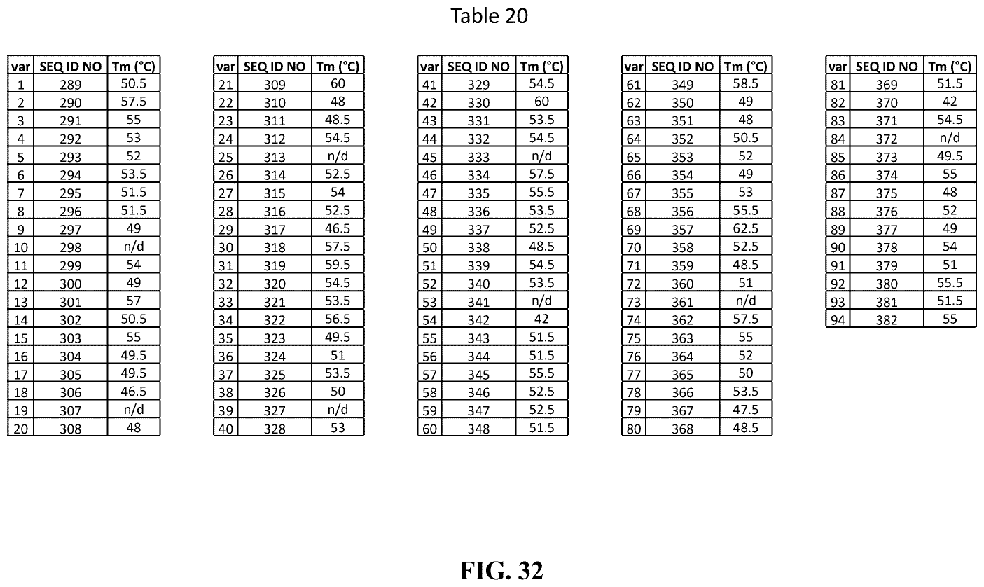

Polynucleotides encoding HC21-Fab and one light chain variant were co-transfected into HEK 293 cells, and the cells were cultured for 7 days in THERMO FISHER SCIENTIFIC Expi293™ media. Expressed proteins were purified using nickel NTA resin, and Tm measured by DSF. The results are shown in FIG. 32 Table 20, which has columns for the SEQ ID NO corresponding to the amino acid sequence of the modified kappa region (“SEQ ID NO”), and for melting temperature in degrees Celsius (Tm). In a few cases, the sequence modifications resulted in an antibody that was not expressed. In those cases no Tm measurement was made, this is indicated as n/d (“not done”) in the Tm column.

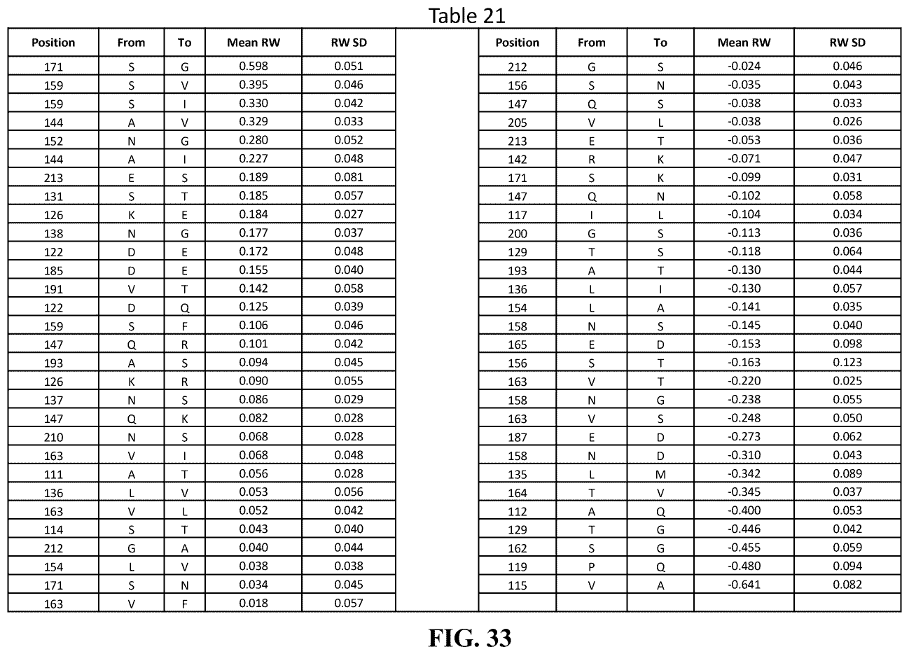

The contributions of different amino acid substitutions to Fab melting temperature was modelled as described previously in U.S. Pat. No. 8,635,029, and mean values and standard deviations for the regression weights were calculated for each substitution. These are shown in FIG. 33 Table 21 which shows the position in the kappa pairing region by EU numbering (“Position”), the amino acid being changed (“From”), the amino acid change being made (“To”), the mean regression weight (“Mean RW”) and the regression weight standard deviation (“RW SD”). Substitutions with positive model weights are those that contribute positively to Tm. Particularly advantageous substitutions include S171G, S159V, S159I, A144V, N152G, A1441, E213S, S131T, K126E, N138G, D122E, D185E, V191T, D122Q, S159F, Q147R, A193S, K126R, N137S, Q147K, N210S, V1631 and A111T.

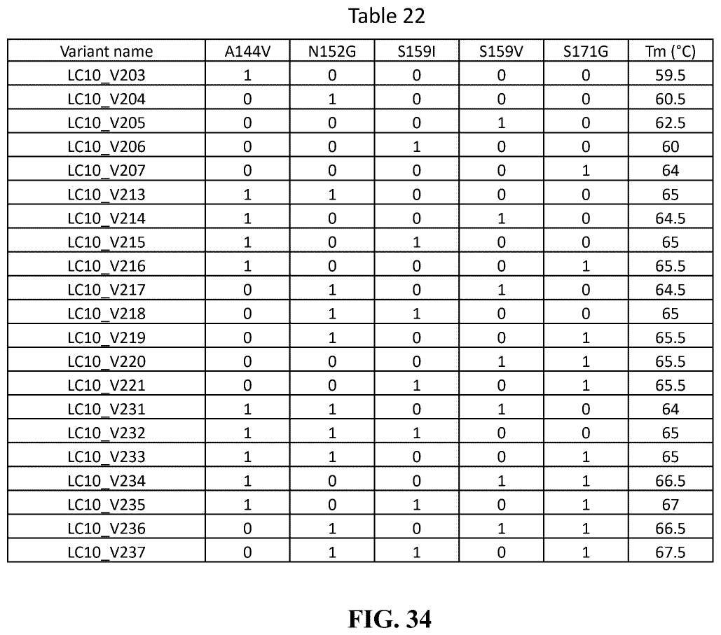

Various combinations of the substitutions predicted to have the most positive effect on Fab antibody Tm we tested using the procedure described above to synthesize Fab antibodies comprising heavy chain HC21-fab and light chain LC10, wherein LC10 further comprised one or more substitutions selected from S171G, S159V, S159I, A144V and N152G. FIG. 34 Table 22 shows a column for each of these substitutions, a 1 indicates that the substitution was present in a variant, a 0 indicates its absence. The Tm column indicates the measured Tm in degrees Celsius. All combinations of these five substitutions substantially increased the melting temperature of the Fab, demonstrating that the model predictions were accurate and that one or more substitution selected from S171G, S159V, S159I, A144V, N152G in the kappa pairing region are particularly advantageous in increasing the melting temperature when the kappa region is paired with an IgM Cμ4 pairing region.

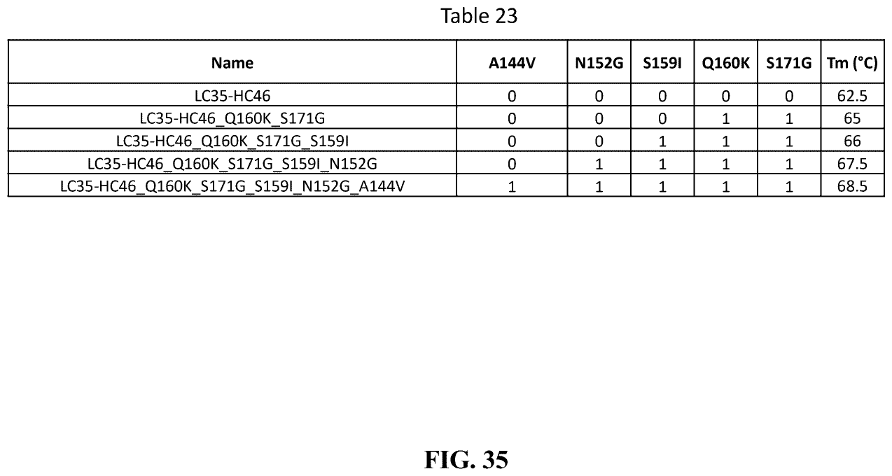

LC10 is a kappa chain comprising a Q124C glutamine to cysteine substitution. This forms a disulfide bond with the engineered cysteine in HC21, which contains an IgM Cμ4 pairing region comprising a Y455C tyrosine to cysteine substitution. LC10 further comprises an E123K substitution, which forms an electrostatic bond with K220E in the upper hinge region of HC21. To verify that the substitutions to increase Tm would function similarly in other contexts, these substitutions were tested with another set of pairing regions: LC35 and HC46 whose amino acid sequences are SEQ ID NOs:255 and 267 respectively. LC35 and HC46 comprise the same pair of engineered cysteines as LC10 and HC21 (a Q124C glutamine to cysteine substitution in the kappa pairing region of LC35, a Y455C tyrosine to cysteine substitution in the IgM Cμ4 pairing region of HC47). However instead of the E123K substitution in LC10, LC35 comprises an S131K substitution, which forms an electrostatic bond with a corresponding T477D substitution in the IgM Cμ4 pairing region of HC46. To the LC35 kappa pairing region was added the substitution Q160K, and then added sequentially S171G, S159I, N152G and A144V. Proteins were expressed and analyzed as described above, the mature amino acid sequence of HC46-Fab is SEQ ID NO:383, the mature amino acid sequence of LC35 is SEQ ID NO:242. FIG. 35 Table 23 shows the name of each variant (“Name”) and the presence (indicated by a 1) or absence (indicated by a 0) of Q160K, S171G, S159I, N152G or A144V in their respective columns. The melting temperature measured by DSF is shown in the Tm column. As can be seen in FIG. 35 Table 23, the starting Tm of LC35-HC46 is 62.5° C. Addition of Q160K and S171G increases this to 65° C., further addition of S159I increases this to 66° C., further addition of N152G increases this to 67.5° C., further addition of A144V increases this to 68.5° C. Thus, incorporation of each of the substitutions S171G, S159I, N152G and A144V into a kappa pairing region each increases the thermostability of that kappa region paired with an IgM Cμ4 pairing region.

### Example 12

### Thermostability Optimization Between Kappa and IgM Cμ4 Pairing Regions Using Alternative Disulfide Bonds

Example 11 describes substitutions in a kappa pairing region that increase thermostability when paired with an IgM Cμ4 pairing region. The kappa and Cμ4 pairing regions in Example 11 comprised an engineered cysteine pair resulting in a disulfide bond between the two pairing regions: These were glutamine to cysteine substitution Q124C in the kappa pairing region and a tyrosine to cysteine substitution Y455C in the IgM Cμ4 pairing region. In an effort to identify kappa pairing region substitutions that would increase the thermostability of a kappa-IgM Cμ4 pair with an alternative engineered disulfide bond, this time between a kappa pairing region comprising a glutamine to cysteine Q160C substitution, and an IgM Cμ4 pairing region comprising a phenylalanine to cysteine F516C substitution.

A Fab region was prepared consisting of:
(a) light chain LC33 with mature amino acid sequence SEQ ID NO:240 (see FIG. 25 Table 13) comprising a light chain variable region with mature amino acid sequence SEQ ID NO:28 fused to a modified kappa pairing region with amino acid sequence SEQ ID NO:253, and
(b) heavy chain HC47-Fab with mature amino acid sequence SEQ ID NO:384 comprising a heavy chain variable region with mature amino acid sequence SEQ ID NO:29 fused to a modified IgM Cμ4 pairing region, upper hinge and polyhistidine tract.

The protein was expressed in HEK cells, purified over nickel NTA resin and Tm of this Fab protein was measured by Differential Scanning Fluorimetry (DSF) and determined to be 69° C.

Approximately 500 sequence homologs (with amino acid identity >40%) of the human kappa light chain pairing region were identified, and these sequences were used to identify a set of 60 candidate amino acid substitutions using methods described previously in U.S. Pat. No. 8,635,029. A set of 94 variant kappa pairing regions comprising one or mor of these substitutions was designed. The sequences of the variant kappa regions had amino acid sequences SEQ ID NOs: 385-478.

A polynucleotide comprising an open reading frame encoding a light chain comprising a secretion signal, a light chain variable region with mature amino acid sequence SEQ ID NO:28 fused to one of the variant kappa pairing regions was prepared for each of these variant light chain pairing regions. An HC47-Fab-encoding polynucleotide comprised an open reading frame encoding a heavy chain comprising a secretion signal and a heavy variable region fused to a modified mature amino acid sequence IgM Cμ4 pairing region, upper hinge and polyhistidine tract, the mature heavy chain Fab chain consisting of amino acid sequence SEQ ID NO:384. The open reading frame in each polynucleotide was operably linked to a CMV promoter and rabbit globin polyadenylation sequence such that the open reading frame was expressible in a cultured mammalian cell.

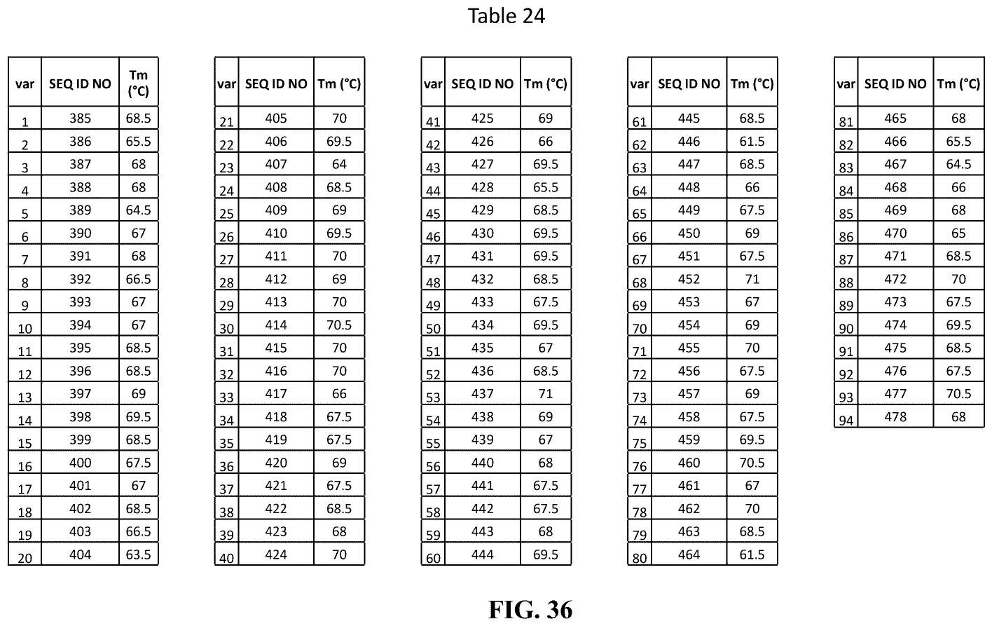

Polynucleotides encoding HC47-fab and one light chain variant were co-transfected into HEK 293 cells, and the cells were cultured for 7 days in THERMO FISHER SCIENTIFIC Expi293^{T}M media. Expressed proteins were purified using nickel NTA resin, and Tm measured by DSF. The results are shown in FIG. 36 Table 24, which has columns for the SEQ ID NO corresponding to the amino acid sequence of the modified kappa region (“SEQ ID NO”), and for melting temperature in degrees Celsius (Tm).

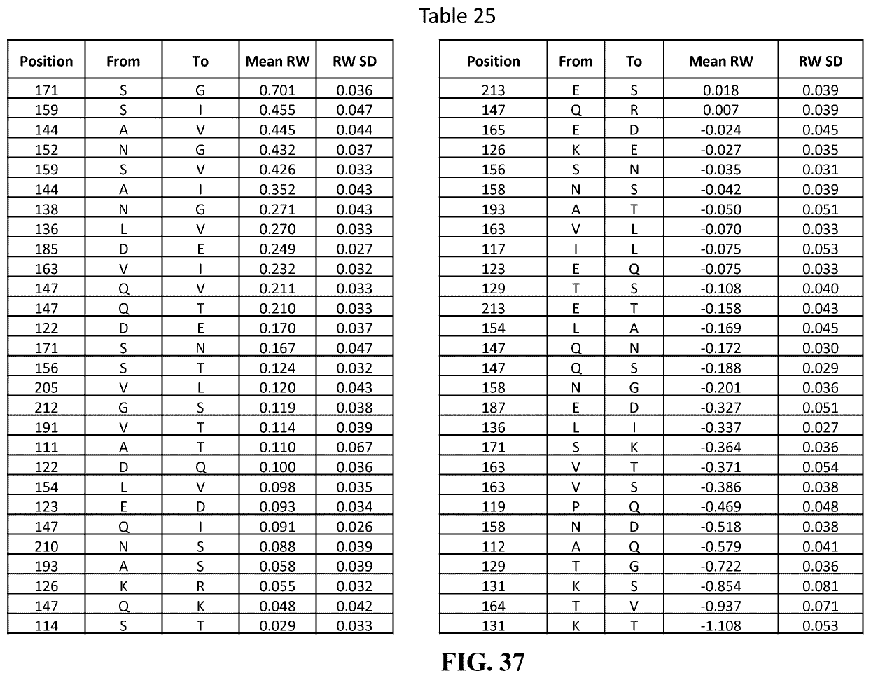

The contributions of different amino acid substitutions to Fab melting temperature was modelled as described previously in U.S. Pat. No. 8,635,029, and mean values and standard deviations for the regression weights were calculated for each substitution. These are shown in FIG. 37 Table 25 which shows the position in the kappa pairing region by EU numbering (“Position”), the amino acid being changed (“From”), the amino acid change being made (“To”), the mean regression weight (“Mean RW”) and the regression weight standard deviation (“RW SD”). Substitutions with positive model weights are those that contribute positively to Tm. Particularly advantageous kappa pairing region substitutions for increasing the thermostability of kappa when paired with an IgM Cμ4 pairing region include S171G, S159I, A144V, N152G, S159V, A1441, N138G, L136V, D185E, V1631, Q147V, Q147T, D122E, S171N, S156T, V205L, A111T, D122Q, L154V, E123D, Q147I, N210S and A193S. The majority of these positive substitutions were also positive for the LC10-HC21 pair: A111T, D122E, D122Q, K126R, N138G, A144V, A1441, N152G, S159I, S159V, V1631, S171G, D185E, V191T, A193S and N210S were all positive in both cases.

Two additional kappa substitutions were also tested on the thermostability of one of the best performing LC33variant-HC47 pairs. The kappa pairing region with amino acid sequence SEQ ID NO:452 comprised substitutions Q147V, D185E and S159V and had a Tm of 71° C. Additional substitutions N137L and S171G were added to the kappa region using the procedure described above to synthesize a Fab antibody comprising this new variant light chain and heavy chain HC47-Fab. The Tm of this new Fab was increased to 73.5° C. As with Example 11, this shows that adding more of the kappa substitutions identified to have a positive effect on thermostability can be used to further increase the melting temperature of the Fab portion of these antibodies.

### Example 13

### Orthogonal Pairing of Two Different Kappa-IgM Cμ4 Pairing Partners

Example 9 describes the addition of substitutions to kappa and IgM Cμ4 pairing regions to create electrostatic interactions to promote pairing. Substitutions can also be incorporated to create electrostatic repulsions, thereby disfavoring unwanted pairing. One example of such substitutions that can create two orthogonal kappa—IgM Cμ4 pairs comprise changes at position 131 in the kappa pairing region and position 477 in the IgM Cμ4 pairing region. As described above in Example 9, LC33 and LC35 each comprise a serine to lysine substitution S131K in the kappa pairing region (as shown in FIG. 25 Table 13). LC33 and LC35 pair respectively with HC47 and HC46, which each comprising a threonine to aspartate substitution T477D in the IgM Cμ4 pairing region (as shown in FIG. 26 Table 14). Although they share the same S131K-T477D electrostatic interactions, the LC33-HC47 pair and the LC35-HC46 pair differ in the positions of their engineered cysteines.

To further increase specificity of pairing between light and heavy chains, residues were engineered in both heavy and light chains to create additional electrostatic interactions. The objective was to introduce mutually attractive changes into corresponding pairing regions that would at the same time result in mutually repulsive changes in non-corresponding pairing regions. These changes were combinations of one of the following in each pairing region:
(i) mutation of serine to lysine S131K in the kappa pairing region, and mutation of threonine to aspartate T477D in the corresponding IgM Cμ4 pairing region; and
(ii) mutation of serine to aspartate or glutamate (S131D or S131E) in the kappa pairing region, and mutation of threonine to lysine T477K in the corresponding IgM Cμ4 pairing region.

Kappa pairing regions also comprised substitution of the C-terminal cysteine to alanine (C214A), and either introduction of a new cysteine at position 124 (Q124C) or at position 160 (Q160C). IgM Cμ4 pairing regions also comprised either introduction of a new cysteine at position 455 (Y455C) or at position 516 (F516C). Kappa and IgM Cμ4 pairing regions also comprised substitutions to increase thermostability or to reduce IgM receptor binding, as described in previous examples.

Polynucleotides encoding antibody chains were constructed, each comprised an open reading frame encoding an antibody chain operably linked to a CMV promoter and rabbit globin polyadenylation sequence such that the open reading frame was expressible in a cultured mammalian cell.

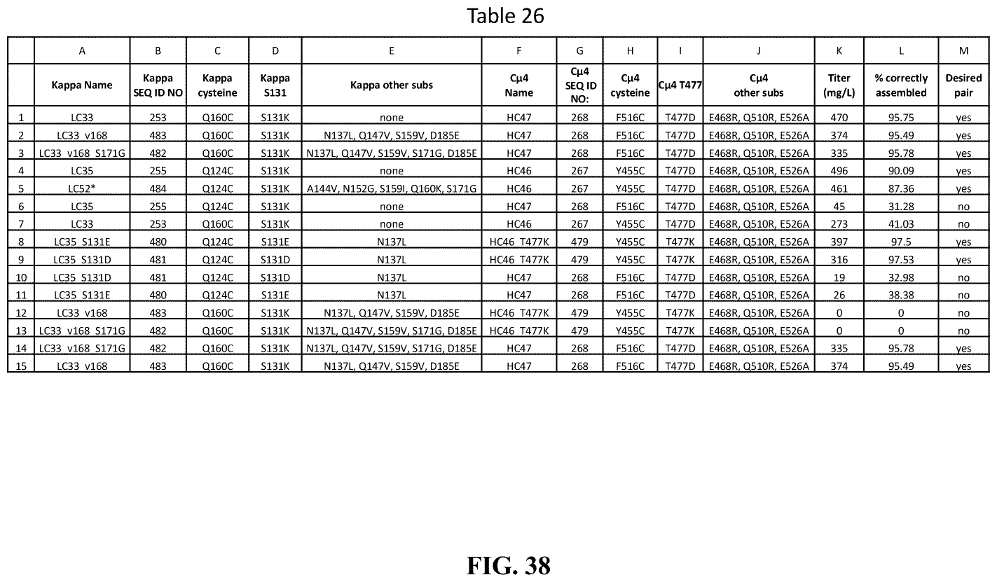

Polynucleotides for expression of antibody light chains using a kappa pairing region each comprised a sequence encoding, from N-terminus to C-terminus: a secretion signal, the light chain variable region of the antibody (with amino acid sequence SEQ ID NO:28) and a kappa pairing region (with an amino acid sequence corresponding to a SEQ ID NO shown in FIG. 38 Table 26, column B). The positions of engineered cysteines are indicated in FIG. 38 Table 26 column C (as well as the amino acid that is replaced), the substitution at serine S131C is shown in FIG. 38 Table 26 column D, other mutations are indicated in column E of FIG. 38 Table 26.

Polynucleotides for expression of antibody heavy chains each comprised a sequence encoding, from N-terminus to C-terminus: a secretion signal, the heavy chain variable region of the antibody (with amino acid sequence SEQ ID NO:29), a human IgM Cμ4 constant region or a human IgG CH1 region (with amino acid sequence corresponding to a SEQ ID NO shown in FIG. 38 Table 26, column G, a hinge region (with amino acid sequence SEQ ID NO:117) and an Fc region (human IgG1 CH2 plus CH3 regions) with amino acid sequence SEQ ID NO:118. The position of engineered cysteines are indicated in column H (as well as the amino acid that is replaced), the substitution at position 477 is shown in FIG. 38 Table 26 column I, other mutations are indicated in column J of FIG. 38 Table 26.

Polynucleotides encoding one heavy chain (or modified heavy chain) and one light chain (or modified light chain) were co-transfected into HEK 293 cells, and the cells were cultured for 7 days in THERMO FISHER SCIENTIFIC Expi293™ media. Protein was purified by affinity chromatography on protein A resin, and the antibody yield was used to calculate the titer in the original culture. FIG. 38 Table 26 shows the different chain combinations tested and the resulting antibody titers in mg/L (FIG. 38 Table 26 column K). Protein was also run over an SEC column, and the % of product running at the correct size for a properly assembled antibody is shown in column M of FIG. 38 Table 26.

FIG. 38 Table 26, rows 1-7 shows the titers of antibodies (in mg/L) produced with various combinations of light chains comprising a kappa pairing region comprising an S131K mutation combined with heavy chains all comprising an IgM Cμ4 pairing region comprising a T477D mutation. Engineered cysteines are appropriately matched in antibodies shown in rows 1-5 (i.e., kappa Q124C is matched with IgM Cμ4 Y455C and kappa Q160C is matched with IgM Cμ4 F516C). Each of these antibodies shows high titers of production (>300 mg/L) and high percentages of correctly assembled antibody (>87%). FIG. 38 Table 26 rows 6 and 7 show antibodies produced with mismatched engineered cysteines: these are the light chain from the antibody in row 4 (LC35) paired with the heavy chain from the antibody in row 1 (HC47), and the light chain for the antibody in row 1 (LC33) paired with the heavy chain from the antibody in row 4 (HC46). LC35, with Q124C yields less than 10% of the amount of antibody (45 mg/L) when it is paired with HC47 comprising an engineered cysteine F516C (FIG. 38 Table 26 row 6), compared with the yield with its matched partner HC46 (496 mg/L), which is an acceptably low level of pairing for pairing regions that should be orthogonal. However LC33 with Q160C yields over 50% of the amount of antibody (273 mg/L) when it is paired with HC46 comprising an engineered cysteine Y455C (FIG. 38 Table 26 row 7), compared with the yield with its matched partner HC47 (470 mg/L), which is an unacceptably high level of pairing for pairing regions that should be orthogonal. A bispecific antibody comprising pairing regions LC33, HC47, LC35 and HC47 would produce correct pairing (FIG. 38 Table 26 rows 1 and 4), but would also have significant light chain mispairing between LC33 and HC46 (FIG. 38 Table 26 row 7).

To reduce this light chain mispairing, the charges on one of the engineered electrostatic pairs was reversed. FIG. 38 Table 26, rows 8 and 9 show pairing between a light chain with a kappa pairing region comprising a serine 131 replacement by either aspartate (LC35_S131E, row 8) or glutamate (LC35_S131E, row 9), and a heavy chain with an IgM Cμ4 pairing region comprising a threonine 477 replacement by lysine (HC46_T477K). Both of these combinations result in high levels of production (>300 mg/L) of correctly folded antibody (>95%). When these kappa light chains were instead paired with HC47, less than half the amount of antibody was produced (<27 mg/L) compared with the amount produced with LC35 and HC47 (compare FIG. 38 Table 26, row 6 with rows 10 and 11), showing that the light chain charge switch reduced this already low light chain mispairing further. We also tested pairing between different heavy chains and versions of LC33 to which additional mutations had been added to increase thermostability, but in which the charge resulting from the substitution S131K remained. These light chains (LC33_v168_S171G and LC33_v168) both paired with heavy chain HC47 in a comparable way to LC33 (compare FIG. 38 Table 26, row 1 with rows 14 and 15), all of which produce high titers (>300 mg/L) of properly assembled antibodies (>95%). In contrast, when paired with the charge-switched heavy chain HC46_T477K, no detectable antibody was produced (FIG. 38 Table 26 rows 12 and 13), showing that the charge switch on the IgM Cμ4 pairing region from T477D to T477K eliminated light chain mispairing.

Thus an orthogonal set of heavy and light chains, in which the light chains are able to interact well with their corresponding heavy chains, enabling their folding, secretion and correct disulfide bonding, but wherein the light chains do not interact with their non-corresponding heavy chains, even sufficient to enable efficient secretion of the non-corresponding heavy chain comprises:
(i) a first chain comprising a first IgM Cμ4 pairing region comprising mutations Y455C and T477K within the IgM Cμ4 region,
(ii) a corresponding second chain comprising a first kappa pairing region comprising mutations Q124C, C214A and either (a) S131D, or (b) S131E,
(iii) a third chain comprising a second IgM Cμ4 pairing region comprising mutations F516C and T477D within the IgM Cμ4 region, and
(iv) a fourth chain corresponding to the third chain, the fourth chain comprising a second kappa pairing region comprising mutations Q160C, C214A and S131K.

Optionally one of the first or second chains and one of the third or fourth chains further comprises an IgG1 hinge region and joined to an IgG Fc (CH2 and CH3) region.

### Example 14

### Additional Mutations to Confer Preferential Pairing Between Pairing Partners

As described in Example 9, the addition of electrostatic steering mutations into the kappa, CH1 and IgM Cμ4 pairing regions improves the specificity of pairing. However, a low level of residual unwanted pairing was observed between the kappa chains engineered to pair specifically with Cμ4 “Cμ4-engineered kappas”, and the CH1 pairing region. Additional tests were undertaken to identify additional mutations that could be incorporated into either the Cμ4-engineered kappa domains or into the CH1 pairing regions to further reduce their pairing. These tests also expanded the number of antibody variable regions tested, to ensure a general solution. First, pairing between corresponding Cμ4 and Cμ4-engineered kappa paring regions was assessed, and between corresponding kappa and CH1 pairing regions, by fusing them to eight different corresponding heavy and light chain variable regions. This was done by measuring titer (which is an indication of pairing sufficient for the light chain to pair, chaperone and enable secretion of the corresponding heavy and chain, and which is also important for antibody production), and then by using size exclusion chromatography to measure the fraction of the produced antibody that was correctly assembled into a tetramer of two light chains and two heavy chains with a molecular weight of approximately 160 kDa. The same techniques were used to assess mispairing between kappa and non-corresponding Cμ4 pairing regions, and between Cμ4-engineered kappa and non-corresponding CH1 paring regions, using combinations of light and heavy variable regions that showed a high tendency to associate regardless of the pairing region to which they were fused. Folding of the CH1 region can be made more dependent on pairing with the cognate pairing region by incorporation of the substitution P151A into the CH1 region.

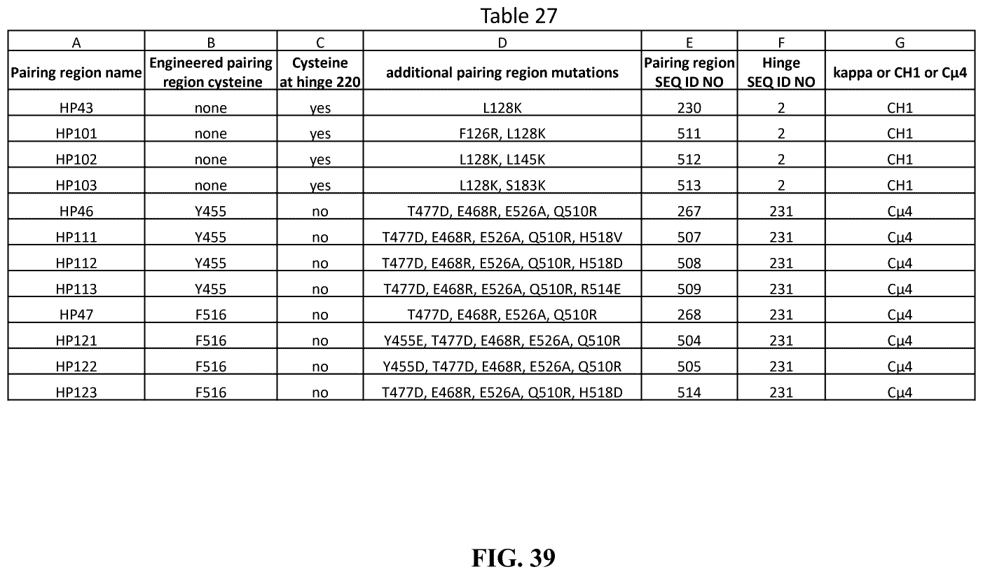

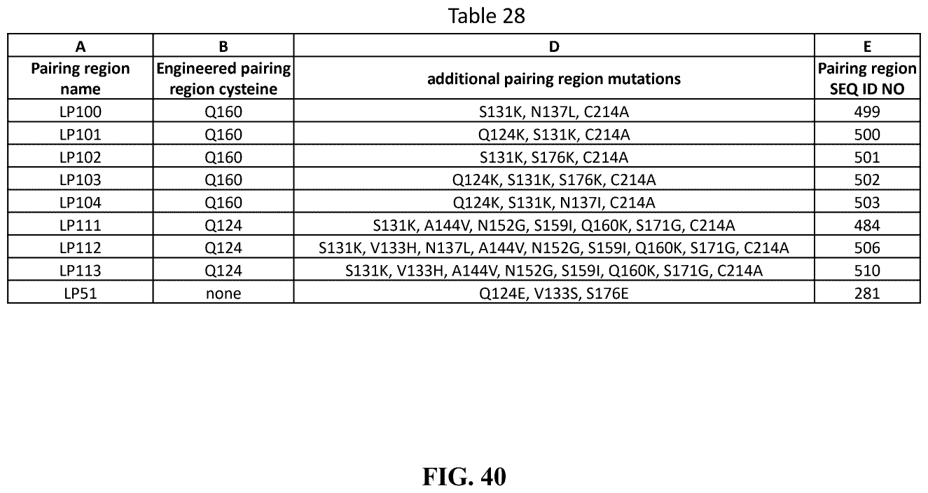

New CH1 pairing regions comprising L128K were constructed, these pairing regions comprised one of the following additional substitutions: F126R, L145K or S183K. These positively charged substitutions were selected to introduce additional electrostatic repulsions against the positively charged Cμ4-engineered kappas comprising a positively charged substitution at S131. The substitutions in these pairing regions are shown in FIG. 39 Table 27. New substitutions were introduced into Cμ4-engineered kappas. These were either (i) additional positively charged substitutions (Q124K, S176K or V133H) designed to introduce additional electrostatic repulsions against the positively charged CH1 L128K substitution, (ii) N1371 or N137L designed to disrupt H-bonding with histidine at position 168 and threonine at position 187 in CH1 (EU numbering), and (iii) substitutions designed to increase the thermostability of the kappa-IgM Cμ4 pairing (A144V, N152G, S159I and S171G). The substitutions in these pairing regions are shown in FIG. 40 Table 28. New IgM Cμ4 pairing regions comprising T477D were constructed. These pairing regions either comprised an additional negatively charged substitution (H518D, R514E, Y455D, Y455E) designed to increase pairing with the positively charged IgM Cμ4-engineered kappas, or H518V. The substitutions in these pairing regions are shown in FIG. 39 Table 27.

Polynucleotides encoding antibody chains were constructed, each comprised an open reading frame encoding an antibody chain operably linked to a CMV promoter and rabbit globin polyadenylation sequence such that the open reading frame was expressible in a cultured mammalian cell.

Polynucleotides were as follows. Polynucleotides for expression of antibody light chains using a kappa pairing region each comprised a sequence encoding, from N-terminus to C-terminus: a secretion signal, the light chain variable region of the antibody (with an amino acid sequence selected from SEQ ID NO:28, 486, 488, 490, 492, 494, 496 or 498) and a kappa pairing region (with an amino acid sequence selected from SEQ ID NO 499, 500, 501, 502, 503, 484, 506, 510 or 281, with further details shown in FIG. 40 Table 28).

Polynucleotides for expression of antibody heavy chains each comprised a sequence encoding, from N-terminus to C-terminus: a secretion signal, the heavy chain variable region of the antibody (with an amino acid sequence selected from SEQ ID NO:29, 485, 487, 489, 491, 493, 495 or 497), and either a human IgG CH1 region (with amino acid sequence selected from SEQ ID NO:230, 511, 512 or 513 and a hinge region with amino acid sequence SEQ ID NO:2), or a human IgM Cμ4 constant region (with amino acid sequence selected from SEQ ID NO:267, 507, 508, 509, 268, 504, 505 or 514 and a hinge region with amino acid sequence SEQ ID NO:231) and an Fc region (human IgG1 CH2 plus CH3 regions) with amino acid sequence SEQ ID NO:118.

Polynucleotides encoding one heavy chain and one light chain were co-transfected into HEK 293 cells, and the cells were cultured for 7 days in THERMO FISHER SCIENTIFIC Expi293™ media. Protein was purified by affinity chromatography on protein A resin, and the antibody yield was used to calculate the titer in the original culture. Purified proteins were analyzed by size exclusion chromatography to assess the percentage of correctly assembled antibody.

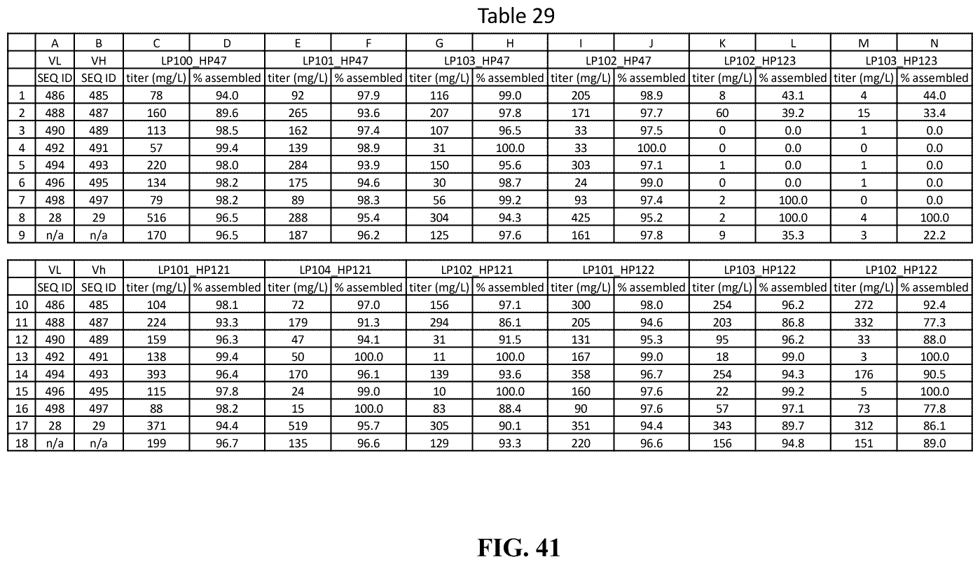

Various combinations of Cμ4-engineered kappa pairing regions with corresponding Cμ4 pairing regions were tested, wherein the Cμ4-engineered kappa pairing regions comprised an engineered cysteine at position Q160C, and the Cμ4 pairing regions comprised an engineered cysteine at position F516C. The results are shown in FIG. 41 Table 29. Columns A and B provide the SEQ ID NOs corresponding the amino acid sequences of the light and heavy pairing regions respectively. Subsequent pairs of columns give, in the left hand column of each pair, the titer of antibody produced (in mg/L), and in the right hand column of each pair, the percentage of the purified antibody that is correctly assembled as measured by size exclusion chromatography. Above each pair of columns is indicated the pairing regions used, first the light chain pairing region, then an underscore, then the heavy chain pairing region. Details about each pairing region, including the substitutions in each and the SEQ ID NO corresponding to the amino acid sequence of the pairing region are shown in FIG. 40 Table 28 for the light chain pairing regions and in FIG. 39 Table 27 for the heavy chain pairing regions. FIG. 41 Table 29, rows 1-8 and 10-17 give titers or percentage of correctly assembled antibody for each combination, and rows 9 and 18 give average values for a single pairing region combination with eight different antibody variable regions. FIG. 41 Table 29 shows that HP123, comprising substitution H518D, yielded very low titers (FIG. 41 Table 29, columns K and M, rows 1-9). The highest titer combinations were LP101 (comprising substitutions S131K and Q124K) combined with either HP47 (comprising T477D: average titer 187 mg/L, FIG. 41 Table 29 rows 1-9 column E), HP121 (comprising T477D and Y455E: average titer 199 mg/L, FIG. 41 Table 29 rows 10-18 column C) and HP122 (comprising T477D and Y455D: average titer 220 mg/L, FIG. 41 Table 29 rows 10-18 column I). The fraction of these antibodies that were correctly assembled was excellent at >95% for all 3 combinations (FIG. 41 Table 29 rows 1-9 column F, Table 29 rows 10-18 column D and Table 29 rows 10-18 column J). These results illustrate that a kappa pairing region comprising substitutions Q124K, S131K and Q160C pairs well with a IgM Cμ4 pairing region comprising substitutions T477D and F516C, and optionally further comprising substitution Y455E or Y455D, most preferably Y455D.

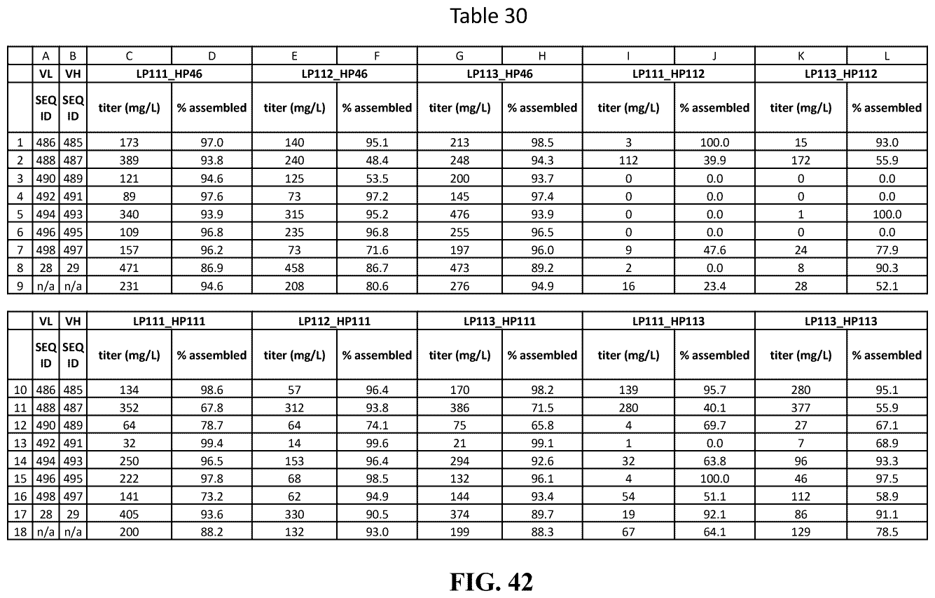

Various combinations of IgM Cμ4-engineered kappa pairing regions were tested with corresponding IgM Cμ4 pairing regions, wherein the Cμ4-engineered kappa pairing regions comprised an engineered cysteine at position Q124C, and the IgM Cμ4 pairing regions comprised an engineered cysteine at position Y455C. The results are shown in FIG. 42 Table 30. Columns A and B provide the SEQ ID NOs corresponding to the amino acid sequences of the light and heavy pairing regions respectively. Subsequent pairs of columns give, in the left hand column of each pair, the titer of antibody produced (in mg/L), and in the right hand column of each pair, the percentage of the purified antibody that is correctly assembled as measured by size exclusion chromatography. Above each pair of columns is indicated the pairing regions used, first the light chain pairing region, then an underscore, then the heavy chain pairing region. Details about each pairing region, including the substitutions in each and the SEQ ID NO corresponding to the amino acid sequence of the pairing region are shown in FIG. 40 Table 28 for the light chain pairing regions and in FIG. 39 Table 27 for the heavy chain pairing regions. Rows 1-8 and 10-17 give titers or % of correctly assembled antibody for each combination, and rows 9 and 18 give average values for a single pairing region combination with eight different antibody variable regions. FIG. 42 Table 30 shows that HP112, comprising substitution H518D, yielded very low titers (FIG. 42 Table 30 columns I and K rows 1-9), as did HP113 comprising substitution R514E (FIG. 42 Table 30 columns I and K rows 10-18). The highest titer combinations were LP111, LP112 and LP113 (each comprising electrostatic substitutions S131K and Q160K, and each further comprising thermostabilizing substitutions A144V, N152G, S159I and S171G, with LP112 and LP113 further comprising electrostatic substitution V133H and LP112 further comprising substitution N137L) combined with HP46 (comprising T477D). The average titer for LP111 with HP46 was 231 mg/L (FIG. 42 Table 30 rows 1-9 column C); the average titer for LP112 with HP46 was 208 mg/L (FIG. 42 Table 30 rows 1-9 column E) and the average titer for LP113 with HP46 was 276 mg/L (FIG. 42 Table 30 rows 1-9 column G). The fraction of antibodies that were correctly assembled for LP111 and LP113 with HP46 was excellent at ˜95% (FIG. 42 Table 30 rows 1-9 column D and, FIG. 42 Table 30 rows 1-9 column H). The combination of LP112 with HP46 did not perform as well, with an average of \hat{˜}˜80% (FIG. 42 Table 30 rows 1-9 column F). These results illustrate that a kappa pairing region comprising substitutions Q124C, S131K, Q160K pairs well with a Cμ4 pairing region comprising substitutions Y455C and T477D, wherein the kappa pairing region optionally further comprises on or more substitutions selected from V133H, A144V, N152G, S159I and S171G and preferably including V133H.

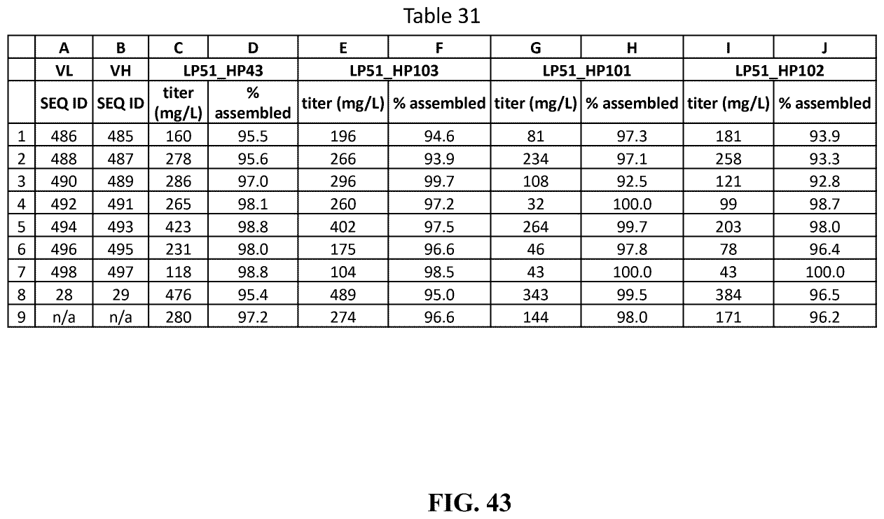

Various combinations of kappa pairing regions with corresponding CH1 pairing regions were also tested, wherein the kappa pairing regions comprised substitutions Q124E, V133S and S176E, and the CH1 pairing region comprised substitution L128K and optionally a further substitution selected from F126R, L145K or S183K. The results are shown in FIG. 43 Table 31. Columns A and B provide the SEQ ID NOs corresponding to the amino acid sequences of the light and heavy pairing regions respectively. Subsequent pairs of columns give, in the left hand column of each pair, the titer of antibody produced (in mg/L), and in the right hand column of each pair, the percentage of the purified antibody that is correctly assembled as measured by size exclusion chromatography. Above each pair of columns is indicated the pairing regions used, first the light chain pairing region, then an underscore, then the heavy chain pairing region. Details about each pairing region, including the substitutions in each and the SEQ ID NO corresponding to the amino acid sequence of the pairing region are shown in FIG. 40 Table 28 for the light chain pairing regions and in FIG. 39 Table 27 for the heavy chain pairing regions. FIG. 43, Table 31, rows 1-8 give titers or percentage of correctly assembled antibody for each combination, and row 9 gives average values for a single pairing region combination with eight different antibody variable regions. FIG. 43 Table 31 shows that HP101 and HP102, comprising substitutions F126R and L145K, yielded relatively lower titers (FIG. 43 Table 31 columns G and I respectively). The highest titer combinations were LP51 combined with HP43 (comprising L128K) or HP103 (comprising L128K and S183K). The average titer for LP51 with HP43 was 280 mg/L (FIG. 43 Table 31, column C); the average titer for LP51 with HP103 was 274 mg/L (FIG. 43 Table 31 column E). The fraction of antibodies that were correctly assembled for LP51 with HP43 and HP103 were both excellent at >95% (FIG. 43 Table 31 columns D and F). LP51 thus pairs equally well with HP43 and HP103.

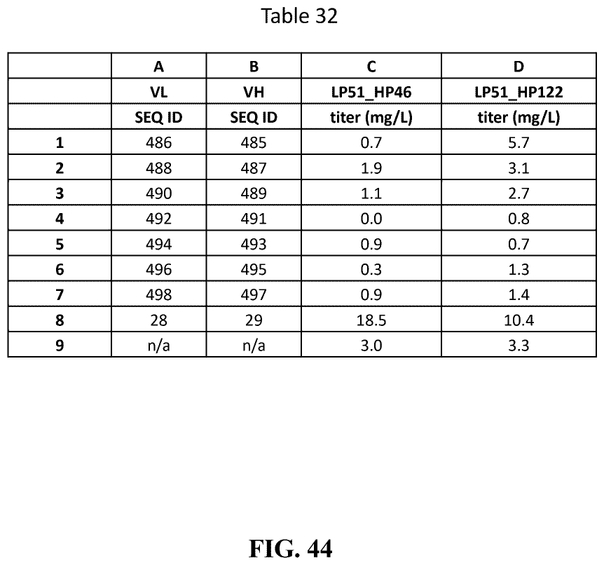

Having identified preferred corresponding pairing regions, their propensity to mispair was measured. As described above in this Example 14, LP51, a kappa pairing region comprising substitutions Q124E, V133S and S176E, pairs well with HP43, a CH1 pairing region comprising substitution L128K. The pairing of LP51 with two high performing IgM Cμ4 pairing regions was tested. These chains were (i) HP46 comprising electrostatic substitution T477D and engineered cysteine Y455C, and (ii) HP122 comprising electrostatic substitution T477D and engineered cysteine F516C and further comprising electrostatic substitution Y455D (see FIG. 39 Table 27 for a full list of substitutions). The results are shown in FIG. 44 Table 32. Columns A and B provide the SEQ ID NOs corresponding to the amino acid sequences of the light and heavy pairing regions respectively. Subsequent columns give the titer of antibody produced (in mg/L) Above each of columns C-D is indicated the pairing regions used, first the light chain pairing region, then an underscore, then the heavy chain pairing region. Details about each pairing region, including the substitutions in each and the SEQ ID NO corresponding to the amino acid sequence of the pairing region are shown in FIG. 40 Table 28 for the light chain pairing regions and in FIG. 39 Table 27 for the heavy chain pairing regions. FIG. 44 Table 32, rows 1-8 give titers for each combination, and row 9 gives average values for a single pairing region combination. FIG. 44 Table 32 shows that almost no antibody is produced for any of the variable regions when LP51 is mispaired with either HP46 of HP112, showing excellent orthogonality between this kappa pairing region and either of the IgM Cμ4 pairing regions.

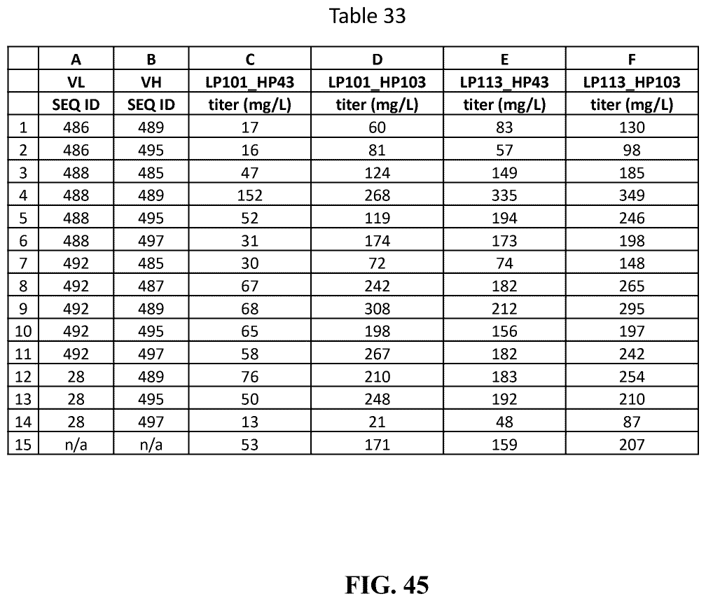

The propensity of Cμ4-engineered kappas to mispair with CH1 was also measured. As described above, earlier in this Example 14, the most productive pairings were between LP101 and HP112 (FIG. 41 Table 29 columns I and J, rows 10-18), and between LP113 and HP46 (FIG. 42 Table 30 columns G and H rows 1-9). It was previously observed that there was higher mispairing between Cμ4-engineered kappas and CH1 pairing regions than between kappa and Cμ4 pairing regions, so test set of variable regions was expanded to include combinations that were particularly prone to unwanted association. This data is shown in FIG. 45 Table 33.

FIG. 45 Table 33, columns A and B provide the SEQ ID NOs corresponding the amino acid sequences of the light and heavy pairing regions respectively. Subsequent columns give the titer of antibody produced (in mg/L). Above each of columns C-F is indicated the pairing regions used, first the light chain pairing region, then an underscore, then the heavy chain pairing region. Details about each pairing region, including the substitutions in each and the SEQ ID NO corresponding to the amino acid sequence of the pairing region are shown in FIG. 40 Table 28 for the light chain pairing regions and in FIG. 39 Table 27 for the heavy chain pairing regions. FIG. 44 Table 32, rows 1-14 give titers for each combination, and row 15 gives average values for a single pairing region combination. FIG. 45 Table 33 shows that the average titer is about a third as high when LP101 (comprising electrostatic substitutions S131K and Q124K as well as engineered cysteine Q160C) is paired with HP43 (comprising L128K) as compared to the titer when it is paired with HP103 (comprising L128K and S183K) (compare FIG. 45 Table 33 columns C and D), showing that LP101 and HP43 are a preferred orthogonal combination. None of the antibodies comprising these two pairing regions were properly assembled as seen by SEC. FIG. 45 Table 33 shows that the average titer is about a 160 mg/L when LP113 (comprising electrostatic substitutions S131K, V133H and Q160K as well as engineered cysteine Q124C and thermostabilizing substitutions A144V, N152G, S159I and S171G) is paired with HP43 (comprising L128K) compared with 207 mg/L when it is paired with HP103 (comprising L128K and S183K) (compare FIG. 45 Table 33 columns E and F), showing that LP113 and HP43 are a preferred orthogonal combination. None of the antibodies comprising these two pairing regions were properly assembled as seen by SEC.

1-113. (canceled)

114. An antibody, comprising:
(a) a first chain comprising a first variable region and a first pairing region; and
(b) a second chain comprising a second variable region and a second pairing region;
wherein the first and second variable regions are heavy and light chain variable regions, or vice versa;
wherein the first and second chains pair to each other via association of the first and second pairing regions, thereby forming a first binding site that specifically binds a first target epitope;
wherein the first pairing region is a first IgM Cμ4 region, and the second pairing region is selected from a first kappa light chain constant region and a first CH1 region.

115. The antibody of claim 114, further comprising at least one amino acid insertion, deletion or substitution in the first or the second pairing region, or both the first and second pairing regions, wherein the at least one amino acid insertion, deletion or substitution results in enhanced affinity of the first and second pairing regions.

116. The antibody of claim 114, wherein the first and second pairing regions each includes an engineered cysteine residue, which form a disulfide bond linking the first chain and second chain.

117. The antibody of claim 116, wherein the second pairing region is a kappa light chain constant region, wherein:
(a) the IgM Cμ4 region comprises an engineered cysteine at amino acid position 455 (by Kabat numbering), and the first kappa light chain constant region comprises an engineered cysteine residue at amino acid position 121, 124 or 131 (by EU numbering); or
(b) the IgM Cμ4 region comprises an engineered cysteine at amino acid position 516 (by Kabat numbering), and the first kappa light chain constant region comprises a cysteine at amino acid position 160 by EU numbering.

118. The antibody of claim 117, wherein the kappa light chain constant regions of (a) or (b) further comprise removing or substituting the cysteine at position C214 by EU numbering with an amino acid incapable of forming a disulfide bond.

119. The antibody of claim 115, wherein the at least one amino acid addition, deletion or substitution enhances electrostatic attraction between the first and second pairing regions.

120. The antibody of claim 119, wherein the second pairing region is the first kappa light chain constant region, wherein:
(a) the first IgM Cμ4 pairing region comprises T477H, T477K or T477R (Kabat numbering); and the second pairing region is a first kappa light chain constant region comprising S131D or S131E (EU numbering); or
(b) the first IgM Cμ4 pairing region comprises T477D or T477E (Kabat numbering); and the first kappa light chain constant region comprises S131H or S131K or S131R (EU numbering).

121. The antibody of claim 117(a), wherein the first kappa light chain constant region further comprises at least one amino acid substitution selected from V133H and Q160K.

122. The antibody of claim 117(b), wherein the first kappa light chain constant region further comprises Q124K.

123. The antibody of claim 115, wherein the second pairing region is the first kappa light chain constant region, and where the first kappa light chain constant region comprises at least one amino acid substitution selected from S171G, S159I, A144V, N152G, S159V, S159I, S159V, A1441, N138G, L136V, D185E, V1631, Q147V, Q147T, D122E, S171N, S156T, V205L, A111T, D122Q, L154V, E123D, Q147I, N210S and A193S.

124. The antibody of claim 116, wherein the second pairing region is a CH1 region, wherein:
(a) the IgM Cμ4 region comprises Y455C by Kabat numbering, and the CH1 region comprises A141C by EU numbering; or
(b) the IgM Cμ4 region comprises F516C by Kabat numbering, and the CH1 region comprises H168C by EU numbering; or
(c) the IgM Cμ4 region comprises Q463C by Kabat numbering, and the CH1 region comprises F126C by EU numbering; or
(d) the IgM Cμ4 region comprises L457C by Kabat numbering, and the CH1 region comprises L128C or G143C by EU numbering.

125. The antibody of claim 115, wherein the second pairing region is the first CH1 constant region, and wherein the IgM Cμ4 region includes one or more of:
(a) A482P,
(b) T477Y,
(c) L456V,
(d) V476I,
(e) T556I or T556A,
(f) E549Q,
(g) V523I,
(h) L495V,
(i) L475V,
(j) L457F, and
(k) R546H,
all by Kabat numbering.

126. The antibody of claim 114, wherein the IgM Cμ4 region comprises at least one mutation elected from:
(i) E468del or E468R,
(ii) E525del,
(iii) E527del,
(iv) E526del or E526A, and
(v) Q510R.

127. The antibody of claim 114, the first pairing region N-terminus is coupled directly to the first variable region C-terminus.

128. The antibody of claim 114, the first pairing region N-terminus is coupled indirectly to the first variable region C-terminus by a non-native coupling sequence.

129. At least one polynucleotide encoding the first chain and the second chain of the antibody of claim 114.

130. The antibody of claim 114, further comprising:
(a) a third chain comprising a third variable region and a third pairing region; and
(b) a fourth chain comprising a fourth variable region and a fourth pairing region;
wherein the third and fourth variable regions are heavy and light chain variable regions, or vice versa;
wherein the first and second chains are preferentially paired to each other via association of the first and second pairing regions, and
wherein the third and fourth chains are preferentially paired to each other via association of the third and fourth pairing regions, thereby forming a second binding site that specifically binds a second target epitope that is different from the first target epitope;
wherein
(i) the third pairing region is selected from:
(A) a second IgM Cμ4 region,
(B) a second light chain constant region, and
(C) a first modified CH3 region; and
(ii) where the third pairing region is (A), (B) or (C), the fourth pairing region is, correspondingly, selected from:
(A′) a second kappa light chain constant region or a second-CH1 region;
(B′) a third CH1 region, and
(C′) a second modified CH3 region, wherein the first modified CH3 region pairs preferentially with the second modified CH3 region.

131. The antibody of claim 130, wherein the first, second, third and fourth pairing regions collectively comprise a plurality of amino acid deletions, insertions or substitutions such that the first and second pairing regions preferentially pair with each other relative to their pairing with either the third or fourth pairing regions, and the third and fourth pairing regions preferentially pair with each other relative to their pairing with either the first or second pairing regions.

132. The antibody of claim 130, wherein the third and fourth chains are covalently coupled to the first and second chains.

133. The antibody of claim 130, comprising at least one amino acid addition, deletion or substitution in the first pairing region, second pairing region, third pairing region, fourth pairing region, or any plurality combination thereof, wherein the addition, deletion or substitution:
(a) promotes the pairing of the first and second pairing regions; or
(b) disfavors the pairing of either the first or second pairing regions with either the third or fourth pairing regions.
(c) promotes the pairing of the third and fourth pairing regions; or
(d) disfavors the pairing of either the third or fourth pairing regions with either the first or second pairing regions.

134. The antibody of claim 133, wherein the at least one amino acid addition, deletion or substitution results in the formation of a disulfide bond, thereby covalently linking the first and second pairing regions or the third and fourth pairing regions.

135. The antibody of claim 133, wherein the at least one amino acid addition, deletion or substitution prevents the formation of a disulfide bond, thereby preventing covalent linkage between either the first or second pairing regions and either the third or fourth pairing regions.

136. The antibody of claim 135, wherein the second pairing region is the first CH1 region and the C-terminus of the first CH1 region is linked to an N-terminal IgG1 hinge segment, and the naturally present cysteine at position 220 of the N-terminal hinge segment (by EU numbering) is substituted with an amino acid incapable to forming a disulfide bond or is deleted.

137. The antibody of claim 133, wherein the at least one amino acid addition, deletion or substitution increases the electrostatic attraction between (i) the first and second pairing regions, or (ii) the third and fourth pairing regions, thereby promoting pairing between the first and second pairing regions or the third and fourth pairing regions.

138. The antibody of claim 133, wherein the at least one amino acid addition, deletion or substitution results in electrostatic repulsion between (i) either the first or second pairing region, and (ii) either the third or fourth pairing region, thereby suppressing pairing between either the first or second pairing regions and either the third or fourth pairing regions.

139. The antibody of claim 133, wherein the third pairing region is (B) a kappa light chain constant region comprising one or more substitutions selected from
(i) S176D or S176E,
(ii) V133S and
(iii) Q124D or Q124E; and
the fourth pairing region (B′) is an IgG1 CH1 constant region comprising a substitution selected from L128K or L128R (all EU numbering).

140. The antibody of claim 130, wherein the first or second pairing region has a native cysteine substituted with an amino acid incapable of forming a disulfide bond or deleted to prevent disulfide bonding of that pairing region to the third or fourth chains.

141. The antibody of claim 140, wherein the second pairing region is the kappa light chain constant region and the native cysteine at the C-terminal portion of the kappa light chain constant region is substituted with an amino acid incapable of forming a disulfide bond or is deleted, to prevent disulfide bonding of the second pairing region to the third or fourth chains.

142. The antibody of claim 140, wherein the first pairing region is the IgM Cμ4 region, wherein said IgM Cμ4 region native cysteine at position 556 by Kabat numbering is (i) substituted with an amino acid that is incapable of forming a disulfide bond, (ii) is deleted, or where the IgM Cμ4 region is truncated at a position at or before cysteine 556.

143. The antibody of claim 130, wherein
(a) the first variable region is the light chain variable region of the first binding site and the first pairing region is the IgM Cμ4 region
(b) the second variable region is the heavy chain variable region of the first binding site and the second pairing region is the first kappa light chain constant region or the first CH1 region,
(c) the third variable region is the light chain variable region of the second binding site and the third pairing region is the second light chain constant region, and
(d) the fourth variable region is the heavy chain variable region of the second binding site and the fourth pairing region is a CH1 constant region selected from an IgG CH1 constant region and an IgA CH1 constant region.

144. The antibody of claim 130, wherein
(a) the first variable region is the heavy chain variable region of the first binding site and the first pairing region is the IgM Cμ4 region,
(b) the second variable region is the light chain variable region of the first binding site and the second pairing region is the kappa light chain constant region or the first CH1 region,
(c) the third variable region is the heavy chain variable region of the second binding site and the third pairing region is the second light chain constant region, and
(d) the fourth variable region is the light chain variable region of the second binding site and the fourth pairing region is a CH1 constant region selected from an IgG CH1 constant region and an IgA CH1 constant region.

145. The antibody of claim 130, wherein:
(a) the first pairing region is a first IgM Cμ4 region comprising Y455C by Kabat numbering,
(b) the second pairing region is a first kappa light chain constant region comprising S121C, Q124C or K131C, all by EU numbering,
(c) the third pairing region is (A) a second IgM Cμ4 region comprising F516C by Kabat numbering, and
(d) the fourth pairing region is (A′) a second kappa light chain constant region comprising a cysteine at amino acid position Q160C by EU numbering.

146. The antibody of claim 130, said antibody comprising:
(a) a first chain comprising a first IgM C□4 pairing region comprising amino acid substitutions Y455C and T477K,
(b) a second chain that pairs with the first chain, the second chain comprising a first kappa pairing region comprising amino acid substitutions Q124C, C214A, and either (i) S131D, or (ii) S131E,
(c) a third chain comprising a second IgM C□4 pairing region comprising amino acid substitutions F516C and T477D, and
(d) a fourth chain that pairs with the third chain, the fourth chain comprising a second kappa pairing region comprising mutations Q160C, C214A and S131K.

147. At least one polynucleotide encoding the first chain, second chain, third chain and fourth chain of the antibody of claim 130.

148. The antibody of claim 130, further wherein
(a) either the first or second chain further comprises:
(i) a first at least a portion of a hinge region,
(ii) a first CH2 region, and
(iii) a first CH3 region, and
(b) either the third or fourth chain further comprises:
(i′) a second at least a portion of a hinge region,
(ii′) a second CH2 region, and
(iii′) a second CH3 region,
wherein the first at least a portion of a hinge region is positioned between the first or second pairing region and the first CH2 and CH3 regions,
wherein the second at least a portion of a hinge region is positioned between the third or fourth pairing region and the second CH2 and CH3 regions,
wherein the paired first and second chains and the paired third and fourth chains are associated with each other via at least the first and second CH3 regions, thereby forming a tetramer.

149. The antibody of claim 148, wherein the first CH2 region, the first CH3 region, the second CH2 region, and the second CH3 region are, independently, an IgG isotype or an IgA isotype.

150. The antibody of claim 148, wherein the associated first and second chains and the associated third and fourth chains are associated with each other, at least in part, by at least one disulfide bond.

151. The antibody of claim 148, wherein the first or second hinge regions or portions thereof comprise removing or substituting the cysteine at the position analogous to C220 of the IgG1 hinge amino acid sequence by EU numbering with an amino acid incapable of forming a disulfide bond.

152. The antibody of claim 148, said antibody comprising a chain comprising an IgG1 CH1 pairing region or a IgM Cm4 pairing region, said chain further comprising at least a portion of a hinge and CH2 and CH3 regions of human IgG1 isotype, wherein a cysteine residue at EU position 220 of the at least a portion of a hinge of that chain is substituted with an amino acid that can not form a disulfide bond or is deleted to prevent disulfide bonding with the C-terminal cysteine of a light chain constant region.

153. The antibody of claim 148, wherein:
(a) the third pairing region is the second CH1 constant region,
(b) the fourth pairing region is the second light chain constant region, and
(c) the first chain comprises a IgM Cμ4 pairing region, at least a portion of a hinge and CH2 and CH3 regions of human IgG1 isotype, wherein a cysteine residue at EU position 220 of the at least a portion of a hinge of the first chain is substituted with an amino acid that can not form a disulfide bond or deleted to prevent disulfide bonding with the second light chain constant region.

154. The antibody of claim 148, wherein:
(a) the third pairing region is the second CH1 constant region,
(b) the fourth pairing region is the second light chain constant region, and
(c) the second chain comprises a CH1 pairing region, at least a portion of a hinge and CH2 and CH3 regions of human IgG1 isotype, wherein a cysteine residue at EU position 220 of the at least a portion of a hinge of the second chain is substituted with an amino acid that can not form a disulfide bond is or deleted to prevent disulfide bonding with the second light chain constant region.

155. The antibody of claim 148, wherein the antibody comprises a chain comprising a CH1 region that is an IgG2, IgG3 or IgG4 isotype and subclass, said chain further comprising at least a portion of a hinge and CH2 and CH3 regions of human IgG2, IgG3 or IgG4 isotype, and wherein the CH1 region comprises a cysteine residue at EU position 131 that is substituted with an amino acid that can not form a disulfide bond or is deleted to prevent disulfide bonding with the C-terminal cysteine of a light chain constant region.

156. The antibody of claim 148, wherein:
(a) the third pairing region is the second CH1 constant region,
(b) the fourth pairing region is the second light chain constant region, and
(c) the second chain comprises a CH1 pairing region, at least a portion of a hinge and CH2 and CH3 regions of human IgG2, IgG3 or IgG4 isotype, wherein the CH1 pairing region comprises a cysteine residue at EU position 131 that is substituted with an amino acid that can not form a disulfide bond or is deleted to prevent disulfide bonding with the second light chain constant region.

157. The antibody of claim 148, wherein the first or second IgM Cμ4 pairing region comprises an amino acid sequence selected from SEQ ID NOS: 23-25, 53, 54, 56-59, 74-78, 86-92, 119-190, 218-220, 222-229, 265-269, 479, 504-505, and 507-509.

158. The antibody of claim 148, wherein the first and second at least a portion of a hinge region each comprises, independently, a sequence selected from CDKTHTCPPCP (SEQ ID NO: 516), CVECPPCP (SEQ ID NO: 517) and SEQ ID NOs: 2, 6, 10, 14, 117, 196-199, 231 and 232.

159. The antibody of claim 148, wherein the chains comprising the first and second CH2 and CH3 regions comprise at least one pair of complementary knob and hole amino acid substitutions to promote their association.

160. The antibody of claim 159, wherein said complementary knob and hole amino acid substitutions are selected from:
(a) Y407T in one chain and T366Y in the other chain,
(b) Y407A in one chain and T366W in the other chain,
(c) F405A in one chain and T394W in the other chain,
(d) F405W in one chain and T394S in the other chain,
(e) Y407T in one chain and T366Y in the other chain,
(f) T366Y and F405A in one chain and T394W and Y407T in the other chain,
(g) T366W and F405W in one chain and T394S and Y407A in the other chain,
(h) F405W and Y407A in one chain and T366W and T394S in the other chain, and
(i) T366W in one chain and T366S, L368A, and Y407V in the other chain;
all by EU numbering.

161. The antibody of claim 148, wherein the chains comprising the first and second CH2 and CH3 regions comprise at least one pair of complementary amino acid substitutions that create interchain disulfide bonds and thereby promote heterodimer formation.

162. The antibody of claim 161, wherein the pair of complementary amino acid substitutions that create interchain disulfide bonds is selected from:
(a) Y349C in one chain and S354C in the other chain,
(b) Y349C in one chain and E356C in the other chain,
(c) Y349C in one chain and E357C in the other chain,
(d) L351C in one chain and S354C in the other chain,
(e) T394C in one chain and E397C in the other chain, and
(f) D399C in one chain and K392C in the other chain,
by EU numbering.

163. The antibody of claim 148, wherein the first or the second at least a portion of a hinge region or the first or the second CH2 or CH3 regions include a mutation modulating effector function.

164. The antibody of claim 148, wherein the first or the second CH2 or CH3 regions include at least one mutation increasing FcRn binding or that increases half-life of the antibody.

165. The antibody of claim 148, wherein the first or the second CH2 or CH3 regions comprise at least one mutation that eliminates or reduces binding to protein A.

166. A composition comprising the antibody of claim 148, the composition further comprising a pharmaceutically acceptable excipient.

167. At least one polynucleotide encoding the first chain, second chain, third chain and fourth chain of the antibody of claim 148.

168. The at least one polynucleotide of claim 167, wherein the at least one polynucleotide comprises:
(a) a first polynucleotide encoding the first chain,
(b) a second polynucleotide encoding the second chain,
(c) a third polynucleotide encoding the third chain, and
(d) a fourth polynucleotide encoding the fourth chain.

169. An expression system for producing an antibody in a host cell, the system comprising:
(a) a first polynucleotide encoding first polypeptide, said first polypeptide comprising a first variable region and a first pairing region;
(b) a second polynucleotide encoding second polypeptide, said second polypeptide comprising a second variable region and a second pairing region;
(c) a host cell suitable for expressing the first and second polypeptides following delivery of said first and second polynucleotides into said host cell;
wherein the first and second variable regions are heavy and light chain variable regions, or vice versa;
wherein the first and second polypeptides pair to each other via association of the first and second pairing regions, thereby forming a first binding site that specifically binds a first target epitope;
wherein the first pairing region is a first IgM Cμ4 region, and the second pairing region is selected from a first kappa light chain constant region and a first CH1 region.

170. A method for producing an antibody in a host cell, the method comprising:
(a) providing:
(i) a first polynucleotide encoding a first polypeptide, said first polypeptide comprising a first variable region and a first pairing region;
(ii) a second polynucleotide encoding a second polypeptide, said second polypeptide comprising a second variable region and a second pairing region; wherein
(A) the first and second variable regions are heavy and light chain variable regions, or vice versa,
(B) the first and second polypeptides pair to each other via association of the first and second pairing regions, thereby forming a first binding site that specifically binds a first target epitope, and
(C) the first pairing region is a first IgM Cμ4 region, and the second pairing region is selected from a first kappa light chain constant region and a first CH1 region;
(iii) a host cell suitable for expressing the first and second polypeptides following delivery of said first and second polynucleotides into said host cell;
(b) delivering said first and second polynucleotides in said host cell;
(c) culturing said host cell under conditions for the expression of said first and second polypeptides, thereby producing said antibody.
